(19) United States
(12) Patent Application Publication
(10) Pub. No.: US 2026/0028403 A1
(43) Pub. Date: Jan. 29, 2026

(54) TRIVALENT AND TRISPECIFIC ANTIBODY CONSTRUCTS AND METHODS OF USE THEREOF

(71) Applicant: Zymeworks BC Inc., Vancouver (CA)

(72) Inventors: Nicole Afacan, Vancouver (CA); Chayne L. Piscitelli, Vancouver (CA); Lisa Newhook, Vancouver (CA); Purva Prashant Bhojane, Vancouver (CA); Patricia Zwierzchowski, Vancouver (CA); Peter Repenning, Vancouver (CA); Nina E. Weisser, Vancouver (CA); Thomas Spreter Von Kreudenstein, Vancouver (CA); Paula Tao, Vancouver (CA); David Douda, Vancouver (CA); Kelsey Ferns, Vancouver (CA); Sylwia Jancowski, Vancouver (CA); Sukhbir Singh Kang, Vancouver (CA); Lee Freiburger, Vancouver (CA); Nichole K. Escalante, Vancouver (CA)

(21) Appl. No.: 19/180,055

(22) Filed: Apr. 15, 2025

Related U.S. Application Data
(63) Continuation of application No. PCT/CA2023/051391, filed on Oct. 19, 2023.
(60) Provisional application No. 63/417,542, filed on Oct. 19, 2022.
(60) Provisional application No. 63/458,852, filed on Apr. 12, 2023.
(60) Provisional application No. 63/465,137, filed on May 9, 2023.
(60) Provisional application No. 63/591,311, filed on Oct. 18, 2023.

(51) Int. Cl. C07K 16/28; A61K 39/00; A61P 35/00

(52) U.S. Cl. CPC C07K 16/2809; A61P 35/00; C07K 16/28; C07K 16/2818; A61K 2039/505; C07K 2317/24; C07K 2317/31; C07K 2317/35; C07K 2317/55; C07K 2317/622; C07K 2317/73; C07K 2317/92; C07K 2317/94

(57) ABSTRACT

The present disclosure provides trivalent and trispecific antibody constructs capable of binding two different antigens on one or more cytotoxic effector cell(s) and a tumor-associated antigen (TAA) on a tumor cell. Pharmaceutical compositions comprising such antibody constructs and methods of preparing and using such constructs and compositions, e.g., for the treatment of cancer, are also disclosed.

### CROSS-REFERENCE

This application claims priority to and the benefit of U.S. Provisional Application No. 63/591,311, filed Oct. 18, 2023, U.S. Provisional Application No. 63/465,137, filed May 9, 2023, U.S. Provisional Application No. 63/458,852, filed Apr. 12, 2023, and U.S. Provisional Application No. 63/417,542, filed Oct. 19, 2022, the entire contents of each of which are incorporated by reference herein for all purposes.

### SEQUENCE LISTING

The instant application contains a Sequence Listing which is hereby incorporated by reference in its entirety Said XML copy, created on Nov. 9, 2023, is named ZWI-096WOC1_SL, and is 214,954 bytes in size.

### TECHNICAL FIELD

The present disclosure generally relates to trivalent and trispecific T cell engaging antibody constructs that can comprise a first binding domain capable of binding a first antigen on a first cytotoxic effector cell, a second binding domain capable of binding a second antigen on a second cytotoxic effector cell, and a third binding domain capable of binding a tumor-associated antigen (TAA) on a tumor cell.

### BACKGROUND

Cancer continues to pose a major unmet medical need, despite the considerable progress that has been made in its treatment over the past decades. While the current standard of care, as well as more recently developed anti-cancer therapies, have shown some clinical progress, various indications such as those with low T cell tumor infiltration still present a major clinical challenge.

### SUMMARY

In various embodiments, the present disclosure describes trispecific and trivalent antibody constructs that are capable of engaging two different antigens on one or more immune cell(s) (e.g., T cell(s)), as well as an antigen (e.g., a TAA) on a tumor cell. In certain embodiments of the present disclosure, described herein are trivalent and trispecific antibody constructs that comprise three binding domains, wherein a first binding domain is capable of binding a first antigen on a first cytotoxic effector cell, a second binding domain capable of binding a second antigen on a second cytotoxic effector cell, and a third binding domain capable of binding a tumor-associated antigen (TAA) on a tumor cell.

In one embodiment, described herein is an antibody construct, comprising: (i) a Fab domain capable of binding a first antigen on a first cytotoxic effector cell; (ii) a first scFv domain and a second scFv domain, wherein one of the scFv domains is capable of binding a second antigen on a second cytotoxic effector cell and the other scFv domain is capable of binding a tumor-associated antigen (TAA) on a tumor cell, and (iii) an Fc domain comprising a first Fc polypeptide and a second Fc polypeptide, wherein: (a) the first and second antigens on the one or more cytotoxic effector cells are different, (b) the Fab domain is coupled to the N-terminus of the first Fc polypeptide, and (c) each of the first and second scFv domains is independently coupled to either an N-terminus of the Fab domain, a C-terminus of the Fab domain, or the N-terminus of the second Fc polypeptide.

In one embodiment, described herein is an antibody construct, comprising: (i) a Fab domain capable of binding a CD3 on a first cytotoxic effector cell; (ii) a first scFv domain and a second scFv domain, wherein one of the scFv domains is capable of binding CD28 on a second cytotoxic effector cell and the other scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, and (b) each of the first and second scFv domains is independently coupled to either an N-terminus of the Fab domain, a C-terminus of the Fab domain, the C-terminus of one of the Fc polypeptides, or the N-terminus of the second Fc polypeptide.

In one embodiment, described herein is an antibody construct, comprising: (i) a Fab domain capable of binding a CD28 on a first cytotoxic effector cell; (ii) a first scFv domain and a second scFv domain, wherein one of the scFv domains is capable of binding CD3 on a second cytotoxic effector cell and the other of the scFv domains is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, and (b) each of the first and second scFv domains is independently coupled to either an N-terminus of the Fab domain, a C-terminus of the Fab domain, the C-terminus of one of the Fc polypeptides, or the N-terminus of the second Fc polypeptide.

In one embodiment, described herein is an antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the N-terminus of the V_{H} domain of the Fab domain, and (c) the second scFv domain is coupled to the N-terminus of the second Fc polypeptide. An antibody construct according to such an embodiment is shown in, e.g., FIG. 1A.

In one embodiment, described herein is an antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the N-terminus of the V_{H} domain of the Fab domain, and (c) the second scFv domain is coupled to the N-terminus of the second Fc polypeptide. An antibody construct according to such an embodiment is shown in, e.g., FIG. 1A.

In one embodiment, described herein is an antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the CL domain of the light chain of the Fab domain, and (c) the second scFv domain is coupled to the N-terminus of the second Fc polypeptide. An antibody construct according to such an embodiment is shown in, e.g., FIG. 1B.

In one embodiment, described herein is an antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the CL domain of the light chain of the Fab domain, and (c) the second scFv domain is coupled to the N-terminus of the second Fc polypeptide. An antibody construct according to such an embodiment is shown in, e.g., FIG. 1B.

In one embodiment, described herein is an antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the second Fc polypeptide. An antibody construct according to such an embodiment is shown in, e.g., FIG. 1F.

In one embodiment, described herein is an antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the second Fc polypeptide. An antibody construct according to such an embodiment is shown in, e.g., FIG. 1F.

In one embodiment, described herein is an antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the V_{H} domain of the Fab domain. An antibody construct according to such an embodiment is shown in, e.g., FIG. 1C.

In one embodiment, described herein is an antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the V_{H} domain of the Fab domain. An antibody construct according to such an embodiment is shown in, e.g., FIG. 1C.

In some embodiments, described herein is a pharmaceutical composition comprising a trivalent and trispecific antibody construct of the present disclosure, and a pharmaceutically acceptable carrier, excipient, diluent, or combination thereof.

In some embodiments, described herein is a nucleic acid molecule or a set of nucleic acid molecules encoding one or more, two or more, or three or more polypeptide chains that form a trivalent and trispecific antibody construct of the present disclosure.

In some embodiments, described herein is a vector or a set of vectors comprising the nucleic acid molecule or the set of nucleic acid molecules that encode the one or more, two or more, or three or more polypeptide chains of a trivalent and trispecific antibody construct of the present disclosure.

In certain embodiments, the present disclosure relates to a method of producing a trivalent and trispecific antibody construct, the method comprising: (a) obtaining a host cell culture comprising at least one host cell comprising one or more nucleic acid molecules encoding one or more, two or more, or three or more polypeptide chains that form the antibody construct; and (b) recovering the antibody construct from the host cell culture.

In certain embodiments, the present disclosure relates to a method of eliciting an anti-tumor immune response in a cell population comprising immune cells and tumor cells expressing a TAA, the method comprising contacting the cell population with an effective amount of a trivalent and trispecific antibody construct of the present disclosure, wherein the immune cells express a first and a second antigens and the tumor cells express the TAA.

In certain embodiments, the present disclosure relates to a method of inhibiting the proliferation of tumor cells expressing a TAA, the method comprising contacting a cell population comprising the tumor cells and immune cells with an effective amount of a trivalent and trispecific antibody construct of the present disclosure, wherein the immune cells express the first and second antigens and the tumor cells express the TAA.

In certain embodiments, the present disclosure relates to a method of killing tumor cells expressing a TAA, the method comprising contacting a cell population comprising the tumor cells and immune cells with an effective amount of a trivalent and trispecific antibody construct of the present disclosure, wherein the immune cells express the first and second antigens and the tumor cells express the TAA.

In some of these embodiments, the first antigen can be CD3 or CD28, and the second antigen can be CD3 or CD28, wherein the first and second antigen are different antigens. Furthermore, in some embodiments, the TAA can be MSLN or Cldn18.2.

In certain embodiments, the present disclosure relates to a method treating a cancer in a subject in need thereof, the method comprising administering to the subject a trivalent and trispecific antibody construct of the present disclosure, or a pharmaceutical comprising a trivalent and trispecific antibody construct of the present disclosure. In some embodiments, such method can further comprise eliciting a cytotoxic immune response against the cancer in the subject by the trivalent and trispecific antibody construct, thereby treating the cancer in the subject.

In some embodiments, the disclosure relates to a trivalent and trispecific antibody construct for use in the treatment of cancer.

In some embodiments, the disclosure relates to a trivalent and trispecific antibody construct in the manufacture of a medicament for the treatment of cancer.

In some embodiments, described herein is an antibody construct, comprising a binding domain capable of binding CD28, wherein the binding domain comprises a V_{H} sequence comprising a HCDR1 having the sequence SX₁GVH (SEQ ID NO: 302), a HCDR2 having the sequence VIWX₂GGX₃TNFNSALMS (SEQ ID NO: 306), and a HCDR3 having the sequence DRAX₄GX₅YX₆X₇AMDY (SEQ ID NO: 312), and a V_{L} sequence comprising a LCDR1 having the sequence RASESVEYYX₈TSLMQ (SEQ ID NO: 315), a LCDR2 having the sequence AASX₉VX₁₀S (SEQ ID NO: 319), and a LCDR3 having the sequence QQSRKVPFT (SEQ ID NO: 320), having one or more of the following amino acid substitutions at the positions as identified in the CDR sequences: X₁: Y to A (i.e., residue Y is substituted with residue A), X₂: P to A, X₃: G to S, X₄: S to Y, X₅: N to A, X₆: L to N, X₇: S to Y, X₈: G to V, X₉: N to A, or X₁₀: E to D.

In some embodiments, described herein is an antibody construct, comprising a binding domain capable of binding Cldn18.2, wherein the binding domain comprises a V_{H} sequence comprising a HCDR1 having the sequence SNPMI (SEQ ID NO: 333), a HCDR2 having the sequence IIDTDGSTYYADWAKG (SEQ ID NO: 334), and a HCDR3 having the sequence RLHGSSNGYYDDL (SEQ ID NO: 335), and a V_{L} sequence comprising a LCDR1 having the sequence QASQSIYSYLS (SEQ ID NO: 336), a LCDR2 having the sequence KASTLAS (SEQ ID NO: 337), and a LCDR3 having the sequence QQGYTVTNVDKNT (SEQ ID NO: 338).

### BRIEF DESCRIPTION OF THE DRAWINGS

The implementations disclosed herein are illustrated by way of example, and not by way of limitation, in the accompanying drawings. The description and drawings are only for the purpose of illustration and as an aid to understanding and are not intended as a definition of the limits of the antibody constructs, pharmaceutical compositions, and methods of the present disclosure.

FIGS. 1A-1G show schematic representations of the format and geometry of trivalent and trispecific antibody constructs that comprise binding domains capable of engaging CD3, CD28, and a TAA, according to embodiments of the present disclosure. FIGS. 1H-1L show schematic representations of bispecific control constructs used and described in this disclosure.

FIGS. 2A-2B show binding of certain anti-CD3 (Fab)/anti-CD28 (scFv)/anti-MSLN (scFv) trivalent and trispecific constructs (v34914, v34915, v34916 and v34917, FIG. 2A), and anti-CD3 (scFv)/anti-CD28 (Fab)/anti-MSLN (scFv) trivalent and trispecific constructs (v34913 and v34918, FIG. 2B) to CD4+ (left) and CD8+ (right) T cells, as measured by flow cytometry.

FIGS. 3A-3B show that certain anti-CD3 (Fab)/anti-CD28 (scFv)/anti-MSLN (scFv) trivalent and trispecific constructs (v34914, v34915, v34916 and v34917, FIG. 3A), and anti-CD3 (scFv)/anti-CD28 (Fab)/anti-MSLN (scFv) trivalent and trispecific constructs (v34913 and v34918, FIG. 3B) directed T cells from healthy donors to toward MSLN+ H292 target cells, inducing T cell mediated killing of the tumor cells.

FIGS. 4A-4B show that certain anti-CD3 (Fab)/anti-CD28 (scFv)/anti-MSLN (scFv) trivalent and trispecific constructs (v34914, v34915, v34916 and v34917, FIG. 4A), and anti-CD3 (scFv)/anti-CD28 (Fab)/anti-MSLN (scFv) trivalent and trispecific constructs (v34913 and v34918, FIG. 4B) induced Interleukin-2 (IL-2, left) and/or tumor necrosis factor alpha (TNFα, right) production from T cells when co-incubated with MSLN+ H292 cells (E:T of 2:1) for 72 hours (hrs).

FIGS. 5A-5D show in vitro T cell-dependent cytotoxicity induced by the trivalent and trispecific antibody constructs v34914 (FIG. 5A), v34916 (FIG. 5B), v34913 (FIG. 5C) and v34918 (FIG. 5D) and compared to the bispecific anti-(MSLNxCD3) benchmark control construct v34919 against MSLN^{high} H292 cells (152,986 MSLN/cell) and MSLN^{low} OVTOKO cells (9,752 MSLN/cell).

FIGS. 6A-6B show that the trivalent and trispecific antibody constructs v34913, v34916 and v34918 directed T cells from healthy donors to kill MSLN+H292 target cells expressing moderate levels of MSLN. This long-term T cell-dependent cellular cytotoxicity (TDCC) study utilized a low E:T ratio of 1:5 and an incubation period of either 3 days (FIG. 6A) or 7 days (FIG. 6B), e.g., long-term co-culture at low E:T ratios to reflect conditions more accurately in certain (e.g., solid) tumor types.

FIGS. 7A-7B show that the trivalent and trispecific antibody constructs comprising an anti-CD28 Fab domain v34913 and v34918 (FIG. 7B), as well as the trivalent and trispecific antibody constructs comprising an anti-CD3 Fab domain v34914, v34916 and v34917 (FIG. 7A), induced proliferation of T cells co-incubated with MSLN+ OVCAR3 cells (E:T of 10:1) for 5 days, and when compared to the control constructs v34919, v34927 and v31926. FIG. 7C shows T cell proliferation data measured after 3 days, 5 days, and 7 days of co-culture with H292 cells (E:T ratio=2:1) for the potent trivalent and trispecific construct, v34913, a corresponding bispecific anti-(MSLNxCD3) control construct, v34919, as well as the negative control v22277.

FIGS. 8A-8B show binding of certain trivalent and trispecific anti-CD3/anti-CD28/anti-Cldn 18.2 antibody constructs to CLDN18.2+ SNU 601 cells as measured by flow cytometry. Both, the constructs that comprised an anti-CD3 Fab domain and an anti-CD28 scFv domain (v37633, v37634 and v37635) as well as those that comprised an anti-CD28 Fab domain and an anti-CD3 scFv domain (v37638, v37640 and v37642), were compared against an anti-(Cldn18.2×CD3) bispecific construct (v37663), a one-armed anti-Cldn18.2 antibody (v37675), a benchmark control (v35923, also known as AMG910), an anti-Cldn18.2 monoclonal antibody (mAb), and the anti-RSV protein F mAb palivizumab.

FIGS. 9A-9B show binding of certain anti-CD3 (Fab)/anti-CD28 (scFv)/anti-Cldn18.2 (scFv) trivalent and trispecific constructs (v37633-v37637, FIG. 9A), and anti-CD3 (scFv)/anti-CD28 (Fab)/anti-Cldn18.2 (scFv) trivalent and trispecific constructs (v37638-v37642, FIG. 9B) to CD4+ (left) and CD8+ (right) T cells, as measured by flow cytometry.

FIGS. 10A-10B show that certain anti-CD3 (Fab)/anti-CD28 (scFv)/anti-Cldn18.2 (scFv) trivalent and trispecific constructs (v37633-v37637, FIG. 10A), and anti-CD3 (scFv)/anti-CD28 (Fab)/anti-Cldn18.2 (scFv) trivalent and trispecific constructs (v37638-v37642, FIG. 10B) directed T cells from healthy donors to kill CLDN18.2+ SNU 601 target cells. These trispecific constructs were compared against a bispecific anti-(Cldn18.2×CD3) control (v37663), a bispecific anti-(Cldn18.2×CD28) control (v37665) and a negative control (Het-Fc version of palivizumab, v22277).

FIGS. 11A-11B show that certain anti-CD3 (Fab)/anti-CD28 (scFv)/anti-Cldn18.2 (scFv) trivalent and trispecific constructs (v37633-v37637, FIG. 11A), and anti-CD3 (scFv)/anti-CD28 (Fab)/anti-Cldn18.2 (scFv) trivalent and trispecific constructs (v37638-v37642, FIG. 11B) induced IL-2 (top) and/or TNFα (bottom) production from T cells co-incubated with CLDN18.2+ SNU 601 cells (E:T of 2:1) for 72 hrs.

FIGS. 12A-12D show in vitro T cell-dependent cytotoxicity induced by the trivalent and trispecific antibody constructs v37633 (FIG. 12A), v37634 (FIG. 12B), v37638 (FIG. 12C) and v37642 (FIG. 12D) and compared to the bispecific anti-(MSLNxCD3) benchmark control construct v35923 against CLDN18.2^{high} SNU 601 and CLDN18.2^{low} SKOV-3 target cells.

FIGS. 13A-13B show concentration response curves for a long-term (7 days) TDCC study for the tested anti-CLDN18.2 trivalent and trispecific constructs that either comprised anti-CD3 (Fab)/anti-CD28 (scFv)/anti-Cldn18.2 (scFv) (v37633-v36735, FIG. 13A) or anti-CD3 (scFv)/anti-CD28 (Fab)/anti-Cldn18.2 (scFv) (v37638, v37640, v37642, FIG. 13B) and show that the constructs directed T cells from healthy donors to kill CLDN18.2+ target cells expressing varying levels of CLDN18.2 using an E:T ratio of 1:1. Top left: SNU-601 (276,125 CLDN18.2/cell); top right: KATO-III (63,566 CLDN18.2/cell); bottom: DAN-G (33,164 CLDN18.2/cell).

FIGS. 14A-14B show that certain anti-CLDN18.2 trivalent and trispecific constructs that either comprised anti-CD3 (Fab)/anti-CD28 (scFv)/anti-Cldn18.2 (scFv) (v37633-v36735, FIG. 14A) or anti-CD3 (scFv)/anti-CD28 (Fab)/anti-Cldn18.2 (scFv) (v37638, v37640, v37642, FIG. 14B) were capable of inducing the production of several cytokines (i.e., IFNγ (top left), IL-2 (top right), TNFα (bottom)) in T cells in the presence of CLDN18.2+ target SNU 601 using a 72 hours incubation period and an ET ratio of 2:1, and in comparison to certain bispecific and benchmark control constructs.

FIG. 15 shows the binding curves to CD3+CD28+ Jurkat cells for several anti-CLDN18.2 trivalent and trispecific constructs, v37638, v37683, v37689, v37692 and v37694, comprising anti-CD28 binding domains with mutation(s) in their V_{H} or V_{L} domain and thus varying affinity for CD28 as measured by flow cytometry.

FIG. 16 shows that several tested anti-CLDN18.2 trivalent and trispecific constructs with varying affinity for CD28, v37638, v37683, v37689, v37692 and v37694, directed T cells from healthy donors to kill CLDN18.2+ SNU 601 cells and compared to bispecific (v37663) and negative (v22277) control constructs.

FIGS. 17A-17B show that several tested anti-CLDN18.2 trivalent and trispecific constructs with varying affinity for CD28, v37638, v37683, v37689, v37692 and v37694, induced IL-2 (FIG. 17A) or TNFα (FIG. 17B) production from T cells co-incubated with CLDN18.2+ SNU 601 cells (E:T of 2:1) for 72 hrs, and in comparison to bispecific (v37663) and negative (v22277) control constructs.

FIG. 18 shows upregulation of Bcl-xL expression in activated T-cells using either a trivalent and trispecific antibody construct of the present disclosure, v37634, or certain bispecific controls constructs.

FIG. 19 shows upregulation of T cell proliferation in activated T-cells using either a trivalent and trispecific antibody construct of the present disclosure, v37634, or certain bispecific controls constructs.

FIG. 20 shows in vivo anti-tumor activities in donor X-engrafted mice treated with either the bispecific control constructs, v35923 or v38417, or the trispecific and trivalent antibody construct, v37634.

FIG. 21 shows the body weights of mice treated over the course of the in vivo efficacy study referenced in FIG. 20.

FIGS. 22A-22B show libraries of (i) conventional agonist paratope variants with a range of CD28 binding affinities (FIG. 22A), and (ii) agonist paratope variants with a range of CD3 binding affinities (FIG. 22B), determined by SPR. FIG. 22C shows a representation of the impact of paratope format (scFv vs. Fab) and geometry of the antibody construct on the binding affinities to CD3 and CD28 for a subset of formats with the same CD3 and CD28 paratopes (left), as well as a representation of affinities following CD3 and CD28 paratope engineering for one antibody construct format, which can be transferred among formats to create a large panel of antibody constructs (right).

FIG. 23A shows that the representative trivalent and trispecific antibody construct v37634 showed no significant change in purity post 5 cycles freezing to −80° C. followed by thawing to 4° C., and FIG. 23B shows that v37634 showed only minimal change in purity post 14 days incubation at 40° C. in an accelerated stress test. The construct concentration was 1 mg/mL. FIGS. 23C-23F show that the trivalent and trispecific antibody construct v37634 showed no significant change in purity after storage at −80° C. for 10 weeks (FIG. 23C), after storage at 4° C. for 14 days (FIG. 23D), and under low (pH 3.5, FIG. 23E) and high (pH 9, FIG. 23F) pH conditions for 3 hours.

### DETAILED DESCRIPTION

In various embodiments, the present disclosure describes trivalent and trispecific antibody constructs capable of binding two different antigens located either on a cytotoxic effector cell (e.g., a T cell) or on two different cytotoxic effector cells (i.e., each antigen is located on a different cell), and a TAA on a tumor cell. Such antibody construct can comprise a first binding domain capable of binding a first antigen on a first cytotoxic effector cell, a second binding domain capable of binding a second antigen on a second cytotoxic effector cell, and a third binding domain capable of binding a TAA on a tumor cell. Hence, in certain embodiments, described herein are trivalent and trispecific T cell engaging antibody constructs capable of co-stimulating one or more effector cells (e.g., T cells) by engaging two effector cell antigens (e.g., CD3 and CD28).

In some embodiments, and as further described herein, a trivalent and trispecific antibody construct of the present disclosure can comprise (i) a fragment antigen binding (Fab) domain (i.e., a first binding domain) that is capable of binding a first antigen on a first cytotoxic effector cell, (ii) a first single-chain variable fragment (scFv) domain (i.e., a second binding domain) that is capable binding a second antigen on a second cytotoxic effector cell, and (iii) a second scFv domain (i.e., a third binding domain) capable of binding a TAA on a tumor cell. Such trivalent and trispecific antibody construct can further comprise an Fc domain (e.g., a heterodimeric Fc domain) comprising a first Fc polypeptide and a second Fc polypeptide and to which the various binding domains are coupled, either directly (e.g., without a linker) or indirectly, e.g., via a linker and/or via another binding domain (e.g., in instances in which a first binding domain is coupled indirectly to an Fc polypeptide via a second binding domain, e.g., a C-terminus of the first binding domain is coupled to an N-terminus of the second binding domains, and such second binding domains is in turn coupled to the N-terminus of the Fc polypeptide, thereby indirectly coupling the first binding domain to the Fc polypeptide).

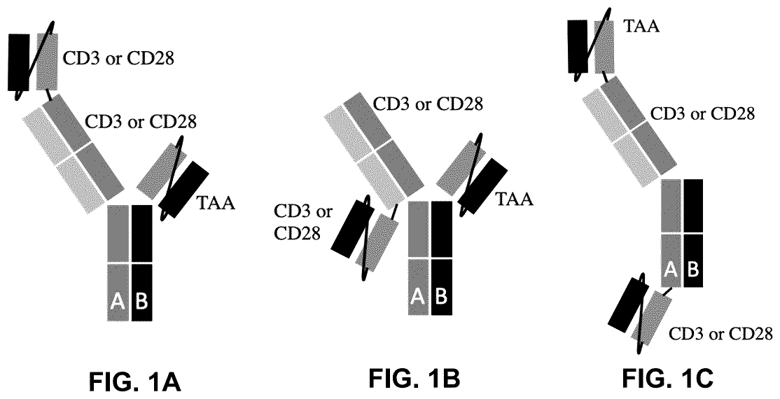

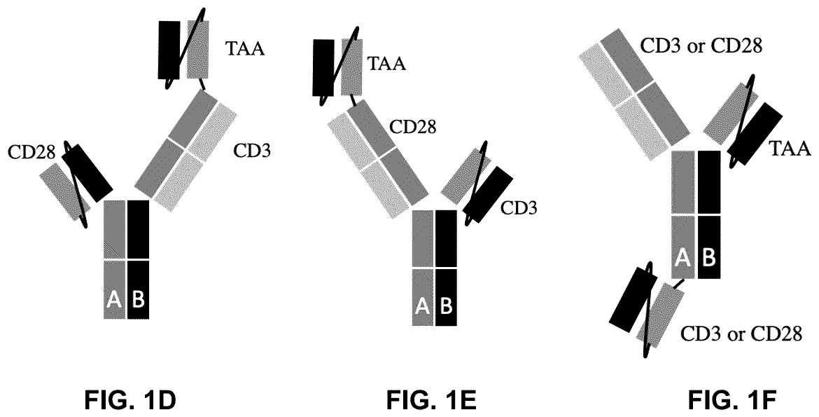

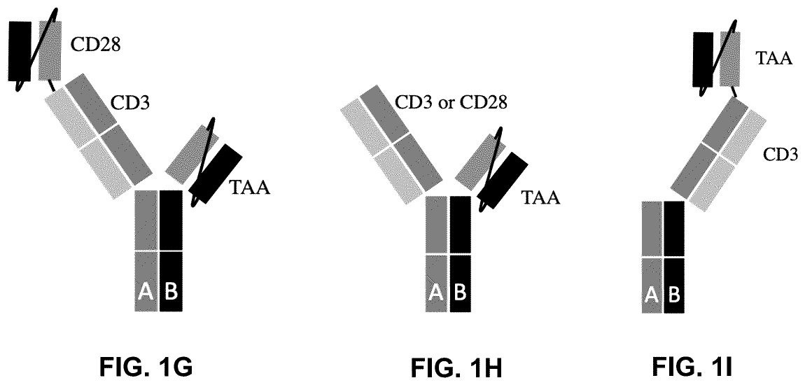

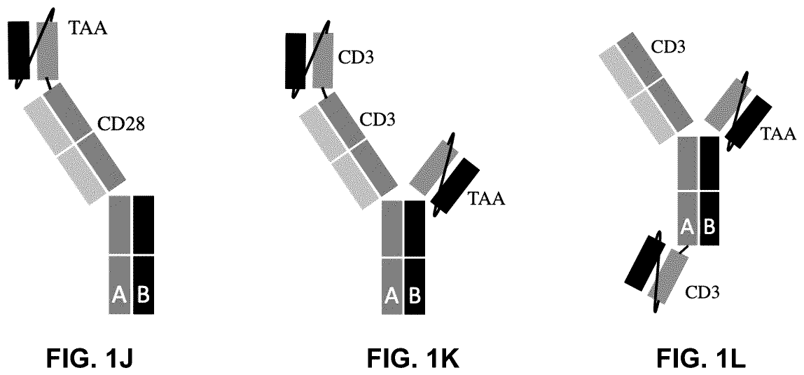

As further described herein, FIGS. 1A-1G depict certain antibody construct geometries with different relative orientation of the three binding domains, according to certain embodiments of this disclosure.

Further described herein are pharmaceutical compositions comprising one or more of the trivalent and trispecific antibody constructs disclosed herein.

Other embodiments of this disclosure relate to a nucleic acid molecule or a set of nucleic acid molecules that encode the one or more (e.g., 2, 3, or more) polypeptide chains of an antibody construct described herein. Some embodiments relate to a vector or a set of vectors that comprise a nucleic acid molecule or a set of nucleic acid molecules encoding an antibody construct of the present disclosure.

Other embodiments of this disclosure describe methods of producing and using the trivalent and trispecific antibody constructs described herein, e.g., for the treatment of a cancer in a subject (such as a rodent or a human).

### I. DEFINITIONS

Unless defined otherwise, all technical and scientific terms used herein have the same meaning as commonly understood by one of ordinary skill in the art.

The term “about,” as used herein in the context of a numerical value or range, generally refers to ±10%, 9%, 8%, 7%, 6%, 5%, 4%, 3%, 2%, or +1% of the numerical value or range recited or claimed, unless otherwise specified. In various embodiments, the term “about” refers to an approximately ±10% variation from a given value or range. In other embodiments, the term “about” refers to an approximately ±5% variation from a given value or range. In yet other embodiments, the term “about” refers to an approximately ±1% variation from a given value or range. It is to be understood that such a variation is always included in any given value provided herein, whether it is specifically referred to or not.

The use of the word “a” or “an,” when used herein in conjunction with the term “comprising,” can mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a construct, composition, use or method, denotes that additional features, elements and/or method steps can be present, but that these additions do not materially affect the manner in which the recited construct, composition, method or use functions. The term “consisting of,” when used herein in connection with a construct, composition, use or method, excludes the presence of additional elements and/or method steps. An antibody construct, composition, use, or method described herein as comprising certain elements and/or steps can also, in certain embodiments, consist essentially of those elements and/or steps, and in other embodiments, consist of those elements and/or steps, whether or not these embodiments are specifically referred to.

The terms “subject” and “patient” can be used interchangeably herein and generally refer to an animal in need of treatment. An animal in need of treatment can be a human or a non-human animal, such as a mammal, bird, or fish. In certain embodiments, the subject or patient is a mammal. In some embodiments, the subject is a human. In other embodiments, the subject is a rodent or a non-human primate.

An “effective amount” of a trivalent and trispecific antibody construct described herein, or a pharmaceutical composition comprising such antibody construct, in respect of a particular result to be achieved is an amount sufficient to achieve the desired result. For example, an “effective amount” of an antibody construct or pharmaceutical composition when referred to in respect of the killing of cancer cells, refers to an amount of antibody construct or composition comprising the antibody construct sufficient to produce a killing effect.

Unless specified otherwise, the terms “Fc region,” “Fc” and “Fc domain” are used interchangeably herein and refer to a C-terminal region of an immunoglobulin (Ig) heavy chain that contains at least a portion of a constant region. In various embodiments, an Fc domain herein can be dimeric. Such dimeric Fc domain can comprise a first Fc polypeptide and a second Fc polypeptide, wherein each Fc polypeptide can comprise a CH2 domain and a CH3 domain. Such dimeric Fc can either be homodimeric, i.e., comprising first and second Fc polypeptides that have identical amino acid sequences, or heterodimeric, i.e., comprising first and second Fc polypeptides that have different amino acid sequences, e.g., sequences that share about 95%, 96%, 97%, 98%, or about 99% sequence identity. In some embodiments, an antibody construct of the present disclosure comprises a homodimeric Fc domain. In yet other embodiments, and as further described herein, an antibody construct comprises a heterodimeric Fc domain in which at least one of the CH2 and/or CH3 domains of the first and second Fc polypeptides have amino acid sequences that share about 99% or less, 98% or less, or about 97% or less sequence identity.

The term “multispecific,” as used herein in the context of an antibody construct, refers to a biologically functional protein (e.g., an antibody construct as described herein) which is “at least bispecific,” i.e., it comprises at least a first binding domain and a second binding domain, wherein such first and second binding domain can bind specifically two distinct epitopes, e.g., a first epitope and a second epitope. Such first and second epitopes can be located on the same antigen or on different antigens, e.g., a first epitope on cluster of differentiation 3 (CD3) or cluster of differentiation 28 (CD28) and a second epitope on a TAA. Accordingly, in some embodiments, antibody constructs according to the present disclosure can comprise specificities for at least two different antigens or for at least three different antigens or targets. Hence, the term “multispecific” in the context of an antibody construct herein encompasses antibody constructs that are at least bispecific (i.e., comprising two binding domains with specificities for two different antigens or targets), or at least trispecific (i.e., comprising three binding domains with specificities for three different antigens or targets, e.g., CD3, CD28, and a TAA).

The term “trispecific,” as used herein in the context of an antibody construct, refers to a biologically functional protein (e.g., an antibody construct as described herein) which is “at least trispecific,” i.e., it comprises at least a first binding domain, a second binding domain and a third binding domain, wherein such first, second and third binding domains can bind specifically three distinct epitopes, e.g., a first epitope, a second epitope and a third epitope. Such first, second and third epitopes can be located on the same antigen or on different antigens, e.g., a first epitope on CD3, a second epitope on CD28, and a third epitope on a TAA (e.g., a Claudin (Cldn) such as Cldn6 or Cldn18.2, or mesothelin (MSLN), etc.). Accordingly, in some embodiments, trispecific antibody constructs according to the present disclosure can comprise specificities for at least three different antigens or targets. Hence, the specificity in the context of an antibody construct herein describes the total number of different epitopes and/or antigens an antibody construct can specifically bind to, e.g., a monospecific antibody construct comprises one or more binding domain(s) with a specificity for one epitope or antigen, a trispecific antibody construct comprises three or more binding domains with specificities for three different epitopes and/or antigens, and so forth.

The term “trivalent,” as used herein in the context of an antibody construct, refers to a biologically functional protein (e.g., an antibody construct as described herein) which is “at least trivalent,” i.e., it comprises three binding domains, e.g., at least a first binding domain, a second binding domain and a third binding domain, wherein each of the first, second, and third binding domains is capable of specifically binding an epitope and/or antigen, e.g., CD3, CD28, or a TAA. The three binding domains can either have specificities for three different epitopes or antigens, or two or more of the three binding domains have a specificity for the same epitope or antigen. Hence, the valency in the context of an antibody construct herein, e.g., being mono-, bi-, or trivalent, describes the total number of antigen binding domains of an antibody construct. Accordingly, the valency of an antibody construct has to be at least equal to its specificity, i.e., a trispecific antibody construct has to be at least trivalent. In embodiments in which the antibody construct is trivalent and trispecific, each of the construct's three binding domains is capable of binding a different epitope or antigen, and thus the construct engages each of the three epitopes and/or antigens monovalently.

As used herein, the term “format” in the context of an antibody construct described herein generally describes attributes of the antibody construct including its antigen valency (e.g., a construct being mono- or bivalent for a given antigen), the type(s) of binding domain(s) (e.g., possessing one or more scFv domain(s), Fab domain(s), etc.) present in an antibody construct, as well as the presence, absence, and/or type of an Fc domain (e.g., homodimeric, heterodimeric, containing one or more constant heavy domains, CH2, CH3, etc.). As an example, in some embodiments, an antibody construct of the present disclosure can be trivalent and trispecific in a 1+1+1 format illustrating that the construct contains three binding domains, wherein each binding domain has an affinity for a different antigen (e.g., CD3, CD28 and a TAA), i.e., the trivalent construct is monovalent (as indicated by “1”) for each of the three antigens.

As used herein, the term “geometry” in the context of an antibody construct described herein generally describes the overall structure of an antibody construct, including the relative spatial localization and arrangement and/or connectivity of the various domains of an antibody construct, e.g., the relative arrangement and connectivity of binding and Fc domains, as further described herein and as illustrated in FIGS. 1A-1G according to certain embodiments of the present disclosure.

Generally, and unless specified otherwise, an amino acid sequence of a polypeptide described herein is described and defined in the direction from N- to C-terminus. As an example, a polypeptide described as comprising an scFv domain coupled to an Fc polypeptide is defined herein as a polypeptide in which the C-terminus of the scFv domain is coupled, either with or without a linker, to the N-terminus of the Fc polypeptide and the domain structure can be described as: scFv-Fc, or with the inclusion of a linker as: scFv-Linker^{scFv-Fc}-Fc.

As used herein, abbreviations such as “H1” and “H2,” or “A” and “B,” are generally used as generic heavy chain identifiers and broadly refer to a first heavy chain and a second heavy chain of an antibody construct, respectively, and thus are not intended to be limited to any specific heavy chain amino acid (or polynucleotide) sequences.

The term “amino acid modification,” as used herein in the context of an amino acid sequence of a polypeptide, generally refers to an amino acid sequence of a polypeptide in which one or more amino acid substitution(s), one or more amino acid insertion(s), and/or one or more amino acid deletion(s) have been introduced relative to a corresponding unmodified (e.g., WT or reference) amino acid sequence of the polypeptide.

Descriptions of antibody constructs such as “anti-(Cldn18.2×CD28×CD3)” and “anti-CD3/anti-CD28/anti-Cldn18.2” can be used interchangeably herein and generally refer to an antibody construct that is at least trispecific and thus contains at least three binding domains that are capable of binding an epitope on Cldn18.2, CD28 and CD3, respectively. In various embodiments, such description refers to a trivalent and trispecific antibody construct having three binding domains that are capable of binding an epitope on Cldn18.2, CD28 and CD3, respectively.

Generally, it is to be understood that the positive recitation of a feature in one embodiment serves as a basis for excluding the feature in an alternative embodiment. In particular, where a list of options is presented for a given embodiment or claim, it is to be understood that one or more option can be deleted from the list and the shortened list can form an alternative embodiment, whether or not such an alternative embodiment is specifically referred to.

It is further contemplated that any embodiment discussed herein can be implemented with respect to any antibody construct, method, use, or composition disclosed herein, and vice versa.

Furthermore, modifications of the specific embodiments described herein that would be apparent to those skilled in the art are intended to be included within the scope of the claims recited herein.

### II. ANTIBODY CONSTRUCTS

In various embodiments, the present disclosure relates to trivalent and trispecific T cell engaging antibody constructs comprising three binding domains capable of engaging two different antigens on one or more cytotoxic effector cell(s) and a TAA on a tumor cell.

In some embodiments, a trivalent and trispecific antibody construct of the present disclosure comprises: (i) a first binding domain capable of binding a first antigen on a first cytotoxic effector cell, (ii) a second binding domain capable of binding a second antigen on a second cytotoxic effector cell, (iii) a third binding domain capable of binding a TAA on a tumor cell, and (iv) an Fc domain comprising a first Fc polypeptide and a second Fc polypeptide. The antibody constructs described herein can have various formats and geometries, e.g., the binding domains and the Fc domain can have various relative spatial localizations and arrangements as further described herein.

### A. Format and Geometry of Antibody Constructs

In various embodiments, the antibody constructs described in the present disclosure are trivalent and trispecific and comprise three antigen binding domains, with each of the three binding domains being capable of binding a different antigen. Hence, in various embodiments, the trivalent and trispecific antibody constructs of the present disclosure can have a format of 1+1+1, indicating that each of the three binding domains binds a different antigen, and hence such constructs bind each antigen in a monovalent manner.

In some embodiments, such trivalent and trispecific antibody constructs can comprise one or more different types of binding domains. Types of binding domains that can be used in the antibody constructs described herein include scFv domains, Fab domains, single domain antibodies (sdAbs), etc. Thus, in certain embodiments, an antibody construct described herein can comprise one or more scFv domain(s) and/or one or more Fab domain(s). In some embodiments, an antibody construct can comprise one or more scFv domain(s) and one or more Fab domain(s).

In some embodiments, a trivalent and trispecific antibody construct of the present disclosure comprises two scFv domains, e.g., a first scFv domain and a second scFv domain, and one Fab domain. Such antibody construct can further comprise an Fc domain. Thus, the two scFv domains, the Fab domain, and the Fc domain can be coupled to one another in various relative spatial arrangements to yield various construct geometries, e.g., as shown in FIGS. 1A-1G. In some embodiments, the Fc domain is a heterodimeric Fc domain.

In certain embodiments herein, a trivalent and trispecific antibody construct of the present disclosure does not contain a polypeptide chain which comprises two or more scFv domains coupled to one another in tandem, e.g., according to the domain structure, from either N- to C-terminus or C- to N-terminus of: (V_{H/L}-V_{L/H})_{scFv1}-(V_{H/L}-V_{L/H})_{scFv2}, wherein such chain may optionally contain one or more linkers coupling the V_{H} and V_{L} domains together, both, within an scFv domain or between the two scFv domains. Expression of antibody constructs that comprise a polypeptide chain containing two or more scFv domains coupled in tandem can reduce the producibility and stability (e.g., thermostability) of the antibody construct. Hence, in various embodiments, the antibody constructs described herein can have a higher producibility and/or thermal stability compared to conventional constructs comprising polypeptide chains with two or more scFv domains coupled in tandem.

In some embodiments, described herein is an antibody construct, comprising: (i) a Fab domain capable of binding a first antigen on a first cytotoxic effector cell; (ii) a first scFv domain and a second scFv domain, wherein one of the scFv domains is capable of binding a second antigen on a second cytotoxic effector cell and the other scFv domain is capable of binding a tumor-associated antigen (TAA) on a tumor cell, and (iii) an Fc domain comprising a first Fc polypeptide and a second Fc polypeptide, wherein: (a) the first and second antigens are different, (b) the Fab domain is coupled to the N-terminus of the first Fc polypeptide, and (c) the first and second scFv domains are independently coupled to either an N-terminus of the Fab domain, a C-terminus of the Fab domain, a C-terminus of one of the Fc polypeptides, or the N-terminus of the second Fc polypeptide.

In some embodiments, described herein is an antibody construct, comprising: (i) a Fab domain capable of binding a first antigen on a first cytotoxic effector cell; (ii) a first scFv domain and a second scFv domain, wherein one of the scFv domains is capable of binding a second antigen on a second cytotoxic effector cell and the other scFv domain is capable of binding a tumor-associated antigen (TAA) on a tumor cell, and (iii) an Fc domain comprising a first Fc polypeptide and a second Fc polypeptide, wherein: (a) the first and second antigens are different, (b) the Fab domain is coupled to the N-terminus of the first Fc polypeptide, and (c) the first and second scFv domains are independently coupled to either an N-terminus of the Fab domain, a C-terminus of the Fab domain, or the N-terminus of the second Fc polypeptide.

In one embodiment, the present disclosure describes a trivalent and trispecific antibody construct, comprising: (i) a Fab domain capable of binding a first antigen on a first cytotoxic effector cell; (ii) a first scFv domain capable of binding a second antigen on a second cytotoxic effector cell; (iii) a second scFv domain capable of binding a tumor-associated antigen (TAA); and (iv) a heterodimeric Fc domain comprising a first Fc polypeptide and a second Fc polypeptide, wherein: (a) the Fab domain is coupled to the N-terminus of the first Fc polypeptide or the N-terminus of the second Fc polypeptide, (b) the first scFv domain is coupled to either an N-terminus of the Fab domain, a C-terminus of the Fab domain, the C-terminus of the first Fc polypeptide, or the N-terminus of the second Fc polypeptide, and (c) the second scFv domain is coupled to either the N-terminus of the first Fc polypeptide, the N-terminus of the second Fc polypeptide, or an N-terminus of the Fab domain.

In one embodiment, the present disclosure describes a trivalent and trispecific antibody construct, comprising: (i) a Fab domain capable of binding a first antigen on a first cytotoxic effector cell; (ii) a first scFv domain capable of binding a second antigen on a second cytotoxic effector cell; (iii) a second scFv domain capable of binding a tumor-associated antigen (TAA); and (iv) a heterodimeric Fc domain comprising a first Fc polypeptide and a second Fc polypeptide, wherein: (a) the Fab domain is coupled to the N-terminus of the first Fc polypeptide, (b) the first scFv domain is coupled to either an N-terminus of the Fab domain, a C-terminus of the Fab domain, or the C-terminus of the first Fc polypeptide, and (c) the second scFv domain is coupled to the N-terminus of the second Fc polypeptide.

In some embodiments, the first antigen is CD3 or CD28, wherein the first antigen and the second antigen are different antigens.

In some embodiments, the second antigen is CD3 or CD28, wherein the first antigen and the second antigen are different antigens.

In certain embodiments of the present disclosure, the first antigen on the first cytotoxic effector cell is CD28, and the second antigen on the second cytotoxic effector cell is CD3. In other embodiments, the first antigen is CD3, and the second antigen is CD28.

In one embodiment, the present disclosure describes a trivalent and trispecific antibody construct, comprising: (i) a Fab domain capable of binding CD28 on a first cytotoxic effector cell; (ii) a first scFv domain capable of binding CD3 on a second cytotoxic effector cell; (iii) a second scFv domain capable of binding a TAA on a tumor cell; and (iv) a heterodimeric Fc domain comprising a first Fc polypeptide and a second Fc polypeptide, wherein: (a) the Fab domain is coupled to the N-terminus of the first Fc polypeptide, (b) the first scFv domain is coupled to either an N-terminus of the Fab domain, a C-terminus of the Fab domain, or the C-terminus of the first Fc polypeptide, and (c) the second scFv domain is coupled to the N-terminus of the second Fc polypeptide.

In one embodiment, the present disclosure describes a trivalent and trispecific antibody construct, comprising: (i) a Fab domain capable of binding CD3 on a first cytotoxic effector cell; (ii) a first scFv domain capable of binding CD28 on a second cytotoxic effector cell; (iii) a second scFv domain capable of binding a TAA on a tumor cell; and (iv) a heterodimeric Fc domain comprising a first Fc polypeptide and a second Fc polypeptide, wherein: (a) the Fab domain is coupled to the N-terminus of the first Fc polypeptide, (b) the first scFv domain is coupled to either an N-terminus of the Fab domain, a C-terminus of the Fab domain, or the C-terminus of the first Fc polypeptide, and (c) the second scFv domain is coupled to the N-terminus of the second Fc polypeptide.

In some embodiments, described herein is an antibody construct, comprising: (i) a Fab domain capable of binding a first antigen on a first cytotoxic effector cell; (ii) a first scFv domain and a second scFv domain, wherein one of the scFv domains is capable of binding a second antigen on a second cytotoxic effector cell and the other scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, and (b) the first and second scFv domains are independently coupled to either (i) an N-terminus of the Fab domain, (ii) a C-terminus of the Fab domain, (iii) the C-terminus of one of the Fc polypeptides, or (iv) the N-terminus of the second Fc polypeptide, provided that when one of the scFv domains is coupled to the C-terminus of one of the Fc polypeptides, the first antigen is CD3 and the second antigen is CD28, or the first antigen is CD28 and the second antigen is CD3, and that the first scFv domain and the second scFv domain are not coupled to each other in tandem.

In some embodiments of a trivalent and trispecific antibody construct described herein, the first scFv domain is coupled to an N-terminus of the Fab domain. In such embodiments, the first scFv domain can be coupled to the N-terminus of the V_{H} sequence of the heavy chain of the Fab domain. In other embodiments, the first scFv domain can be coupled to the N-terminus of the V_{L} sequence of the light chain of the Fab domain.

In certain embodiments, a trivalent and trispecific antibody construct of the present disclosure comprises: a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, and (iii) the first Fc polypeptide; b) a second heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), and (ii) the second Fc polypeptide; and c) a light chain polypeptide comprising, from N-terminus to C-terminus: a Fab V_{L} sequence coupled to a Fab CL sequence, wherein: the heavy chain Fab sequence and the light chain polypeptide form the Fab domain, and the first Fc polypeptide and the second Fc polypeptide form the heterodimeric Fc domain.

In other embodiments of a trivalent and trispecific antibody construct, the first scFv domain is coupled to the C-terminus of the first Fc polypeptide. In such embodiments, the trivalent and trispecific antibody construct can comprise: a) a first heavy chain polypeptide comprising, from N-terminus to C-terminus: (i) a heavy chain Fab sequence comprising a Fab V_{H} sequence coupled to a Fab C_{H1} sequence, (ii) the first Fc polypeptide, and (iii) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}); b) a second heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), and (ii) the second Fc polypeptide; and c) a light chain polypeptide comprising a Fab V_{L} sequence coupled to a Fab C_{L} sequence, wherein: the heavy chain Fab sequence and the light chain polypeptide form the Fab domain, and the first Fc polypeptide and the second Fc polypeptide form the heterodimeric Fc domain. One embodiment of such trivalent and trispecific antibody construct is depicted in FIG. 1F.

In yet other embodiments of a trivalent and trispecific antibody construct described herein, the first scFv domain is coupled to the C-terminus of the C_{L} sequence of the light chain of the Fab domain. In such embodiments, the antibody construct can comprise: a) a first heavy chain polypeptide comprising, from N-terminus to C-terminus: (i) a heavy chain Fab sequence comprising a Fab V_{H} sequence coupled to a Fab C_{H1} sequence, and (ii) the first Fc polypeptide; b) a second heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), and (ii) the second Fc polypeptide; and c) a light chain polypeptide comprising, from N-terminus to C-terminus: (i) a light chain Fab sequence comprising a Fab V_{L} sequence coupled to a Fab C_{L} sequence, and (ii) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), wherein: the heavy chain Fab sequence and the Fab light chain polypeptide sequence form the Fab domain, and the first Fc polypeptide and the second Fc polypeptide form the heterodimeric Fc domain. One embodiment of such trivalent and trispecific antibody construct is depicted in FIG. 1B.

In one embodiment, the present disclosure describes a trivalent and trispecific antibody construct, comprising: (i) a Fab domain capable of binding CD28 on a first cytotoxic effector cell; (ii) a first scFv domain capable of binding CD3 on a second cytotoxic effector cell; (iii) a second scFv domain capable of binding a TAA on a tumor cell; and (iv) a heterodimeric Fc domain comprising a first Fc polypeptide and a second Fc polypeptide, wherein: (a) the Fab domain is coupled to the N-terminus of either the first Fc polypeptide or the second Fc polypeptide, (b) the first scFv domain is coupled to either the N-terminus of the first Fc polypeptide, the C-terminus of the first Fc polypeptide, or the N-terminus of the second Fc polypeptide, and (c) the second scFv domain is coupled to an N-terminus of the Fab domain. Certain embodiments of such trivalent and trispecific antibody construct are depicted in FIGS. 1D and 1F.

In another embodiment, the present disclosure describes a trivalent and trispecific antibody construct, comprising: (i) a Fab domain capable of binding CD3 on a first cytotoxic effector cell; (ii) a first scFv domain capable of binding CD28 on a second cytotoxic effector cell; (iii) a second scFv domain capable of binding a TAA on a tumor cell; and (iv) a heterodimeric Fc domain comprising a first Fc polypeptide and a second Fc polypeptide, wherein: (a) the Fab domain is coupled to the N-terminus of either the first Fc polypeptide or the second Fc polypeptide, (b) the first scFv domain is coupled to either the N-terminus of the first Fc polypeptide, the C-terminus of the first Fc polypeptide, or the N-terminus of the second Fc polypeptide, and (c) the second scFv domain is coupled to an N-terminus of the Fab domain. Certain embodiments of such trivalent and trispecific antibody construct are depicted in FIGS. 1D and 1F.

In a certain embodiment, the Fab domain is coupled to the N-terminus of the first Fc polypeptide and the first scFv domain is coupled to the C-terminus of the first Fc polypeptide. Exemplary embodiments are depicted in FIGS. 1C and 1F. In one such embodiment, e.g., as shown in FIG. 1C, the antibody construct can comprise: a) a first heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), (ii) a heavy chain Fab sequence comprising a Fab V_{H} sequence coupled to a Fab C_{H1} sequence, (iii) the first Fc polypeptide, and (iv) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}); b) a second heavy chain polypeptide comprising or consisting of the second Fc polypeptide; and c) a light chain polypeptide comprising, from N-terminus to C-terminus: a light chain Fab sequence comprising a Fab V_{L} sequence coupled to a Fab C_{L} sequence, wherein: the heavy chain Fab sequence and the light chain polypeptide form the Fab domain, and the first Fc polypeptide and the second Fc polypeptide form the heterodimeric Fc domain.

In another embodiment of a trivalent and trispecific antibody construct described herein, the Fab domain is coupled to the N-terminus of the first Fc polypeptide and the first scFv domain is coupled to the N-terminus of the second Fc polypeptide, e.g., as shown in FIG. 1E. In such an embodiment, the antibody construct can comprise: a) a first heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), (ii) a heavy chain Fab sequence comprising a Fab V_{H} sequence coupled to a Fab C_{H1} sequence, and (iii) the first Fc polypeptide; b) a second heavy chain polypeptide comprising, from N-terminus to C-terminus: (i) the first scFv domain comprising cither a first scFv V_{H} sequence coupled to a first scFv V_{L} sequence (V_{H}-V_{L}), or a first scFv V_{L} sequence coupled to a first scFv V_{H} sequence (V_{L}-V_{H}), and (ii) the second Fc polypeptide; and c) a light chain polypeptide comprising, from N-terminus to C-terminus: a light chain Fab sequence comprising a Fab V_{L} sequence coupled to a Fab C_{L} sequence, wherein: the heavy chain Fab sequence and the light chain polypeptide form the Fab domain, and the first Fc polypeptide and the second Fc polypeptide form the heterodimeric Fc domain.

In another embodiment, the Fab domain is coupled to the N-terminus of the second Fc polypeptide and the first scFv domain is coupled to the N-terminus of the first Fc polypeptide, e.g., as shown in FIG. 1D. In such an embodiment, the antibody construct can comprise: a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), and (iii) the first Fc polypeptide; b) a second heavy chain polypeptide comprising, from N-terminus to C-terminus: (i) the second scFv domain comprising either a second scFv V_{H} sequence coupled to a second scFv V_{L} sequence (V_{H}-V_{L}.), or a second scFv V_{L} sequence coupled to a second scFv V_{H} sequence (V_{L}-V_{H}), (ii) a heavy chain Fab sequence comprising a Fab V_{H} sequence coupled to a Fab C_{H1} sequence, and (iii) the second Fc polypeptide; and c) a light chain polypeptide comprising, from N-terminus to C-terminus: a light chain Fab sequence comprising a Fab V_{L} sequence coupled to a Fab C_{L} sequence, wherein: the heavy chain Fab sequence and the light chain polypeptide form the Fab domain, and the first Fc polypeptide and the second Fc polypeptide form the heterodimeric Fc domain.

In some embodiments, described herein is an antibody construct in which each of the first and second scFv domains can be independently coupled to an N-terminus of the Fab domain, a C-terminus of the Fab domain, the C-terminus of one of the Fc polypeptide, or the N-terminus of the second Fc polypeptide.

In such embodiments, an antibody construct can comprise: (i) a Fab domain capable of binding a CD3 on a first cytotoxic effector cell; (ii) a first scFv domain and a second scFv domain, wherein one of the scFv domains is capable of binding CD28 on a second cytotoxic effector cell and the other scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, and (b) each of the first and second scFv domains is independently coupled to an N-terminus of the Fab domain, a C-terminus of the Fab domain, the C-terminus of one of the Fc polypeptide, or the N-terminus of the second Fc polypeptide.

In other embodiments, an antibody construct can comprise: (i) a Fab domain capable of binding a CD28 on a first cytotoxic effector cell; (ii) a first scFv domain and a second scFv domain, wherein one of the scFv domains is capable of binding CD3 on a second cytotoxic effector cell and the other scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, and (b) the first and second scFv domains are each independently coupled to either an N-terminus of the Fab domain, a C-terminus of the Fab domain, the C-terminus of one of the Fc polypeptide, or the N-terminus of the second Fc polypeptide.

In some of these embodiments, the first scFv domain is coupled to an N-terminus of the Fab domain and the second scFv is coupled to the N-terminus of the second Fc polypeptide. In certain embodiments, the first scFv domain is coupled to the N-terminus of the V_{H} sequence of the heavy chain of the Fab domain. In other embodiments, the first scFv domain is coupled to the N-terminus of the V_{L} sequence of the light chain of the Fab domain.

In some embodiments, a trivalent and trispecific antibody construct can comprise or consist of three polypeptide chains that can associate and form the antibody construct. In some embodiments, such trivalent and trispecific antibody construct comprises: a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, and (iii) the first Fc polypeptide; b) a second heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), and (ii) the second Fc polypeptide; and c) a light chain polypeptide comprising, from N-terminus to C-terminus: a Fab V_{L} sequence coupled to a Fab C_{L} sequence, wherein: the heavy chain Fab sequence and the light chain polypeptide form the Fab domain, and the first Fc polypeptide and the second Fc polypeptide form the Fc domain.

In some embodiments, the first scFv domain is coupled to the C-terminus of the Fab domain and the second scFv domain is coupled to the N-terminus of the second Fc polypeptide. In such embodiments, a trivalent and trispecific antibody construct can comprise the following polypeptide chains: a) a first heavy chain polypeptide comprising, from N-terminus to C-terminus: (i) a heavy chain Fab sequence comprising a Fab V_{H} sequence coupled to a Fab C_{H1} sequence, and (ii) the first Fc polypeptide; b) a second heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), and (ii) the second Fc polypeptide; and c) a light chain polypeptide comprising, from N-terminus to C-terminus: (i) a Fab V_{L} sequence coupled to a Fab C_{L} sequence, and (ii) the first scFv domain comprising either a first scFv V_{H} sequence coupled to a first scFv V_{L} sequence (V_{H}-V_{L}.), or a first scFv V_{L}, sequence coupled to a first scFv V_{H} sequence (V_{L}-V_{H}), wherein: the heavy chain Fab sequence and the Fab sequence of the light chain polypeptide form the Fab domain, and the first Fc polypeptide and the second Fc polypeptide form the Fc domain.

In some embodiments of trivalent and trispecific antibody constructs described herein, the first scFv domain is coupled to an N-terminus of the Fab domain and the second scFv domain is coupled to the C-terminus of one of the Fc polypeptides. In such embodiments, the first scFv domain can be coupled to the N-terminus of the V_{H} domain of the Fab domain. In other embodiments, the first scFv domain is coupled to the N-terminus of the V_{L} domain of the Fab domain. In some of these embodiments, the second scFv domain is coupled to the C-terminus of the first Fc polypeptide. In yet other such embodiments, the second scFv domain is coupled to the C-terminus of the first Fc polypeptide.

In some of these embodiments, a trivalent and trispecific antibody construct can comprise the following polypeptide chains: a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, (iii) the first Fc polypeptide, and (iv) the second scFv domain comprising either a second scFv V_{H} sequence coupled to a second scFv V_{L} sequence (V_{H}-V_{L}.), or a second scFv V_{L} sequence coupled to a second scFv V_{H} sequence (V_{L}-V_{H}); b) a second heavy chain polypeptide comprising the second Fc polypeptide; and c) a light chain polypeptide comprising a Fab V_{L} sequence coupled to a Fab C_{L} sequence, wherein: the heavy chain Fab sequence and the light chain polypeptide form the Fab domain, and the first Fc polypeptide and the second Fc polypeptide form the Fc domain.

In other embodiments, a trivalent and trispecific antibody construct can comprise the following polypeptide chains: a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, and (iii) the first Fc polypeptide; b) a second heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), and (ii) the second Fc polypeptide; and c) a light chain polypeptide comprising a Fab V_{L} sequence coupled to a Fab C_{L} sequence, wherein: the heavy chain Fab sequence and the light chain polypeptide form the Fab domain, and the first Fc polypeptide and the second Fc polypeptide form the Fc domain.

In some embodiments of the trivalent and trispecific antibody constructs described herein that comprise a first scFv domain and a second scFv domain, the first scFv domain is capable of binding the TAA, and the second scFv domain is capable of binding CD28 or CD3. In yet other embodiments, the second scFv domain is capable of binding the TAA, and the first scFv domain is capable of binding CD28 or CD3.

In one embodiment, described herein is an antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the N-terminus of the V_{H} domain of the Fab domain, and (c) the second scFv domain is coupled to the N-terminus of the second Fc polypeptide.

In one embodiment, described herein is an antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the N-terminus of the V_{H} domain of the Fab domain, and (c) the second scFv domain is coupled to the N-terminus of the second Fc polypeptide.

In one embodiment, described herein is an antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the C_{L} domain of the Fab domain, and (c) the second scFv domain is coupled to the N-terminus of the second Fc polypeptide.

In one embodiment, described herein is an antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the C_{L} domain of the Fab domain, and (c) the second scFv domain is coupled to the N-terminus of the second Fc polypeptide.

In one embodiment, described herein is an antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the second Fc polypeptide.

In one embodiment, described herein is an antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the second Fc polypeptide.

In one embodiment, described herein is an antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the V_{H} domain of the Fab domain.

In one embodiment, described herein is an antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the V_{H} domain of the Fab domain.

In various embodiments, the dimeric Fc domain of a trivalent and trispecific antibody construct is a heterodimeric Fc domain.

In some of the embodiments, the first scFv domain has the domain structure, from N- to C-terminus, of: V_{H}-V_{L}. As further described herein, the first scFv domain can further comprise a linker^{scFv1} which couples the first scFv V_{H} sequence to the first scFv V_{L} sequence.

In certain other embodiments, the first scFv domain has the domain structure, from N- to C-terminus, of: V_{L}-V_{H}. As further described herein, the first scFv domain can further comprise a linker^{scFv1} which couples the first scFv V_{L} sequence to the first scFv V_{H} sequence.

In some embodiments, the second scFv domain has the domain structure, from N- to C-terminus, of: V_{H}-V_{L}. As further described herein, the second scFv domain can further comprise a linker^{scFv2} which couples the second scFv V_{H} sequence to the second scFv V_{L} sequence.

In certain embodiments, the second scFv domain has the domain structure, from N- to C-terminus, of: V_{L}-V_{H}. As further described herein, the second scFv domain can further comprise a linker^{scFv2} which couples the second scFv V_{L} sequence to the second scFv V_{H} sequence.

In some embodiments, the first scFv domain has the domain structure, from N- to C-terminus, of: V_{L}-V_{H}, and the second scFv domain has the domain structure, from N- to C-terminus, of: V_{L}-V_{H}.

In some embodiments, the first scFv domain has the domain structure, from N- to C-terminus, of: V_{H}-V_{L}, and the second scFv domain has the domain structure, from N- to C-terminus, of: V_{H}-V_{L}.

In some embodiments, the first scFv domain has the domain structure, from N- to C-terminus, of: V_{L}-V_{H}, and the second scFv domain has the domain structure, from N- to C-terminus, of: V_{H}-V_{L}.

In some embodiments, the first scFv domain has the domain structure, from N- to C-terminus, of: V_{H}-V_{L}, and the second scFv domain has the domain structure, from N- to C-terminus, of: V_{L}-V_{H}.

In various embodiments of the trivalent and trispecific antibody construct of the present disclosure, the TAA is MSLN or Cldn18.2, as further described herein.

As further described herein, an antibody construct of the present disclosure can further comprise one or more linkers. Such one or more linkers can be one or more peptide linkers. The one or more linkers can couple one, two, or more domains and/or sequences of an antibody construct to each other. As an example, a linker^{scFv} herein can couple an scFv V_{H} sequence to an scFv V_{L} sequence.

### B. Domains of an Antibody Construct

A trivalent and trispecific antibody construct of the present disclosure can comprise one or more antibody domains. In various embodiments, a trivalent and trispecific antibody construct of the present disclosure comprises a plurality of (i.e., two or more) antibody domains. Such plurality of antibody domains can comprise (i) one or more Fc domain(s), wherein an Fc domain can comprise a first Fc polypeptide and a second Fc polypeptide, and can be either homodimeric or heterodimeric (ii) one or more Fab domain(s), wherein a Fab domain can comprise a heavy chain polypeptide comprising a heavy variable domain (V_{H}) sequence and a heavy constant domain (C_{H1}) sequence and a light chain polypeptide comprising a light variable domain (V_{L}) sequence and light constant domain (C_{L}) sequence, and (iii) one or more scFv domains, wherein an scFv domain can comprise an scFv V_{H} sequence coupled to an scFv V_{L} sequence. The various domains an antibody construct can comprise are further described herein.

An Ig structural unit is typically composed of two pairs of polypeptide chains, each pair having one “light” chain (about 25 kilodalton (kD)) and one “heavy” chain (about 50-70 kD). Light chains can be classified as either kappa or lambda. The “class” of an Ig refers to the type of constant domain possessed by its heavy chain. There are five major classes of antibodies: IgA, IgD, IgE, IgG and IgM, and several of these can be further divided into subclasses (isotypes), for example, IgG1, IgG2, IgG3, IgG4, IgA1 and IgA2. The heavy chain constant domains that correspond to the different classes of immunoglobulins (Igs) are called alpha (α), delta (δ), epsilon (ε), gamma (γ) and mu (μ), respectively.

In various embodiments, a trivalent and trispecific antibody construct described herein is based on an IgG class immunoglobulin, for example, an IgG1, IgG2, IgG3 or IgG4 immunoglobulin. In some embodiments, an antibody construct described herein is based on an IgG1, IgG2 or IgG4 immunoglobulin. In certain embodiments, an antibody construct described herein is based on an IgG1 immunoglobulin. In the context of the present disclosure, when an antibody construct is based on a specified Ig isotype, it refers to an antibody construct that comprises either all or a portion of the constant region (i.e., Fc domain) of the specified Ig isotype. It is to be understood that an antibody construct can also comprise hybrids of isotypes and/or subclasses, according to certain embodiments of this disclosure.

Generally, in antibodies, the N-terminal domain of each polypeptide chain usually defines a variable region (e.g., V_{H} or V_{L}) of about 100 to 110 or more amino acids in length that is primarily responsible for antigen recognition. The terms variable light chain (V_{L}) and variable heavy chain (V_{H}) refer to these domains in the light and heavy chain, respectively. As described herein, in various embodiments, a trivalent and trispecific antibody construct of the present disclosure can comprise two or more variable domain sequences. In various embodiments, a trivalent and trispecific antibody construct of the present disclosure comprises two variable domain sequence per binding domain, and thus by being trivalent (i.e., containing three binding domains) and trispecific (i.e., each of the three binding domains targets a different antigen) can comprise a total of six variable domain sequences, e.g., three V_{H} domain sequences and three V_{L} domain sequences.

In some embodiments, two or more of such variable domains are coupled to one another in tandem and in a single polypeptide chain format, e.g., as described for scFv-type binding domains which contain (from either N- or C-terminus) a V_{H} domain coupled to a V_{L} domain, or as described for constructs in which an scFv domain (which contains two variable domain sequences coupled to one another in tandem) is coupled to a Fab domain via a Fab variable domain sequence, e.g., either the Fab V_{H} or the Fab V_{L} sequence. In some embodiments, such construct can comprise the heavy chain domain structure, from N- to C-terminus: [(V_{H}-V_{L}) or (V_{L}-V_{H})]_{scFv}-V_{H}-C_{H1}-Fc.

Accordingly, an antibody construct of the present disclosure that is derived from an Ig molecule can comprise different Ig domains within its heavy and light chain(s). Heavy chain domains can include the Fc domain (or Fc region), e.g., comprising a CH2 domain and CH3 domain, a hinge domain (or hinge region), a heavy chain Fab domain comprising a variable heavy domain (V_{H}) and a constant heavy domain (C_{H1}), and light chain domains can include the variable light domain (V_{L}) and the light constant domain (C_{L}). In some embodiments, and according to certain nomenclatures, the “Fc domain” can include the CH2 and CH3 domains as well as a hinge domain (or hinge region).

### B.1 Complementarity Determining Regions (CDRs) and Binding Domains

In each of the V_{H} and V_{L} domains of an antibody construct herein are three loops which are hypervariable in sequence and form an antigen-binding site. Each of these loops is referred to as a “hypervariable region” or “HVR,” or “complementarity determining region” or “CDR.” The terms hypervariable region (HVR) and complementarity determining region (CDR) are used herein interchangeably in reference to the portions of the variable domain (e.g., V_{H} or V_{L}) that form the antigen-binding site. With the exception of CDR1 in V_{H}, CDRs generally comprise the amino acid residues that form the hypervariable loops. The V_{H} and V_{L} domains consist of relatively invariant stretches called framework regions (FRs) of between about 15 to 30 amino acids in length separated by the shorter CDRs, which are each typically between about 5 and 15 amino acids in length, although can occasionally be longer or shorter. The three CDRs and four FRs that make up each V_{H} and V_{L} domain are arranged from N- to C-terminus as follows: FR1-CDR1-FR2-CDR2-FR3-CDR3-FR4.

Several different definitions and numbering conventions of the CDR regions in Ig molecules are in common use, including those described by Kabat et al. (1983, Sequences of Proteins of Immunological Interest, NIH Publication No. 369-847, Bethesda, MD), by Chothia et al. (1987, J Mol Biol, 196:901-917), as well as the IMGT, AbM and Contact definitions. These different definitions include overlapping or subsets of amino acid residues when compared against each other. By way of example, CDR definitions according to Kabat, Chothia, IMGT, AbM and Contact are provided in TABLE 1 below.

Accordingly, as can be readily apparent to one skilled in the art, the exact numbering and placement of CDRs can differ based on the numbering system employed. However, it is to be understood that the disclosure herein of a variable heavy domain (V_{H}) includes the disclosure of the associated (inherent) heavy chain CDRs (HCDRs) as defined by any of the known numbering systems. Similarly, disclosure herein of a variable light domain (V_{L}) includes the disclosure of the associated (inherent) heavy chain CDRs (HCDRs) as defined by any of the known numbering systems. One skilled in the art can appreciate that a limited number of amino acid substitutions can be introduced into the CDR sequences or to the V_{H} or V_{L} sequences of known antibodies without the antibody losing its ability to bind its target, e.g., a reduction in binding affinity of at least about 1000-fold or more. Candidate amino acid substitutions can be identified by computer modeling or by techniques such as alanine scanning, with the resulting variants being tested for binding activity (e.g., expressed as binding affinity, e.g., given as the measured EC₅₀ value) by standard techniques. As an example, in certain embodiments, the CD3 binding domain(s) of antibody constructs described herein can comprise a set of CDRs (i.e., heavy chain CDR1, CDR2 and CDR3, and light chain CDR1, CDR2 and CDR3) that have 90% or greater, 95% or greater, 98% or greater, 99% or greater, or 100% sequence identity to the amino acid sequences set forth in SEQ ID NOs: 321-326, respectively, wherein the binding domain retains or substantially retains the ability to bind CD3. In this context, the term “substantially” refers to a change in binding affinity of less than about 5%, less than about 4%, less than about 3%, less than about 2%, or less than about 1%.

TABLE 1
Common CDR Definitions¹

| | Heavy Chain | | | Light Chain | | |
|---|---|---|---|---|---|---|
| Definition | CDR1² | CDR2 | CDR3 | CDR1 | CDR2 | CDR3 |
| Kabat | H31-H35B | H50-H65 | H95-H102 | L24-L34 | L50-L56 | L89-L97 |
| Chothia | H26-H32, | H52-H56 | H95-H102 | L24-L34 | L50-L56 | L89-L97 |
| | H33 or H34 | | | | | |
| IMGT | H26-H33, H34, H35, | H51-H57 | H93-102 | L27-L32 | L50-L52 | L89-L97 |
| | H35A or H35B | | | | | |
| AbM | H26-H35B | H50-H58 | H95-H102 | L24-L34 | L50-L56 | L89-L97 |
| Contact | H30-H35B | H47-H58 | H95-H101 | L30-L36 | L46-L55 | L89-L96 |

¹Either the Kabat or Chothia numbering system can be used for HCDR2, HCDR3 and the light chain CDRs for all definitions except Contact, which uses Chothia numbering.
²Using Kabat numbering. The position in the Kabat numbering scheme that demarcates the end of the Chothia and IMGT CDR-H1 loop varies depending on the length of the loop due to the placement of insertions outside of those CDR definitions at positions 35A and 35B in Kabat. The IMGT and Chothia CDR-H1 loop can be unambiguously defined using Chothia numbering. CDR-H1 definitions using Chothia numbering are: Kabat H31-H35, Chothia H26-H32, AbM H26-H35, IMGT H26-H33, Contact H30-H35.

In some embodiments, the antibody constructs described herein comprise at least one Ig domain from a mammalian Ig, such as a bovine Ig, a human Ig, a camelid Ig, a rat Ig, or a mouse Ig. In some embodiments, an antibody construct herein can be a chimeric construct comprising two or more Ig domains, in which at least one domain is from a first mammalian Ig, for example a human Ig, and at least a second domain is from a second mammalian Ig, for example, a mouse or rat Ig. In other embodiments, an antibody construct can be derived from Igs that are from different species, for example, an antibody construct can be chimeric or humanized. A “chimeric antibody construct” refers to an antibody that typically comprises at least one variable domain from a rodent antibody (usually a murine antibody) and at least one constant domain from a human antibody. A “humanized antibody construct” is a type of chimeric antibody that contains minimal sequence derived from a non-human antibody. In some embodiments, an antibody construct herein can comprise at least one Ig constant domain from a human Ig. In various embodiments, all domains of an antibody construct described herein can be (or be derived from) from a human Ig.

In some embodiments, and as further described herein, modifications (e.g., to the amino acid sequence) to one or more domains of an antibody construct can be made to further refine the antibody construct's properties and performance (e.g., antigen affinity, stability, and/or pharmacokinetics, etc.). For example, framework region (FR) residues of a human Ig can be replaced by corresponding non-human residues, or the humanized antibodies can comprise residues that are not found in either the recipient antibody or the donor antibody. In general, a variable domain in a humanized antibody or a humanized antibody domain comprises all or substantially all of the hypervariable regions from a non-human Ig and all or substantially all of the FRs from a human Ig sequence. As further described herein, modifications in the Fc domain can enable preferential pairing of the Fc polypeptides to form a heterodimeric Fc domain rather than a homodimeric Fc domain.

In some embodiments, the present disclosure relates to antibody constructs that can have different valencies, e.g., can be bivalent or trivalent. Hence, in various embodiments, an antibody construct herein comprises two or three antigen binding domains, i.e., is at least bivalent or at least trivalent. In various embodiments of this disclosure, an antibody construct can be trispecific and trivalent, and thus such antibody construct can comprise three binding domains. Each of the three binding domains can have a unique binding specificity for an antigen (either the same epitope/antigen or different ones). In some of these embodiments, a trispecific and trivalent antibody construct of the present disclosure comprises three binding domains, e.g., one or more Fab domain(s) and/or one or more scFv domain(s), capable of binding three different antigens (e.g., CD3, CD28, and a TAA).

In some embodiments, an antibody construct of the present disclosure can comprise (i) one or more Fab domain(s), (ii) one or more scFv domain(s), and (iii) an Fc domain comprising a first Fc polypeptide and a second Fc polypeptide. In various embodiments, a trivalent and trispecific antibody construct as described herein comprises (i) a Fab domain capable of binding to a first antigen, (ii) a first scFv domain capable of binding to a second antigen, and (iii) a second scFv domain capable of binding to a third antigen, and (iv) a heterodimeric Fc domain comprising a first Fc polypeptide and a second Fc polypeptide.

Generally, a “Fab domain,” as used herein, comprises a constant region comprising the constant domain (C_{L}) of the light chain and the first constant domain (C_{H1}) of the heavy chain, and a variable region comprising the variable domains V_{L} and V_{H} on the light and heavy chains, respectively, which comprise the CDRs as described herein. In some embodiments, a Fab domain can be a single chain Fab. A single chain Fab can be a Fab molecule in which the Fab light chain and the Fab heavy constant chain are connected by a peptide linker to form a single polypeptide chain. In such embodiments, typically, the C-terminus of the Fab light chain is connected to the N-terminus of the Fab heavy chain in the single-chain Fab molecule, however, other formats are also encompassed herein. In various embodiments herein, however, a Fab domain of an antibody construct is formed by two separately expressed polypeptide chains, i.e., a light chain and a heavy chain (or portion thereof). The heavy chain and light chain parts of the Fab domain can, however, be interconnected by covalent bonds, such as disulfide bonds.

An “scFv domain,” as used herein, generally comprises a heavy chain variable domain (V_{H}) and a light chain variable domain (V_{L}.) in a single polypeptide chain format. The scFv can optionally comprise a peptide linker between the V_{H} and V_{L} domains which can assist the scFv in forming a functional structure for antigen binding. Hence, in various embodiments, an scFv domain herein can include a V_{L} domain that is coupled via its C-terminus to the N-terminus of a V_{H} domain by a linker^{scFv}, i.e., an scFv domain can have the domain structure: V_{L}-linker^{scFv}-V_{H}, or alternatively, an scFv can comprise a V_{H} connected by its C-terminus to the N-terminus of a V_{L} by a linker^{scFv}, i.e., having the domain structure: V_{H}-linker^{scFv}-V_{L}.

In some embodiments, an antibody construct described herein can further comprise another domain or moiety that may not be derived from an Ig molecule. Such a non-Ig domain can be referred to as a moiety. Such moiety can be a detectable label (e.g., a radiolabel or fluorescent label), a low molecular weight (e.g., <750 Da) drug molecule, another peptide (e.g., signal peptide(s)) or polypeptide molecule, or combinations thereof.

### B.2 Binding Domains Against Antigens on Cytotoxic Effector Cells

As further described herein, an antibody construct of the present disclosure can comprise at least two binding domains capable of binding to one or more molecule(s), e.g., a polypeptide(s), on the surface of one or more cytotoxic effector cell(s). Such one or more cytotoxic effector cell(s) can be one or more immune cell(s). Such one or more immune cell(s) can comprise a T cell, a macrophage, a dendritic cell, a neutrophil, a B-cell, an NK cell, or a combination thereof.

In various embodiments, a trivalent and trispecific antibody construct of the present disclosure comprises at least one binding domain capable of binding a first antigen on a first cytotoxic effector cell, and at least one binding domain capable of binding a second antigen on a second cytotoxic effector cell.

In various embodiments, the first and second cytotoxic effector cells are different cells, i.e., the first and the second antigens are located on surfaces of different cells. In other embodiments, the first and second cytotoxic effector cells are the same cell, e.g., the first antigen and the second antigen that an antibody construct is engaging are expressed by the same cell, i.e., are located on the same cell surface. In some embodiments, the first and second cytotoxic effector cell(s) comprises or consists of T cell(s).

In some embodiments, the first antigen is cluster of differentiation 3 (CD3) and the second antigen is CD28. In such embodiments, both CD3 and CD28 can be engaged by a trivalent and trispecific antibody construct when located on the same cytotoxic effector cell surface. In other embodiments, both antigens CD3 and CD28 can be engaged by a trivalent and trispecific antibody construct when located on the surfaces of different cytotoxic effector cells.

Hence, in some embodiments, the present disclosure relates to trivalent and trispecific antibody constructs comprising a first binding domain capable of binding CD3, a second binding domain capable of binding CD28, and a third binding domain capable of binding a TAA. Hence, the antibody constructs described herein can also be referred to as “T cell engagers,” “TCEs” or “T cell engager molecules,” describing the construct's ability to bind both, antigens on one or more T cell(s) as well as a TAA on a tumor cell. In some embodiments, the engagement of a trivalent and trispecific antibody construct of two different antigens on one or more T cell(s) and an antigen on a tumor cell, e.g., in a tumor (micro) environment, can be—at least temporarily—simultaneous, thereby establish a TCR-independent immune synapse, and direct T cell-mediated cytotoxic activity to a tumor environment which contains tumor cells expressing the TAA. In various embodiments, and as further described herein, a trivalent and trispecific antibody construct may cause a significantly reduced immune cell (e.g., T cell) activation in the absence of a TAA, e.g., when the immune synapse cannot be fully formed due to an absence of the TAA.

In various embodiments, the first antigen binding domain capable of binding a CD3 on a first cytotoxic effector cell can be a Fab domain or an scFv domain, as described herein. In these embodiments, the second antigen binding domain capable of binding CD28 on a second cytotoxic effector cell can also be a Fab domain or an scFv domain, as described herein.

Hence, in some embodiments, both binding domains capable of binding CD3 and CD28 on the first and second cytotoxic effector cells, respectively, are Fab domains.

In other embodiments, both binding domains capable of binding CD3 and CD28 on the first and second cytotoxic effector cells, respectively, are scFv domains.

In yet other embodiments, the first binding domain capable of binding CD28 on a first cytotoxic effector cell is a Fab domain, and the second binding domain capable of binding CD3 on a second cytotoxic effector cell is an scFv domain.

In another embodiment, the first binding domain capable of binding CD28 on a first cytotoxic effector cell is an scFv domain, and the second binding domain capable of binding CD3 on a second cytotoxic effector cell is a Fab domain.

In various embodiments, the Fab domain that is capable of binding either CD3 or CD28 can comprise a heavy chain constant domain (C_{H1}) comprising or consisting of an amino acid sequence having at least about 90%, 95%, 97%, 99%, or 100% sequence identity to the amino acid sequence set forth in SEQ ID NO: 107. In some embodiments, such C_{H1} domain sequence comprises or consists of an amino acid sequence having at least about 90% sequence identity to the amino acid sequence set forth in SEQ ID NO: 107. In some embodiments, such C_{H1} domain sequence comprises or consists of an amino acid sequence having at least about 95% sequence identity to the amino acid sequence set forth in SEQ ID NO: 107. In some embodiments, such C_{H1} domain sequence comprises or consists of an amino acid sequence having at least about 97% sequence identity to the amino acid sequence set forth in SEQ ID NO: 107. In some embodiments, such C_{H1} domain sequence comprises or consists of an amino acid sequence having at least about 99% sequence identity to the amino acid sequence set forth in SEQ ID NO: 107. In some embodiments, such C_{H1} domain sequence comprises or consists of the amino acid sequence set forth in SEQ ID NO: 107.

### B.2.1 Binding Domains Against CD3

As described herein, in some embodiments, a trivalent and trispecific antibody construct of the present disclosure comprises a binding domain capable of binding CD3.

In various embodiments, such anti-CD3 binding domain (e.g., an scFv domain or a Fab domain) of an antibody construct herein can have an affinity for CD3 (given as an EC₅₀ value for binding CD3) that is not more than about 1 nM, 5 nM, 10 nM, 20 nM, or not more than about 30 nM. Thus, in various embodiments, an anti-CD3 binding domain herein has an EC₅₀ value for binding CD3 that is not more than about 20 nM to about 80 nM, from about 30 nM to about 60 nM, or from about 40 nM to about 50 nM. In some embodiments, an anti-CD3 binding domain herein has an EC₅₀ value for binding CD3 that is not more than about 30 nM, 40 nM, 50 nM, or about 60 nM. In various embodiments, an antibody construct herein comprises an anti-CD3 binding domain that has an EC₅₀ value for binding CD3 from about 20 nM to about 40 nM, e.g., of about 30 nM.

In some embodiments, the anti-CD3 binding domain is capable of binding CD3 with a dissociation constant from about 20 nM to about 200 nM, from about 30 nM to about 150 nM, from about 40 nM to about 100 nM, or from 50 nM to about 80 nM.

In various embodiments, an antibody construct herein comprises a binding domain capable of binding CD3 on a T cell, wherein such binding domain has an EC₅₀ value for binding CD3 from about 20 nM to about 40 nM, e.g., of about 30 nM, and comprises the CDR sequences of the V_{H} sequence as set forth in SEQ ID NOS: 321-323, and the CDR sequences of the V_{L} sequence as set forth in SEQ ID NO: 324-326.

In various embodiments, an antibody construct herein comprises a binding domain capable of binding CD3 on a T cell, wherein such binding domain has an EC₅₀ value for binding CD3 from about 20 nM to about 40 nM, e.g., of about 30 nM, and comprises a V_{H} domain comprising HCDR1 comprising the sequence GVTFNYYG (SEQ ID NO: 321), HCDR2 comprising the sequence ITSSGGRI (SEQ ID NO: 322), and HCDR3 comprising the sequence TLDGRDGWVAY (SEQ ID NO: 323), and a V_{L} domain comprising LCDR1 comprising the sequence TGNIGSNY (SEQ ID NO: 324), LCDR2 comprising the sequence RND (SEQ ID NO: 325), and LCDR3 comprising the sequence QSYSSGFI (SEQ ID NO: 326).

In some embodiments, the anti-CD3 binding domain comprises a V_{H} domain comprising or consisting of an amino acid sequence having at least about 80%, 90%, 95%, 97%, 99%, or 100% sequence identity to the sequence set forth in SEQ ID NO: 102, and a V_{L} domain comprising or consisting of an amino acid sequence having at least about 80%, 90%, 95%, 97%, 99%, or 100% sequence identity to the sequence set forth in SEQ ID NO: 103. In certain embodiments, the anti-CD3 binding domain of an antibody construct herein comprises a V_{H} domain comprising or consisting of the amino acid sequence set forth in SEQ ID NO: 102, and a V_{L} domain comprising or consisting of the amino acid sequence set forth in SEQ ID NO: 103.

In some embodiments, the CDRs of an anti-CD3 paratope used in an antibody construct of the present disclosure comprise one or more amino acid modifications in one or more of the CDR sequences set forth in SEQ ID NOs: 321-326, wherein at least about 80%, 90%, or 95% binding affinity to CD3 is retained compared to the paratope without such amino acid modifications.

In some embodiments, the anti-CD3 binding domain that an antibody construct described herein comprises can comprise or consist of an scFv domain or a Fab domain.

The V_{H} domain of an anti-CD3 scFv or Fab domain of an antibody construct herein can comprise or consist of an amino acid sequence having at least about 80%, 90%, 95%, 97%, 99%, or 100% sequence identity to the amino acid sequence set forth in SEQ ID NO: 102. In some embodiments, the V_{H} domain of such scFv or Fab domain comprises or consists of an amino acid sequence having at least about 90% sequence identity to the amino acid sequence set forth in SEQ ID NO: 102. In some embodiments, the V_{H} domain of such scFv or Fab domain comprises or consists of an amino acid sequence having at least about 95% sequence identity to the amino acid sequence set forth in SEQ ID NO: 102. In some embodiments, the V_{H} domain of such scFv or Fab domain comprises or consists of an amino acid sequence having at least about 97% sequence identity to the amino acid sequence set forth in SEQ ID NO: 102. In some embodiments, the V_{H} domain of such scFv or Fab domain comprises or consists of an amino acid sequence having at least about 99% sequence identity to the amino acid sequence set forth in SEQ ID NO: 102. In yet other embodiments, the V_{H} domain of such scFv or Fab domain comprises or consists of the amino acid sequence set forth in SEQ ID NO: 102.

The V_{L} domain of an anti-CD3 scFv or Fab domain of an antibody construct herein, which, in case of a Fab domain, can be part of a light chain (e.g., L1) that pairs with an anti-CD3 Fab domain sequence of a heavy chain, can comprise or consist of an amino acid sequence having at least about 80%, 90%, 95%, 97%, 99%, or 100% sequence identity to the amino acid sequence set forth in SEQ ID NO: 103. In some embodiments, the V_{L} domain of such scFv or Fab domain comprises or consists of an amino acid sequence having at least about 90% sequence identity to the amino acid sequence set forth in SEQ ID NO: 103. In some embodiments, the V_{L} domain of such scFv or Fab domain comprises or consists of an amino acid sequence having at least about 95% sequence identity to the amino acid sequence set forth in SEQ ID NO: 103. In some embodiments, the V_{L} domain of such scFv or Fab domain comprises or consists of an amino acid sequence having at least about 97% sequence identity to the amino acid sequence set forth in SEQ ID NO: 103. In some embodiments, the V_{L} domain of such scFv or Fab domain comprises or consists of an amino acid sequence having at least about 99% sequence identity to the amino acid sequence set forth in SEQ ID NO: 103. In various embodiments, the V_{L} domain of such scFv or Fab domain comprises or consists of the amino acid sequence set forth in SEQ ID NO: 103.

In embodiments in which the anti-CD3 domain is a Fab domain, which further comprises a C_{H1} domain and a C_{L} domain in its heavy and light chains, respectively, the C_{H1} domain of an anti-CD3 Fab domain of an antibody construct herein can comprise or consist of an amino acid sequence having at least about 80%, 90%, 95%, 97%, 99%, or 100% sequence identity to the amino acid sequence set forth in SEQ ID NO: 107. In some embodiments, the C_{H1} domain of such Fab domain comprises or consists of an amino acid sequence having at least about 90% sequence identity to the amino acid sequence set forth in SEQ ID NO: 107. In some embodiments, the C_{H1} domain of such Fab domain comprises or consists of an amino acid sequence having at least about 95% sequence identity to the amino acid sequence set forth in SEQ ID NO: 107. In some embodiments, the C_{H1} domain of such Fab domain comprises or consists of an amino acid sequence having at least about 97% sequence identity to the amino acid sequence set forth in SEQ ID NO: 107. In some embodiments, the C_{H1} domain of such Fab domain comprises or consists of an amino acid sequence having at least about 99% sequence identity to the amino acid sequence set forth in SEQ ID NO: 107. In some embodiments, the C_{H1} domain of such Fab domain comprises or consists of the amino acid sequence set forth in SEQ ID NO: 107.

Moreover, the C_{L} domain of an anti-CD3 Fab domain of an antibody construct herein can comprise or consist of an amino acid sequence having at least about 80%, 90%, 95%, 97%, 99%, or 100% sequence identity to the amino acid sequence set forth in SEQ ID NO: 121. In some embodiments, the CI domain of such Fab domain comprises or consists of an amino acid sequence having at least about 90% sequence identity to the amino acid sequence set forth in SEQ ID NO: 121. In some embodiments, the C_{L} domain of such Fab domain comprises or consists of an amino acid sequence having at least about 95% sequence identity to the amino acid sequence set forth in SEQ ID NO: 121. In some embodiments, the CI domain of such Fab domain comprises or consists of an amino acid sequence having at least about 97% sequence identity to the amino acid sequence set forth in SEQ ID NO: 121. In some embodiments, the C_{L} domain of such Fab domain comprises or consists of an amino acid sequence having at least about 99% sequence identity to the amino acid sequence set forth in SEQ ID NO: 121. In yet other embodiments, the C_{L} domain of such Fab domain comprises or consists of the amino acid sequence set forth in SEQ ID NO: 121.

In some embodiments, an anti-CD3 binding domain of an antibody construct is an scFv domain. In some embodiments, such anti-CD3 scFv domain has the domain structure, from N- to C-terminus, of: V_{H}-LinkerSF-V_{L} In other embodiments, the anti-CD3 scFv domain has the domain structure, from N- to C-terminus, of: V_{L}-Linker^{scFv}-V_{H}. In any of these embodiments, and as further described herein, the V_{H} domain can comprise or consist of an amino acid sequence having at least about 95%, 97%, 99%, or 100% sequence identity to the amino acid sequence set forth in SEQ ID NO: 102, the V_{L} domain can comprise or consist of an amino acid sequence having at least about 95%, 97%, 99%, or 100% sequence identity to the amino acid sequence set forth in SEQ ID NO: 102, and the Linker^{scFv} can comprise or consist of the amino acid sequence set forth in SEQ ID NO: 104.

Hence, in some embodiments, an anti-CD3 scFv domain can have the domain structure, from N- to C-terminus, of: V_{H}-Linker^{scFv}-V_{L} or V_{L}-Linker^{scFv}-V_{H}, wherein the V_{H} domain comprises or consists of the amino acid sequence set forth in SEQ ID NO: 102, the V_{L} domain comprises or consists of the amino acid sequence set forth in SEQ ID NO: 103, and the Linker^{scFv} comprises or consists of the amino acid sequence set forth in SEQ ID NO: 104. In some of these embodiments, the anti-CD3 scFv domain has the domain structure, from N- to C-terminus, of: V_{H}-Linker^{scFv}-V_{L}. In other embodiments, the anti-CD3 scFv domain has the domain structure, from N- to C-terminus, of: V_{L}-Linker^{scFv}-V_{H}.

In some embodiments, an anti-CD3 binding domain of an antibody construct is a Fab domain. In some embodiments, such anti-CD3 Fab domain comprises a heavy chain, or a portion thereof (e.g., in cases in which the heavy chain further comprises an Fc portion, etc.), and a light chain. The heavy chain of the anti-CD3 Fab domain can comprise or consist of a V_{H} domain coupled to a C_{H1} domain, from N- to C-terminus. In various embodiments, the anti-CD3 Fab domain comprises (i) a V_{H} domain comprising or consisting of an amino acid sequence having at least about 95%, 97%, 99%, or 100% sequence identity to the amino acid sequence set forth in SEQ ID NO: 102, (ii) a V_{L} domain comprising or consisting of an amino acid sequence having at least about 95%, 97%, 99%, or 100% sequence identity to the amino acid sequence set forth in SEQ ID NO: 103, (iii) a C_{H1} domain comprising or consisting of an amino acid sequence having at least about 95%, 97%, 99%, or 100% sequence identity to the amino acid sequence set forth in SEQ ID NO: 107, and (iv) C_{H1} domain comprising or consisting of an amino acid sequence having at least about 95%, 97%, 99%, or 100% sequence identity to the amino acid sequence set forth in SEQ ID NO: 121.

In certain embodiments, the anti-CD3 Fab domain comprises a V_{H} domain comprising or consisting of the amino acid sequence set forth in SEQ ID NO: 102, a C_{H1} domain comprising or consisting of the amino acid sequence set forth in SEQ ID NO: 107, a V_{L} domain comprising or consisting of the amino acid sequence set forth in SEQ ID NO: 103, and a C_{L} domain comprising or consisting of the amino acid sequence set forth in SEQ ID NO: 121.

In various embodiments, and as further described herein, an anti-CD3 Fab domain of an antibody construct can form when a portion of a heavy chain (e.g., H1) that comprises a V_{H} domain and a C_{H1} domain pairs with a light chain (L1) that comprises a V_{L} domain and a C_{L} domain. In some embodiments, the heavy chain, or portion thereof, that comprises the V_{H} and C_{H1} domains and forms an anti-CD3 Fab domain can have the amino acid sequence set forth in SEQ ID NO: 154, and the corresponding light chain that pairs with the anti-CD3 portion of the heavy chain to form the anti-CD3 Fab domain can have the amino acid sequence set forth in SEQ ID NO: 120.

In some embodiments, an anti-CD3 binding domain of an antibody construct herein comprises or consists of an scFv domain. Such scFv domain can comprise or consist of a V_{H} domain and V_{L} domain. In some embodiments, the V_{H} domain is coupled to the V_{L} domain via a linker^{scFv}. In some embodiments, the V_{H} domain comprises or consists of the amino acid sequence set forth in SEQ ID NO: 102, and the V_{L} domain comprises or consists of the amino acid sequence set forth in SEQ ID NO: 103. Hence, in certain embodiments, an anti-CD3 binding domain of an antibody construct herein is an scFv domain comprising or consisting of a V_{H} domain comprising or consisting of the amino acid sequence set forth in SEQ ID NO: 102 coupled to a V_{L} domain comprising or consisting of the amino acid sequence set forth in SEQ ID NO: 103 via a linker^{scFv} comprising or consisting of the amino acid sequence set forth in SEQ ID NO: 104.

As further described herein, in some embodiments, the binding affinity of a trivalent and trispecific antibody construct of the present disclosure for one or more specific targets (e.g., CD3) can—at least in part—be dependent on its format and/or geometry. As an example, the relative orientation and vicinity of an anti-CD3 binding domain in an antibody construct can affect the binding domain's ability to interact with the target epitope it is binding, e.g., through steric hindrance, conformational changes occurring when the construct interacts with one or more of its targets (e.g., conditional degrees of freedom for a binding domain such as its steric flexibility), etc.

In further embodiments, the CD3 binding affinity of an anti-CD3 binding domain of an antibody construct herein can be engineered and altered (e.g., increased/decreased relative to unmodified domains), e.g., by using one or more amino acid modifications. In some embodiments, a trispecific and trivalent antibody construct of the present disclosure can comprise a variant anti-CD3 binding domain that comprises one or more amino acid modifications in its VH and/or VL domain(s) compared to the anti-CD3 binding domains described herein which comprise a VH sequence set forth in, e.g., SEQ ID NO: 102 and a VL sequence set forth in, e.g., SEQ ID NO: 103. Such one or more amino acid modifications can reduce or increase the binding affinity of the variant anti-CD3 binding domain to CD3 when compared to the binding affinity of a corresponding anti-CD3 binding domain that does not comprise such one or more amino acid modifications.

In some embodiments, the one or more amino acid modifications used to alter the binding affinity of an anti-CD3 binding domain can include one or more amino acid substitution(s), one or more amino acid addition(s), and/or one or more amino acid deletion(s). In certain embodiments, the one or more amino acid modifications used to alter the binding affinity of an anti-CD3 binding domain comprise or consist of one or more amino acid substitution(s) relative to an unmodified binding domain sequence (e.g., an anti-CD3 VH or VL sequence).

In some embodiments, the anti-CD3 affinity of an affinity-altered CD3 binding domain can be about ±2-fold, 3-fold, 4-fold, 5-fold, 6-fold, 7-fold, 8-fold, 9-fold, or 10-fold higher or lower than a corresponding parental anti-CD3 binding domain.

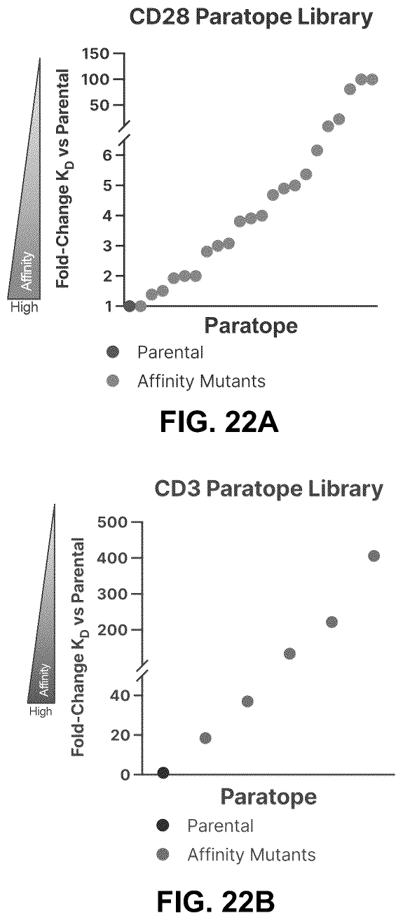

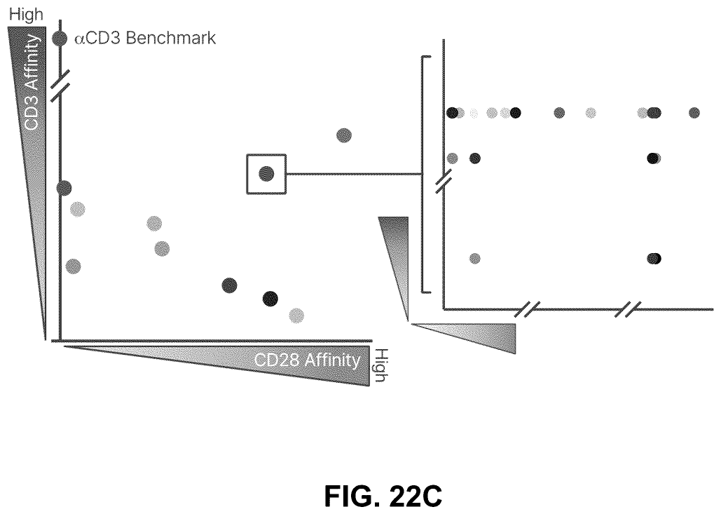

In some embodiments, a trivalent and trispecific antibody construct of this disclosure can have a variety of different anti-CD3 and anti-CD28 (further described below) binding affinities, providing a range of constructs with differing target engagement profiles, particularly when considering that the construct format and geometry may further impact antigen binding (see, e.g., FIGS. 22A-22C).

### B.2.2 Binding Domains Against CD28

As described herein, in various embodiments, a trivalent and trispecific antibody construct of the present disclosure comprises a binding domain capable of binding CD28.

In various embodiments, such anti-CD28 binding domain (e.g., an scFv domain or a Fab domain) of an antibody construct herein can have an affinity for CD28 (given as an EC₅₀, i.e., K_{D}) value for binding CD28) that is from about 10 nM to about 500 nM or from about 20 nM to about 250 nM.

In some embodiments, an antibody construct herein comprises the CDRs of the V_{H} sequence set forth in SEQ ID NOS: 300, 303, and 307, and the CDRs of the V_{L} sequence set forth in SEQ ID NOS: 313, 316, and 320.

In some embodiments, an antibody construct herein comprises a binding domain (e.g., an scFv domain or a Fab domain) capable of binding CD28 on a T cell, wherein such binding domain has an affinity for CD28 that is from about 15 nM to about 35 nM and comprises an anti-CD28 V_{H} sequence comprising a HCDR1 having the sequence SYGVH (SEQ ID NO: 300), a HCDR2 having the sequence VIWPGGGTNFNSALMS (SEQ ID NO: 303), and a HCDR3 having the sequence DRAYGNYLYAMDY (SEQ ID NO: 307), and an anti-CD28 V_{L} sequence comprising a LCDR1 having the sequence RASESVEYYVTSLMQ (SEQ ID NO: 313), a LCDR2 having the sequence AASNVDS (SEQ ID NO: 316), and a LCDR3 having the sequence QQSRKVPFT (SEQ ID NO: 320).

In such embodiments, an antibody construct herein comprises a binding domain (e.g., an scFv domain or a Fab domain) capable of binding CD28 on a T cell, wherein such binding domain comprises a V_{H} domain comprising or consisting of an amino acid sequence having at least about 80%, 90%, 95%, 97%, 99%, or 100% sequence identity to the sequence set forth in SEQ ID NO: 106, and a V_{L} domain comprising or consisting of an amino acid sequence having at least about 80%, 90%, 95%, 97%, 99%, or 100% sequence identity to the sequence set forth in SEQ ID NO: 116. In some of these embodiments, the anti-CD28 binding domain comprises a V_{H} domain comprising or consisting of the sequence set forth in SEQ ID NO: 106, and a V_{L} domain comprising or consisting of the sequence set forth in SEQ ID NO: 116.

In other embodiments, a trivalent and trispecific antibody construct of the present disclosure comprises an anti-CD28 binding domain comprising (i) a V_{H} domain that comprises a sequence having one or more amino acid substitution(s) compared to the sequence set forth in SEQ ID NO: 106, and/or (ii) a V_{L} domain that comprises a sequence having one or more amino acid substitution(s) compared to the sequence set forth in SEQ ID NO: 116, wherein the position of such amino acid substitution is provided according to the IMGT numbering system.

In some embodiments, such one or more amino acid substitutions in either the anti-CD28 V_{H} domain and/or the anti-CD28 V_{L} domain can reduce the binding affinity of the corresponding anti-CD28 binding domain by about 1.5-fold, 1.6-fold, 1.7-fold, 1.8-fold, 2.0-fold, 3.0-fold, 3.3-fold, 3.5-fold, 3.7-fold, 3.9-fold, 5.0-fold, 5.2-fold, 5.5-fold, 6.0-fold, 7.0-fold, 8.0-fold, 8.5-fold, 9.0-fold, 10-fold, 20-fold, or 25-fold, or from about 1.5-fold to about 25-fold, from about 2.0-fold to about 20-fold, from about 3.0-fold to about 20-fold, or from about 5.0-fold to about 10-fold, when compared to the binding affinity of an anti-CD28 binding domain that does not contain a V_{H} and/or V_{L} sequence with such amino acid substitutions (e.g., a binding domain comprising the V_{H} and V_{L} sequence set forth in SEQ ID NOs: 106 and 116, respectively).

Hence, in some embodiments, an anti-CD28 binding domain (e.g., an scFv domain or a Fab domain) of an antibody construct herein can have an affinity for CD28 (given as an EC₅₀, i.e., K_{D} value for binding CD28) that is from about 10 nM to about 500 nM, from about 20 nM to about 600 nM, from about 20 nM to about 250 nM, from about 20 nM to about 150 nM, from about 20 nM to about 100 nM, or from about 20 nM to about 50 nM.

In some embodiments, an anti-CD28 binding domain herein comprises a V_{H} domain comprising the amino acid substitution P1058A relative to the amino acid sequence set forth in SEQ ID NO: 106. In some of these embodiments, the anti-CD28 V_{H} domain comprises the amino acid sequence set forth in SEQ ID NO: 204.

In some embodiments, an anti-CD28 binding domain herein comprises a V_{H} domain comprising the amino acid substitution G1064S relative to the amino acid sequence set forth in SEQ ID NO: 106. In some of these embodiments, the anti-CD28 V_{H} domain comprises the amino acid sequence set forth in SEQ ID NO: 207.

In some embodiments, an anti-CD28 binding domain herein comprises a V_{L} domain comprising the amino acid substitution V1035G relative to the amino acid sequence set forth in SEQ ID NO: 116. In some of these embodiments, the anti-CD28 V_{L} domain comprises the amino acid sequence set forth in SEQ ID NO: 200.

In some embodiments, an anti-CD28 binding domain herein comprises a V_{L} domain comprising the amino acid substitution D1068E relative to the amino acid sequence set forth in SEQ ID NO: 116. In some of these embodiments, the anti-CD28 V_{L} domain comprises the amino acid sequence set forth in SEQ ID NO: 209.

In some embodiments, an anti-CD28 binding domain herein comprises a V_{H} domain comprising the amino acid substitution E1080K relative to the amino acid sequence set forth in SEQ ID NO: 106. In some of these embodiments, the anti-CD28 V_{H} domain comprises the amino acid sequence set forth in SEQ ID NO: 203.

In some embodiments, an anti-CD28 binding domain herein comprises a V_{H} domain comprising the amino acid substitution Y1110S relative to the amino acid sequence set forth in SEQ ID NO: 106. In some of these embodiments, the anti-CD28 V_{H} domain comprises the amino acid sequence set forth in SEQ ID NO: 201.

In some embodiments, an anti-CD28 binding domain herein comprises a V_{H} domain comprising the amino acid substitution N1111aA relative to the amino acid sequence set forth in SEQ ID NO: 106. In some of these embodiments, the anti-CD28 V_{H} domain comprises the amino acid sequence set forth in SEQ ID NO: 208.

In some embodiments, an anti-CD28 binding domain herein comprises a V_{H} domain comprising the amino acid substitution Y1112S relative to the amino acid sequence set forth in SEQ ID NO: 106. In some of these embodiments, the anti-CD28 V_{H} domain comprises the amino acid sequence set forth in SEQ ID NO: 113.

In some embodiments, an anti-CD28 binding domain herein comprises a V_{H} domain comprising the amino acid substitution L1112aN relative to the amino acid sequence set forth in SEQ ID NO: 106. In some of these embodiments, the anti-CD28 V_{H} domain comprises the amino acid sequence set forth in SEQ ID NO: 206.

In some embodiments, an anti-CD28 binding domain herein comprises a V_{H} domain comprising the amino acid substitution Y1113S relative to the amino acid sequence set forth in SEQ ID NO: 106. In some of these embodiments, the anti-CD28 V_{H} domain comprises the amino acid sequence set forth in SEQ ID NO: 210.

In some embodiments, an anti-CD28 binding domain herein comprises a V_{H} domain comprising the amino acid substitution Y1037A relative to the amino acid sequence set forth in SEQ ID NO: 106. In some of these embodiments, the anti-CD28 V_{H} domain comprises the amino acid sequence set forth in SEQ ID NO: 205.

In some embodiments, an anti-CD28 binding domain herein comprises a V_{L} domain comprising the amino acid substitution Y1031A relative to the amino acid sequence set forth in SEQ ID NO: 116. In some of these embodiments, the anti-CD28 V_{L} domain comprises the amino acid sequence set forth in SEQ ID NO: 151.

In some embodiments, an anti-CD28 binding domain herein comprises a V_{L} domain comprising the amino acid substitution N1066A relative to the amino acid sequence set forth in SEQ ID NO: 116. In some of these embodiments, the anti-CD28 V_{L} domain comprises the amino acid sequence set forth in SEQ ID NO: 202.

In some embodiments, an antibody construct comprising an anti-CD28 binding domain comprising one or more of the V_{H} and/or V_{L} domain substitutions can exhibit a reduced non-specific activity in vitro and/or in vivo. In one such embodiment, an antibody construct comprising such mutated (relative to the huTN228 wildtype sequence) anti-CD28 binding domain can induce less non-specific immune cell activity, e.g., having reduced non-specific cytokine production by the immune cells. In certain embodiments, an antibody construct comprising an anti-CD28 binding domain that carries the N1066A substitution in the V_{L} domain relative to huTN228 wildtype can induce a reduced non-specific T cell activity, e.g., cytokine production. In another embodiment, an antibody construct comprising an anti-CD28 binding domain that carries the Y1031A substitution in the V_{L} domain relative to huTN228 wildtype can induce a reduced non-specific T cell activity, e.g., cytokine production. In some embodiments, non-specific T cell activity, e.g., cytokine production such as production of TNFα, IL-2, etc., can be reduced by about 10-fold, 20-fold, 30-fold, 50-fold, 60-fold, 70-fold, or about 100-fold.

In various embodiments, an antibody construct herein comprises a binding domain capable of binding CD28 on a T cell, wherein such binding domain has an EC₅₀ value for binding CD28 from about 20 nM to about 600 nM and comprises a V_{H} domain comprising a HCDR1 having the sequence SX₁GVH (SEQ ID NO: 302), a HCDR2 having the sequence VIWX₂GGX₃TNFNSALMS (SEQ ID NO: 306), and a HCDR3 having the sequence DRAX₄GX₅YX₆X₇AMDY (SEQ ID NO: 312) and a V_{L} sequence comprising a LCDR1 having the sequence RASESVEYYX₈TSLMQ (SEQ ID NO: 315), a LCDR2 having the sequence AASX₉ VX₁₀S (SEQ ID NO: 319), and a LCDR3 having the sequence QQSRKVPFT (SEQ ID NO: 320), and wherein X₁=Y, A; X₂=P, A; X₃=G, S; X₄=S, Y; X₅=N, A; X₆=L, N; X₇=S, Y; X₈=G, V; X₉=N, A; and X₁₀=E, D.

In some embodiments, an anti-CD28 binding domain (e.g., an scFv domain or a Fab domain) of an antibody construct herein comprises a V_{H} domain comprising or consisting of an amino acid sequence having at least about 80%, 90%, 95%, 97%, 99%, or 100% sequence identity to the sequence set forth in SEQ ID NO: 106, and a V_{L} domain comprising or consisting of an amino acid sequence having at least about 80%, 90%, 95%, 97%, 99%, or 100% sequence identity to the sequence set forth in SEQ ID NO: 116. In some of these embodiments, the anti-CD28 binding domain of an antibody construct herein comprises a V_{H} domain comprising or consisting of the amino acid sequence set forth in SEQ ID NO: 106, and a V_{L} domain comprising or consisting of the amino acid sequence set forth in SEQ ID NO: 116.

In some embodiments, the anti-CD28 binding domain (e.g., an scFv domain or a Fab domain) of an antibody construct herein comprises a V_{H} domain comprising or consisting of the amino acid sequence set forth in SEQ ID NO: 204, and a V_{L} domain comprising or consisting of the amino acid sequence set forth in SEQ ID NO: 116.

In some embodiments, the anti-CD28 binding domain (e.g., an scFv domain or a Fab domain) of an antibody construct herein comprises a V_{H} domain comprising or consisting of the amino acid sequence set forth in SEQ ID NO: 207, and a V_{L} domain comprising or consisting of the amino acid sequence set forth in SEQ ID NO: 116.

In some embodiments, the anti-CD28 binding domain (e.g., an scFv domain or a Fab domain) of an antibody construct herein comprises a V_{H} domain comprising or consisting of the amino acid sequence set forth in SEQ ID NO: 106, and a V_{L} domain comprising or consisting of the amino acid sequence set forth in SEQ ID NO: 200.

In some embodiments, the anti-CD28 binding domain (e.g., an scFv domain or a Fab domain) of an antibody construct herein comprises a V_{H} domain comprising or consisting of the amino acid sequence set forth in SEQ ID NO: 106, and a V_{L} domain comprising or consisting of the amino acid sequence set forth in SEQ ID NO: 209.

In some embodiments, the anti-CD28 binding domain (e.g., an scFv domain or a Fab domain) of an antibody construct herein comprises a V_{H} domain comprising or consisting of the amino acid sequence set forth in SEQ ID NO: 203, and a V_{L} domain comprising or consisting of the amino acid sequence set forth in SEQ ID NO: 116.

In some embodiments, the anti-CD28 binding domain (e.g., an scFv domain or a Fab domain) of an antibody construct herein comprises a V_{H} domain comprising or consisting of the amino acid sequence set forth in SEQ ID NO: 201, and a V_{L} domain comprising or consisting of the amino acid sequence set forth in SEQ ID NO: 116.

In some embodiments, the anti-CD28 binding domain (e.g., an scFv domain or a Fab domain) of an antibody construct herein comprises a V_{H} domain comprising or consisting of the amino acid sequence set forth in SEQ ID NO: 208, and a V_{L} domain comprising or consisting of the amino acid sequence set forth in SEQ ID NO: 116.

In some embodiments, the anti-CD28 binding domain (e.g., an scFv domain or a Fab domain) of an antibody construct herein comprises a V_{H} domain comprising or consisting of the amino acid sequence set forth in SEQ ID NO: 113, and a V_{L} domain comprising or consisting of the amino acid sequence set forth in SEQ ID NO: 116.

In some embodiments, the anti-CD28 binding domain (e.g., an scFv domain or a Fab domain) of an antibody construct herein comprises a V_{H} domain comprising or consisting of the amino acid sequence set forth in SEQ ID NO: 206, and a V_{L} domain comprising or consisting of the amino acid sequence set forth in SEQ ID NO: 116.

In some embodiments, the anti-CD28 binding domain (e.g., an scFv domain or a Fab domain) of an antibody construct herein comprises a V_{H} domain comprising or consisting of the amino acid sequence set forth in SEQ ID NO: 210, and a V_{L} domain comprising or consisting of the amino acid sequence set forth in SEQ ID NO: 116.

In some embodiments, the anti-CD28 binding domain (e.g., an scFv domain or a Fab domain) of an antibody construct herein comprises a V_{H} domain comprising or consisting of the amino acid sequence set forth in SEQ ID NO: 205, and a V_{L} domain comprising or consisting of the amino acid sequence set forth in SEQ ID NO: 116.

In some embodiments, the anti-CD28 binding domain (e.g., an scFv domain or a Fab domain) of an antibody construct herein comprises a V_{H} domain comprising or consisting of the amino acid sequence set forth in SEQ ID NO: 106, and a V_{L} domain comprising or consisting of the amino acid sequence set forth in SEQ ID NO: 151.

In some embodiments, the anti-CD28 binding domain (e.g., an scFv domain or a Fab domain) of an antibody construct herein comprises a V_{H} domain comprising or consisting of the amino acid sequence set forth in SEQ ID NO: 106, and a V_{L} domain comprising or consisting of the amino acid sequence set forth in SEQ ID NO: 202.

In embodiments in which the anti-CD28 binding domain of an antibody construct is an scFv domain, such anti-CD28 scFv domain can comprise, from either N- to C-terminus of C- to N-terminus, a V_{H} domain comprising the amino acid sequence set forth in any one of SEQ ID NOs: 106, 113, 201, 203, 204, 205, 206, 207, 208, or 210, coupled to a V_{L} domain comprising the amino acid sequence set forth in any one of SEQ ID NOs: 116, 151, 200, 202, or 209, via a linker^{scFv} which sequence is (GₙS)ₘ wherein n, m can independently be 1, 2, 3, 4 or 5, and as set forth in SEQ ID NO: 348. In some of these instances, the linker^{scFv} comprises or consists of the amino acid sequence set forth in SEQ ID NO: 104. And in some embodiments, the anti-CD28 scFv domain has a domain structure, from N- to C-terminus, of: V_{H}-linker^{scFv}-V_{L}. In other embodiments, the anti-CD28 scFv domain has a domain structure, from N- to C-terminus, of: V_{L}-linker^{scFv}-V_{H}.

In some embodiments, an anti-CD28 scFv domain of an antibody construct herein comprises or consists of an amino acid sequence having at least about 90%, 95%, 97%, 99%, or 100% sequence identity to the amino acid sequence set forth in SEQ ID NO: 119. In some embodiments, an anti-CD28 scFv domain of an antibody construct herein comprises or consists of the amino acid sequence set forth in SEQ ID NO: 119.

In embodiments in which the anti-CD28 binding domain of an antibody construct is a Fab domain, such anti-CD28 Fab domain can comprise a heavy chain comprising a V_{H} sequence having at least about 90%, 95%, 97%, 99%, or 100% sequence identity to the amino acid sequence set forth in SEQ ID NO: 106, coupled to a C_{H1} sequence having at least about 90%, 95%, 97%, 99%, or 100% sequence identity to the amino acid sequence set forth in SEQ ID NO: 107, paired with a light chain comprising a V_{L} sequence having at least about 90%, 95%, 97%, 99%, or 100% sequence identity to the amino acid sequence set forth in SEQ ID NO: 116, coupled to a C_{L} sequence having at least about 90%, 95%, 97%, 99%, or 100% sequence identity to the amino acid sequence set forth in SEQ ID NO: 117.

In some embodiments, an anti-CD28 Fab domain of an antibody construct herein can comprise a heavy chain comprising a V_{H} sequence comprising or consisting of the amino acid sequence set forth in any one of SEQ ID NOs: 106, 113, 201, 203, 204, 205, 206, 207, 208, or 210, coupled to a C_{H1} sequence comprising or consisting of the amino acid sequence set forth in SEQ ID NO: 107, paired with a light chain comprising a V_{L} sequence comprising or consisting of the amino acid sequence set forth in any one of SEQ ID NOs: 116, 151, 200, 202, or 209, coupled to a C_{L} sequence comprising or consisting of the amino acid sequence set forth in SEQ ID NO: 117.

In some embodiments, an anti-CD28 Fab domain of an antibody construct herein can comprise a heavy chain comprising a V_{H} sequence comprising or consisting of the amino acid sequence set forth in SEQ ID NO: 106, coupled to a C_{H1} sequence comprising or consisting of the amino acid sequence set forth in SEQ ID NO: 107, paired with a light chain comprising a V_{L} sequence comprising or consisting of the amino acid sequence set forth in SEQ ID NO: 116, coupled to a C_{L} sequence comprising or consisting of the amino acid sequence set forth in SEQ ID NO: 117.

In certain embodiments, described herein is an antibody construct comprising a binding domain capable of binding CD28, wherein the binding domain comprises a V_{H} sequence comprising a HCDR1 having the sequence SX₁GVH (SEQ ID NO: 302), a HCDR2 having the sequence VIWX₂GGX₃TNFNSALMS (SEQ ID NO: 306), and a HCDR3 having the sequence DRAX₄GX₅YX₆X₇AMDY (SEQ ID NO: 312), and a V_{L} sequence comprising a LCDR1 having the sequence RASESVEYYX₈TSLMQ (SEQ ID NO: 315), a LCDR2 having the sequence AASX₉VX₁₀S (SEQ ID NO: 319), and a LCDR3 having the sequence QQSRKVPFT (SEQ ID NO: 320), and comprises one or more of the following amino acid substitutions at the positions as identified in the CDR sequences: X₁: Y to A, X₂: P to A, X₃: G to S, X₄: S to Y, X₅: N to A, X₆: L to N, X₇: S to Y, X₈: G to V, X₉: N to A, or X₁₀: E to D.

In some embodiments, the antibody construct comprises an anti-CD28 binding domain comprising the substitution X₁: Y to A. In some embodiments, the antibody construct comprises an anti-CD28 binding domain comprising the substitution X₂: P to A. In some embodiments, the antibody construct comprises an anti-CD28 binding domain comprising the substitution X₃: G to S. In some embodiments, the antibody construct comprises an anti-CD28 binding domain comprising the substitution X₄: S to Y. In some embodiments, the antibody construct comprises an anti-CD28 binding domain comprising the substitution X₅: N to A. In some embodiments, the antibody construct comprises an anti-CD28 binding domain comprising the substitution X₆: L to N. In some embodiments, the antibody construct comprises an anti-CD28 binding domain comprising the substitution X₇: S to Y. In some embodiments, the antibody construct comprises an anti-CD28 binding domain comprising the substitution X₈: G to V. In some embodiments, the antibody construct comprises an anti-CD28 binding domain comprising the substitution X₉: N to A. In some embodiments, the antibody construct comprises an anti-CD28 binding domain comprising the substitution X₁₀: E to D.

In some embodiments, such antibody construct can have a binding affinity for CD28 that is reduced by about 1.5-fold to about 25-fold, by about 2.0-fold to about 20-fold, by about 3.0-fold to about 20-fold, or by about 5.0-fold to about 10-fold, when compared to the binding affinity of an antibody construct comprising an anti-CD28 binding domain that does not contain the one or more amino acid substitutions in one or more of the CDR sequences.

In some embodiments, such antibody construct can comprise an anti-CD28 binding domain that comprises the CDRs of a V_{H} domain as set forth in SEQ ID NOS: 300, 303, and 307, and the CDRs of the V_{L} sequence set forth in SEQ ID NOS: 313, 316, and 320.

In some embodiments, such antibody construct can comprise an anti-CD28 binding domain that comprises an anti-CD28 V_{H} sequence comprising a HCDR1 having the sequence SYGVH (SEQ ID NO: 300), a HCDR2 having the sequence VIWPGGGTNFNSALMS (SEQ ID NO: 303), and a HCDR3 having the sequence DRAYGNYLYAMDY (SEQ ID NO: 307), and an anti-CD28 V_{L} sequence comprising a LCDR1 having the sequence RASESVEYYVTSLMQ (SEQ ID NO: 313), a LCDR2 having the sequence AASNVDS (SEQ ID NO: 316), and a LCDR3 having the sequence QQSRKVPFT (SEQ ID NO: 320).

### C. Binding Domains Against a Tumor-Associated Antigen (TAA)

As described herein, in various embodiments, a trivalent and trispecific antibody construct of the present disclosure comprises a binding domain capable of binding a TAA. In various embodiments, and as further described herein, an antibody construct herein can be trivalent and trispecific and comprises a first binding domain capable of binding a first antigen on a first cytotoxic effector cell, a second binding domain capable of binding a second antigen on a second cytotoxic effector cell, and a third binding domain, wherein such third binding domain is capable of binding the TAA. The TAA can be any antigenic substance expressed on a tumor cell surface.

As described herein, in various embodiments, the anti-TAA binding domain of an antibody construct can be capable of binding mesothelin (MSLN).

In other embodiments, the anti-TAA binding domain of an antibody construct can be capable of binding Claudin 18.2 (Cldn18.2).

Generally, the anti-TAA binding domain of an antibody construct herein can be an scFv domain or a Fab domain.

In various embodiments, the anti-TAA binding domain of a trivalent and trispecific antibody construct herein is an scFv domain.

In various embodiments, the anti-TAA binding domain (e.g., scFv domain) of an antibody construct herein is capable of binding Cldn18.2. In some embodiments, such anti-Cldn18.2 binding domain comprises a HCDR1 having the sequence SNPMI (SEQ ID NO: 333), a HCDR2 having the sequence IIDTDGSTYYADWAKG (SEQ ID NO: 334), and a HCDR3 having the sequence RLHGSSNGYYDDL (SEQ ID NO: 335) and a V_{L} sequence comprising a LCDR1 having the sequence QASQSIYSYLS (SEQ ID NO: 336), a LCDR2 having the sequence KASTLAS (SEQ ID NO: 337), and a LCDR3 having the sequence QQGYTVTNVDKNT (SEQ ID NO: 338).

In some embodiments, an anti-Cldn 18.2 binding domain (e.g., scFv domain) of an antibody construct herein comprises a V_{H} sequence comprising or consisting of an amino acid sequence having at least about 90%, 95%, 97%, 99%, or 100% sequence identity to the amino acid sequence set forth in SEQ ID NO: 127, and a V_{L} sequence comprising or consisting of an amino acid sequence having at least about 90%, 95%, 97%, 99%, or 100% sequence identity to the amino acid sequence set forth in SEQ ID NO: 128.

In various embodiments, the anti-Cldn18.2 binding domain of an antibody construct herein is an scFv domain and comprises, either from N- to C-terminus or from C- to N-terminus, a V_{H} sequence comprising or consisting of the amino acid sequence set forth in SEQ ID NO: 127, coupled to a V_{L} sequence comprising or consisting of the amino acid sequence set forth in SEQ ID NO: 128, via a linker_{scFv} having the amino acid sequence set forth in SEQ ID NO: 104.

In various embodiments, the anti-TAA binding domain (e.g., scFv domain) of an antibody construct herein is capable of binding MSLN. In some embodiments, such anti-MSLN binding domain comprises a V_{H} sequence comprising a HCDR1 having the sequence GYTMN (SEQ ID NO: 327), a HCDR2 having the sequence LITPYSGASSYAQKFQG (SEQ ID NO: 328), and a HCDR3 having the sequence GGYDGRGFDY (SEQ ID NO: 329) and a V_{L} sequence comprising a LCDR1 having the sequence SASSSVSYMH (SEQ ID NO: 330), a LCDR2 having the sequence DTSKLAS (SEQ ID NO: 331), and a LCDR3 having the sequence QQWSGHPLT (SEQ ID NO: 332).

In some embodiments, the anti-MSLN binding domain of an antibody construct herein is an scFv domain and comprises or consists of an amino acid sequence having at least about 90%, 95%, 97%, 99%, or 100% sequence identity to the amino acid sequence set forth in SEQ ID NO: 2. In some embodiments, the anti-MSLN scFv domain of an antibody construct herein comprises or consists of the amino acid sequence set forth in SEQ ID NO: 2.

In certain embodiments, described herein is an antibody construct comprising an anti-Cldn18.2 binding domain comprising the CDRs of a V_{H} sequence as set forth in SEQ ID NOS: 333-335, and the CDRs of a V_{L} sequence as set forth in SEQ ID NOS: 336-338.

In some embodiments, such antibody construct can comprise an anti-Cldn18.2 binding domain comprising a V_{H} sequence comprising a HCDR1 having the sequence SNPMI (SEQ ID NO: 333), a HCDR2 having the sequence IIDTDGSTYYADWAKG (SEQ ID NO: 334), and a HCDR3 having the sequence RLHGSSNGYYDDL (SEQ ID NO: 335), and a V_{L} sequence comprising a LCDR1 having the sequence QASQSIYSYLS (SEQ ID NO: 336), a LCDR2 having the sequence KASTLAS (SEQ ID NO: 337), and a LCDR3 having the sequence QQGYTVTNVDKNT (SEQ ID NO: 338).

In some embodiments, such antibody construct can comprise an anti-Cldn18.2 binding domain comprising or consisting of an amino acid sequence having at least about 90%, 95%, 97%, 99%, or 100% sequence identity to the amino acid sequence set forth in SEQ ID NO: 127, and a V_{L} sequence comprising or consisting of an amino acid sequence having at least about 90%, 95%, 97%, 99%, or 100% sequence identity to the amino acid sequence set forth in SEQ ID NO: 128. In certain embodiments, such anti-Cldn18.2 binding domain comprises a V_{H} sequence comprising or consisting of the amino acid sequence set forth in SEQ ID NO: 127, and a V_{L} sequence comprising or consisting of the amino acid sequence set forth in SEQ ID NO: 128.

### D. Fc Domains

As described herein, a trivalent and trispecific antibody construct of this disclosure can comprise an Fc domain (or Fc region) comprising a first Fc polypeptide and a second Fc polypeptide. In various embodiments, the Fc domain is a heterodimeric Fc domain comprising a first Fc polypeptide and a second Fc polypeptide, wherein the first Fc polypeptide and the second Fc polypeptide share about 90%, 95%, 97%, or about 99% amino acid sequence identity, e.g., each comprising one or more asymmetric amino acid substitutions that can promote preferential pairing for the Fc polypeptides to form the heterodimeric Fc domain compared to formation of a respective homodimeric Fc domain.

The term “Fc domain,” as used herein, includes native (or wildtype) sequence Fc domains as well as variant Fc domains comprising one or more amino acid modifications relative to a corresponding native or wildtype Fc domain. Unless otherwise specified herein, numbering of amino acid residues in the Fc domain or constant region is according to the EU numbering system, also called the EU index, as described, e.g., in Kabat et al., Sequences of Proteins of Immunological Interest, 5th Ed. Public Health Service, National Institutes of Health, Bethesda, MD (1991). An “Fc polypeptide” of a dimeric (e.g., heterodimeric) Fc domain refers to one of the two polypeptide chains (e.g., a first and second Fc polypeptide) forming the dimeric (e.g., heterodimeric) Fc domain. In some embodiments, Fc polypeptides can comprise a C-terminal constant region of an Ig heavy chain that is capable of stable self-association. In various embodiments, and as further described herein, an Fc polypeptide (e.g., a first or a second Fc polypeptide) comprises at least one of a CH2 domain and/or a CH3 domain. In certain embodiments, an Fc polypeptide of an antibody construct described herein comprises a CH2 domain and a CH3 domain.

As disclosed herein, a trivalent and trispecific antibody construct can comprise an Fc domain, wherein such Fc domain can be a heterodimeric Fc domain. The Fc domain, e.g., heterodimeric Fc domain, of an antibody construct, unless otherwise specified, comprises a first Fc polypeptide and a second Fc polypeptide. Generally, each Fc polypeptide of a (e.g., heterodimeric) Fc domain can comprise a CH2 domain, a CH3 domain, or, as described in various embodiments herein, both a CH2 domain and a CH3 domain.

In certain embodiments, an antibody construct comprises an Fc domain based on a human IgG Fc domain. In some embodiments, an antibody construct comprises an Fc domain based on a human IgG1 Fc domain. In various embodiments, an antibody construct comprises a heterodimeric IgG Fc domain comprising two different Fc polypeptides, e.g., a first Fc polypeptide and a second polypeptide, wherein the first and second Fc polypeptides have different amino acid sequences, e.g., amino acid sequences that have about 90%, 95%, 97%, or 99% sequence identity when compared and aligned to one another, e.g., as further described herein. In some embodiments, the differences in the amino acid sequences of a first and second Fc polypeptide can be due to asymmetric amino acid substitutions that can be introduced into each Fc polypeptide chain to promote preferential paring of the heavy chains to form the heterodimeric Fc domain, compared to a corresponding homodimeric Fc domain.

In various embodiments, a trivalent and trispecific antibody construct herein comprises an Fc domain that is a modified IgG Fc domain, and in which at least the CH3 domain of at least one Fc polypeptide comprises one or more amino acid modifications compared to a respective wildtype CH3 domain. In some embodiments, an antibody construct herein comprises an Fc domain that is a modified IgG Fc domain in which at least the CH2 domain of at least one Fc polypeptide comprises one or more amino acid modifications compared to a respective wildtype CH2 domain. In some embodiments, an antibody construct comprises an Fc domain that is a modified IgG Fc domain in which both the CH3 domain and the CH2 domain of at least one Fc polypeptide comprises one or more amino acid modifications compared to respective wildtype CH3 and CH2 domains. In various embodiments, both Fc polypeptides of a heterodimeric Fc domain can comprise one or more amino acid modifications in their CH3 domains. In some embodiments, both Fc polypeptides of a heterodimeric Fc domain can comprise one or more amino acid modifications in their CH2 domains. In yet other embodiments, both Fc polypeptides of a heterodimeric Fc domain can comprise one or more amino acid modifications in their CH2 domains and CH3 domains.

### Modified Fc Domains

In some embodiments, the present disclosure relates to trivalent and trispecific antibody constructs that can comprise a heterodimeric Ig Fc domain comprising a modified heterodimeric CH3 domain, wherein the modified heterodimeric CH3 domain comprises one or more asymmetric amino acid modifications, i.e., one or both the first and the second Fc polypeptide each comprise one or more amino acid modifications in their CH3 domain sequences compared to respective wildtype sequences. As used herein, the term “asymmetric amino acid modification” generally refers to a modification in which an amino acid at a specific position on the first Fc polypeptide is different to the amino acid at the corresponding position on the second Fc polypeptide. These asymmetric amino acid modifications can comprise modifications of only one of the two amino acids at the corresponding position on each Fc polypeptide, or they can comprise modifications of both amino acids at the corresponding positions on each of the first and second Fc polypeptides. In various embodiments, an “asymmetric amino acid modification” is an asymmetric amino acid substitution.

In some embodiments, an antibody construct herein comprises a heterodimeric Fc domain comprising a modified CH3 domain (i.e., a heterodimeric CH3 domain consisting of the two CH3 domains of the first and second Fc polypeptides), wherein the modified CH3 domain comprises one or more asymmetric amino acid modifications that promote formation of the heterodimeric Fc domain (e.g., pairing of a first Fc polypeptide with a second Fc polypeptide) over formation of a corresponding homodimeric Fc domain (e.g., pairing of a first Fc polypeptide with another first Fc polypeptide). Amino acid modifications that can be made to the CH3 domain of an Fc domain in order to promote formation of a heterodimeric Fc domain are known in the art and include, for example, those described in International Publication No. WO 96/027011 (“knobs into holes”), Gunasekaran et al., 2010, J Biol Chem, 285, 19637-46 (“electrostatic steering”), Davis et al., 2010, Prot Eng Des Sel, 23 (4): 195-202 (strand exchange engineered domain (SEED) technology) and Labrijn et al., 2013, Proc Natl Acad Sci USA, 110 (13): 5145-50 (Fab-arm exchange). Other examples include approaches combining positive and negative design strategies to produce stable asymmetrically modified Fc regions as described in International Publication Nos. WO 2012/058768 and WO 2013/063702.

In certain embodiments, an antibody construct described herein comprises a heterodimeric Fc domain comprising a modified heterodimeric CH3 domain in which at least one, or both of the Fc polypeptide chains comprise one or more amino acid modifications, as described in International Publication No. WO 2012/058768 or International Patent Publication No. WO 2013/063702.

In some embodiments, an antibody construct described herein comprises a heterodimeric human IgG1 Fc domain having a modified CH3 domain. TABLE 2 herein provides the amino acid sequence of a human IgG1 Fc domain sequence (e.g., a sequence that a first and/or a second Fc polypeptide can be derived from), corresponding to amino acids 231 to 447 of a full-length human IgG1 heavy chain (e.g., one that comprises V_{H}, C_{H1}, Hinge, CH2 and CH3 domains), and identified by SEQ ID NO: 1. The CH2 domain is typically defined as comprising amino acids 231-340 of the full-length human IgG1 heavy chain and the CH3 domain is typically defined as comprising amino acids 341-447 of the full-length human IgG1 heavy chain.

As described herein, an antibody construct can comprise a heterodimeric Fc domain having a modified CH3 domain comprising one or more asymmetric amino acid modifications that promote formation of the heterodimeric Fc domain over formation of a homodimeric Fc domain, and in which the modified CH3 domain comprises a first Fc polypeptide including amino acid modifications at positions F405 and Y407, relative to SEQ ID NO: 1, and a second Fc polypeptide including amino acid modifications at positions T366 and T394, relative to SEQ ID NO: 1. In various embodiments, the one or more amino acid modifications comprise one or more amino acid substitutions. Hence, in some embodiments, the amino acid modification at position F405 of the first Fc polypeptide of the modified CH3 domain is F405A, F405I, F405M, F405S, F405T or F405V. In some embodiments, the amino acid modification at position Y407 of the first Fc polypeptide of the modified CH3 domain is Y407I or Y407V. In some embodiments, the amino acid modification at position T366 of the second Fc polypeptide of the modified CH3 domain is T366I, T366L or T366M. In some embodiments, the amino acid modification at position T394 of the second Fc polypeptide of the modified CH3 domain is T394W. In some embodiments, the modified CH3 domain of a first Fc polypeptide further includes an amino acid modification at position L351, relative to SEQ ID NO: 1. In some embodiments, the amino acid modification at position L351 in the first Fc polypeptide of the modified CH3 domain is L351Y. In some embodiments, the second Fc polypeptide of the modified CH3 domain further includes an amino acid modification at position K392, relative to SEQ ID NO: 1. In some embodiments, the amino acid modification at position K392 in the second Fc polypeptide of the modified CH3 domain is K392F, K392L or K392M. In some embodiments, one or both of the first and second Fc polypeptides of the modified CH3 domain further comprises the amino acid modification T350V.

In certain embodiments, an antibody construct herein comprises a heterodimeric Fc domain having a modified CH3 domain comprising one or more asymmetric amino acid modifications that promote formation of the heterodimeric Fc domain over formation of a homodimeric Fc domain, and in which the modified CH3 domain comprises a first Fc polypeptide including the amino acid modification F405A, F405I, F405M, F405S, F405T or F405V together with the amino acid modification Y407I or Y407V, and relative to SEQ ID NO: 1, and a second Fc polypeptide including the amino acid modification T366I, T366L or T366M, together with the amino acid modification T394W, and relative to SEQ ID NO: 1. In some embodiments, the first Fc polypeptide of the modified CH3 domain further includes the amino acid modification L351Y. In some embodiments, the second Fc polypeptide of the modified CH3 domain further includes the amino acid modification K392F, K392L or K392M. In some embodiments, one or both of the first and second Fc polypeptides having a modified CH3 domain further comprises the amino acid modification T350V.

TABLE 2
Exemplary Human IgG1 Fc Domain Sequences and Variants Thereof

| | |
|---|---|
| Human IgG1 Fc | APELLGGPSVFLFPPKPKDTLMISRTPEVTCVVVDVSHEDPE |
| Domain sequence | VKFNWYVDGVEVHNAKTKPREEQYNSTYRVVSVLTVLHQD |
| of amino acid | WLNGKEYKCKVSNKALPAPIEKTISKAKGQPREPQVYTLPPS |
| residues 231-447 | RDELTKNQVSLTCLVKGFYPSDIAVEWESNGQPENNYKTTP |
| (EU-numbering) | PVLDSDGSFFLYSKLTVDKSRWQQGNVFSCSVMHEALHNH |
| | YTQKSLSLSPGK |
| | (SEQ ID NO: 1) |

| Variant # | Polypeptide Chain* | Mutations |
|---|---|---|
| 1 | A | L351Y_F405A_Y407V |
| | B | T366L_K392M_T394W |
| 2 | A | L351Y_F405A_Y407V |
| | B | T366L_K392L_T394W |
| 3 | A | T350V_L351Y_F405A_Y407V |
| | B | T350V_T366L_K392L_T394W |
| 4 | A | T350V_L351Y_F405A_Y407V |
| | B | T350V_T366L_K392M_T394W |
| 5 | A | T350V_L351Y_S400E_F405A_Y407V |
| | B | T350V_T366L_N390R_K392M_T394W |

*“A” corresponds to a first Fc polypeptide chain and ″B″ to corresponds to a second Fc polypeptide chain.

In certain embodiments, an antibody construct herein comprises a heterodimeric Fc domain comprising a modified CH3 domain having a first Fc polypeptide that comprises amino acid modifications at positions F405 and Y407, and optionally further comprises an amino acid modification at position L351, and a second Fc polypeptide that comprises amino acid modifications at positions T366 and T394, and optionally further comprises an amino acid modification at position K392, as described above, and the first Fc polypeptide further comprises an amino acid modification at one or both of positions S400 or Q347 and/or the second Fc polypeptide further comprises an amino acid modification at one or both of positions K360 or N390, wherein the amino acid modification at position S400 is S400E, S400D, S400R or S400K; the amino acid modification at position Q347 is Q347R, Q347E or Q347K; the amino acid modification at position K360 is K360D or K360E, and the amino acid modification at position N390 is N390R, N390K or N390D, relative to SEQ ID NO: 1.

In some embodiments, an antibody construct comprises a heterodimeric Fc domain comprising a modified CH3 domain comprising the modifications of any one of Variant 1, Variant 2, Variant 3, Variant 4 or Variant 5, as shown in TABLE 2.

In various embodiments, an antibody construct of the present disclosure can comprise a heterodimeric Fc domain comprising a first Fc polypeptide (A) and a second Fc polypeptide (B), wherein the first Fc polypeptide (A) and the second Fc polypeptide (B) comprise the amino acid substitutions in their CH3 domains according to variant #1 as shown in TABLE 2. In other embodiments, an antibody construct of the present disclosure can comprise a heterodimeric Fc domain comprising a first Fc polypeptide (A) and a second Fc polypeptide (B), wherein the first Fc polypeptide (A) and the second Fc polypeptide (B) comprise the amino acid substitutions in their CH3 domains according to variant #2 as shown in TABLE 2. In some embodiments, an antibody construct of the present disclosure can comprise a heterodimeric Fc domain comprising a first Fc polypeptide (A) and a second Fc polypeptide (B), wherein the first Fc polypeptide (A) and the second Fc polypeptide (B) comprise the amino acid substitutions in their CH3 domains according to variant #3 as shown in TABLE 2. In some embodiments, an antibody construct of the present disclosure can comprise a heterodimeric Fc domain comprising a first Fc polypeptide (A) and a second Fc polypeptide (B), wherein the first Fc polypeptide (A) and the second Fc polypeptide (B) comprise the amino acid substitutions in their CH3 domains according to variant #4 as shown in TABLE 2. In yet other embodiments, an antibody construct of the present disclosure can comprise a heterodimeric Fc domain comprising a first Fc polypeptide (A) and a second Fc polypeptide (B), wherein the first Fc polypeptide (A) and the second Fc polypeptide (B) comprise the amino acid substitutions in their CH3 domains according to variant #5 as shown in TABLE 2.

In certain embodiments, the CH3 domain of a first Fc polypeptide of an antibody construct herein has an amino acid sequence that is at least about 80%, about 85%, about 90%, about 95%, about 97%, or at least about 99% identical to the amino acid sequence set forth in SEQ ID NO: 110. In certain embodiments, the CH3 domain of a second Fc polypeptide of an antibody construct herein has an amino acid sequence that is at least about 80%, about 85%, about 90%, about 95%, about 97%, or at least about 99% identical to the amino acid sequence set forth SEQ ID NO: 114. In some embodiments, the CH3 domain of a first Fc polypeptide of an antibody construct herein has the amino acid sequence set forth in SEQ ID NO: 110 and a second Fc polypeptide of the antibody construct herein has the amino acid sequence set forth SEQ ID NO: 114.

In certain embodiments, an antibody construct herein comprises a heterodimeric Fc domain based on an IgG Fc domain having a modified CH2 domain (i.e., a heterodimeric CH2 domain consisting of the two CH2 domain sequences of the respective first and second Fc polypeptides). In some embodiments, an antibody construct comprises a heterodimeric Fc domain based on an IgG Fc domain having a modified CH2 domain, wherein the modification(s) of the CH2 domain result(s) in altered (e.g., reduced or abated) binding to one or more Fc receptors (FcRs) such as receptors of the FcγRI, FcγRII and FcγRIII subclasses.

Several amino acid modifications to the CH2 domain of the first and/or second Fc polypeptide(s) of an Fc domain that selectively alter the affinity of such Fc domain for different Fcγ receptors are known in the art. Amino acid modifications that result in increased binding and amino acid modifications that result in decreased binding can both be useful in certain indications. For example, increasing binding affinity of an Fc for FcγRIIIa (an activating receptor) can result in increased antibody dependent cell-mediated cytotoxicity (ADCC), which in turn can result in increased lysis of the target cell. Decreased binding to FcγRIIb (an inhibitory receptor) likewise can be beneficial in some circumstances. In certain indications, a decrease in, or elimination of, ADCC and complement-mediated cytotoxicity (CDC) can be desirable. In such embodiments, modified CH2 domains comprising amino acid modifications that result in increased binding to FcγRIIb or amino acid modifications that can decrease or eliminate binding of the Fc region to all of the Fcγ receptors (“knock-out” variants) can be useful.

Non-limiting examples of amino acid modifications to the CH2 domain that alter binding of the Fc domain by Fcγ receptors include, but are not limited to, the following: S298A/E333A/K334A and S298A/E333A/K334A/K326A (increased affinity for FcγRIIIa) (Lu, et al., 2011, J Immunol Methods, 365 (1-2): 132-41); F243L/R292P/Y300L/V305I/P396L (increased affinity for FcγRIIIa) (Stavenhagen, et al., 2007, Cancer Res, 67 (18): 8882-90); F243L/R292P/Y300L/L235V/P396L (increased affinity for FcγRIIIa) (Nordstrom J L, et al., 2011, Breast Cancer Res, 13 (6): R123); F243L (increased affinity for FcγRIIIa) (Stewart, et al., 2011, Protein Eng Des Sel., 24 (9): 671-8); S298A/E333A/K334A (increased affinity for FcγRIIIa) (Shields, et al., 2001, J Biol Chem, 276 (9): 6591-604); S239D/1332E/A330L and S239D/1332E (increased affinity for FcγRIIIa) (Lazar, et al., 2006, Proc Natl Acad Sci USA, 103 (11): 4005-10), and S239D/S267E and S267E/L328F (increased affinity for FcγRIIb) (Chu, et al., 2008, Mol Immunol, 45 (15): 3926-33). Additional modifications that affect Fc domain binding to Fcγ receptors are described in Therapeutic Antibody Engineering (Strohl & Strohl, Woodhead Publishing series in Biomedicine No 11, ISBN 1 907568 37 9, October 2012, page 283).

In various embodiments, an antibody construct of the present disclosure comprises a heterodimeric Fc domain based on an IgG Fc domain having a modified CH2 domain, in which one or both of the CH2 sequences (i.e., of the first/second Fc polypeptide) of the modified dimeric CH2 domain comprise one or more amino acid modifications that can result in decreased or eliminated binding of the Fc domain to one or more, or all of the Fcγ receptors (i.e., a “knock-out” or “KO” variant).

Various publications describe strategies that have been used to engineer antibodies to produce “knock-out” Fc variants (see, for example, Strohl, 2009, Curr Opin Biotech 20:685-691, and Strohl & Strohl, “Antibody Fc engineering for optimal antibody performance” In Therapeutic Antibody Engineering, Cambridge: Woodhead Publishing, 2012, pp 225-249). These strategies include reduction of effector function through modification of glycosylation, use of IgG2/IgG4 scaffolds, or the introduction of mutations in the hinge or CH2 domain of the Fc (see also, U.S. Patent Publication No. 2011/0212087, International Publication No. WO 2006/105338, U.S. Patent Publication No. 2012/0225058, U.S. Patent Publication No. 2012/0251531, and Strop et al., 2012, J. Mol. Biol., 420:204-219).

In some embodiments, an antibody construct's Fc domain can comprise one or more of known amino acid modifications to reduce FcγR and/or complement binding of the Fc domain. In some embodiments, such modifications can include those identified in TABLE 3.

TABLE 3
Modifications to Reduce Fcγ Receptor or Complement Binding

| Company | Mutations |
|---|---|
| GSK | N297A |
| Ortho Biotech | L234A/L235A |
| Protein Design labs | IgG2 V234A/G237A |
| Wellcome Labs | IgG4 L235A/G237A/E318A |
| GSK | IgG4 S228P/L236E |
| Merck | IgG2 H268Q/V309L/A330S/A331S |
| Bristol-Myers | C220S/C226S/C229S/P238S |
| Seattle Genetics | C226S/C229S/E3233P/L235V/L235A |
| Medimmune | L234F/L235E/P331S |

Additional examples herein include Fc domains engineered to include the amino acid modifications L235A/L236A/D265S, e.g., based on the sequence set forth in SEQ ID NO: 1. In addition, asymmetric amino acid modifications in the CH2 domain that decrease binding of the Fc to all Fcγ receptors are described in International Publication No. WO 2014/190441.

In certain embodiments, the CH2 domain of a first and a second Fc polypeptide herein comprises or consists of an amino acid sequence having at least about 80%, about 85%, about 90%, about 95%, about 97%, or at least about 99% sequence identity to the sequence set forth in SEQ ID NO: 109. In some embodiments, the CH2 domain of a first and/or a second Fc polypeptide herein comprises or consists of the sequence set forth in SEQ ID NO: 109.

In certain embodiments, an antibody construct herein comprises a heterodimeric Fc domain in which native glycosylation has been modified. As is known in the art, glycosylation of an Fc can be modified to increase or decrease effector function. For example, mutation of the conserved asparagine residue at position 297 to alanine, glutamine, lysine, or histidine (i.e., N297A, Q, K or H) results in an aglycoslated Fc that lacks all effector function (Bolt et al., 1993, Eur. J. Immunol., 23:403-411; Tao & Morrison, 1989, J. Immunol., 143:2595-2601). Conversely, removal of fucose from heavy chain N297-linked oligosaccharides has been shown to enhance ADCC, based on improved binding to FcγRIIIa (see, for example, Shields et al., 2002, J Biol Chem., 277:26733-26740, and Niwa et al., 2005, J. Immunol. Methods, 306:151-160). Such low fucose antibody constructs can be produced, for example in knockout Chinese hamster ovary (CHO) cells lacking fucosyltransferase (FUT8) (Yamane-Ohnuki et al., 2004, Biotechnol. Bioeng., 87:614-622), in the variant CHO cell line, Lec 13, that has a reduced ability to attach fucose to N297-linked carbohydrates (International Publication No. WO 03/035835), or in other cells that generate afucosylated antibodies (see, for example, Li et al., 2006, Nat Biotechnol, 24:210-215; Shields et al., 2002, ibid, and Shinkawa et al., 2003, J. Biol. Chem., 278:3466-3473). In addition, International Publication No. WO 2009/135181 describes the addition of fucose analogs to culture medium during antibody production to inhibit incorporation of fucose into the carbohydrate on the antibody.

### E. Linkers

In various embodiments of this disclosure, a trivalent and trispecific antibody construct described herein can comprise one or more linkers. In some embodiments, such one or more linkers are peptide (also referred to herein as “peptitic”) linkers comprising or consisting of an amino acid sequence of about 1, 2, 3, 5, 10, 15, 20, 25, 30, 40, or about 50 consecutive amino acid residues in length. The one or more peptide linker of an antibody construct can comprise or consist of an amino acid sequence from 1 to about 50, from 2 to about 40, from 3 to about 30, or from 5 to about 25 consecutive amino acid residues in length.

Such peptide linkers can couple, or link, two or more polypeptide sequences and/or domains of an antibody construct to each other. In various embodiments, a linker herein can couple a first polypeptide chain, e.g., a heavy chain constant domain (C_{H1}), to an Fc polypeptide. Thus, a linker can be used to couple one domain of an antibody construct to another domain, from N- to C-terminus, e.g., a Fab domain to an Fc domain, e.g., a linker^{Fab-Fc}, an scFv domain to a Fab domain, e.g., a linker^{scFv-Fab}, a V_{H} domain to a V_{L} domain, e.g., linker^{scfv}, and so forth. In embodiments in which both scFv domains of an antibody construct contain a linker^{scFv} with identical amino acid sequence, such antibody construct can be described as comprising a linker^{scFv}, instead of specifying that it contains a linker^{scFv1} and a linker^{scFv2}. In embodiments, however, in which the linker^{scFv} of both scFv domains have different amino acid sequences, such antibody construct can be described as comprising a linker^{scFv1} and a linker^{scFv2}.

In embodiments in which a linker couples, e.g., a heavy chain variable domain (V_{H}) to, e.g., a light chain variable domain (V_{L}), the linker can be of sufficient length to allow both domains to elicit their biological function. In addition to providing a spacing function, a linker herein (e.g., a peptide linker) can provide flexibility or rigidity suitable for properly orienting the one or more domains of an antibody construct, both within the antibody construct itself and between the antibody construct and its target(s).

Further, a linker herein (e.g., a peptide linker) can support (i) expression of a full-length fusion protein, e.g., a full-length polypeptide chain H1, L1, H2, etc. of an antibody construct, and (ii) provide increased stability of the purified protein both in vitro and in vivo, e.g., following administration to a subject in need thereof, such as a human. The one or more linkers used in antibody constructs herein are generally non-immunogenic or poorly immunogenic in mammalian subjects that a construct may be administered to. In certain embodiments, one or more of the linker used in an antibody construct herein can comprise part or all of a human Ig hinge region, a stalk region of C-type lectins, a family of type II membrane proteins, or combinations thereof. In certain embodiments, one or more of the linker used in an antibody construct herein can comprise part or all of a human Ig hinge region, such as an IgG1 hinge region, such as a linker^{Fab-Fc} or a linker^{scFv-Fc}.

In certain embodiments, each linker used in an antibody construct herein can comprise or consist of an amino acid sequence having a length of 2 to about 50 amino acids. In some embodiments, each linker used in an antibody construct herein can comprise or consist of an amino acid sequence having a length from about 3 to about 40 amino acids, from about 10 to about 50 amino acids, from about 2 to about 40 amino acids, from about 5 to about 30 amino acids, from about 5 to about 25 amino acids, from about 4 to about 30 amino acids, from about 10 to about 30 amino acids, or from about 15 to about 25 amino acids. In some embodiments, the one or more linkers of an antibody construct can each comprise an amino acid sequence comprising or consisting of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive amino acids.

In certain embodiments, a linker (e.g., a linker^{scFv}, linker^{scFv-Fab}, linker^{Fab-Fc}, etc.) of an antibody construct herein comprises of consists of the amino acid sequence (EAAAK)ₙ wherein n is an integer from 1 to 5 (SEQ ID NO: 339). In some embodiments, a linker comprises or consists of the sequence EAAAK (SEQ ID NO: 340). In some embodiments, a linker comprises or consists of the sequence EAAAKEAAAK (SEQ ID NO: 341). In some embodiments, a linker comprises a polyproline linker, e.g., having an amino acid sequence of PPP (SEQ ID NO: 342) or PPPP (SEQ ID NO: 343). In certain embodiments, a linker is a glycine (G)-proline (P) polypeptide linker, e.g., comprising or consisting of one or more of GPPPG (SEQ ID NO: 344), GGPPPGG (SEQ ID NO: 345), GPPPPG (SEQ ID NO: 346), or GGPPPPGG (SEQ ID NO: 347). In some embodiments, a linker herein is a (GₙS)ₘ linker, wherein n and m are independently integers from 1 to 5 (SEQ ID NO: 348). In certain embodiments, a linker comprises or consists of an amino acid sequence of (G₃S)ₙ(G₄S)₁ (SEQ ID NO: 349), (G3S) (G4S), (SEQ ID NO: 350), (G₃S)ₙ(G₄S)ₙ (SEQ ID NO: 351), or (G₄S)ₙ (SEQ ID NO: 355) wherein each n is an integer from 1 to 5. In certain embodiments, a linker herein is suitable for connecting two different domains of an antibody construct and comprises a sequence comprising glycine-serine linkers, for example, but not limited to, (GₘS)ₙ-GG (SEQ ID NO: 352), wherein m and n are independently integers from 0 to 20, (SGₙ)ₘ (SEQ ID NO: 353), or (SEGₙ)ₘ (SEQ ID NO: 354), wherein m and n are independently integers from 0 to 20 but not 0 at the same time.

In some embodiments, an antibody construct described herein comprises one or more any one or more of the linkers described herein. In some embodiments, an antibody construct comprises, e.g., 1, 2, 3, 4, or 5 linkers, which can include one or more linker^{Fab-Fc}, one or more linker^{scFv-Fab}, and/or one or more linker^{scFv}.

In certain embodiments, a linker^{scFv} of an antibody construct herein comprises or consists of the amino acid sequence (GₙS)ₘ linker, wherein n and m are independently integers from 1 to 5 (SEQ ID NO: 348). In such embodiments, n and m can both be 4, and thus the one or more linker^{scFv} of an antibody construct can comprise or consist of the sequence (G4S) 4 (SEQ ID NO: 104). In some embodiments, the one or more linker^{scFv} of an antibody construct (e.g., a linker^{scFv1} and a linker^{scFv2} that have an identical amino acid sequence) can comprise or consist of an amino acid sequence having about 80%, 90%, or 100% sequence identity to the amino acid sequence set forth in SEQ ID NO: 104.

In some embodiments, an antibody construct of the present disclosure can comprise a linker^{scFv-Fab} that couples the C-terminus of a scFv domain to an N-terminus of a Fab domain. Such linker^{scFv-Fab} can comprise or consist of the amino acid sequence set forth in SEQ ID NO: 105, or a sequence having about 80%, 90%, or 100% sequence identity thereto.

In some embodiments, an antibody construct of the present disclosure can comprise a linker^{Fc-scFv} that couples the C-terminus of an Fc polypeptide to the N-terminus of an scFv domain. In some embodiments, such linker^{Fc-scFv} can also comprise or consist of the amino acid sequence set forth in SEQ ID NO: 105, or a sequence having about 80%, 90%, or 100% sequence identity thereto.

In certain embodiments herein, one or more of the linkers an antibody construct comprises can be an amino acid sequence obtained, derived, or designed from an antibody hinge region sequence. In some embodiments, such linker can have at least one cysteine capable of participating in at least one disulfide bond under physiological conditions or other standard peptide conditions (e.g., peptide purification conditions, conditions for peptide storage, etc.). In certain embodiments, a linker corresponding to, or similar to, an Ig hinge peptide retains a cysteine that corresponds to the hinge cysteine disposed toward the amino (or N-) terminus of that hinge. In further embodiments, a linker is derived from an IgG1 hinge and can be modified to remove any cysteine residues, or the linker is an IgG1 hinge that has one cysteine or two cysteines corresponding to hinge cysteines.

In certain embodiments, a linker of an antibody construct described herein can comprise an “altered wildtype Ig hinge region” or an “altered Ig hinge region”. Such altered hinge regions can refer to (a) a wild type Ig hinge region with up to 30 percent amino acid changes (e.g., up to 25 percent, 20 percent, 15 percent, 10 percent, or 5 percent amino acid substitutions, insertions or deletions), (b) a portion of a wild type Ig hinge region that is at least 10 amino acids (e.g., at least 12, 13, 14 or 15 amino acids) in length with up to 30 percent amino acid changes (e.g., up to 25 percent, 20 percent, 15 percent, 10 percent, or 5 percent amino acid substitutions or deletions), (c) a portion of a wild type Ig hinge region that comprises the core hinge region, which portion can be 4, 5, 6, 7, 8, 9, 10, 11, 12, 13, 14, or 15, or at least 4, 5, 6, 7, 8, 9, 10, 11, 12, 13, 14, or 15 amino acids in length, or (d) a combination of any of (a)-(c). In certain embodiments, one or more cysteine residues in a wildtype Ig hinge region, such as an IgG1 hinge comprising the upper and core regions, can be substituted by one or more other amino acid residues (e.g., one or more serine residues). An altered Ig hinge region can alternatively or additionally have a proline residue of a wildtype Ig hinge region, such as an IgG1 hinge comprising the upper and core regions, substituted by another amino acid residue (e.g., a serine residue).

Hence, in some embodiments, an antibody construct of this disclosure comprises a linker comprising or consisting of an amino acid sequence having at least about 80%, 90%, 95%, 97%, 99%, or 100% sequence identity to the sequence set forth in SEQ ID NO: 108. In some embodiments, an antibody construct comprises a linker comprising or consisting of an amino acid sequence having at least about 80%, 90%, 95%, 97%, 99%, or 100% sequence identity to the sequence set forth in SEQ ID NO: 111. In some embodiments, an antibody construct comprises a linker comprising or consisting of an amino acid sequence having at least about 80%, 90%, 95%, 97%, 99%, or 100% sequence identity to the sequence set forth in SEQ ID NO: 112.

In various embodiments, an antibody construct of this disclosure comprises a linker that couples a Fab domain to a first Fc polypeptide, linker^{Fab-fc}, wherein the linker^{Fab-Fc} comprises or consists of the amino acid sequence set forth in SEQ ID NO: 108, and another linker coupling a first or second scFv domain to a first or second Fc polypeptide, linker^{scFv-Fc}, wherein the linker^{scFv-Fc} comprises or consists of the amino acid sequence set forth in SEQ ID NO: 111 or SEQ ID NO: 112.

### F. Certain Embodiments of Antibody Constructs

In one embodiment, described herein is a trivalent and trispecific antibody construct, comprising: (i) a Fab domain capable of binding CD3 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L}sequence; (ii) a first scFv domain capable of binding CD28 on a second immune cell, wherein the first scFv domain comprises, from N- to C-terminus, a first scFv V_{H} sequence coupled to a first scFv V_{L} sequence via a first linker^{scFv}, (iii) a second scFv domain capable of binding MSLN on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-Fc}, b) the C-terminus of the first scFv domain is coupled to the N-terminus of the Fab V_{H} sequence via a linker^{scFv-Fab}, and c) the C-terminus of the second scFv domain is coupled to the N-terminus of the second Fc polypeptide via a linker^{scFv-Fc}.

In one embodiment, described herein is a trivalent and trispecific antibody construct, comprising: (i) a Fab domain capable of binding CD3 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD28 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding MSLN on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-Fc}, b) the N-terminus of the first scFv domain is coupled to the C-terminus of the Fab C_{L} sequence via a linker^{Fab-scFv}, and c) the C-terminus of the second scFv domain is coupled to the N-terminus of the second Fc polypeptide via a linker^{scFv-Fc}.

In one embodiment, described herein is a trivalent and trispecific antibody construct, comprising: (i) a Fab domain capable of binding CD3 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD28 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding MSLN on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-fc}, b) the N-terminus of the first scFv domain is coupled to the C-terminus of the first Fc polypeptide via a linker^{Fc-scFv}, and c) the C-terminus of the second scFv domain is coupled to the N-terminus of the second Fc polypeptide via a linker^{scFv-Fc}.

In one embodiment, described herein is a trivalent and trispecific antibody construct, comprising: (i) a Fab domain capable of binding CD3 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a CI sequence; (ii) a first scFv domain capable of binding CD28 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding MSLN on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-Fc}, b) the N-terminus of the first scFv domain is coupled to the C-terminus of the first Fc polypeptide via a linker^{Fc-scFv}, and c) the C-terminus of the second scFv domain is coupled to the N-terminus of the Fab V_{H} sequence via a linker^{scFv-Fab}.

In one embodiment, described herein is a trivalent and trispecific antibody construct, comprising: (i) a Fab domain capable of binding CD3 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD28 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding MSLN on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the second Fc polypeptide via the C-terminus of the Fab C_{H1} sequence and via a linker^{Fab-Fc}, b) the C-terminus of the first scFv domain is coupled to the N-terminus of the first Fc polypeptide via a linker^{scFv-Fc}, and c) the C-terminus of the second scFv domain is coupled to the N-terminus of the Fab V_{H} sequence via a linker^{scFv-Fab}.

In one embodiment, described herein is a trivalent and trispecific antibody construct, comprising: (i) a Fab domain capable of binding CD28 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD3 on a second immune cell, wherein the first scFv domain comprises, from N- to C-terminus, a first scFv V_{H} sequence coupled to a first scFv V_{L} sequence via a first linker^{scFv}, (iii) a second scFv domain capable of binding MSLN on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-Fc}, b) the C-terminus of the first scFv domain is coupled to the N-terminus of the Fab V_{H} sequence via a linker^{scFv-Fab}, and c) the C-terminus of the second scFv domain is coupled to the N-terminus of the second Fc polypeptide via a linker^{scFv-Fc}.

In one embodiment, described herein is a trivalent and trispecific antibody construct, comprising: (i) a Fab domain capable of binding CD28 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD3 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding MSLN on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-fc}, b) the N-terminus of the first scFv domain is coupled to the C-terminus of the Fab C_{L} sequence via a linker^{Fab-scFv}, and c) the C-terminus of the second scFv domain is coupled to the N-terminus of the second Fc polypeptide via a linker^{scFv-Fc}.

In one embodiment, described herein is a trivalent and trispecific antibody construct, comprising: (i) a Fab domain capable of binding CD28 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD3 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding MSLN on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-fc}, b) the N-terminus of the first scFv domain is coupled to the C-terminus of the first Fc polypeptide via a linker^{Fc-scFv}, and c) the C-terminus of the second scFv domain is coupled to the N-terminus of the second Fc polypeptide via a linker^{scFv-Fc}.

In one embodiment, described herein is a trivalent and trispecific antibody construct, comprising: (i) a Fab domain capable of binding CD28 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD3 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding MSLN on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-fc}, b) the N-terminus of the first scFv domain is coupled to the C-terminus of the first Fc polypeptide via a linker^{Fc-scFv}, and c) the C-terminus of the second scFv domain is coupled to the N-terminus of the Fab V_{H} sequence via a linker^{scFv-Fab}.

In one embodiment, described herein is a trivalent and trispecific antibody construct, comprising: (i) a Fab domain capable of binding CD28 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD3 on a second immune cell, wherein the first scFv domain comprises, from N- to C-terminus, a first scFv V_{H} sequence coupled to a first scFv V_{L} sequence via a first linker^{scFv}, (iii) a second scFv domain capable of binding MSLN on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-Fc}, b) the C-terminus of the first scFv domain is coupled to the N-terminus of the second Fc polypeptide via a linker^{scFv-fc}, and c) the C-terminus of the second scFv domain is coupled to the N-terminus of the Fab V_{H} sequence via a linker^{scFv-Fab}.

In one embodiment, described herein is a trivalent and trispecific antibody construct, comprising: (i) a Fab domain capable of binding CD3 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD28 on a second immune cell, wherein the first scFv domain comprises, from N- to C-terminus, a first scFv V_{H} sequence coupled to a first scFv V_{L} sequence via a first linker^{scFv}, (iii) a second scFv domain capable of binding Cldn 18.2 on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-Fc}, b) the C-terminus of the first scFv domain is coupled to the N-terminus of the Fab V_{H} sequence via a linker^{scFv-Fab}, and c) the C-terminus of the second scFv domain is coupled to the N-terminus of the second Fc polypeptide via a linker^{scFv-Fc}.

In one embodiment, described herein is a trivalent and trispecific antibody construct, comprising: (i) a Fab domain capable of binding CD3 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD28 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding Cldn 18.2 on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-fc}, b) the N-terminus of the first scFv domain is coupled to the C-terminus of the Fab C_{L} sequence via a linker^{Fab-scFv}, and c) the C-terminus of the second scFv domain is coupled to the N-terminus of the second Fc polypeptide via a linker^{scFv-Fc}.

In one embodiment, described herein is a trivalent and trispecific antibody construct, comprising: (i) a Fab domain capable of binding CD3 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD28 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding Cldn 18.2 on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{fab-Fc}, b) the N-terminus of the first scFv domain is coupled to the C-terminus of the first Fc polypeptide via a linker^{Fc-scFv}, and c) the C-terminus of the second scFv domain is coupled to the N-terminus of the second Fc polypeptide via a linker^{scFv-Fc}.

In one embodiment, described herein is a trivalent and trispecific antibody construct, comprising: (i) a Fab domain capable of binding CD3 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD28 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding Cldn 18.2 on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-Fc}, b) the N-terminus of the first scFv domain is coupled to the C-terminus of the first Fc polypeptide via a linker^{Fc-scFv}, and c) the C-terminus of the second scFv domain is coupled to the N-terminus of the Fab V_{H} sequence via a linker^{scFv-Fab}

In one embodiment, described herein is a trivalent and trispecific antibody construct, comprising: (i) a Fab domain capable of binding CD3 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD28 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding Cldn 18.2 on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the second Fc polypeptide via the C-terminus of the Fab C_{H1} sequence and via a linker^{Fab-Fc}, b) the C-terminus of the first scFv domain is coupled to the N-terminus of the first Fc polypeptide via a linkers^{scFv-Fc}, and c) the C-terminus of the second scFv domain is coupled to the N-terminus of the Fab V_{H} sequence via a linker^{scFv-Fab}.

In one embodiment, described herein is a trivalent and trispecific antibody construct, comprising: (i) a Fab domain capable of binding CD28 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD3 on a second immune cell, wherein the first scFv domain comprises, from N- to C-terminus, a first scFv V_{H} sequence coupled to a first scFv V_{L} sequence via a first linker^{scFv}, (iii) a second scFv domain capable of binding Cldn 18.2 on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-Fc}, b) the C-terminus of the first scFv domain is coupled to the N-terminus of the Fab V_{H} sequence via a linker^{scFv-Fab}, and c) the C-terminus of the second scFv domain is coupled to the N-terminus of the second Fc polypeptide via a linker^{scFv-Fc}.

In one embodiment, described herein is a trivalent and trispecific antibody construct, comprising: (i) a Fab domain capable of binding CD28 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD3 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding Cldn 18.2 on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-Fc}, b) the N-terminus of the first scFv domain is coupled to the C-terminus of the Fab C_{L} sequence via a linker^{Fab-scFv}, and c) the C-terminus of the second scFv domain is coupled to the N-terminus of the second Fc polypeptide via a linker^{scFv-Fc}.

In one embodiment, described herein is a trivalent and trispecific antibody construct, comprising: (i) a Fab domain capable of binding CD28 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD3 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding Cldn 18.2 on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-fc}, b) the N-terminus of the first scFv domain is coupled to the C-terminus of the first Fc polypeptide via a linker^{Fc-scFv}, and c) the C-terminus of the second scFv domain is coupled to the N-terminus of the second Fc polypeptide via a linker^{scFv-Fc}.

In one embodiment, described herein is a trivalent and trispecific antibody construct, comprising: (i) a Fab domain capable of binding CD28 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD3 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding Cldn 18.2 on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-fc}, b) the N-terminus of the first scFv domain is coupled to the C-terminus of the first Fc polypeptide via a linker^{Fc-scFv}, and c) the C-terminus of the second scFv domain is coupled to the N-terminus of the Fab V_{H} sequence via a linker^{scFv-Fab}.

In one embodiment, described herein is a trivalent and trispecific antibody construct, comprising: (i) a Fab domain capable of binding CD28 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD3 on a second immune cell, wherein the first scFv domain comprises, from N- to C-terminus, a first scFv V_{H} sequence coupled to a first scFv V_{L} sequence via a first linker^{scFv}, (iii) a second scFv domain capable of binding Cldn 18.2 on a tumor cell, wherein the second scFv domain comprises, from N- to C-terminus, a second scFv V_{H} sequence coupled to a second scFv V_{L}, sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-Fc}, b) the C-terminus of the first scFv domain is coupled to the N-terminus of the second Fc polypeptide via a linker^{scFv-Fc}, and c) the C-terminus of the second scFv domain is coupled to the N-terminus of the Fab V_{H} sequence via a linker^{scFv-Fab}.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 100 or 118, (ii) a second heavy chain polypeptide (H2) that comprises or consists of an amino acid sequence having at least about 90%, 95%, 97%, 99%, or 100% sequence identity to the amino acid sequence set forth in SEQ ID NO: 111 or 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115 or 120.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 111, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 118, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 122, (ii) a second heavy chain polypeptide (H2) that comprises or consists of an amino acid sequence having at least about 90%, 95%, 97%, 99%, or 100% sequence identity to the amino acid sequence set forth in SEQ ID NO: 111, and (iii) a light chain polypeptide (L1) that comprises or consists of an amino acid sequence having at least about 90%, 95%, 97%, 99%, or 100% sequence identity to the amino acid sequence set forth in SEQ ID NO: 123.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 124, (ii) a second heavy chain polypeptide (H2) that comprises or consists of an amino acid sequence having at least about 90%, 95%, 97%, 99%, or 100% sequence identity to the amino acid sequence set forth in SEQ ID NO: 111, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 124, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 129, (ii) a second heavy chain polypeptide (H2) that comprises or consists of an amino acid sequence having at least about 90%, 95%, 97%, 99%, or 100% sequence identity to the amino acid sequence set forth in SEQ ID NO: 111, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 122, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 123.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 129, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 134, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 135.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 130, (ii) a second heavy chain polypeptide (H2) that comprises or consists of an amino acid sequence having at least about 90%, 95%, 97%, 99%, or 100% sequence identity to the amino acid sequence set forth in SEQ ID NO: 131, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 132, (ii) a second heavy chain polypeptide (H2) that comprises or consists of an amino acid sequence having at least about 90%, 95%, 97%, 99%, or 100% sequence identity to the amino acid sequence set forth in SEQ ID NO: 133, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 136, (ii) a second heavy chain polypeptide (H2) that comprises or consists of an amino acid sequence having at least about 90%, 95%, 97%, 99%, or 100% sequence identity to the amino acid sequence set forth in SEQ ID NO: 131, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 137, (ii) a second heavy chain polypeptide (H2) that comprises or consists of an amino acid sequence having at least about 90%, 95%, 97%, 99%, or 100% sequence identity to the amino acid sequence set forth in SEQ ID NO: 138, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 139, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 140, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 141.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 142.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in any one of SEQ ID NOs: 143-149, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 143, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 144, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 145, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 146, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 147, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 148, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 149, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

In one embodiment, described herein is a trivalent and trispecific antibody construct, comprising: (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 150 or SEQ ID NO: 152.

In certain embodiments, a trivalent and trispecific antibody constructs of the present disclosure is one in which the first scFv domain and the second scFv domain are not coupled to each other in tandem, i.e., in a structure of scFv1-scFv2 or scFv2-scFv, from N- to C-terminus, and either with or without a linker sequence between scFv1 and scFv2.

### G. Certain Properties of Trivalent and Trispecific Antibody Constructs

The trivalent and trispecific antibody constructs of the present disclosure can have several specific properties due to their format, geometry and antigen affinities.

Thus, in some embodiments, the engagement of a trivalent and trispecific antibody construct of two different antigens on one or more T cell(s) and an antigen on a tumor cell, e.g., in a tumor (micro) environment, can be—at least temporarily—simultaneous, thereby establish a TCR-independent immune synapse, and direct T cell-mediated cytotoxic activity to a tumor environment which contains tumor cells expressing the TAA. In various embodiments, and as further described herein, a trivalent and trispecific antibody construct may cause a significantly reduced immune cell (e.g., T cell) activation in the absence of a TAA, e.g., when the immune synapse cannot be fully formed due to an absence of the TAA. Such property can be advantageous over those of conventional constructs as it allows, for example, activation of a subject's immune system in a more TAA-dependent manner, and thus may cause less off-targets effects in the subject compared to conventional constructs of antibodies that act in a less TAA-dependent manner.

In some embodiments, the antibody constructs described herein can posses an enhanced anti-tumor activity in tumors that have a relatively low T cell infiltration when compared to conventional constructs that target only one immune cell antigen (e.g., CD3 or CD28), due to their co-stimulatory activity by being capable of engaging both CD3 and CD28 on either the same immune cell (e.g., T cell) or on two different, e.g., adjacent, immune cells (e.g., T cells).

Generally, and as it may be appreciated by a person of ordinary skill in the art, natural T cell activation can require both TCR (e.g., involving CD3) and CD28 stimulation. The antibody constructs of the present disclosure have been specifically designed, e.g., through their format and geometry, to provide both CD3 and CD28 co-stimulation. Furthermore, and according to various embodiments of the present disclosure, the anti-CD3 and anti-CD28 binding affinities of the anti-CD3 and anti-CD28 binding domains (e.g., scFv's, Fab's, etc.) of antibody construct described herein, in combination with their relative positioning within the construct, have been specifically selected and engineered to generate signals for immune cell (e.g., T cell) activation with appropriate strength to reduce both T cell anergy on the one side and T cell overreaction and dysfunction on the other side of the spectrum, and to provide an improved ratio of anti-tumor, on-target to healthy tissue, off-target activity. In various embodiments, such improved on-target- to-off-target activities was achieved by optimizing the format and geometry of the antibody constructs in a way such that engagement of all three antigens, namely CD3, CD28 and the TAA, at the same time allows for the most potent anti-tumor activity, compared to instances in which, e.g., only CD3 and CD28 are engaged and bound by the construct.

As further described herein, an antibody construct of the present disclosure can be trivalent and trispecific and bind each antigen, e.g., CD3, CD28, TAA, monovalently via one of its three antigen binding domains.

In some embodiments, a trivalent and trispecific antibody construct has a binding affinity for the TAA of at least about 40 nM, 30 nM, 20 nM, 10 nM or 5 nM, or from about 40 nM to about 5 nM or from about 30 nM to about 10 nM, e.g., using SPR or other methods known in the art. In one embodiment, the TAA is MSLN. In another embodiment, the TAA is Cldn18.2.

In some embodiments, a trivalent and trispecific antibody construct has a melting temperature at Tm1, Tm2, and/or Tm3 that is within 10° C., 5° C., 2° C., or within 1° C. degree of that of a bivalent and monospecific IgG1 monoclonal antibody, as determined using, e.g., differential scanning calorimetry (DSC) or differential scanning fluorometry (DSF). In some embodiments, such bivalent and monospecific IgG1 monoclonal antibody can be any conventional IgG1 antibody capable of binding a specific antigen. In certain embodiments, such monoclonal, monospecific and bivalent IgG1 antibody is one that comprises two Fab domains as described herein in the context of trispecific antibody constructs. In some embodiments, the bivalent and monospecific IgG1 monoclonal antibody comprises two anti-CD3 Fab domains as described herein, two anti-CD28 Fab domains as described herein, two anti-MSLN Fab domains comprising the anti-MSLN VH and VL sequences as described herein, or two anti-Cldn18.2 Fab domains comprising the anti-Cldn 18.2 VH and VL sequences as described herein.

In some embodiments, a trivalent and trispecific antibody construct of the present disclosure binds a cytotoxic effector cell (e.g., a T cell) that expresses CD3 and CD28 with an affinity from about 5 nM to about 100 pM, from about 1 nM to about 100 pM, from about 1 nM to about 250 pM, from about 1 nM to about 500 pM, or from about 1 nM to about 750 pM. In certain embodiments, such antibody construct binds the cytotoxic effector cell that expresses CD3 and CD28 with an affinity that is about 2-fold, 5-fold, 10-fold, 20-fold, 50-fold, 100-fold, or about 200-fold higher, and/or from about 2-fold to about 200-fold, from about 2-fold to about 150-fold, from about 2-fold to about 100-fold, or from about 20-fold to about 200-fold higher than that of a corresponding bispecific anti-CD3×TAA and/or anti-CD28×TAA antibody construct.

In some embodiments, using the engineered anti-CD28 binding domains with reduced CD28 affinity, e.g., compared to the parent huTN228 paratope, can result in trivalent and trispecific antibody constructs that may induce less CD28-mediated toxicities.

In some embodiments, a trivalent and trispecific antibody construct of the present disclosure exhibits an IC₅₀ value from about 50 pM to about 0.01 pM, from about 25 pM to about 0.01 pM, from about from about 10 pM to about 0.05 pM, from about 10 pM to about 0.1 pM, from about 10 pM to about 1 pM, from about 5 pM to about 1 pM for killing TAA-expressing tumor cells that express at least about 100,000 TAA/cell by TDCC in the presence of the cytotoxic effector cell and using an E:T ratio of 2:1 and an incubation period of 72 hours. In some of such embodiments, the antibody construct achieves a maximum killing of TAA-expressing tumor cells of at least about 60%, 65%, 70%, 75%, or 80%, 85%, or 90%, 100%, or from about 60% to about 100%, from about 70% to about 90%, or from about 75% to about 85%.

In some embodiments, a trivalent and trispecific antibody construct of the present disclosure is capable of inducing the production of one or more cytokines by the cytotoxic effector cell ranging from about 300 pg/mL to about 9000 pg/mL, when TAA-expressing cells expressing at least about 100,000 TAA/cell are present and using an E:T ratio of 2:1 and an incubation period of 72 hours.

Furthermore, in various embodiments, the trivalent and trispecific antibody constructs of the present disclosure may provide a strictly target cell (e.g., tumor cell) dependent cytotoxicity profile, e.g., as shown herein when cytokine release is significantly reduced in the presence of only isolated T cells compared to conditions in which the T cells are in co-culture with TAA-expressing tumor cells, see, e.g., EXAMPLE 25.

In some embodiments, the trivalent and trispecific antibody constructs disclosed herein can have a thermal stability when measured at 40° C. and over a time period of about 2, 3, 5, 7, 10, or 14 days of at least about 90%, 95%, 97%, 98% or 99% intact construct, when measured using, e.g., size-exclusion chromatography, or other methods known in the art. In certain embodiments, such constructs can comprise one Fab domain capable of binding either CD3 or CD28, and two scFv domains, wherein one such scFv domain is capable of binding either CD3 or CD28 (e.g., whichever the Fab domain doesn't bind), and one scFv domain is capable of binding a TAA, such as in the construct v37634. In some embodiments, the stability of a trispecific and trivalent antibody construct over a period of 14 days at 40° C. is at least about 97% or 98% percent (i.e., at least about 97% or 98% of construct is intact as measured, e.g., using size-exclusion chromatography). In some embodiments, the concentration of the construct in such stability experiment is about 1 mg/mL.

H. Trivalent and Trispecific Antibody Constructs Comprising Light Chains Comprising a Fab Portion and an scFv Portion

Certain embodiments of the present disclosure relate to trivalent and trispecific antibody constructs comprising a light chain comprising (from either N- to C-terminus or C- to N-terminus) a (i) Fab portion comprising a first VL sequence and a CL sequence, coupled to (ii) an scFv domain comprising a second VL sequence and a VH sequence.

In some embodiments, such trivalent and trispecific antibody construct comprises a light chain having the following domain structure, from N- to C-terminus, of VL-CL-scFv, wherein the scFv domain can comprise, from N- to C-terminus, a VH coupled to a VL sequence, or a VL sequence coupled to a VH sequence. Thus, in certain embodiments, the light chain has the domain structure, from N- to C-terminus, of (VL-CL)_{Fab}-(VL-VH)_{scFv}. In other embodiments, the light chain has the domain structure, from N- to C-terminus, of (VL-CL)_{Fab}-(VH-VL)_{scFv}.

In certain embodiments, described herein is a trivalent and trispecific antibody construct comprising a light chain having the following domain structure, from N- to C-terminus, of (VL-CL)_{Fab}-(VL-VH)_{scFv}. In some embodiments, such light chain can further comprise one or more linkers, as further described herein. In some embodiments, the light chain comprises a peptide linker^{Fab-scfv} between the Fab portion and the scFv portion which couples the Fab portion to the scFv portion to yield the light chain with the domain structure (VL-CL)_{Fab}-Linker^{Fab-scFv}-(VL-VH)_{scFv}. The linker^{Fab-scFv} can comprise or consist of the amino acid sequence set forth in SEQ ID NO: 105. As further described herein, the scFv domain of the light chain can also comprise a linker^{scFv} that couples the VL sequence to the VH sequence. Thus, in certain embodiments, the trivalent and trispecific antibody construct comprises a light chain with the domain structure (VL-CL)_{Fab}-Linker^{Fab-scFv}-(VL-Linker^{scFv}-VH)_{scFv}. The linker_{scFv} can comprise or consist of the amino acid sequence set forth in SEQ ID NO: 104.

In certain embodiments, described herein is a trivalent and trispecific antibody construct comprising a light chain comprising an amino acid sequence having at least about 80%, 85%, 90%, 95%, 97%, 99%, or 100% sequence identity to the amino acid sequence set forth in SEQ ID NO: 123.

In some embodiments, described herein is a trivalent and trispecific antibody construct comprising a light chain comprising a Fab portion and an scFv portion, wherein the antibody construct comprises: (i) a Fab domain comprising a heavy chain comprising a VH sequence and a CH1 sequence and a light chain comprising a VL sequence and CL sequence, wherein the Fab domain is capable of binding CD3; (ii) a first scFv domain comprising a first VH sequence and a first VL sequence, wherein the first scFv domain is capable of binding CD28; (iii) a second scFv domain comprising a second VH sequence and a second VL sequence, wherein the second scFv domain is capable of binding Cldn18.2; and (iv) a dimeric Fc domain comprising a first Fc polypeptide and a second Fc polypeptide, wherein: a) the Fab domain is coupled via its CH1 sequence to the N-terminus of the first Fc polypeptide, b) the first scFv domain is coupled to the C-terminus of the CL sequence of the Fab light chain, and c) the second scFv domain is coupled to the N-terminus of the second Fc polypeptide.

In some embodiments, described herein is a trivalent and trispecific antibody construct comprising a light chain comprising a Fab portion and an scFv portion, wherein the antibody construct comprises: (i) a Fab domain capable of binding CD3 on a first immune cell, wherein the Fab domain comprises a heavy chain portion comprising, from N- to C-terminus, a V_{H} sequence coupled to a C_{H1} sequence, paired with a light chain comprising, from N- to C-terminus, a V_{L} sequence coupled to a C_{L} sequence; (ii) a first scFv domain capable of binding CD28 on a second immune cell, wherein the first scFv domain comprises, from N- to C-terminus, a first scFv V_{L} sequence coupled to a first scFv V_{H} sequence via a first linker^{scFv}, (iii) a second scFv domain capable of binding Cldn 18.2 on a tumor cell, wherein the second scFv domain comprises, from N- to C-terminus, a second scFv V_{H} sequence coupled to a second scFv V_{L} sequence via a second linker^{scFv}, and (iv) a heterodimeric Fc domain comprising a first Fc polypeptide and a second Fc polypeptide, wherein a) the Fab domain is coupled to the N-terminus of the first Fc polypeptide via the C-terminus of the Fab C_{H1} sequence and via a linker^{Fab-Fc}, b) the N-terminus of the first scFv domain is coupled to the C-terminus of the Fab C_{L} sequence via a linker^{Fab-scFv}, and c) the C-terminus of the second scFv domain is coupled to the N-terminus of the second Fc polypeptide via a linker^{scFv-Fc}.

In various embodiments, such trivalent and trispecific antibody construct comprises a light chain with the domain structure (VL-CL)_{Fab}-Linker^{Fab-scFv}-(VL-Linker^{ScFv}-VH)_{scFv}. In certain embodiments, such light chain comprises an amino acid sequence having at least about 80%, 85%, 90%, 95%, 97%, 99%, or 100% sequence identity to the amino acid sequence set forth in SEQ ID NO: 123. In some embodiments, such construct comprises a light chain that comprises or consists of the amino acid sequence set forth in SEQ ID NO: 123.

In various embodiments, a trivalent and trispecific antibody construct comprising a light chain comprising a Fab portion and an scFv portion can possess certain properties that can be unique to a construct with the specified format and geometry.

In some embodiments, a trivalent and trispecific antibody construct herein that comprises a light chain comprising a Fab portion and an scFv portion does not reduce T cell viability by more than 5%, 4%, 3%, 2% 1%, or by 0% compared to T cells treated with a negative control construct that does not contain a binding domain against Cldn18.2, measured after incubation of the T cells with the respective construct for 48 hours. In some embodiments, the trivalent and trispecific antibody construct does not reduce T cell viability by more than 5%, 3%, 1% or 0%. In some embodiments, the trivalent and trispecific antibody construct does not reduce T cell viability by more than 5% compared to the control construct. In some embodiments, the trivalent and trispecific antibody construct does not reduce T cell viability by more than 4% compared to the control construct. In some embodiments, the trivalent and trispecific antibody construct does not reduce T cell viability by more than 3% compared to the control construct. In some embodiments, the trivalent and trispecific antibody construct does not reduce T cell viability by more than 2% compared to the control construct. In some embodiments, the trivalent and trispecific antibody construct does not reduce T cell viability by more than 1% compared to the control construct. In some embodiments, the trivalent and trispecific antibody construct does not reduce T cell viability compared to the control construct.

In some embodiments, a trivalent and trispecific antibody construct herein that comprises a light chain comprising a Fab portion and an scFv portion reduces T cell viability by about 1.5-fold to about 2-fold, by about 1.5-fold to about 3-fold, or by about 2-fold to about 3-fold less than an antibody construct in which the first scFv domain and the second scFv domain are coupled either to the N-terminus of the Fab heavy chain or the N-terminus of the second Fc polypeptide, and measured when the respective antibody construct is incubated with the T cells for 48 hours. In some embodiments, the trivalent and trispecific antibody construct that comprises a light chain comprising a Fab portion and an scFv portion reduces T cell viability by about 1.5-fold to about 2-fold less than an antibody construct in which the first scFv domain and the second scFv domain are coupled either to the N-terminus of the Fab heavy chain or the N-terminus of the second Fc polypeptide. In some embodiments, the trivalent and trispecific antibody construct that comprises a light chain comprising a Fab portion and an scFv portion reduces T cell viability by about 1.5-fold to about 3-fold less than an antibody construct in which the first scFv domain and the second scFv domain are coupled either to the N-terminus of the Fab heavy chain or the N-terminus of the second Fc polypeptide. In some embodiments, the trivalent and trispecific antibody construct that comprises a light chain comprising a Fab portion and an scFv portion reduces T cell viability by about 2-fold to about 3-fold less than an antibody construct in which the first scFv domain and the second scFv domain are coupled either to the N-terminus of the Fab heavy chain or the N-terminus of the second Fc polypeptide.

In some embodiments, a trivalent and trispecific antibody construct herein that comprises a light chain comprising a Fab portion and an scFv portion induces about 80-fold to about 2000-fold, about 100-fold to about 1000-fold, or about 100-fold to about 500-fold less cytokine in an assay comprising human CD3⁺ T cells only compared to an antibody construct in which the first scFv domain and the second scFv domain are independently coupled to either the N-terminus of the Fab heavy chain or the N-terminus of the second Fc polypeptide, and wherein the respective antibody construct is incubated with the CD3⁺ T cells for 48 hours. In some embodiments, the trivalent and trispecific antibody construct that comprises a light chain comprising a Fab portion and an scFv portion induces about 80-fold to about 2000-fold less cytokine in an assay comprising human CD3⁺ T cells only compared to an antibody construct in which the first scFv domain and the second scFv domain are independently coupled to either the N-terminus of the Fab heavy chain or the N-terminus of the second Fc polypeptide. In some embodiments, the trivalent and trispecific antibody construct that comprises a light chain comprising a Fab portion and an scFv portion induces about 100-fold to about 1000-fold less cytokine in an assay comprising human CD3⁺ T cells only compared to an antibody construct in which the first scFv domain and the second scFv domain are independently coupled to either the N-terminus of the Fab heavy chain or the N-terminus of the second Fc polypeptide. In some embodiments, the trivalent and trispecific antibody construct that comprises a light chain comprising a Fab portion and an scFv portion induces about 100-fold to about 500-fold less cytokine in an assay comprising human CD3⁺ T cells only compared to an antibody construct in which the first scFv domain and the second scFv domain are independently coupled to either the N-terminus of the Fab heavy chain or the N-terminus of the second Fc polypeptide. In some embodiments, the trivalent and trispecific antibody construct that comprises a light chain comprising a Fab portion and an scFv portion induces less than about 10 pg/mL of cytokine, such as between about 10 pg/mL and 0.5 pg/mL of cytokine.

In some embodiments, a trivalent and trispecific antibody construct herein that comprises a light chain comprising a Fab portion and an scFv portion induces from about 5-fold to about 900-fold, from about 5-fold to about 500-fold, or from about 5-fold to about 300-fold less cytokine in an assay comprising human PBMCs only compared to an antibody construct in which the first scFv domain and the second scFv domain are independently coupled to either the N-terminus of the Fab heavy chain or the N-terminus of the second Fc polypeptide, and wherein the respective antibody construct is incubated with the T cells for 48 hours. In some embodiments, the trivalent and trispecific antibody construct that comprises a light chain comprising a Fab portion and an scFv portion induces from about 5-fold to about 900-fold less cytokine in an assay comprising human PBMCs only compared to an antibody construct in which the first scFv domain and the second scFv domain are independently coupled to either the N-terminus of the Fab heavy chain or the N-terminus of the second Fc polypeptide. In some embodiments, the trivalent and trispecific antibody construct that comprises a light chain comprising a Fab portion and an scFv portion induces from about 5-fold to about 500-fold less cytokine in an assay comprising human PBMCs only compared to an antibody construct in which the first scFv domain and the second scFv domain are independently coupled to either the N-terminus of the Fab heavy chain or the N-terminus of the second Fc polypeptide. In some embodiments, the trivalent and trispecific antibody construct that comprises a light chain comprising a Fab portion and an scFv portion induces from about 5-fold to about 300-fold less cytokine in an assay comprising human PBMCs only compared to an antibody construct in which the first scFv domain and the second scFv domain are independently coupled to either the N-terminus of the Fab heavy chain or the N-terminus of the second Fc polypeptide. In some embodiments, the trivalent and trispecific antibody construct that comprises a light chain comprising a Fab portion and an scFv portion induces less than about 20 pg/mL of cytokine, such as between about 20 pg/mL and 0.5 pg/mL of cytokine.

In certain embodiments, the cytokine comprises one or more of IL-2, Interleukin-6 (IL-6), IFNγ and TNFα.

In some embodiments, a trivalent and trispecific antibody construct herein that comprises a light chain comprising a Fab portion and an scFv portion induces different amounts of memory T cell subsets upon stimulation of certain immune cells, when compared to trivalent and trispecific antibody constructs having a different format and/or geometry. In some embodiments, a trivalent and trispecific antibody construct herein that comprises a light chain comprising a Fab portion and an scFv portion induces similar amounts of memory T cell subsets upon stimulation of certain immune cells, when compared to a combination treatment of two bivalent and bispecific antibody constructs that together target the same antigens as the trivalent and trispecific antibody construct that comprises a light chain comprising a Fab portion and an scFv portion.

In some of these embodiments, the trivalent and trispecific antibody construct that comprises a light chain comprising a Fab portion and an scFv portion comprises: (i) a Fab domain capable of binding CD3; (ii) a first scFv domain capable of binding CD28; (iii) a second scFv domain capable of binding Claudin 18.2 (Cldn18.2); and (iv) an Fc domain comprising the first Fc polypeptide and the second Fc polypeptide, wherein: a) the Fab domain is coupled via its C_{H1} sequence to the N-terminus of the first Fc polypeptide, b) the first scFv domain is coupled to the C-terminus of the C_{L} sequence of the Fab light chain, and c) the second scFv domain is coupled to the N-terminus of the second Fc polypeptide.

In certain embodiments, the trivalent and trispecific antibody construct that comprises a light chain comprising a Fab portion and a scFv portion is v37634.

### III. SEQUENCE IDENTITY OF AMINO ACID AND NUCLEIC ACID SEQUENCES

As described in other parts of this disclosure, certain embodiments herein relate to an isolated polypeptide or a set of isolated polypeptides (e.g., polypeptide chains H1, H2, L1, etc., or portions, e.g., domains, thereof) of a trivalent and trispecific antibody construct, as well as to a polynucleotide or a set of polynucleotides encoding the one or more polypeptide chains of an antibody construct described herein. A polynucleotide in this context can encode all or part of an antibody construct, such as one or more polypeptide chains (e.g., H1, H2, L1, etc.) of an antibody construct.

In some embodiments, described herein is a nucleic acid molecule or a set of nucleic acid molecules encoding one or more, two or more, or three or more polypeptide chains that form any of the trivalent and trispecific antibody constructs disclosed herein.

The terms “nucleic acid,” “nucleic acid molecule” and “polynucleotide” are used interchangeably herein and refer to a polymeric form of nucleotides of any length, either deoxyribonucleotides or ribonucleotides, or analogues thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers.

In some embodiments, described herein is a vector or a set of vectors comprising the nucleic acid molecule or the set of nucleic acid molecules that encode the one or more polypeptide chains (e.g., one or more of H1, H2, L1, etc.) of an antibody construct disclosed herein.

A polynucleotide that “encodes” a given polypeptide is a polynucleotide that is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence can be located 3′ to the coding sequence.

In certain embodiments, the present disclosure relates to polynucleotide and/or polypeptide sequences that are identical or substantially identical to another polynucleotide and/or polypeptide sequence. The term “identical,” in the context of two or more polynucleotide or polypeptide sequences, refers to two or more sequences or subsequences that are the same, i.e., have the identical sequence of nucleotide or amino acid monomers (i.e., 100% sequence identity), respectively. Polypeptide or polynucleotide sequences herein share “sequence identity” if they have a percentage or a certain number of amino acid residues or nucleotides, respectively, that are at least about 80%, about 85%, about 90%, about 95%, about 97%, or at least about 99% identity over a specified region when compared and aligned for maximum correspondence over a comparison window or over a designated region as measured using one of the commonly used sequence comparison algorithms as known to persons of ordinary skill in the art or by manual alignment and visual inspection. This definition also refers to the complement of a test polynucleotide sequence. The identity can exist over a region that is at least about 50 amino acids or nucleotides in length, or over a region that is from about 75 to about 100 amino acids or nucleotides in length, or, where not specified, across the entire sequence of a polypeptide or polynucleotide. For sequence comparison, typically test sequences are compared to a designated reference sequence. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent (%) sequence identities for the test sequences relative to the reference sequence, based on the program parameters.

The term “comparison window,” as used herein, refers to a segment of a sequence comprising contiguous amino acid or nucleotide positions which can be from about 20 to about 1000 contiguous amino acid or nucleotide positions, for example from about 50 to about 600 or from about 100 to about 300 or from about 150 to about 200 contiguous amino acid or nucleotide positions over which a test sequence can be compared to a reference sequence of the same number of contiguous positions after the two sequences are optimally aligned. Longer segments up to and including the full-length sequence may also be used as a comparison window in certain embodiments. Methods of alignment of sequences for comparison are known to those of ordinary skill in the art. Optimal alignment of sequences for comparison can be conducted, for example, by the local homology algorithm of Smith & Waterman, 1970, Adv. Appl. Math., 2: 482c; by the homology alignment algorithm of Needleman & Wunsch, 1970, J. Mol. Biol., 48:443; by the search for similarity method of Pearson & Lipman, 1988, Proc. Natl. Acad. Sci. USA, 85:2444, or by computerized implementations of these algorithms (for example, GAP, BESTFIT, FASTA or TFASTA in the Wisconsin Genetics Software Package, Genetics Computer Group, Madison, WI), or by manual alignment and visual inspection (see, for example, Ausubel et al., Current Protocols in Molecular Biology, (1995 supplement), Cold Spring Harbor Laboratory Press). Examples of available algorithms suitable for determining percent sequence identity are the BLAST and BLAST 2.0 algorithms, which are described in Altschul et al., 1997, Nuc. Acids Res., 25:3389-3402, and Altschul et al., 1990, J. Mol. Biol., 215:403-410, respectively. Software for performing BLAST analyses is publicly available through the website for the National Center for Biotechnology Information (NCBI).

Certain embodiments described herein relate to variant sequences (e.g., variant V_{H} domains, variant Fc polypeptides, etc.) that comprise one or more amino acid modification, e.g., one or more amino acid insertions, one or more amino acid deletions, and/or one or more amino acid substitutions, when compared to, e.g., a reference such as a wildtype sequence. In certain embodiments, the one or more amino acid modification of a variant sequence comprises one or more amino acid substitutions when compared to a reference such as a wildtype sequence. In such embodiments, the one or more amino acid substitutions are one or more non-conservative substitutions. In other embodiments, the one or more amino acid substitutions are one or more conservative substitutions. In general, a “conservative substitution,” as used herein, is considered to be a substitution of one amino acid with another amino acid having similar physical, chemical and/or structural properties. Common conservative substitutions are listed under Column 1 of

TABLE 4
Conservative Amino Acid Substitutions

| Original | | |
|---|---|---|
| Amino Acid | Column 1 | Column 2 |
| Ala (A) | Gly, Ile, Leu, Met, | Cys, Gly, Ile, Leu, Met, Norleucine, Phe, |
| | Norleucine, Val | Trp, Tyr, Val |
| Arg (R) | His, Lys | His, Lys |
| Asn (N) | Cys, Gln, Ser, Thr | Asp, Cys, Gln, Glu, Ser, Thr |
| Asp (D) | Glu | Asn, Cys, Gln, Glu, Ser, Thr |
| Cys (C) | Asn, Gln, Ser, Thr | Asn, Asp, Gln, Glu, Ser, Thr |
| Gln (Q) | Asn, Cys, Ser, Thr | Asn, Asp, Cys, Glu, Ser, Thr |
| Glu (E) | Asp | Asp, Asn, Cys, Gln, Ser, Thr |
| Gly (G) | Pro | Ala, Ile, Leu, Met, Norleucine, Pro, Val |
| His (H) | Arg, Lys | Arg, Lys, Phe, Trp, Tyr |
| Ile (I) | Ala, Gly, Leu, Met, | Ala, Cys, Gly, Leu, Met, Norleucine, |
| | Norleucine, Val | Phe, Trp, Tyr, Val |
| Leu (L) | Ala, Gly, Ile, Met, | Ala, Cys, Gly, Ile, Met, Norleucine, Phe, |
| | Norleucine, Val | Trp, Tyr, Val |
| Lys (K) | Arg, His | Arg, His |
| Met (M) | Ala, Gly, Ile, Leu, | Ala, Cys, Gly, Ile, Leu, Norleucine, Phe, |
| | Norleucine, Val | Trp, Tyr, Val |
| Phe (F) | Tyr, Trp | Ala, Cys, Gly, His, Ile, Leu, Met, |
| | | Norleucine, Trp, Tyr, Val |
| Pro (P) | Gly | Gly |
| Ser (S) | Asn, Cys, Gln, Thr | Asp, Asn, Cys, Gln, Glu, Thr |
| Thr (T) | Asn, Cys, Gln, Ser | Asp, Asn, Cys, Gln, Glu, Ser |
| Trp (W) | Phe, Tyr | Ala, Cys, Gly, His, Ile, Leu, Met, |
| | | Norleucine, Phe, Tyr, Val |
| Tyr (Y) | Phe, Trp | Ala, Cys, Gly, His, Ile, Leu, Met, |
| | | Norleucine, Phe, Trp, Val |
| Val (V) | Ala, Gly, Ile, Leu, Met, | Ala, Cys, Gly, Ile, Leu, Met, Norleucine, |
| | Norleucine | Phe, Trp, Tyr |

One skilled in the art will appreciate that the main factors in determining what constitutes a conservative substitution are usually the size of the amino acid side chain and its physical/chemical properties, but that certain environments allow for substitution of a given amino acid with a broader range of amino acids than those listed in Column 1 of TABLE 4. These additional amino acids tend to either have similar properties to the amino acid being substituted but to vary more widely in size or be of similar size but vary more widely in physical/chemical properties. This broader range of conservative substitutions is listed under Column 2 of TABLE 4. The skilled person can readily ascertain the most appropriate group of substituents to select from in view of the particular protein environment in which the amino acid substitution is being made.

### IV. PHARMACEUTICAL COMPOSITIONS

In certain embodiments, the present disclosure relates to pharmaceutical compositions that can comprise one or more of the trivalent and trispecific antibody constructs described herein. In various embodiments, a pharmaceutical composition herein can further comprise a pharmaceutically acceptable excipient, carrier, buffer, stabiliser, or other materials well known to those skilled in the art. Such materials are generally non-toxic and do not interfere with the efficacy of the active ingredient (i.e., antibody construct). The precise nature of a carrier or other material can depend on the route of administration. Hence, a pharmaceutical composition herein can be formulated for various used and administration routes, e.g., for oral, intravenous, cutaneous, subcutaneous, nasal, intramuscular, or intraperitoneal administration routes.

A pharmaceutical composition for oral administration can be in tablet, capsule, powder, or liquid form. A tablet can include a solid carrier such as gelatin or an adjuvant. Liquid pharmaceutical compositions generally include a liquid carrier such as water, petroleum, animal or vegetable oils, mineral oil, or synthetic oil. Physiological saline solution, dextrose or other saccharide solution or glycols such as ethylene glycol, propylene glycol or polyethylene glycol can be included.

For intravenous, cutaneous, or subcutaneous injection, or injection at the site of affliction (e.g., at a tumor site), the active ingredient (i.e., antibody construct) can be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

For antibody constructs according to the present disclosure that are administered to a subject, administration is preferably in a “therapeutically effective amount” that is sufficient to show benefit to the individual, as further described herein. The actual amount administered, and rate and time-course of administration, can depend on the nature and severity of the disease (e.g., cancer) being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

In some embodiments, a pharmaceutical composition can comprise a second active ingredient (e.g., another protein or small molecule) in addition to an antibody construct described herein.

Hence, also described herein is a pharmaceutical composition comprising any one or more of the trivalent and trispecific antibody construct(s) disclosed herein, and a pharmaceutically acceptable carrier, excipient, diluent, or combination thereof.

### V. KITS

The present disclosure also describes kits comprising one or more of the trivalent and trispecific antibody constructs described herein, or a pharmaceutical composition as described herein and that comprises such antibody construct(s), as well as instructions for use. Thus, in certain embodiments, described herein are kits comprising vectors for expressing an antibody construct described herein and instructions for use. In certain embodiments, described herein are kits comprising host cells comprising a vector for expressing an antibody construct and instructions for use. In some embodiments, the present disclosure relates to kits comprising a purified antibody construct and instructions for use. The purified antibody construct can be lyophilized or provided in a dry form, such as a powder or granules, and the kit can additionally contain a suitable solvent for reconstitution of the lyophilized or dried component(s).

A kit can further comprise a container and a label and/or package insert on or associated with the container. The label or package insert contains instructions customarily included in commercial packages of therapeutic products, providing information or instructions about the indications, usage, dosage, administration, contraindications and/or warnings concerning the use of such therapeutic products (e.g., an antibody construct described herein). The label or package insert can further include a notice in the form prescribed by a governmental agency regulating the manufacture, use or sale of pharmaceuticals or biological products, which notice reflects approval by the agency of manufacture, for use or sale for human or animal administration. The container can hold a composition comprising an antibody construct of this disclosure. In some embodiments, the container can have a sterile access port. For example, the container can be an intravenous solution bag or a vial having a stopper that can be pierced by a hypodermic injection needle.

In addition to the container containing a composition comprising an antibody construct, the kit can further comprise one or more additional containers comprising other components of the kit. For example, a pharmaceutically acceptable buffer (such as bacteriostatic water for injection) (BWFI), phosphate-buffered saline, Ringer's solution, or dextrose solution), or other buffers or diluents can be included in such kit.

Suitable containers can include, for example, bottles, vials, syringes, intravenous solution bags, and the like. The containers can be formed from a variety of materials such as glass or plastic. If appropriate, one or more components (e.g., an antibody construct) of the kit can be lyophilized or provided in a dry form, such as a powder or granules, and the kit can additionally contain a suitable solvent for reconstitution of the lyophilized or dried component(s).

A kit herein can further include other materials desirable from a commercial or user standpoint, such as filters, needles, and syringes.

### VI. METHODS

Further described herein are methods of producing and using the trivalent and trispecific antibody constructs of the present disclosure.

### A. Methods of Producing an Antibody Construct

In some embodiments, the present disclosure relates to methods for preparing the trivalent and trispecific antibody constructs described herein. In various embodiments, an antibody construct of the present disclosure can be produced using standard recombinant methods known in the art (see, for example, U.S. Pat. No. 4,816,567 and “Antibodies: A Laboratory Manual,” 2^{nd} Edition, Ed. Greenfield, Cold Spring Harbor Laboratory Press, New York, 2014).

For recombinant production of an antibody construct described herein, a polynucleotide or set of polynucleotides encoding the antibody construct can be generated and inserted into one or more vectors for further cloning and/or expression in a host cell. Polynucleotide(s) encoding the antibody construct can be produced by standard methods known in the art (see, for example, Ausubel et al., Current Protocols in Molecular Biology, John Wiley & Sons, New York, 1994 & update, and “Antibodies: A Laboratory Manual,” 2^{nd} Edition, Ed. Greenfield, Cold Spring Harbor Laboratory Press, New York, 2014). As would be appreciated by one of skill in the art, the number of polynucleotides required for expression of the antibody construct may be dependent on the format and/or geometry of the antibody construct, including, for example, the number of polypeptide chains that the antibody construct is comprised of. For example, when an antibody construct comprises three polypeptide chains (e.g., H1, H2 and L1), three polynucleotides each encoding one polypeptide chain can be used. In embodiments in which two or more polynucleotides are used, such two or more polynucleotides can be incorporated into one vector or into more than one vector (e.g., two or three separate vectors).

Generally, for expression, the polynucleotide or set of polynucleotides encoding an antibody construct herein can be incorporated into an expression vector together with one or more regulatory elements, such as transcriptional elements, which can be used for efficient transcription of the polynucleotide(s). Examples of such regulatory elements include, but are not limited to, promoters, enhancers, terminators, and polyadenylation signals. One skilled in the art will appreciate that the choice of regulatory elements can be dependent on the host cell selected for expression of the polypeptides of the antibody construct and that such regulatory elements can be derived from a variety of sources, including bacterial, fungal, viral, mammalian or insect genes. The expression vector can optionally further contain heterologous nucleic acid sequences that facilitate expression or purification of the expressed protein. Examples include, but are not limited to, signal peptides and affinity tags such as metal-affinity tags, histidine tags, avidin/streptavidin encoding sequences, glutathione-S-transferase (GST) encoding sequences and biotin encoding sequences. The expression vector can be an extrachromosomal vector or an integrating vector. Hence, in some embodiments, the amino acid sequences of the polypeptide chains of an expressed antibody construct described herein, e.g., chains H1, H2, L1, etc., can comprise a signal peptide sequence. Such signal peptide sequences may vary depending on the expression system and conditions used for producing an antibody construct. Exemplary signal peptide sequences can comprise the amino acid sequence METDTLLLWVLLLWVPGSTG (SEQ ID NO: 155) or MRPTWAWWLFLVLLLALWAPARG (SEQ ID NO: 156), e.g., for H1, H2, etc., or MRPTWAWWLFLVLLLALWAPARG (SEQ ID NO: 156) or MGWSCIILFLVATATGVHS (SEQ ID NO: 157), e.g., for L1, L2, etc. In certain embodiments, one or more heavy chains (e.g., H1, H2, etc.) of an antibody construct described herein can comprise a C-terminal lysine residue following expression of the polypeptide chains inside the cell. In various embodiments, such C-terminal lysine residue may be enzymatically cleaved from the polypeptide chains prior to further processing (e.g., purification, formulation, etc.) and prior to use of the corresponding antibody construct, e.g., prior to administration of the construct to a subject in need thereof.

Certain embodiments for producing an antibody construct of the present disclosure relate to vectors (such as expression vectors) comprising one or more polynucleotides encoding at least a portion of an antibody construct described herein. The polynucleotide(s) can be comprised by a single vector or by more than one vector. In some embodiments, the polynucleotides are comprised by a multi-cistronic vector. Expression vectors that can be used to express polynucleotides include but are not limited to pTT5 and pUC15 cells comprising vectors encoding an antibody construct.

Suitable host cells for cloning or expression of the antibody construct polypeptides include various prokaryotic or eukaryotic cells as known in the art. Eukaryotic host cells include, for example, mammalian cells, plant cells, insect cells and yeast cells (such as Saccharomyces or Pichia cells). Prokaryotic host cells include, for example, E. coli, A. salmonicida or B. subtilis cells. In certain embodiments, an antibody construct can be produced in bacteria, in particular when glycosylation and Fc effector function are not needed or desired for the indented purpose of the antibody construct, as described for example in U.S. Pat. Nos. 5,648,237, 5,789,199, and 5,840,523, and in Charlton, Methods in Molecular Biology, Vol. 248, pp. 245-254, B.K.C. Lo, ed., Humana Press, Totowa, N.J., 2003. Eukaryotic microbes such as filamentous fungi or yeast are suitable expression host cells in certain embodiments, in particular fungi and yeast strains whose glycosylation pathways have been “humanized” resulting in the production of an antibody with a partially or fully human glycosylation pattern (see, for example, Gerngross, 2004, Nat. Biotech. 22:1409-1414, and Li et al., 2006, Nat. Biotech. 24:210-215).

Suitable host cells for the expression of glycosylated antibody constructs are, in various embodiments, eukaryotic cells. For example, U.S. Pat. Nos. 5,959,177, 6,040,498, 6,420,548, 7,125,978 and 6,417,429 describe PLANTIBODIES™ technology for producing antibodies and portions thereof (e.g., scFv(s), Fab(s), etc.) in transgenic plants. Mammalian cell lines adapted to grow in suspension are particularly useful for the expression of antibody constructs described herein. Examples include, but are not limited to, monkey kidney CV1 line transformed by SV40 (COS-7), human embryonic kidney (HEK) line 293 or 293 cells (see, for example, Graham et al., 1977, J. Gen Virol., 36:59), baby hamster kidney cells (BHK), mouse sertoli TM4 cells (see, for example, Mather, 1980, Biol Reprod, 23:243-251); monkey kidney cells (CV1), African green monkey kidney cells (VERO-76), human cervical carcinoma (HeLa) cells, canine kidney cells (MDCK), buffalo rat liver cells (BRL 3A), human lung cells (W138), human liver cells (Hep G2), mouse mammary tumour (MMT 060562), TRI cells (see, for example, Mather et al., 1982, Annals N.Y. Acad Sci, 383:44-68), MRC 5 cells, FS4 cells, Chinese hamster ovary (CHO) cells (including DHFR CHO cells, see Urlaub et al., 1980, Proc Natl Acad Sci USA, 77:4216), and myeloma cell lines (such as Y0, NS0 and Sp2/0). Exemplary mammalian host cell lines suitable for production of antibodies are reviewed in Yazaki & Wu, Methods in Molecular Biology, Vol. 248, pp. 255-268 (B.K.C. Lo, ed., Humana Press, Totowa, N.J., 2003).

In certain embodiments, the host cell used to produce a trivalent and trispecific antibody construct herein is a transient or stable higher eukaryotic cell line, such as a mammalian cell line. In some embodiments, the host cell is a mammalian HEK293T, CHO, HeLa, NS0 or COS cell. In some embodiments, the host cell is a stable cell line that allows for mature glycosylation of the antibody construct.

The host cells comprising the expression vector(s) encoding the antibody construct can be cultured using routine methods to produce the antibody construct. Alternatively, in some embodiments, host cells comprising the expression vector(s) encoding the antibody construct can be used therapeutically or prophylactically to deliver the antibody construct to a subject, or polynucleotides or expression vectors can be administered to a cell from a subject ex vivo and the cell then returned to the body of the subject.

In some embodiments, a host cell comprises (for example, has been transformed with) a vector comprising a polynucleotide that encodes a V_{L} and a V_{H} of a binding domain of an antibody construct described herein. In some embodiments, a host cell comprises (for example, has been transformed with) a vector comprising a polynucleotide that encodes a full-length polypeptide chain of an antibody construct described herein, e.g., H1, H2, or L1 as described herein. In another example, a host cell comprises a first vector comprising a polynucleotide that encodes the V_{L} of a binding domain and a second vector comprising a polynucleotide that encodes the corresponding V_{H} of the binding domain. In various embodiments, the host cell is eukaryotic, for example, a Chinese Hamster Ovary (CHO) cell, a human embryonic kidney (HEK) cell or a lymphoid cell (e.g., Y0, NS0, Sp20 cell). In certain embodiments, the host cell is Expi293™ (Thermo Fisher, Waltham, MA). In certain embodiments, the host cell used herein is CHO-S cells (National Research Council Canada) or HEK293 cells.

Certain embodiments of the present disclosure relate to a method of making an antibody construct comprising culturing a host cell into which one or more polynucleotides encoding the antibody construct, or one or more expression vectors encoding the antibody construct, have been introduced, under conditions suitable for expression of the antibody construct. Such method can further comprise recovering the antibody construct from the host cell (or from host cell culture medium). In some embodiments, such method can further comprise purifying the antibody construct.

Cell culture media that can be used include, but are not limited to, DMEM (Thermo Fisher, Waltham, MA), Opti-MEM™ (Thermo Fisher, Waltham, MA), Opti-MEM™ I Reduced Serum Medium (Thermo Fisher, Waltham, MA), RPMI-1640 medium, Expi293™ Expression Medium (Thermo Fisher, Waltham, MA), and FreeStyle CHO expression medium (Thermo Fisher Scientific, Waltham, MA). The cell culture medium can be supplemented with serum, e.g., fetal bovine serum (FBS), amino acids, e.g., L-glutamine, antibiotics, e.g., penicillin, and streptomycin, and/or antimycotics, e.g., amphotericin, or any other supplements routinely used in the to support cell culture.

In various embodiments, an antibody construct of the present disclosure is purified after expression. Proteins, such as an antibody construct of the present disclosure, can be isolated or purified in a variety of ways known to those skilled in the art (see, for example, Protein Purification: Principles and Practice, 3^{rd} Ed., Scopes, Springer-Verlag, NY, 1994). Standard purification methods that can be used for the antibody constructs disclosed herein include chromatographic techniques, including ion exchange, hydrophobic interaction, affinity, sizing or gel filtration, and reverse-phase, carried out at atmospheric pressure or at high pressure using systems such as FPLC and HPLC. Additional purification methods include electrophoretic, immunological, precipitation, dialysis and chromatofocusing techniques. Ultrafiltration and diafiltration techniques, in conjunction with protein concentration, can also be used. As is well known in the art, a variety of natural proteins bind Fc domains and other structural elements of an antibody construct, and, in some embodiments, these proteins can be used for purification of an antibody construct. For example, the bacterial proteins A and G can bind to the Fc domain of some antibody constructs. Likewise, the bacterial protein L can bind to the Fab domain of some antibody constructs. Purification can also be enabled by a particular fusion partner. For example, antibody constructs can be purified using glutathione resin if a GST fusion is employed, Ni⁺² affinity chromatography if a His-tag is employed or immobilized anti-flag antibody if a flag-tag is used. The degree of purification necessary may vary depending on the use of the antibody constructs. Hence, in some embodiments, no purification may be necessary.

In certain embodiments, an antibody construct of this disclosure is substantially pure. The term “substantially pure” (or “substantially purified”) when used in reference to an antibody construct described herein, refers to an antibody construct as substantially or essentially free of components that normally accompany or interact with the protein as found in its naturally occurring environment, such as a native cell, or a host cell in the case of a recombinantly produced antibody construct. In certain embodiments, an antibody construct that is substantially pure is an antibody construct purified to have less than about 30%, less than about 25%, less than about 20%, less than about 15%, less than about 10%, less than about 5%, or less than about 2% (by dry weight) of other contaminating protein species.

Assessment of antibody construct purity and/or homogeneity can be performed by any method known in the art, including, but not limited to, non-reducing/reducing CE-SDS, non-reducing/reducing SDS-PAGE, Ultra-high performance liquid chromatography-size exclusion chromatography (UPLC-SEC), High Performance Liquid Chromatography (HPLC), mass spectrometry, multi angle light scattering (MALS), and dynamic light scattering (DLS).

In certain embodiments, an antibody construct described herein can comprise one or more post-translational modifications. Such post-translational modifications can occur in vivo, or they be conducted in vitro after isolation of the antibody construct from the host cell.

Post-translational modifications can include various modifications as are known in the art (scc, for example, Proteins—Structure and Molecular Properties, 2nd Ed., T. E. Creighton, W. H. Freeman and Company, New York, 1993; Post-Translational Covalent Modification of Proteins, B. C. Johnson, Ed., Academic Press, New York, pgs. 1-12, 1983; Scifter et al., 1990, Meth. Enzymol., 182:626-646, and Rattan et al., 1992, Ann. N.Y. Acad. Sci., 663:48-62). In those embodiments in which an antibody construct comprises one or more post-translational modifications, the antibody construct can comprise the same type of modification at one or several sites (e.g., amino acid residues), or it can comprise different modifications at different sites.

Examples of post-translational modifications can include glycosylation, acetylation, phosphorylation, amidation, derivatization by known protecting/blocking groups, formylation, oxidation, reduction, proteolytic cleavage or specific chemical cleavage by cyanogen bromide, trypsin, chymotrypsin, papain, V8 protease or NaBH₄.

Other examples of post-translational modifications can include, for example, addition or removal of N-linked or O-linked carbohydrate chains, chemical modifications of N-linked or O-linked carbohydrate chains, processing of N-terminal or C-terminal ends, attachment of chemical moieties to the amino acid backbone, and addition or deletion of an N-terminal methionine residue resulting from prokaryotic host cell expression. Post-translational modifications can also include modification with a detectable label, such as an enzymatic, fluorescent, isotopic or affinity label to allow for detection and isolation of the protein. Examples of suitable enzyme labels include, but are not limited to, horseradish peroxidase, alkaline phosphatase, beta-galactosidase and acetylcholinesterase. Examples of suitable prosthetic group complexes include, but are not limited to, streptavidin/biotin and avidin/biotin. Examples of suitable fluorescent materials include, but are not limited to, umbelliferone, fluorescein, fluorescein isothiocyanate, rhodamine, dichlorotriazinylamine fluorescein, dansyl chloride and phycoerythrin. An example of a luminescent material is luminol, examples of bioluminescent materials include luciferase, luciferin and acquorin, and examples of suitable radioactive materials include iodine, carbon, sulfur, tritium, indium, technetium, thallium, gallium, palladium, molybdenum, xenon, and fluorine.

Additional examples of post-translational modifications can include acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, gamma-carboxylation, GPI anchor formation, hydroxylation, iodination, methylation, myristylation, pegylation, prenylation, racemization, selenoylation, sulfation, transfer-RNA mediated addition of amino acids to proteins such as arginylation, and ubiquitination.

In some embodiments, described herein is a method of producing a trivalent and trispecific antibody construct of the present disclosure, the method comprising: (a) obtaining a host cell culture comprising at least one host cell comprising one or more nucleic acid molecules encoding one or more, two or more, or three or more polypeptide chains that form the antibody construct; and (b) recovering the antibody construct from the host cell culture. In some embodiments, such method can further comprise, subsequent to step (b), purifying the antibody construct.

### B. Methods of Using an Antibody Construct of the Present Disclosure

In certain embodiments, the present disclosure relates to methods of using a trivalent and trispecific antibody construct of the present disclosure. In some embodiments, described herein are methods of using an antibody construct described herein for the treatment of a disease or condition in a subject in need thereof.

Such method can comprise administering a trivalent and trispecific antibody construct, or a pharmaceutical composition comprising such antibody construct, to a subject in need thereof. In certain embodiments, the subject is a mammal. In some embodiments, the subject is human.

In some embodiments, the present disclosure relates to a method of treating a cancer in a subject in need thereof, the method comprising administering to the subject a trivalent and trispecific antibody construct of the present disclosure, or a pharmaceutical composition comprising such antibody construct. Cancers that can be treated using the methods and antibody constructs disclosed herein can include, but are not limited to, hematologic neoplasms (including leukemias, myelomas and lymphomas), carcinomas (including adenocarcinomas and squamous cell carcinomas), melanomas and sarcomas. Carcinomas and sarcomas are also frequently referred to as “solid tumors”. In certain embodiments, the cancer is a solid tumor. In certain embodiments, the cancer is leukemia. In certain embodiments, the cancer is lymphoma.

When used in a method described herein, an antibody construct of this disclosure can exert cither a cytotoxic or cytostatic effect and can result in one or more of a reduction in the size of a tumor, the slowing or prevention of an increase in the size of a tumor, an increase in the disease-free survival time between the disappearance or removal of a tumor and its reappearance, prevention of an initial or subsequent occurrence of a tumor (e.g., metastasis), an increase in the time to progression, reduction of one or more adverse symptom associated with a tumor, an increase in the overall survival time of a subject having a tumor, or a combination of the above.

The methods described herein can comprise administering a trivalent and trispecific antibody construct to a subject in need thereof. An antibody construct can be administered to a subject by any appropriate route of administration. As will be appreciated by the person of skill in the art, the route and/or mode of administration can vary depending upon the desired therapeutic results. In various embodiments, antibody constructs of this disclosure can be administered by systemic administration or local administration. Local administration can be at the site of a tumor or into a tumor draining lymph node. Generally, the antibody constructs can be administered by parenteral administration, for example, by intravenous, intramuscular, intradermal, intraperitoneal, subcutaneous, or spinal administration, such as by injection or infusion.

A treatment (e.g., of a cancer in a subject) can be achieved by administration of a therapeutically effective amount of a trivalent and trispecific antibody construct to a subject in need thereof. A “therapeutically effective amount,” as used herein, generally refers to an amount of an antibody construct described herein that is effective, at dosages and for periods of time necessary, to achieve a desired therapeutic result. A therapeutically effective amount can vary according to factors such as the disease state, age, sex, and weight of the subject. A therapeutically effective amount is also one in which any potential toxic or detrimental effects of the antibody constructs are outweighed by the therapeutically beneficial effects. “Sufficient amount” generally refers to an amount sufficient to produce a desired effect, e.g., an amount sufficient to generate an anti-tumor immune response to a target (e.g., tumor) cell or tissue, e.g., by engaging an immune cell (e.g., T cell) using a trivalent and trispecific antibody construct described herein.

A suitable dosage of a trivalent and trispecific antibody construct described herein can be determined by a skilled medical practitioner. The selected dosage level may depend upon a variety of pharmacokinetic factors including the activity (e.g., antigen affinity (ies)) of the particular antibody construct employed, the route of administration, the time of administration, the rate of excretion of the construct, the duration of the treatment, other drugs, compounds and/or materials used in combination with the antibody construct, e.g., anti-cancer agents, the age, sex, weight, condition, general health and prior medical history of the subject being treated, and like factors well known in the medical arts.

In some embodiments, a method of treating a disease (e.g., a cancer) in a subject comprises administering a second active ingredient (e.g., another protein or small molecule) in addition to an antibody construct described herein. Such second active ingredient can be administered simultaneously or sequentially with an antibody construct dependent upon the condition to be treated.

In some embodiments, the present disclosure relates to a method of eliciting an anti-tumor immune response in a cell population comprising immune cells expressing CD3 and CD28 and tumor cells expressing MSLN and/or Cldn18.2, the method comprising contacting the cell population with an effective amount of a trivalent and trispecific antibody construct of the present disclosure. In some embodiments, such trivalent and trispecific antibody construct binds CD3 and CD28 on one or more immune cell(s) and MSLN or Cldn18.2 on the tumor cell, thereby forming a TCR-independent immune synapse, and comprises: (i) a Fab domain capable of binding either CD3 or CD28 on a first immune cell; (ii) a first scFv domain capable of binding either CD3 or CD28, wherein, however, the Fab domain and the first scFv domain do not bind the same antigen, (iii) a second scFv domain capable of binding either MSLN or Cldn18.2 on the tumor cell; and (iv) a heterodimeric Fc domain comprising a first Fc polypeptide and a second Fc polypeptide, wherein: (a) the Fab domain is coupled to the N-terminus of either the first of the second Fc polypeptide, (b) the first scFv domain is coupled to either an N-terminus of the Fab domain, the N-terminus of the first or second Fc polypeptide, the C-terminus of the Fab light chain, or the C-terminus of the first Fc polypeptide, and (c) the second scFv domain is coupled either to an N-terminus of the Fab domain, or the N-terminus of the first or second Fc polypeptide.

In some embodiments, the present disclosure relates to a method of inhibiting the proliferation of tumor cells expressing MSLN and/or Cldn.18.2 in a cell population comprising the tumor cells and immune cells expressing CD3 and CD28, the method comprising contacting the cell population with an effective amount of a trivalent and trispecific antibody construct of the present disclosure. In some embodiments, such trivalent and trispecific antibody construct binds CD3 and CD28 on one or more immune cell(s) and MSLN or Cldn18.2 on the tumor cell, thereby forming a TCR-independent immune synapse, and comprises: (i) a Fab domain capable of binding cither CD3 or CD28 on a first immune cell; (ii) a first scFv domain capable of binding either CD3 or CD28, wherein, however, the Fab domain and the first scFv domain do not bind the same antigen, (iii) a second scFv domain capable of binding either MSLN or Cldn18.2 on the tumor cell; and (iv) a heterodimeric Fc domain comprising a first Fc polypeptide and a second Fc polypeptide, wherein: (a) the Fab domain is coupled to the N-terminus of either the first of the second Fc polypeptide, (b) the first scFv domain is coupled to either an N-terminus of the Fab domain, the N-terminus of the first or second Fc polypeptide, the C-terminus of the Fab light chain, or the C-terminus of the first Fc polypeptide, and (c) the second scFv domain is coupled either to an N-terminus of the Fab domain, or the N-terminus of the first or second Fc polypeptide.

In some embodiments, the present disclosure relates to a method of killing tumor cells expressing MSLN and/or Cldn.18.2, such method comprising contacting a cell population comprising the tumor cells and immune cells expressing CD3 and CD28 with an effective amount of a trivalent and trispecific antibody construct of the present disclosure. In various embodiments, such trivalent and trispecific antibody construct binds CD3 and CD28 on one or more immune cell(s) and MSLN or Cldn18.2 on the tumor cell, thereby forming a TCR-independent immune synapse, and comprises: (i) a Fab domain capable of binding either CD3 or CD28 on a first immune cell; (ii) a first scFv domain capable of binding cither CD3 or CD28, wherein, however, the Fab domain and the first scFv domain do not bind the same antigen, (iii) a second scFv domain capable of binding either MSLN or Cldn18.2 on the tumor cell; and (iv) a heterodimeric Fc domain comprising a first Fc polypeptide and a second Fc polypeptide, wherein: (a) the Fab domain is coupled to the N-terminus of either the first of the second Fc polypeptide, (b) the first scFv domain is coupled to cither an N-terminus of the Fab domain, the N-terminus of the first or second Fc polypeptide, the C-terminus of the Fab light chain, or the C-terminus of the first Fc polypeptide, and (c) the second scFv domain is coupled either to an N-terminus of the Fab domain, or the N-terminus of the first or second Fc polypeptide.

In any of the methods described herein, the immune cells comprise or consist of one or more types of T cells.

As described herein, in various embodiments, an antibody construct described herein can be administered to a subject in need thereof, for example, a subject having cancer, in order to modulate an immune response in the subject. The immune response that can be modulated using an antibody construct of this disclosure can be an anti-tumor immune response in the subject, e.g., in various embodiments, such modulated immune response can occur locally at a tumor site. Thus, in certain embodiments, an antibody construct described herein can initiate and/or upregulate a local immune response, e.g., an anti-tumor response of a subject's immune system in order to elicit a localized cytotoxic effect against the tumor at the tumor site.

In various embodiments, an antibody construct described herein, e.g., a trivalent and trispecific antibody construct capable of monovalent binding to CD3 (e.g., via one scFv or Fab domain), monovalent binding of CD28 (e.g., via one scFv or Fab domain), and monovalent binding to MSLN or Cldn18.2 (e.g., via an scFv domain), can have a broader therapeutic window, compared to comparable conventional molecules, that can allow administration of higher doses of the herein described constructs, leading potentially to increased anti-tumor effects without inducing side effects and/or off-target. Such broader therapeutic window can be due to certain properties of the antibody constructs described herein, including higher ratios of anti-tumor activity compared to cytokine induction, i.e., higher tumor cell killing activities can be achieved at lower cytokine induction levels.

In some embodiments, the present disclosure relates to a method of inhibiting the growth of a MSLN and/or Cldn 18.2-expressing tumor and/or reducing the volume of the tumor in a subject in need thereof, the method comprising administering to the subject an effective amount of a trivalent and trispecific antibody construct of the present disclosure. In various embodiments, such in vivo anti-tumor is elicited by the trivalent and trispecific antibody construct that binds (e.g., simultaneously) CD3 and CD28 on one or more the immune cell(s) and MSLN or Cldn 18.2 on the tumor cell and comprises: (i) a Fab domain capable of binding either CD3 or CD28 on a first immune cell; (ii) a first scFv domain capable of binding either CD3 or CD28, wherein, however, the Fab domain and the first scFv domain do not bind the same antigen, (iii) a second scFv domain capable of binding either MSLN or Cldn 18.2 on the tumor cell; and (iv) a heterodimeric Fc domain comprising a first Fc polypeptide and a second Fc polypeptide, wherein: (a) the Fab domain is coupled to the N-terminus of either the first of the second Fc polypeptide, (b) the first scFv domain is coupled to either an N-terminus of the Fab domain, the N-terminus of the first or second Fc polypeptide, the C-terminus of the Fab light chain, or the C-terminus of the first Fc polypeptide, and (c) the second scFv domain is coupled either to an N-terminus of the Fab domain, or the N-terminus of the first or second Fc polypeptide.

In various embodiments, and as further described herein, inhibition of tumor growth and/or a reduction in tumor volume in a subject can be elicited by simultaneous binding of the antibody construct to CD3 and CD28 on one or more immune cell(s) and to MSLN or Cldn18.2 on a tumor cell and formation of a TCR-independent artificial immune synapse within a tumor environment in the subject, thereby eliciting an anti-tumor cytotoxic effect mediated by the activated immune cell and directed against the tumor cell. The immune cell can be a T cell.

In certain embodiments of the methods described herein, the trivalent and trispecific antibody constructs targets MSLN, and hence binds MSLN-positive tumor cells.

In other embodiments of the methods described herein, the trivalent and trispecific antibody constructs targets Cldn18.2, and hence binds Cldn18.2-positive tumor cells.

In various embodiments of the methods described herein, the trivalent and trispecific antibody constructs binds CD3 and CD28 on the same immune cell (e.g., T cell). In other embodiments, the trivalent and trispecific antibody constructs binds CD3 on a first immune cell (e.g., T cell) and CD28 on a second immune cell (e.g., T cell), wherein the first and the second immune cells are different cells.

In various embodiments of the methods described herein, the subject is a rodent, a non-human primate, or a human.

In further embodiments, and in relation to a method described herein, administration of a sufficient amount of a trivalent and trispecific antibody construct to a subject in need thereof can provide one or more of the following to activate or upregulate an immune response in the subject: (i) modulation of T-cell receptor signaling, (ii) modulation of T-cell activation, (iii) modulation of pro-inflammatory cytokines, (iv) modulation of interferon-γ (IFNγ) production by T cells, (v) modulation of T-cell suppression, (vi) modulation of M2-type tumor associated macrophages (TAM) or myeloid-derived suppressor cell (MDSC) survival and/or differentiation, and/or (vii) modulation of cytotoxic or cytostatic effects on cells, e.g., cancer cells.

In some embodiments, the present disclosure relates to methods of modulating an immune response in a cell or in a subject using one or more of the trivalent and trispecific antibody construct(s) of the present disclosure, wherein such modulation can comprise one or more of (i) immune cell activation, (ii) stimulation of T-cell receptor signaling, (iii) stimulation of antibody-dependent cellular cytotoxicity (ADCC), (iv) T cell-dependent cytotoxicity (TDCC), (v) cell-dependent cytotoxicity (CDC), (vi) antibody-dependent cellular phagocytosis (ADCP), and combinations of the above. As described herein, in certain embodiments, an antibody construct activates T effector cells. In some embodiments, and as demonstrated herein, an antibody construct increases production of one or more cytokines and/or signalling molecules, such as GM-CSF, TNFα, MIP-1β, IFN-γ, IL-2, IL-12, IL-17, IL-21 and/or C-X-C motif ligand 13 (CXCL13) by T effector cells.

As described herein, in various embodiments, a trivalent and trispecific antibody construct of the present disclosure comprises an Fc domain comprising a first and a second Fc polypeptide, wherein one or more of the Fc polypeptides can comprise a modified CH2 domain (e.g., compared to a WT domain) that comprises one or more amino acid modifications that can result in a decrease or elimination of binding of the Fc domain to one or more, or to all of the Fcγ receptors (also referred to herein as an Fc “knock-out” or “KO” variant).

### C. Experimental Methods

In some embodiments, the present disclosure relates to experimental methods for analyzing and/or detecting a trivalent and trispecific antibody construct of the present disclosure. Such methods can be used, for example, to assess in vitro and/or in vivo properties of such antibody construct such as its pharmacokinetic (PK) and pharmacodynamic (PD) properties. Other properties and characteristics of an antibody construct can be evaluated such as its stability under certain conditions (e.g., temperature, pH, etc.), its solubility, or its behaviour in the presence of certain other chemical components such as other proteins or cells.

Specific binding of a trivalent and trispecific antibody construct described herein to an antigen (e.g., CD3, CD28, TAA) can be measured, for example, through an enzyme-linked immunosorbent assay (ELISA), a surface plasmon resonance (SPR) technique (employing, for example, a BIAcore instrument) (Liljeblad et al., 2000, Glyco J, 17:323-329), or a traditional binding assay (Heeley, 2002, Endocr Res, 28:217-229). In certain embodiments, specific binding is defined as the extent of binding to an unrelated protein being less than about 10% of the binding to the target antigen (e.g., CD3, CD28, TAA, etc.) as measured by SPR, for example. In certain embodiments, specific binding of an antibody construct herein for a particular antigen, or an epitope is defined by a dissociation constant (K_{D}) of ≤1 μM, for example, ≤500 nM, ≤200 nM, ≤100 nM, ≤50 nM, ≤25 nM, or ≤5 nM. In some embodiments, specific binding of an antibody construct for a particular antigen or an epitope is defined by a dissociation constant (K_{D}) from about 10⁻⁶ M to about 10⁻¹⁰ M, from about 10⁻⁶ M to about 10⁻⁹ M, from about 10⁻⁸ M to about 10⁻¹⁰ M, or from about 10⁻⁷ M to about 10⁻⁹ M.

Additional experimental methods are described in, e.g., EXAMPLES 1-37 herein.

### VII. CERTAIN EMBODIMENTS OF THE DISCLOSURE

Certain embodiments of the present disclosure relate to embodiments 1-393 below, as well as to any combination of embodiments 1-393.

Embodiment 1. An antibody construct, comprising: (i) a Fab domain capable of binding a first antigen on a first cytotoxic effector cell; (ii) a first scFv domain and a second scFv domain, wherein one of the scFv domains is capable of binding a second antigen on a second cytotoxic effector cell and the other of the scFv domains is capable of binding a tumor-associated antigen (TAA), and (iii) a Fc domain comprising a first Fc polypeptide and a second Fc polypeptide, wherein: (a) the first and second antigens are different, (b) the Fab domain is coupled to the N-terminus of the first Fc polypeptide, and (c) each of the first and second scFv domains is independently coupled to an N-terminus of the Fab domain, a C-terminus of the Fab domain, or the N-terminus of the second Fc polypeptide.

Embodiment 2. The antibody construct of embodiment 1, wherein the first antigen is cluster of differentiation 3 (CD3) or cluster of differentiation 28 (CD28).

Embodiment 3. The antibody construct of embodiment 1 or embodiment 2, wherein the second antigen is CD3 or CD28.

Embodiment 4. The antibody construct of any one of embodiments 1-3, wherein the first antigen is CD28, and the second antigen is CD3.

Embodiment 5. The antibody construct of any one of embodiments 1-3, wherein the first antigen is CD3, and the second antigen is CD28.

Embodiment 6. An antibody construct, comprising: (i) a Fab domain capable of binding a CD3 on a first cytotoxic effector cell; (ii) a first scFv domain and a second scFv domain, wherein one of the scFv domains is capable of binding CD28 on a second cytotoxic effector cell and the other of the scFv domains is capable of binding a tumor-associated antigen (TAA), and (iii) a Fc domain comprising a first Fc polypeptide and a second Fc polypeptide, wherein: (a) the Fab domain is coupled to the N-terminus of the first Fc polypeptide, and (b) each of the first and second scFv domains is independently coupled to an N-terminus of the Fab domain, a C-terminus of the Fab domain, the C-terminus of one of the Fc polypeptide, or the N-terminus of the second Fc polypeptide.

Embodiment 7. An antibody construct, comprising: (i) a Fab domain capable of binding a CD28 on a first cytotoxic effector cell; (ii) a first scFv domain and a second scFv domain, wherein one of the scFv domains is capable of binding CD3 on a second cytotoxic effector cell and the other of the scFv domains is capable of binding a tumor-associated antigen (TAA), and (iii) a Fc domain comprising a first Fc polypeptide and a second Fc polypeptide, wherein: (a) the Fab domain is coupled to the N-terminus of the first Fc polypeptide, and (b) each of the first and second scFv domains is independently coupled to an N-terminus of the Fab domain, a C-terminus of the Fab domain, the C-terminus of one of the Fc polypeptide, or the N-terminus of the second Fc polypeptide.

Embodiment 8. The antibody construct of any one of embodiments 1-7, wherein the first scFv domain is coupled to an N-terminus of the Fab domain and the second scFv is coupled to the N-terminus of the second Fc polypeptide.

Embodiment 9. The antibody construct of embodiment 8, wherein the first scFv domain is coupled to the N-terminus of the V_{H} sequence of the heavy chain of the Fab domain.

Embodiment 10. The antibody construct of embodiment 8, wherein the first scFv domain is coupled to the N-terminus of the V_{L} sequence of the light chain of the Fab domain.

Embodiment 11. The antibody construct of embodiments 8 or 9, wherein the antibody construct comprises: a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, and (iii) the first Fc polypeptide; b) a second heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), and (ii) the second Fc polypeptide; and c) a light chain polypeptide comprising, from N-terminus to C-terminus: a Fab V_{L} sequence coupled to a Fab C_{L} sequence, wherein: the heavy chain Fab sequence and the light chain polypeptide form the Fab domain, and the first Fc polypeptide and the second Fc polypeptide form the Fc domain.

Embodiment 12. The antibody construct of any one of embodiments 1-7, wherein the first scFv domain is coupled to the C-terminus of the Fab domain and the second scFv domain is coupled to the N-terminus of the second Fc polypeptide.

Embodiment 13. The antibody construct of embodiment 12, wherein the antibody construct comprises: a) a first heavy chain polypeptide comprising, from N-terminus to C-terminus: (i) a heavy chain Fab sequence comprising a Fab V_{H} sequence coupled to a Fab C_{H1} sequence, and (ii) the first Fc polypeptide; b) a second heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), and (ii) the second Fc polypeptide; and c) a light chain polypeptide comprising, from N-terminus to C-terminus: (i) a Fab V_{L} sequence coupled to a Fab C_{L} sequence, and (ii) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), wherein: the heavy chain Fab sequence and the Fab sequence of the light chain polypeptide form the Fab domain, and the first Fc polypeptide and the second Fc polypeptide form the Fc domain.

Embodiment 14. The antibody construct of any one of embodiments 6-7, wherein the first scFv domain is coupled to an N-terminus of the Fab domain and the second scFv domain is coupled to the C-terminus of one of the Fc polypeptides.

Embodiment 15. The antibody construct of embodiment 14, wherein the first scFv domain is coupled to the N-terminus of the V_{H} domain of the Fab domain.

Embodiment 16. The antibody construct of embodiment 14, wherein the first scFv domain is coupled to the N-terminus of the V_{L} domain of the Fab domain.

Embodiment 17. The antibody construct of any one of embodiments 14-16, wherein the second scFv domain is coupled to the C-terminus of the first Fc polypeptide.

Embodiment 18. The antibody construct of any one of embodiments 14-16, wherein the second scFv domain is coupled to the C-terminus of the second Fc polypeptide.

Embodiment 19. The antibody construct of any one of embodiments 14-15 or 17, wherein the antibody construct comprises: a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, (iii) the first Fc polypeptide, and (iv) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}); b) a second heavy chain polypeptide comprising the second Fc polypeptide; and c) a light chain polypeptide comprising a Fab V_{L} sequence coupled to a Fab C_{L} sequence, wherein: the heavy chain Fab sequence and the light chain polypeptide form the Fab domain, and the first Fc polypeptide and the second Fc polypeptide form the Fc domain.

Embodiment 20. The antibody construct of any one of embodiments 14-15, or 18, wherein the antibody construct comprises: a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, and (iii) the first Fc polypeptide; b) a second heavy chain polypeptide comprising, from N-terminus to C-terminus: (i) the second Fc polypeptide, and (ii) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}); and c) a light chain polypeptide comprising a Fab V_{L} sequence coupled to a Fab C_{L} sequence, wherein: the heavy chain Fab sequence and the light chain polypeptide form the Fab domain, and the first Fc polypeptide and the second Fc polypeptide form the Fc domain.

Embodiment 21. The antibody construct of any one of embodiments 1-20, wherein the first scFv domain is capable of the binding the TAA.

Embodiment 22. The antibody construct of any one of embodiments 1-20, wherein the second scFv domain is capable of the binding the TAA.

Embodiment 23. An antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA), and (iii) a Fc domain comprising a first Fc polypeptide and a second Fc polypeptide, wherein: (a) the Fab domain is coupled to the N-terminus of the first Fc polypeptide, (b) the first scFv domain is coupled to the N-terminus of the V_{H} domain of the Fab domain, and (c) the second scFv domain is coupled to the N-terminus of the second Fc polypeptide.

Embodiment 24. An antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA), an (iii) a Fc domain comprising a first Fc polypeptide and a second Fc polypeptide, wherein: (a) the Fab domain is coupled to the N-terminus of the first Fc polypeptide, (b) the first scFv domain is coupled to the N-terminus of the V_{H} domain of the Fab domain, and (c) the second scFv domain is coupled to the N-terminus of the second Fc polypeptide.

Embodiment 25. An antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA), and (iii) a Fc domain comprising a first Fc polypeptide and a second Fc polypeptide, wherein: (a) the Fab domain is coupled to the N-terminus of the first Fc polypeptide, (b) the first scFv domain is coupled to the C-terminus of the C_{L} domain of the Fab domain, and (c) the second scFv domain is coupled to the N-terminus of the second Fc polypeptide.

Embodiment 26. An antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA), and (iii) a Fc domain comprising a first Fc polypeptide and a second Fc polypeptide, wherein: (a) the Fab domain is coupled to the N-terminus of the first Fc polypeptide, (b) the first scFv domain is coupled to the C-terminus of the C_{L} domain of the Fab domain, and (c) the second scFv domain is coupled to the N-terminus of the second Fc polypeptide.

Embodiment 27. An antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA), and (iii) a Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the second Fc polypeptide.

Embodiment 28. An antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA), and (iii) a Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the second Fc polypeptide.

Embodiment 29. An antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA), and (iii) a Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the V_{H} domain of the Fab domain.

Embodiment 30. An antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA), and (iii) a Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the V_{H} domain of the Fab domain.

Embodiment 31. The antibody construct of any one of embodiments 1-30, wherein the first scFv domain has the domain structure, from N- to C-terminus, of: V_{H}-V_{L}.

Embodiment 32. The antibody construct of any one of embodiments 1-30, wherein the first scFv domain has the domain structure, from N- to C-terminus, of: V_{L}-V_{H}.

Embodiment 33. The antibody construct of any one of embodiments 1-32, wherein the second scFv domain has the domain structure, from N- to C-terminus, of: V_{H}-V_{L}.

Embodiment 34. The antibody construct of any one of embodiments 1-32, wherein the second scFv domain has the domain structure, from N- to C-terminus, of: V_{L}-V_{H}.

Embodiment 35. The antibody construct of any one of embodiments 1-34, wherein the antibody construct comprises one or more linkers.

Embodiment 36. The antibody construct of embodiment 35, wherein the one or more linkers are peptide linkers that each comprise or consist of an amino acid sequence from 1 to about 50, from 2 to about 40, from 3 to about 30, or from 5 to about 25 consecutive amino acid residues in length.

Embodiment 37. The antibody construct of any one of embodiments 35-36, wherein the first scFv domain comprises a linker^{scFv1}.

Embodiment 38. The antibody construct of embodiment 37, wherein the linker^{scFv1} couples the N- or C-terminus of the V_{H} domain to the N- or C-terminus of the V_{L} domain and comprises or consists of an amino acid sequence having about 80%, 90%, or 100% sequence identity to the amino acid sequence set forth in SEQ ID NO: 104.

Embodiment 39. The antibody construct of any one of embodiments 1-11, 14-24, or 29-30, wherein the first scFv domain or the second scFv domain is coupled to the Fab domain via a linker^{scFv-Fab}.

Embodiment 40. The antibody construct of embodiment 39, wherein the linker^{scFv-Fab} comprises or consists of an amino acid sequence having about 60%, 80%, or 100% sequence identity to the amino acid sequence set forth in SEQ ID NO: 105.

Embodiment 41. The antibody construct of any one of embodiments 12-13 or 25-26, wherein the first scFv domain is coupled to the C-terminus of the first Fc polypeptide via a linker^{Fc-scFv}.

Embodiment 42. The antibody construct of embodiment 41, wherein the linker^{Fc-scFv} comprises or consists of an amino acid sequence having about 60%, 80%, or 100% sequence identity to the amino acid sequence set forth in SEQ ID NO: 105.

Embodiment 43. The antibody construct of any one of embodiments 1-42, wherein the C-terminus of the heavy chain of the Fab domain is coupled to the N-terminus of the first Fc polypeptide via a linker^{Fab-Fc}.

Embodiment 44. The antibody construct of embodiment 43, wherein the linker^{Fab-Fc} comprises or consists of an amino acid sequence having about 80%, 90%, or 100% sequence identity to the amino acid sequence set forth in SEQ ID NO: 108.

Embodiment 45. The antibody construct of any one of embodiments 1-13 or 23-28, wherein the C-terminus of the first scFv domain or the second scFv domain is coupled to N-terminus of the second Fc polypeptide via a linker^{scFv-Fc}.

Embodiment 46. The antibody construct of embodiment 45, wherein the linker^{scFv-Fc} comprises or consists of an amino acid sequence having about 80%, 90%, or 100% sequence identity to the amino acid sequence set forth in SEQ ID NO: 111 or SEQ ID NO: 112.

Embodiment 47. The antibody construct of any one of embodiments 1-46, wherein the Fab domain that is capable of binding the first antigen on the first cytotoxic effector cell comprises a heavy chain constant domain (C_{H1}) comprising or consisting of an amino acid sequence having at least about 90%, 95%, 97%, 99%, or 100% sequence identity to the amino acid sequence set forth in SEQ ID NO: 107.

Embodiment 48. The antibody construct of any one of embodiments 2-47, wherein the Fab domain or the first scFv domain is capable of binding CD28 with a dissociation constant from about 10 nM to about 500 nM, from about 20 nM to about 600 nM, from about 20 nM to about 250 nM, from about 20 nM to about 150 nM, from about 20 nM to about 100 nM, or from about 20 nM to about 50 nM.

Embodiment 49. The antibody construct of any one of embodiments 2-48, wherein the Fab domain or the first scFv domain that is capable of binding CD28 comprises a V_{H} sequence comprising a HCDR1 having the sequence SX₁GVH (SEQ ID NO: 302), a HCDR2 having the sequence VIWX₂GGX₃TNFNSALMS (SEQ ID NO: 306), and a HCDR3 having the sequence DRAX₄GX₅YX₆X₇AMDY (SEQ ID NO: 312) and a V_{L} sequence comprising a LCDR1 having the sequence RASESVEYYX₈TSLMQ (SEQ ID NO: 315), a LCDR2 having the sequence AASX₉ VX₁₀S (SEQ ID NO: 319), and a LCDR3 having the sequence QQSRKVPFT (SEQ ID NO: 320), and wherein X₁=Y, A; X₂=P, A; X₃=G, S; X₄=S, Y; X₅=N, A; X₆=L, N; X₇=S, Y; X₈=G, V; X₉=N, A; and X₁₀=E, D.

Embodiment 50. The antibody construct of any one of embodiments 2-49, wherein the Fab domain or the first scFv domain that is capable of binding CD28 comprises a V_{H} sequence having at least about 90%, 95%, 97%, 99%, or 100% sequence identity to the amino acid sequence set forth in SEQ ID NO: 106, and a V_{L} sequence having at least about 90%, 95%, 97%, 99%, or 100% sequence identity to the amino acid sequence set forth in SEQ ID NO: 116.

Embodiment 51. The antibody construct of any one of embodiments 48-50, wherein the anti-CD28 V_{H} sequence comprises or consists of an amino acid sequence selected from the group consisting of SEQ ID NOs: 201, 203-208, and 210, and the anti-CD28 V_{L} sequence comprises or consists of an amino acid sequence selected from the group consisting of SEQ ID NOs: 200, 202, and 209.

Embodiment 52. The antibody construct of any one of embodiments 2-48, wherein the anti-CD28 V_{H} sequence comprises the HCDR1 having the sequence SYGVH (SEQ ID NO: 300), the HCDR2 having the sequence VIWPGGGTNFNSALMS (SEQ ID NO: 303), and the HCDR3 having the sequence DRAYGNYLYAMDY (SEQ ID NO: 307) and the anti-CD28 V_{L} sequence comprises the LCDR1 having the sequence RASESVEYYVTSLMQ (SEQ ID NO: 313), the LCDR2 having the sequence AASNVDS (SEQ ID NO: 316), and the LCDR3 having the sequence QQSRKVPFT (SEQ ID NO: 320).

Embodiment 53. The antibody construct of any one of embodiments 2-4, 7, 24, 26, 28 or 30, wherein the anti-CD28 Fab domain comprises a heavy chain comprising a V_{H} sequence having at least about 90%, 95%, 97%, 99%, or 100% sequence identity to the amino acid sequence set forth in SEQ ID NO: 106, coupled to a C_{H1} sequence having at least about 90%, 95%, 97%, 99%, or 100% sequence identity to the amino acid sequence set forth in SEQ ID NO: 107, and a light chain comprising a V_{L} sequence having at least about 90%, 95%, 97%, 99%, or 100% sequence identity to the amino acid sequence set forth in SEQ ID NO: 116, coupled to a C_{L} sequence having at least about 90%, 95%, 97%, 99%, or 100% sequence identity to the amino acid sequence set forth in SEQ ID NO: 117.

Embodiment 54. The antibody construct of any one of embodiments 3-6, 23, 25, 27 or 29, wherein the first scFv domain or second scFv domain that is capable of binding CD28 comprises, either from N- to C-terminus or C- to N-terminus, a V_{H} sequence having at least about 90%, 95%, 97%, 99%, or 100% sequence identity to the amino acid sequence set forth in SEQ ID NO: 106, coupled to a V_{L} sequence having at least about 90%, 95%, 97%, 99%, or 100% sequence identity to the amino acid sequence set forth in SEQ ID NO: 116.

Embodiment 55. The antibody construct of embodiment 54, wherein the first scFv domain or second scFv domain that is capable of binding CD28 comprises or consists of an amino acid sequence having at least about 90%, 95%, 97%, 99%, or 100% sequence identity to the amino acid sequence set forth in SEQ ID NO: 119.

Embodiment 56. The antibody construct of any one of embodiments 2-55, wherein the Fab domain or the first or second scFv domain is capable of binding CD3 with a dissociation constant from about 20 nM to about 200 nM, from about 30 nM to about 150 nM, from about 40 nM to about 100 nM, or from 50 nM to about 80 nM.

Embodiment 57. The antibody construct of any one of embodiments 2-56, wherein the Fab domain or the first or second scFv domain that is capable of binding CD3 comprises the CDR sequences of the V_{H} sequence as set forth in SEQ ID NOS: 321-323, and the CDR sequences of the V_{L} sequence as set forth in SEQ ID NO: 324-326.

Embodiment 58. The antibody construct of any one of embodiments 2-57, wherein the Fab domain or the first or second scFv domain that is capable of binding CD3 comprises a V_{H} sequence comprising a HCDR1 having the sequence GVTFNYYG (SEQ ID NO: 321), a HCDR2 having the sequence ITSSGGRI (SEQ ID NO: 322), and a HCDR3 having the sequence TLDGRDGWVAY (SEQ ID NO: 323) and a V_{L} sequence comprising a LCDR1 having the sequence TGNIGSNY (SEQ ID NO: 324), a LCDR2 having the sequence RND (SEQ ID NO: 325), and a LCDR3 having the sequence QSYSSGFI (SEQ ID NO: 326).

Embodiment 59. The antibody construct of any one of embodiments 2-58, wherein the Fab domain or the first or second scFv domain that is capable of binding CD3 comprises a V_{H} sequence having at least about 90%, 95%, 97%, 99%, or 100% sequence identity to the amino acid sequence set forth in SEQ ID NO: 102, and a V_{L} sequence having at least about 90%, 95%, 97%, 99%, or 100% sequence identity to the amino acid sequence set forth in SEQ ID NO: 103.

Embodiment 60. The antibody construct of any one of embodiments 2-3, 5-6, 23, 25, 27 or 29, wherein the anti-CD3 Fab domain comprises a heavy chain comprising a V_{H} sequence having at least about 90%, 95%, 97%, 99%, or 100% sequence identity to the amino acid sequence set forth in SEQ ID NO: 102, coupled to a C_{H1} sequence having at least about 90%, 95%, 97%, 99%, or 100% sequence identity to the amino acid sequence set forth in SEQ ID NO: 107, and a light chain comprising a V_{L} sequence having at least about 90%, 95%, 97%, 99%, or 100% sequence identity to the amino acid sequence set forth in SEQ ID NO: 103, coupled to a C_{L} sequence having at least about 90%, 95%, 97%, 99%, or 100% sequence identity to the amino acid sequence set forth in SEQ ID NO: 121.

Embodiment 61. The antibody construct of any one of embodiments 3-4, 7, 24, 26, 28 or 30, wherein the first or second scFv domain that is capable of binding CD3 comprises, either from N- to C-terminus or C- to N-terminus, a V_{H} sequence having at least about 90%, 95%, 97%, 99%, or 100% sequence identity to the amino acid sequence set forth in SEQ ID NO: 106, coupled to a V_{L} sequence having at least about 90%, 95%, 97%, 99%, or 100% sequence identity to the amino acid sequence set forth in SEQ ID NO: 116.

Embodiment 62. The antibody construct of embodiment 61, the first or second scFv domain that is capable of binding CD3 comprises or consists of an amino acid sequence having at least about 90%, 95%, 97%, 99%, or 100% sequence identity to the amino acid sequence set forth in SEQ ID NO: 101.

Embodiment 63. The antibody construct of any one of embodiments 1-62, wherein the first and second antigens are on different cytotoxic effector cells.

Embodiment 64. The antibody construct of any one of embodiments 1-62, wherein the first and second antigens are on the same cytotoxic effector cell.

Embodiment 65. The antibody construct of any one of embodiments 1-64, wherein the first and second antigens are on a T cell.

Embodiment 66. The antibody construct of any one of embodiments 1-65, wherein the TAA is Claudin 18.2 (Cldn18.2) or mesothelin (MSLN).

Embodiment 67. The antibody construct of embodiment 66, wherein the TAA is Cldn18.2.

Embodiment 68. The antibody construct of embodiment 66, wherein the TAA is MSLN.

Embodiment 69. The antibody construct of embodiment 67, wherein the first or second scFv domain capable of binding Cldn18.2 comprises the CDR sequences of the V_{H} sequence as set forth in SEQ ID NOS: 333-335, and the CDR sequences of the V_{L} sequence as set forth in SEQ ID NO: 336-338.

Embodiment 70. The antibody construct of embodiment 69, wherein the first or second scFv domain capable of binding Cldn18.2 comprises a V_{H} sequence comprising a HCDR1 having the sequence SNPMI (SEQ ID NO: 333), a HCDR2 having the sequence IIDTDGSTYYADWAKG (SEQ ID NO: 334), and a HCDR3 having the sequence RLHGSSNGYYDDL (SEQ ID NO: 335) and a V_{L} sequence comprising a LCDR1 having the sequence QASQSIYSYLS (SEQ ID NO: 336), a LCDR2 having the sequence KASTLAS (SEQ ID NO: 337), and a LCDR3 having the sequence QQGYTVTNVDKNT (SEQ ID NO: 338).

Embodiment 71. The antibody construct of embodiment 69, wherein the V_{H} sequence comprises or consists of an amino acid sequence having at least about 90%, 95%, 97%, 99%, or 100% sequence identity to the amino acid sequence set forth in SEQ ID NO: 127, and the V_{L} sequence comprises or consists of an amino acid sequence having at least about 90%, 95%, 97%, 99%, or 100% sequence identity to the amino acid sequence set forth in SEQ ID NO: 128.

Embodiment 72. The antibody construct of embodiment 68, wherein the first or second scFv domain capable of binding MSLN comprises the CDR sequences of the V_{H} sequence as set forth in SEQ ID NOS: 327-329, and the CDR sequences of the V_{L} sequence as set forth in SEQ ID NO: 330-332.

Embodiment 73. The antibody construct of embodiment 72, wherein the first or second scFv domain capable of binding MSLN comprises a V_{H} sequence comprising a HCDR1 having the sequence GYTMN (SEQ ID NO: 327), a HCDR2 having the sequence LITPYSGASSYAQKFQG (SEQ ID NO: 328), and a HCDR3 having the sequence GGYDGRGFDY (SEQ ID NO: 329) and a V_{L} sequence comprising a LCDR1 having the sequence SASSSVSYMH (SEQ ID NO: 330), a LCDR2 having the sequence DTSKLAS (SEQ ID NO: 331), and a LCDR3 having the sequence QQWSGHPLT (SEQ ID NO: 332).

Embodiment 74. The antibody construct of any one of embodiments 1-73, wherein the first Fc polypeptide and the second Fc polypeptide of the Fc domain each comprise or consist of a CH2 sequence and a CH3 sequence.

Embodiment 75. The antibody construct of embodiment 74, wherein at least one of the CH2 sequences of the first and second Fc polypeptide comprises one or more amino acid modifications when compared to an unmodified wildtype IgG1 or IgG4 CH2 sequence.

Embodiment 76. The antibody construct of embodiment 75, wherein both CH2 sequences of the first and second Fc polypeptide comprise the one or more amino acid modifications.

Embodiment 77. The antibody construct of embodiment 75-76, wherein the one or more amino acid modifications to the CH2 sequences reduce or ablate interactions of the Fc domain with one or more Fc receptors.

Embodiment 78. The antibody construct of any one of embodiments 74-77, wherein the CH2 sequence of both the first and the second Fc polypeptide comprises or consists of an amino acid sequence having at least about 90%, 95%, 97%, 99%, or 100% sequence identity to the amino acid sequence set forth in SEQ ID NO: 109.

Embodiment 79. The antibody construct of any one of embodiments 74-78, wherein at least one of the CH3 sequences of the first and second Fc polypeptide comprises one or more amino acid modifications when compared to an unmodified wildtype IgG1 or IgG4 CH3 sequence, and wherein the first and second Fc polypeptides have different amino acid sequences, e.g., a relative amino acid sequence identity of about 97%, 98%, or 99%, and form a heterodimeric Fc domain.

Embodiment 80. The antibody construct of embodiment 79, wherein both CH3 sequences of the first and second Fc polypeptide comprise one or more amino acid modifications that promote preferential pairing of the first and second Fc polypeptide to form a heterodimeric Fc domain compared to the formation of a corresponding homodimeric Fc domain.

Embodiment 81. The antibody construct of any one of embodiments 79-80, wherein the CH3 sequence of one of the first and second Fc polypeptide comprises a set of amino acid substitutions selected from the group consisting of: L351Y_F405A_Y407V, T350V_L351Y_F405A_Y407V and T350V_L351Y_S400E_F405A_Y407V, and the CH3 sequence of the other Fc polypeptide comprises a set of amino acid substitutions selected from the group consisting of: T366L_K392M_T394W, T366L_K392L_T394W, T350V_T366L_K392L_T394W, T350V_T366L_K392M_T394W and T350V_T366L_N390R_K392M_T394W, and wherein the numbering of amino acid residues in the Fc polypeptides is according to the EU numbering system.

Embodiment 82. The antibody construct of any one of embodiments 74-81, wherein the CH3 sequence of the first Fc polypeptide comprises or consists of an amino acid sequence having at least about 90%, 95%, 97%, 99%, or 100% sequence identity to the amino acid sequence set forth in SEQ ID NO: 110.

Embodiment 83. The antibody construct of any one of embodiments 74-82, wherein the CH3 sequence of the second Fc polypeptide comprises or consists of an amino acid sequence having at least about 90%, 95%, 97%, 99%, or 100% sequence identity to the amino acid sequence set forth in SEQ ID NO: 114.

Embodiment 84. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 100 or 118, (ii) a second heavy chain polypeptide (H2) that comprises or consists of an amino acid sequence having at least about 90%, 95%, 97%, 99%, or 100% sequence identity to the amino acid sequence set forth in SEQ ID NO: 153 or 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115 or 120.

Embodiment 85. The antibody construct of embodiment 84, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 153, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 86. The antibody construct of embodiment 84, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 118, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

Embodiment 87. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 122, (ii) a second heavy chain polypeptide (H2) that comprises or consists of an amino acid sequence having at least about 90%, 95%, 97%, 99%, or 100% sequence identity to the amino acid sequence set forth in SEQ ID NO: 153, and (iii) a light chain polypeptide (L1) that comprises or consists of an amino acid sequence having at least about 90%, 95%, 97%, 99%, or 100% sequence identity to the amino acid sequence set forth in SEQ ID NO: 123.

Embodiment 88. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 124, (ii) a second heavy chain polypeptide (H2) that comprises or consists of an amino acid sequence having at least about 90%, 95%, 97%, 99%, or 100% sequence identity to the amino acid sequence set forth in SEQ ID NO: 153, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 89. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 124, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 90. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 129, (ii) a second heavy chain polypeptide (H2) that comprises or consists of an amino acid sequence having at least about 90%, 95%, 97%, 99%, or 100% sequence identity to the amino acid sequence set forth in SEQ ID NO: 153, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

Embodiment 91. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 122, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 123.

Embodiment 92. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 129, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

Embodiment 93. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 94. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 134, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 135.

Embodiment 95. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 130, (ii) a second heavy chain polypeptide (H2) that comprises or consists of an amino acid sequence having at least about 90%, 95%, 97%, 99%, or 100% sequence identity to the amino acid sequence set forth in SEQ ID NO: 131, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

Embodiment 96. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 132, (ii) a second heavy chain polypeptide (H2) that comprises or consists of an amino acid sequence having at least about 90%, 95%, 97%, 99%, or 100% sequence identity to the amino acid sequence set forth in SEQ ID NO: 133, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

Embodiment 97. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 136, (ii) a second heavy chain polypeptide (H2) that comprises or consists of an amino acid sequence having at least about 90%, 95%, 97%, 99%, or 100% sequence identity to the amino acid sequence set forth in SEQ ID NO: 131, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 98. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 137, (ii) a second heavy chain polypeptide (H2) that comprises or consists of an amino acid sequence having at least about 90%, 95%, 97%, 99%, or 100% sequence identity to the amino acid sequence set forth in SEQ ID NO: 138, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 99. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 139, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 100. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 140, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 101. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 141.

Embodiment 102. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 142.

Embodiment 103. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in any one of SEQ ID NOs: 143-149, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 104. The antibody construct of embodiment 1, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 150 or SEQ ID NO: 152.

Embodiment 105. The antibody construct of any one of embodiments 1-104, wherein the antibody construct is trivalent and trispecific and binds each antigen monovalently.

Embodiment 106. The antibody construct of any one of embodiments 1-105, wherein the construct has a binding affinity for the TAA of at least about 40 nM, 30 nM, 20 nM, 10 nM or 5 nM, or from about 40 nM to about 5 nM or from about 30 nM to about 10 nM.

Embodiment 107. The antibody construct of any one of embodiments 1-106, wherein the antibody construct has a melting temperature at Tm1, Tm2, and/or Tm3 that is within 10° C., 5° C., 2° C., or within 1° C. degree of that of a bivalent and monospecific IgG1 monoclonal antibody.

Embodiment 108. The antibody construct of any one of embodiments 1-107, wherein the antibody construct binds the cytotoxic effector cell that expresses CD3 and CD28 with an affinity from about 1 nM to about 500 pM.

Embodiment 109. The antibody construct of any one of embodiments 1-108, wherein the antibody construct binds the cytotoxic effector cell that expresses CD3 and CD28 with an affinity that is about 2-fold, 5-fold, 10-fold, 20-fold, 50-fold, 100-fold, or about 200-fold higher than that of a corresponding bispecific anti-CD3×TAA and/or anti-CD28×TAA antibody construct.

Embodiment 110. The antibody construct of any one of embodiments 1-109, wherein the antibody construct exhibits an IC₅₀ value from about 50 pM to about 0.01 pM, from about 25 pM to about 0.01 pM, from about from about 10 pM to about 0.05 pM, from about 10 pM to about 0.1 pM, from about 10 pM to about 1 pM, from about 5 pM to about 1 pM for killing TAA-expressing tumor cells that express at least about 100,000 TAA/cell by TDCC in the presence of the cytotoxic effector cell and using an E:T ratio of 2:1 and an incubation period of 72 hours.

Embodiment 111. The antibody construct of embodiment 110, wherein the antibody construct achieves a maximum killing of TAA-expressing tumor cells of at least about 60%, 65%, 70%, 75%, or 80%, 85%, or 90%, 100%, or from about 60% to about 100%, from about 70% to about 90%, or from about 75% to about 85%.

Embodiment 112. The antibody construct of any one of embodiments 1-111, wherein the antibody construct (i) is capable of inducing the production of one or more cytokines by the cytotoxic effector cell ranging from about 300 μg/mL to about 9000 μg/mL, when TAA-expressing cells expressing at least about 100,000 TAA/cell are present and using an E:T ratio of 2:1 and an incubation period of 72 hours, and/or (ii) has a stability of least about 95%, 96%, 97%, 98%, or 99%, i.e., at least about 95%, 96%, 97%, 98%, or 99% is measured as being intact, after incubation of the antibody construct for 14 days at 40° C. using an aqueous solution or buffer system comprising sucrose, and as measured using, e.g., size-exclusion chromatography (SEC).

Embodiment 113. A pharmaceutical composition comprising the antibody construct of any one of embodiments 1-112, and a pharmaceutically acceptable carrier, excipient, diluent, or combination thereof.

Embodiment 114. A nucleic acid molecule or a set of nucleic acid molecules encoding one or more, two or more, or three or more polypeptide chains that form the antibody construct of any one of embodiments 1-112.

Embodiment 115. A vector or a set of vectors comprising the nucleic acid molecule or the set of nucleic acid molecules of embodiment 114.

Embodiment 116. A method of producing an antibody construct of any one of embodiments 1-112, the method comprising: (a) obtaining a host cell culture comprising at least one host cell comprising one or more nucleic acid molecules encoding one or more, two or more, or three or more polypeptide chains that form the antibody construct; and (b) recovering the antibody construct from the host cell culture.

Embodiment 117. The method of embodiment 116, further comprising, subsequent to step (b), purifying the antibody construct.

Embodiment 118. A method of eliciting an anti-tumor immune response in a cell population comprising immune cells and tumor cells expressing a TAA, the method comprising contacting the cell population with an effective amount of the antibody construct of any one of embodiments 1-112, wherein the immune cells express the first and second antigen and the tumor cells express the TAA.

Embodiment 119. A method of inhibiting the proliferation of tumor cells expressing a TAA, the method comprising contacting a cell population comprising the tumor cells and immune cells with an effective amount of the antibody construct of any one of embodiments 1-112, wherein the immune cells express the first and second antigen and the tumor cells express the TAA.

Embodiment 120. A method of killing tumor cells expressing a TAA, the method comprising contacting a cell population comprising the tumor cells and immune cells with an effective amount of the antibody construct of any one of embodiments 1-112, wherein the immune cells express the first and second antigen and the tumor cells express the TAA.

Embodiment 121. The method of any one of embodiments 118-120, wherein the immune cells comprise T cells.

Embodiment 122. The method of any one of embodiments 118-121, wherein TAA is MSLN or Cldn18.2.

Embodiment 123. The method of any one of embodiments 118-122, wherein the antibody construct binds CD3 and CD28 on either one T cell or two different T cells, and the TAA on a tumor cell.

Embodiment 124. The method of embodiment 123, wherein the binding of the first and second antigen and the TAA forms a TCR-independent artificial immune synapse between the one or more immune cells and the tumor cell, thereby eliciting a cytotoxic immune response of the immune cell against the tumor cell.

Embodiment 125. The method of any one of embodiments 118-124, wherein the cell population is located in a subject.

Embodiment 126. A method for treating a cancer in a subject in need thereof, the method comprising administering to the subject an antibody construct of any one of embodiments 1-112.

Embodiment 127. The method of embodiment 126, further comprising eliciting a cytotoxic immune response against the cancer in the subject, thereby treating the cancer in the subject.

Embodiment 128. The method of any one of embodiments 125-127, wherein the subject is a rodent, a non-human primate, or a human.

Embodiment 129. An antibody construct of any one of embodiments 1-112 for use in the treatment of cancer.

Embodiment 130. Use of an antibody construct of any one of embodiments 1-112 in the manufacture of a medicament for the treatment of cancer.

Embodiment 131. An antibody construct, comprising a binding domain capable of binding CD28, wherein the binding domain comprises a V_{H} sequence comprising a HCDR1 having the sequence SX₁GVH (SEQ ID NO: 302), a HCDR2 having the sequence VIWX₂GGX₃TNFNSALMS (SEQ ID NO: 306), and a HCDR3 having the sequence DRAX₄GX₅YX₆X₇AMDY (SEQ ID NO: 312), and a V_{L} sequence comprising a LCDR1 having the sequence RASESVEYYX₈TSLMQ (SEQ ID NO: 315), a LCDR2 having the sequence AASX₉VX₁₀S (SEQ ID NO: 319), and a LCDR3 having the sequence QQSRKVPFT (SEQ ID NO: 320), having one or more of the following amino acid substitutions at the positions as identified in the CDR sequences: X₁: Y to A, X₂: P to A, X₃: G to S, X₄: S to Y, X₅: N to A, X₆: L to N, X₇: S to Y, X₈: G to V, X₉: N to A, or X₁₀: E to D.

Embodiment 132. The antibody construct of embodiment 131, wherein the binding domain comprises the substitution X₁: Y to A.

Embodiment 133. The antibody construct of any one of embodiments 131-132, wherein the binding domain comprises the substitution X₂: P to A.

Embodiment 134. The antibody construct of any one of embodiments 131-133, wherein the binding domain comprises the substitution X₃: G to S.

Embodiment 135. The antibody construct of any one of embodiments 131-134, wherein the binding domain comprises the substitution X₄: S to Y.

Embodiment 136. The antibody construct of any one of embodiments 131-135, wherein the binding domain comprises the substitution X₅: N to A.

Embodiment 137. The antibody construct of any one of embodiments 131-136, wherein the binding domain comprises the substitution X₆: L to N.

Embodiment 138. The antibody construct of any one of embodiments 131-137, wherein the binding domain comprises the substitution X₇: S to Y.

Embodiment 139. The antibody construct of any one of embodiments 131-138, wherein the binding domain comprises the substitution X₈: G to V.

Embodiment 140. The antibody construct of any one of embodiments 131-139, wherein the binding domain comprises the substitution X₉: N to A.

Embodiment 141. The antibody construct of any one of embodiments 131-140, wherein the binding domain comprises the substitution X₁₀: E to D.

Embodiment 142. The antibody construct of any one of embodiments 131-141, wherein the antibody construct has a binding affinity for CD28 that is reduced by about 1.5-fold to about 25-fold, by about 2.0-fold to about 20-fold, by about 3.0-fold to about 20-fold, or by about 5.0-fold to about 10-fold, when compared to the binding affinity of an antibody construct comprising an anti-CD28 binding domain that does not contain the one or more amino acid substitutions in one or more of the CDR sequences.

Embodiment 143. The antibody construct of embodiment 131, wherein the binding domain comprises a V_{H} sequence comprising or consisting of an amino acid sequence selected from the group consisting of SEQ ID NOs: 201, 203-208, and 210, and a V_{L} sequence comprising or consisting of an amino acid sequence selected from the group consisting of SEQ ID NOs: 200, 202, and 209.

Embodiment 144. An antibody construct, comprising a binding domain capable of binding Cldn18.2, wherein the binding domain comprises a V_{H} sequence comprising a HCDR1 having the sequence SNPMI (SEQ ID NO: 333), a HCDR2 having the sequence IIDTDGSTYYADWAKG (SEQ ID NO: 334), and a HCDR3 having the sequence RLHGSSNGYYDDL (SEQ ID NO: 335), and a V_{L} sequence comprising a LCDR1 having the sequence QASQSIYSYLS (SEQ ID NO: 336), a LCDR2 having the sequence KASTLAS (SEQ ID NO: 337), and a LCDR3 having the sequence QQGYTVTNVDKNT (SEQ ID NO: 338).

Embodiment 145. The antibody construct of embodiment 144, wherein the binding domain comprises a V_{H} sequence comprising or consisting of an amino acid sequence having at least about 90%, 95%, 97%, 99%, or 100% sequence identity to the amino acid sequence set forth in SEQ ID NO: 127, and a V_{L} sequence comprising or consisting of an amino acid sequence having at least about 90%, 95%, 97%, 99%, or 100% sequence identity to the amino acid sequence set forth in SEQ ID NO: 128.

Embodiment 146. The antibody construct of any one of embodiments 144-145, wherein the binding domain comprises a V_{H} sequence comprising or consisting of the amino acid sequence set forth in SEQ ID NO: 127, and a V_{L} sequence comprising or consisting of the amino acid sequence set forth in SEQ ID NO: 128.

Embodiment 147. An antibody construct, comprising: (i) a Fab domain capable of binding a first antigen on a first cytotoxic effector cell; (ii) a first scFv domain and a second scFv domain, wherein one of the scFv domains is capable of binding a second antigen on a second cytotoxic effector cell and the other scFv domain is capable of binding a tumor-associated antigen (TAA) on a tumor cell, and (iii) an Fc domain comprising a first Fc polypeptide and a second Fc polypeptide, wherein: (a) the first and second antigens on the first and second cytotoxic effector cells are different, (b) the Fab domain is coupled to the N-terminus of the first Fc polypeptide, and (c) the first and second scFv domains are independently coupled to either (i) an N-terminus of the Fab domain, (ii) a C-terminus of the Fab domain, or (iii) the N-terminus of the second Fc polypeptide.

Embodiment 148. The antibody construct of embodiment 147, wherein the first antigen on the first cytotoxic effector cell is cluster of differentiation 3 (CD3) or cluster of differentiation 28 (CD28).

Embodiment 149. The antibody construct of embodiment 147 or embodiment 148, wherein the second antigen on the second cytotoxic effector cell is CD3 or CD28.

Embodiment 150. The antibody construct of any one of embodiments 147-149, wherein the first antigen is CD28, and the second antigen is CD3.

Embodiment 151. The antibody construct of any one of embodiments 147-149, wherein the first antigen is CD3, and the second antigen is CD28.

Embodiment 152. An antibody construct, comprising: (i) a Fab domain capable of binding a CD3 on a first cytotoxic effector cell; (ii) a first scFv domain and a second scFv domain, wherein one of the scFv domains is capable of binding CD28 on a second cytotoxic effector cell and the other scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, and (b) the first and second scFv domains are independently coupled to either (i) an N-terminus of the Fab domain, (ii) a C-terminus of the Fab domain, (iii) the C-terminus of one of the Fc polypeptides, or (iv) the N-terminus of the second Fc polypeptide.

Embodiment 153. An antibody construct, comprising: (i) a Fab domain capable of binding a CD28 on a first cytotoxic effector cell; (ii) a first scFv domain and a second scFv domain, wherein one of the scFv domains is capable of binding CD3 on a second cytotoxic effector cell and the other scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, and (b) the first and second scFv domains are independently coupled to either (i) an N-terminus of the Fab domain, (ii) a C-terminus of the Fab domain, (iii) the C-terminus of one of the Fc polypeptides, or (iv) the N-terminus of the second Fc polypeptide.

Embodiment 154. The antibody construct of any one of embodiments 147-153, wherein the first scFv domain is coupled to an N-terminus of the Fab domain and the second scFv is coupled to the N-terminus of the second Fc polypeptide.

Embodiment 155. The antibody construct of embodiment 154, wherein the first scFv domain is coupled to the N-terminus of the V_{H} sequence of the heavy chain of the Fab domain.

Embodiment 156. The antibody construct of embodiment 154, wherein the first scFv domain is coupled to the N-terminus of the V_{L} sequence of the light chain of the Fab domain.

Embodiment 157. The antibody construct of embodiments 154 or 155, wherein the antibody construct comprises: a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, and (iii) the first Fc polypeptide; b) a second heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), and (ii) the second Fc polypeptide; and c) a light chain polypeptide comprising, from N-terminus to C-terminus: a Fab V_{L} sequence coupled to a Fab C_{L} sequence, wherein: the heavy chain Fab sequence and the light chain polypeptide associate to form the Fab domain, and the first Fc polypeptide and the second Fc polypeptide associate to form the Fc domain.

Embodiment 158. The antibody construct of any one of embodiments 147-153, wherein the first scFv domain is coupled to the C-terminus of the light chain of the Fab domain and the second scFv domain is coupled to the N-terminus of the second Fc polypeptide.

Embodiment 159. The antibody construct of embodiment 158, wherein the antibody construct comprises: a) a first heavy chain polypeptide comprising, from N-terminus to C-terminus: (i) a heavy chain Fab sequence comprising a Fab V_{H} sequence coupled to a Fab C_{H1} sequence, and (ii) the first Fc polypeptide; b) a second heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), and (ii) the second Fc polypeptide; and c) a light chain polypeptide comprising, from N-terminus to C-terminus: (i) a light chain Fab sequence comprising a Fab V_{L} sequence coupled to a Fab C_{L} sequence, and (ii) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), wherein: the heavy chain Fab sequence and the light chain Fab sequence associate to form the Fab domain, and the first Fc polypeptide and the second Fc polypeptide associate to form the Fc domain.

Embodiment 160. The antibody construct of any one of embodiments 152-153, wherein the first scFv domain is coupled to an N-terminus of the Fab domain and the second scFv domain is coupled to the C-terminus of one of the Fc polypeptides.

Embodiment 161. The antibody construct of embodiment 160, wherein the first scFv domain is coupled to the N-terminus of the V_{H} domain of the Fab domain.

Embodiment 162. The antibody construct of embodiment 160, wherein the first scFv domain is coupled to the N-terminus of the V_{L} domain of the Fab domain.

Embodiment 163. The antibody construct of any one of embodiments 160-162, wherein the second scFv domain is coupled to the C-terminus of the first Fc polypeptide.

Embodiment 164. The antibody construct of any one of embodiments 160-162, wherein the second scFv domain is coupled to the C-terminus of the second Fc polypeptide.

Embodiment 165. The antibody construct of any one of embodiments 160-161 or 163, wherein the antibody construct comprises: a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, (iii) the first Fc polypeptide, and (iv) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}); b) a second heavy chain polypeptide comprising the second Fc polypeptide; and c) a light chain polypeptide comprising a Fab V_{L} sequence coupled to a Fab C_{L} sequence, wherein: the heavy chain Fab sequence and the light chain polypeptide associate to form the Fab domain, and the first Fc polypeptide and the second Fc polypeptide associate to form the Fc domain.

Embodiment 166. The antibody construct of any one of embodiments 160-161 or 164, wherein the antibody construct comprises: a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, and (iii) the first Fc polypeptide; b) a second heavy chain polypeptide comprising, from N-terminus to C-terminus: (i) the second Fc polypeptide, and (ii) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}); and c) a light chain polypeptide comprising a Fab V_{L} sequence coupled to a Fab C_{L} sequence, wherein: the heavy chain Fab sequence and the light chain polypeptide associate to form the Fab domain, and the first Fc polypeptide and the second Fc polypeptide associate to form the Fc domain.

Embodiment 167. The antibody construct of any one of embodiments 147-166, wherein the first scFv domain is capable of binding the TAA, and the second scFv domain is capable of binding the second antigen on the second cytotoxic effector cell.

Embodiment 168. The antibody construct of any one of embodiments 147-166, wherein the second scFv domain is capable of binding the TAA, and the first scFv domain is capable of binding the second antigen on the second cytotoxic effector cell.

Embodiment 169. An antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the N-terminus of the V_{H} domain of the Fab domain, and (c) the second scFv domain is coupled to the N-terminus of the second Fc polypeptide.

Embodiment 170. An antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the N-terminus of the V_{H} domain of the Fab domain, and (c) the second scFv domain is coupled to the N-terminus of the second Fc polypeptide.

Embodiment 171. An antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the C_{L} domain of the Fab domain, and (c) the second scFv domain is coupled to the N-terminus of the second Fc polypeptide.

Embodiment 172. An antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the C_{L} domain of the Fab domain, and (c) the second scFv domain is coupled to the N-terminus of the second Fc polypeptide.

Embodiment 173. An antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the second Fc polypeptide.

Embodiment 174. An antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the second Fc polypeptide.

Embodiment 175. An antibody construct, comprising: (i) a Fab domain capable of binding CD3 on a first cytotoxic effector cell; (ii) a first scFv domain and a second scFv domain, wherein the first scFv domain is capable of binding CD28 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the V_{H} domain of the Fab domain.

Embodiment 176. An antibody construct, comprising: (i) a Fab domain capable of binding CD28 on a first cytotoxic effector cell; (ii) a first scFv domain and a second scFv domain, wherein the first scFv domain is capable of binding CD3 on a second cytotoxic effector cell and the second scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, (b) the first scFv domain is coupled to the C-terminus of the first Fc polypeptide, and (c) the second scFv domain is coupled to the N-terminus of the V_{H} domain of the Fab domain.

Embodiment 177. The antibody construct of any one of embodiments 147-176, wherein the first scFv domain has the domain structure, from N- to C-terminus, of: V_{H}-V_{L}.

Embodiment 178. The antibody construct of any one of embodiments 147-176, wherein the first scFv domain has the domain structure, from N- to C-terminus, of: V_{L}-V_{H}.

Embodiment 179. The antibody construct of any one of embodiments 147-178, wherein the second scFv domain has the domain structure, from N- to C-terminus, of: V_{H}-V_{L}.

Embodiment 180. The antibody construct of any one of embodiments 147-178, wherein the second scFv domain has the domain structure, from N- to C-terminus, of: V_{L}-V_{H}.

Embodiment 181. The antibody construct of any one of embodiments 147-180, wherein the antibody construct comprises one or more linkers.

Embodiment 182. The antibody construct of embodiment 181, wherein the one or more linkers are peptide linkers that each comprise or consist of an amino acid sequence from 1 to about 50, from 2 to about 40, from 3 to about 30, or from 5 to about 25 consecutive amino acid residues in length.

Embodiment 183. The antibody construct of any one of embodiments 181-182, wherein the first scFv domain comprises a linker^{scFv1}.

Embodiment 184. The antibody construct of embodiment 183, wherein the linker^{scFv1} couples the N- or C-terminus of the V_{H} domain to the C- or N-terminus of the V_{L} domain, respectively, and comprises or consists of an amino acid sequence having about 80%, 90%, or 100% sequence identity to the amino acid sequence set forth in SEQ ID NO: 104.

Embodiment 185. The antibody construct of any one of embodiments 181-184, wherein the second scFv domain comprises a linker^{scFv2}.

Embodiment 186. The antibody construct of embodiment 185, wherein the linker^{scFv2} couples the N- or C-terminus of the V_{H} domain to the C- or N-terminus of the V_{L} domain, respectively, and comprises or consists of an amino acid sequence having about 80%, 90%, or 100% sequence identity to the amino acid sequence set forth in SEQ ID NO: 104.

Embodiment 187. The antibody construct of any one of embodiments 147-158, 161-171, or 176-177, wherein the first scFv domain or the second scFv domain is coupled to the Fab domain via a linker^{scFv-Fab}.

Embodiment 188. The antibody construct of embodiment 187, wherein the linker^{scFv-Fab} comprises or consists of an amino acid sequence having about 60%, 80%, or 100% sequence identity to the amino acid sequence set forth in SEQ ID NO: 105.

Embodiment 189. The antibody construct of any one of embodiments 173-174, wherein the first scFv domain is coupled to the C-terminus of the first Fc polypeptide via a linker^{Fc-scFv}.

Embodiment 190. The antibody construct of embodiment 189, wherein the linker^{Fc-scFv} comprises or consists of an amino acid sequence having about 60%, 80%, or 100% sequence identity to the amino acid sequence set forth in SEQ ID NO: 105.

Embodiment 191. The antibody construct of any one of embodiments 147-190, wherein the C-terminus of the heavy chain of the Fab domain is coupled to the N-terminus of the first or second Fc polypeptide via a linker^{Fab-Fc}.

Embodiment 192. The antibody construct of embodiment 191, wherein the linker^{Fab-Fc} comprises or consists of an amino acid sequence having about 80%, 90%, or 100% sequence identity to the amino acid sequence set forth in SEQ ID NO: 108.

Embodiment 193. The antibody construct of any one of embodiments 147-159 or 169-174, wherein the C-terminus of the first scFv domain or the second scFv domain is coupled to N-terminus of the second Fc polypeptide via a linker^{scFv-Fc}.

Embodiment 194. The antibody construct of embodiment 193, wherein the linker^{scFv-Fc} comprises or consists of an amino acid sequence having about 80%, 90%, or 100% sequence identity to the amino acid sequence set forth in SEQ ID NO: 111 or SEQ ID NO: 112.

Embodiment 195. The antibody construct of any one of embodiments 147-194, wherein the Fab domain that is capable of binding the first antigen on the first cytotoxic effector cell comprises a heavy chain constant domain (C_{H1}) comprising or consisting of an amino acid sequence having at least about 90%, 95%, 97%, 99%, or 100% sequence identity to the amino acid sequence set forth in SEQ ID NO: 107.

Embodiment 196. The antibody construct of any one of embodiments 148-195, wherein the antibody construct is capable of binding human CD28 with a dissociation constant (K_{D}) for CD28 of from about 10 nM to about 500 nM, from about 20 nM to about 600 nM, from about 20 nM to about 250 nM, from about 20 nM to about 150 nM, from about 20 nM to about 100 nM, or from about 20 nM to about 50 nM, as determined using SPR.

Embodiment 197. The antibody construct of any one of embodiments 148-196, wherein the antibody construct comprises an anti-CD28 V_{H} sequence comprising a HCDR1 having the sequence SX₁GVH (SEQ ID NO: 302), a HCDR2 having the sequence VIWX₂GGX₃TNFNSALMS (SEQ ID NO: 306), and a HCDR3 having the sequence DRAX₄GX₅YX₆X₇AMDY (SEQ ID NO: 312) and an anti-CD28 V_{L} sequence comprising a LCDR1 having the sequence RASESVEYYX₈TSLMQ (SEQ ID NO: 315), a LCDR2 having the sequence AASX₉VX₁₀S (SEQ ID NO: 319), and a LCDR3 having the sequence QQSRKVPFT (SEQ ID NO: 320), and wherein X₁=Y or A; X₂=P or A; X₃=G or S; X₄=S or Y; X₅=N or A; X₆=L or N; X₇=S or Y; X₈=G or V; X₉=N or A; and X₁₀=E or D.

Embodiment 198. The antibody construct of any one of embodiments 147-197, wherein the antibody construct comprises an anti-CD28 V_{H} sequence having at least about 90%, 95%, 97%, 99%, or 100% sequence identity to the amino acid sequence set forth in SEQ ID NO: 106, and an anti-CD28 V_{L} sequence having at least about 90%, 95%, 97%, 99%, or 100% sequence identity to the amino acid sequence set forth in SEQ ID NO: 116.

Embodiment 199. The antibody construct of embodiment 198, wherein the anti-CD28 V_{H} sequence comprises or consists of an amino acid sequence selected from the group consisting of SEQ ID NOs: 201, 203-208, and 210, and the anti-CD28 V_{L} sequence comprises or consists of an amino acid sequence selected from the group consisting of SEQ ID NOs: 200, 202, and 209.

Embodiment 200. The antibody construct of any one of embodiments 148-179, wherein the antibody construct comprises an anti-CD28 V_{H} sequence comprising the HCDR1 having the sequence SYGVH (SEQ ID NO: 300), the HCDR2 having the sequence VIWPGGGTNFNSALMS (SEQ ID NO: 303), and the HCDR3 having the sequence DRAYGNYLYAMDY (SEQ ID NO: 307) and the anti-CD28 V_{L} sequence comprises the LCDR1 having the sequence RASESVEYYVTSLMQ (SEQ ID NO: 313), the LCDR2 having the sequence AASNVDS (SEQ ID NO: 316), and the LCDR3 having the sequence QQSRKVPFT (SEQ ID NO: 320).

Embodiment 201. The antibody construct of any one of embodiments 148-150, 153, 170, 172, 174 or 176, wherein the anti-CD28 Fab domain comprises a heavy chain comprising a V_{H} sequence having at least about 90%, 95%, 97%, 99%, or 100% sequence identity to the amino acid sequence set forth in SEQ ID NO: 106, coupled to a C_{H1} sequence having at least about 90%, 95%, 97%, 99%, or 100% sequence identity to the amino acid sequence set forth in SEQ ID NO: 107, and a light chain comprising a V_{L} sequence having at least about 90%, 95%, 97%, 99%, or 100% sequence identity to the amino acid sequence set forth in SEQ ID NO: 116, coupled to a C_{L} sequence having at least about 90%, 95%, 97%, 99%, or 100% sequence identity to the amino acid sequence set forth in SEQ ID NO: 117.

Embodiment 202. The antibody construct of any one of embodiments 149-152, 169, 171, 173 or 175, wherein the first scFv domain or second scFv domain that is capable of binding CD28 comprises, either from N- to C-terminus or C- to N-terminus, a V_{H} sequence having at least about 90%, 95%, 97%, 99%, or 100% sequence identity to the amino acid sequence set forth in SEQ ID NO: 106, coupled to a V_{L} sequence having at least about 90%, 95%, 97%, 99%, or 100% sequence identity to the amino acid sequence set forth in SEQ ID NO: 116.

Embodiment 203. The antibody construct of embodiment 202, wherein the first scFv domain or second scFv domain that is capable of binding CD28 comprises or consists of an amino acid sequence having at least about 90%, 95%, 97%, 99%, or 100% sequence identity to the amino acid sequence set forth in SEQ ID NO: 119.

Embodiment 204. The antibody construct of any one of embodiments 148-203, wherein the antibody construct is capable of binding human CD3 with a dissociation constant (K_{D}) for CD3 from about 20 nM to about 200 nM, from about 30 nM to about 150 nM, from about 40 nM to about 100 nM, or from 50 nM to about 80 nM, as determined using SPR.

Embodiment 205. The antibody construct of any one of embodiments 148-204, wherein the antibody construct comprises an anti-CD3 V_{H} sequence comprising the CDR sequences set forth in SEQ ID NOS: 321-323, and an anti-CD3 V_{L} sequence comprising the CDR sequences set forth in SEQ ID NO: 324-326.

Embodiment 206. The antibody construct of any one of embodiments 148-205, wherein the antibody construct comprises an anti-CD3 V_{H} sequence comprising a HCDR1 having the sequence GVTFNYYG (SEQ ID NO: 321), a HCDR2 having the sequence ITSSGGRI (SEQ ID NO: 322), and a HCDR3 having the sequence TLDGRDGWVAY (SEQ ID NO: 323), and an anti-CD3 V_{L} sequence comprising a LCDR1 having the sequence TGNIGSNY (SEQ ID NO: 324), a LCDR2 having the sequence RND (SEQ ID NO: 325), and a LCDR3 having the sequence QSYSSGFI (SEQ ID NO: 326).

Embodiment 207. The antibody construct of any one of embodiments 148-206, wherein the antibody construct comprises an anti-CD3 V_{H} sequence having at least about 90%, 95%, 97%, 99%, or 100% sequence identity to the amino acid sequence set forth in SEQ ID NO: 102, and an anti-CD3 V_{L} sequence having at least about 90%, 95%, 97%, 99%, or 100% sequence identity to the amino acid sequence set forth in SEQ ID NO: 103.

Embodiment 208. The antibody construct of any one of embodiments 148-149, 151-152, 169, 171, 173 or 175, wherein the anti-CD3 Fab domain comprises a heavy chain comprising a V_{H} sequence having at least about 90%, 95%, 97%, 99%, or 100% sequence identity to the amino acid sequence set forth in SEQ ID NO: 102, coupled to a C_{H1} sequence having at least about 90%, 95%, 97%, 99%, or 100% sequence identity to the amino acid sequence set forth in SEQ ID NO: 107, and a light chain comprising a V_{L} sequence having at least about 90%, 95%, 97%, 99%, or 100% sequence identity to the amino acid sequence set forth in SEQ ID NO: 103, coupled to a C_{L} sequence having at least about 90%, 95%, 97%, 99%, or 100% sequence identity to the amino acid sequence set forth in SEQ ID NO: 121.

Embodiment 209. The antibody construct of any one of embodiments 149-150, 153, 170, 172, 174 or 176, wherein the first or second scFv domain that is capable of binding CD3 comprises, either from N- to C-terminus or C- to N-terminus, a V_{H} sequence having at least about 90%, 95%, 97%, 99%, or 100% sequence identity to the amino acid sequence set forth in SEQ ID NO: 106, coupled to a V_{L} sequence having at least about 90%, 95%, 97%, 99%, or 100% sequence identity to the amino acid sequence set forth in SEQ ID NO: 116.

Embodiment 210. The antibody construct of embodiment 209, wherein the first or second scFv domain that is capable of binding CD3 comprises or consists of an amino acid sequence having at least about 90%, 95%, 97%, 99%, or 100% sequence identity to the amino acid sequence set forth in SEQ ID NO: 101.

Embodiment 211. The antibody construct of any one of embodiments 147-210, wherein the first cytotoxic effector cell and the second cytotoxic effector cell are different cells.

Embodiment 212. The antibody construct of any one of embodiments 147-210, wherein the first cytotoxic effector cell and the second cytotoxic effector cell are the same cell.

Embodiment 213. The antibody construct of any one of embodiments 147-212, wherein the first and second antigens are on a T cell.

Embodiment 214. The antibody construct of any one of embodiments 147-213, wherein the TAA is Claudin 18.2 (Cldn 18.2) or mesothelin (MSLN).

Embodiment 215. The antibody construct of embodiment 214, wherein the TAA is Cldn18.2.

Embodiment 216. The antibody construct of embodiment 214, wherein the TAA is MSLN.

Embodiment 217. The antibody construct of embodiment 215, wherein the antibody construct comprises an anti-Cldn18.2 V_{H} sequence comprising the HCDR1-3 sequences set forth in SEQ ID NOs: 333-335, respectively, and an anti-Cldn 18.2 V_{L} sequence comprising the LCDR1-3 sequences set forth in SEQ ID NO: 336-338, respectively.

Embodiment 218. The antibody construct of embodiment 217, wherein the antibody construct comprises an anti-Cldn18.2 V_{H} sequence comprising a HCDR1 having the sequence SNPMI (SEQ ID NO: 333), a HCDR2 having the sequence IIDTDGSTYYADWAKG (SEQ ID NO: 334), and a HCDR3 having the sequence RLHGSSNGYYDDL (SEQ ID NO: 335), and an anti-Cldn18.2 V_{L} sequence comprising a LCDR1 having the sequence QASQSIYSYLS (SEQ ID NO: 336), a LCDR2 having the sequence KASTLAS (SEQ ID NO: 337), and a LCDR3 having the sequence QQGYTVTNVDKNT (SEQ ID NO: 338).

Embodiment 219. The antibody construct of embodiment 217, wherein the anti-Cldn18.2 V_{H} sequence comprises or consists of an amino acid sequence having at least about 90%, 95%, 97%, 99%, or 100% sequence identity to the amino acid sequence set forth in SEQ ID NO: 127, and the anti-Cldn 18.2 V_{L} sequence comprises or consists of an amino acid sequence having at least about 90%, 95%, 97%, 99%, or 100% sequence identity to the amino acid sequence set forth in SEQ ID NO: 128, and, optionally, wherein the antibody construct binds human Cldn18.2 with an affinity from about 1 nM to about 80 nM, from about 10 nM to about 60 nM, from about 10 nM to about 50 nM, or from about 20 nM to about 50 nM, as determined by flow cytometry.

Embodiment 220. The antibody construct of embodiment 216, wherein the antibody construct comprises an anti-MSLN V_{H} sequence comprising the HCDR1-3 sequences set forth in SEQ ID NOs: 327-329, respectively, and an anti-MSLN V_{L} sequence comprising the LCDR1-3 sequences set forth in SEQ ID NOs: 330-332, respectively.

Embodiment 221. The antibody construct of embodiment 220, wherein the antibody construct comprises an anti-MSLN V_{H} sequence comprising a HCDR1 having the sequence GYTMN (SEQ ID NO: 327), a HCDR2 having the sequence LITPYSGASSYAQKFQG (SEQ ID NO: 328), and a HCDR3 having the sequence GGYDGRGFDY (SEQ ID NO: 329), and an anti-MSLN V_{L} sequence comprising a LCDR1 having the sequence SASSSVSYMH (SEQ ID NO: 330), a LCDR2 having the sequence DTSKLAS (SEQ ID NO: 331), and a LCDR3 having the sequence QQWSGHPLT (SEQ ID NO: 332).

Embodiment 222. The antibody construct of any one of embodiments 147-221, wherein the first Fc polypeptide and the second Fc polypeptide of the Fc domain each comprise or consist of a CH2 sequence and a CH3 sequence.

Embodiment 223. The antibody construct of embodiment 222, wherein at least one of the CH2 sequences of the first and second Fc polypeptide is an IgG1 or IgG4 CH2 sequence and comprises one or more amino acid modifications when compared to an unmodified wildtype IgG1 or IgG4 CH2 sequence.

Embodiment 224. The antibody construct of embodiment 223, wherein both CH2 sequences of the first and second Fc polypeptide are IgG1 or IgG4 CH2 sequences and comprise the one or more amino acid modifications when compared to an unmodified wildtype IgG1 or IgG4 CH2 sequence.

Embodiment 225. The antibody construct of embodiment 223-224, wherein the one or more amino acid modifications to the CH2 sequences reduce or ablate interactions of the Fc domain with one or more Fc receptors, optionally, one or more Fcγ receptors.

Embodiment 226. The antibody construct of any one of embodiments 222-225, wherein the CH2 sequence of both the first and the second Fc polypeptide comprises or consists of an amino acid sequence having at least about 90%, 95%, 97%, 99%, or 100% sequence identity to the amino acid sequence set forth in SEQ ID NO: 109.

Embodiment 227. The antibody construct of any one of embodiments 222-226, wherein at least one of the CH3 sequences of the first and second Fc polypeptide is an IgG1 or IgG4 CH3 sequence and comprises one or more amino acid modifications when compared to an unmodified wildtype IgG1 or IgG4 CH3 sequence, and, optionally, wherein the first and second Fc polypeptides have different amino acid sequences and form a heterodimeric Fc domain.

Embodiment 228. The antibody construct of embodiment 227, wherein both CH3 sequences of the first and second Fc polypeptide are IgG1 or IgG4 CH3 sequences and comprise one or more amino acid modifications that promote preferential pairing of the first and second Fc polypeptide to form the heterodimeric Fc domain compared to the formation of a corresponding homodimeric Fc domain.

Embodiment 229. The antibody construct of any one of embodiments 227-228, wherein the CH3 sequence of one of the Fc polypeptides comprises a set of amino acid substitutions selected from the group consisting of: L351Y_F405A_Y407V, T350V_L351Y_F405A_Y407V and T350V_L351Y_S400E_F405A_Y407V, and the CH3 sequence of the other Fc polypeptide comprises a set of amino acid substitutions selected from the group consisting of: T366L_K392M_T394W, T366L_K392L_T394W, T350V_T366L_K392L_T394W, T350V_T366L_K392M_T394W and T350V_T366L_N390R_K392M_T394W, and wherein the numbering of amino acid residues in the Fc polypeptides is according to the EU numbering system.

Embodiment 230. The antibody construct of any one of embodiments 222-229, wherein the CH3 sequence of one Fc polypeptide comprises or consists of an amino acid sequence having at least about 90%, 95%, 97%, 99%, or 100% sequence identity to the amino acid sequence set forth in SEQ ID NO: 110.

Embodiment 231. The antibody construct of any one of embodiments 222-230, wherein the CH3 sequence of the other Fc polypeptide comprises or consists of an amino acid sequence having at least about 90%, 95%, 97%, 99%, or 100% sequence identity to the amino acid sequence set forth in SEQ ID NO: 114.

Embodiment 232. The antibody construct of embodiment 147, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 100 or 118, (ii) a second heavy chain polypeptide (H2) that comprises or consists of an amino acid sequence having at least about 90%, 95%, 97%, 99%, or 100% sequence identity to the amino acid sequence set forth in SEQ ID NO: 153 or 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115 or 120.

Embodiment 233. The antibody construct of embodiment 232, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 153, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 234. The antibody construct of embodiment 232, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 118, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

Embodiment 235. The antibody construct of embodiment 147, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 122, (ii) a second heavy chain polypeptide (H2) that comprises or consists of an amino acid sequence having at least about 90%, 95%, 97%, 99%, or 100% sequence identity to the amino acid sequence set forth in SEQ ID NO: 153, and (iii) a light chain polypeptide (L1) that comprises or consists of an amino acid sequence having at least about 90%, 95%, 97%, 99%, or 100% sequence identity to the amino acid sequence set forth in SEQ ID NO: 123.

Embodiment 236. The antibody construct of embodiment 153, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 124, (ii) a second heavy chain polypeptide (H2) that comprises or consists of an amino acid sequence having at least about 90%, 95%, 97%, 99%, or 100% sequence identity to the amino acid sequence set forth in SEQ ID NO: 153, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 237. The antibody construct of embodiment 153, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 124, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 238. The antibody construct of embodiment 152, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 129, (ii) a second heavy chain polypeptide (H2) that comprises or consists of an amino acid sequence having at least about 90%, 95%, 97%, 99%, or 100% sequence identity to the amino acid sequence set forth in SEQ ID NO: 153, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

Embodiment 239. The antibody construct of embodiment 152, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 122, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 123.

Embodiment 240. The antibody construct of embodiment 152, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 129, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

Embodiment 241. The antibody construct of embodiment 147, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 242. The antibody construct of embodiment 147 or embodiment 153, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 134, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 135.

Embodiment 243. The antibody construct of embodiment 152, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 130, (ii) a second heavy chain polypeptide (H2) that comprises or consists of an amino acid sequence having at least about 90%, 95%, 97%, 99%, or 100% sequence identity to the amino acid sequence set forth in SEQ ID NO: 131, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

Embodiment 244. The antibody construct of embodiment 147, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 132, (ii) a second heavy chain polypeptide (H2) that comprises or consists of an amino acid sequence having at least about 90%, 95%, 97%, 99%, or 100% sequence identity to the amino acid sequence set forth in SEQ ID NO: 133, and (iii) a light chain polypeptide (L1) that comprises or consists of an amino acid sequence having at least about 90%, 95%, 97%, 99%, or 100% sequence identity to the amino acid sequence set forth in SEQ ID NO: 120.

Embodiment 245. The antibody construct of embodiment 153, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 136, (ii) a second heavy chain polypeptide (H2) that comprises or consists of an amino acid sequence having at least about 90%, 95%, 97%, 99%, or 100% sequence identity to the amino acid sequence set forth in SEQ ID NO: 131, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 246. The antibody construct of embodiment 147, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 137, (ii) a second heavy chain polypeptide (H2) that comprises or consists of an amino acid sequence having at least about 90%, 95%, 97%, 99%, or 100% sequence identity to the amino acid sequence set forth in SEQ ID NO: 138, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 247. The antibody construct of embodiment 147, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 139, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 248. The antibody construct of embodiment 147, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 140, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 249. The antibody construct of embodiment 147, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 141.

Embodiment 250. The antibody construct of embodiment 147, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 142.

Embodiment 251. The antibody construct of embodiment 147, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in any one of SEQ ID NOs: 143-149, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 115.

Embodiment 252. The antibody construct of embodiment 147, wherein the antibody construct comprises (i) a first heavy chain polypeptide (H1) that comprises or consists of an amino acid sequence having at least about 90%, 95%, 97%, 99%, or 100% sequence identity to the amino acid sequence set forth in SEQ ID NO: 100, (ii) a second heavy chain polypeptide (H2) that comprises or consists of an amino acid sequence having at least about 90%, 95%, 97%, 99%, or 100% sequence identity to the amino acid sequence set forth in SEQ ID NO: 125, and (iii) a light chain polypeptide (L1) that comprises or consists of an amino acid sequence having at least about 90%, 95%, 97%, 99%, or 100% sequence identity to the amino acid sequence set forth in SEQ ID NO: 150 or SEQ ID NO: 152.

Embodiment 253. The antibody construct of any one of embodiments 147-252, wherein the antibody construct is trivalent and trispecific and binds each antigen monovalently.

Embodiment 254. The antibody construct of any one of embodiments 147-253, wherein the construct has a binding affinity for the TAA of at least about 40 nM, 30 nM, 20 nM, 10 nM or 5 nM, or from about 5 nM to about 40 nM or from about 10 nM to about 30 nM.

Embodiment 255. The antibody construct of any one of embodiments 147-254, wherein the antibody construct has a melting temperature at Tm1, Tm2, and/or Tm3 that is within 10° C., 5° C., 2° C., or within 1° C. degree of that of a corresponding bivalent and monospecific IgG1 monoclonal antibody.

Embodiment 256. The antibody construct of any one of embodiments 148-255, wherein the antibody construct binds the cytotoxic effector cell that expresses CD3 and/or CD28 with an affinity from about 1 nM to about 500 pM, as determined by flow cytometry.

Embodiment 257. The antibody construct of any one of embodiments 148-256, wherein the antibody construct binds the cytotoxic effector cell that expresses CD3 and/or CD28 with an affinity that is about 2-fold, 5-fold, 10-fold, 20-fold, 50-fold, 100-fold, or about 200-fold higher than that of a corresponding bispecific anti-CD3×TAA and/or anti-CD28×TAA antibody construct.

Embodiment 258. The antibody construct of any one of embodiments 147-257, wherein the antibody construct exhibits an IC₅₀ value from about 50 pM to about 0.01 pM, from about 25 pM to about 0.01 pM, from about from about 10 pM to about 0.05 pM, from about 10 pM to about 0.1 pM, from about 10 pM to about 1 pM, from about 5 pM to about 1 pM for killing TAA-expressing tumor cells that express at least about 100,000 TAA/cell by TDCC in the presence of the cytotoxic effector cell and using an E:T ratio of 2:1 and an incubation period of 72 hours.

Embodiment 259. The antibody construct of embodiment 258, wherein the antibody construct achieves a maximum killing of TAA-expressing tumor cells of at least about 60%, 65%, 70%, 75%, or 80%, 85%, or 90%, 100%, or from about 60% to about 100%, from about 70% to about 90%, or from about 75% to about 85%.

Embodiment 260. The antibody construct of any one of embodiments 147-259, wherein the antibody construct is capable of inducing the production of one or more cytokines by the cytotoxic effector cell ranging from about 300 μg/mL to about 9000 μg/mL, when TAA-expressing cells expressing at least about 100,000 TAA/cell are present and using an E:T ratio of 2:1 and an incubation period of 72 hours.

Embodiment 261. A pharmaceutical composition comprising the antibody construct of any one of embodiments 147-260, and a pharmaceutically acceptable carrier, excipient, diluent, or combination thereof.

Embodiment 262. A nucleic acid molecule or a set of nucleic acid molecules encoding one or more, two or more, or three or more polypeptide chains that form the antibody construct of any one of embodiments 147-260.

Embodiment 263. A vector or a set of vectors comprising the nucleic acid molecule or the set of nucleic acid molecules of embodiment 262.

Embodiment 264. A cell comprising the nucleic acid molecule or the set of nucleic acid molecules of embodiment 262, or the vector or set of vectors of embodiment 263.

Embodiment 265. A method of producing an antibody construct of any one of embodiments 147-260, the method comprising: (a) obtaining a host cell culture comprising at least one host cell comprising one or more nucleic acid molecules encoding one or more, two or more, or three or more polypeptide chains that form the antibody construct; and (b) recovering the antibody construct from the host cell culture.

Embodiment 266. The method of embodiment 265, further comprising, subsequent to step (b), purifying the antibody construct.

Embodiment 267. A method of eliciting an anti-tumor immune response in a cell population comprising immune cells and tumor cells, the method comprising contacting the cell population with an effective amount of the antibody construct of any one of embodiments 147-260, wherein the immune cells express the first and second antigen and the tumor cells express the TAA.

Embodiment 268. A method of inhibiting the proliferation of tumor cells, the method comprising contacting a cell population comprising the tumor cells and immune cells with an effective amount of the antibody construct of any one of embodiments 147-260, wherein the immune cells express the first and second antigen and the tumor cells express the TAA.

Embodiment 269. A method of killing tumor cells, the method comprising contacting a cell population comprising the tumor cells and immune cells with an effective amount of the antibody construct of any one of embodiments 147-260, wherein the immune cells express the first and second antigen and the tumor cells express the TAA.

Embodiment 270. The method of any one of embodiments 267-269, wherein the immune cells comprise T cells.

Embodiment 271. The method of any one of embodiments 267-270, wherein TAA is MSLN or Cldn18.2.

Embodiment 272. The method of any one of embodiments 267-271, wherein the antibody construct binds CD3 and CD28 on either one T cell or two different T cells, and the TAA on a tumor cell.

Embodiment 273. The method of embodiment 272, wherein the binding of the first and second antigen and the TAA forms a TCR-independent artificial immune synapse between the one or more immune cells and the tumor cell, thereby eliciting a cytotoxic immune response of the immune cell against the tumor cell.

Embodiment 274. The method of any one of embodiments 267-273, wherein the cell population is within a subject.

Embodiment 275. A method for treating a cancer in a subject in need thereof, the method comprising administering to the subject an antibody construct of any one of embodiments 147-260.

Embodiment 276. The method of embodiment 275, wherein a cytotoxic immune response against the cancer is elicited in the subject, thereby treating the cancer in the subject.

Embodiment 277. The method of any one of embodiments 274-276, wherein the subject is a rodent, a non-human primate, or a human.

Embodiment 278. An antibody construct of any one of embodiments 147-260 for use in the treatment of cancer.

Embodiment 279. Use of an antibody construct of any one of embodiments 147-260 in the manufacture of a medicament for the treatment of cancer.

Embodiment 280. An antibody construct, comprising a binding domain capable of binding CD28, wherein the binding domain comprises a V_{H} sequence comprising a HCDR1 having the sequence SX₁GVH (SEQ ID NO: 302), a HCDR2 having the sequence VIWX₂GGX₃TNFNSALMS (SEQ ID NO: 306), and a HCDR3 having the sequence DRAX₄GX₅YX₆X₇AMDY (SEQ ID NO: 312), and a V_{L} sequence comprising a LCDR1 having the sequence RASESVEYYX₈TSLMQ (SEQ ID NO: 315), a LCDR2 having the sequence AASX₉VX₁₀S (SEQ ID NO: 319), and a LCDR3 having the sequence QQSRKVPFT (SEQ ID NO: 320), having one or more of the following amino acid substitutions at the positions as identified in the CDR sequences: X₁: Y to A, X₂: P to A, X₃: G to S, X₄: S to Y, X₅: N to A, X₆: L to N, X₇: S to Y, X₈: G to V, X₉: N to A, and/or X₁₀: E to D.

Embodiment 281. The antibody construct of embodiment 280, wherein the binding domain comprises the substitution X₁: Y to A.

Embodiment 282. The antibody construct of any one of embodiments 280-281, wherein the binding domain comprises the substitution X₂: P to A.

Embodiment 283. The antibody construct of any one of embodiments 280-282, wherein the binding domain comprises the substitution X₃: G to S.

Embodiment 284. The antibody construct of any one of embodiments 280-283, wherein the binding domain comprises the substitution X₄: S to Y.

Embodiment 285. The antibody construct of any one of embodiments 280-284, wherein the binding domain comprises the substitution X₅: N to A.

Embodiment 286. The antibody construct of any one of embodiments 280-285, wherein the binding domain comprises the substitution X₆: L to N.

Embodiment 287. The antibody construct of any one of embodiments 280-286, wherein the binding domain comprises the substitution X₇: S to Y.

Embodiment 288. The antibody construct of any one of embodiments 280-287, wherein the binding domain comprises the substitution X₈: G to V.

Embodiment 289. The antibody construct of any one of embodiments 280-288, wherein the binding domain comprises the substitution X₉: N to A.

Embodiment 290. The antibody construct of any one of embodiments 280-289, wherein the binding domain comprises the substitution X₁₀: E to D.

Embodiment 291. The antibody construct of any one of embodiments 280-290, wherein the antibody construct has a binding affinity for CD28 that is reduced by about 1.5-fold to about 25-fold, by about 2.0-fold to about 20-fold, by about 3.0-fold to about 20-fold, or by about 5.0-fold to about 10-fold, when compared to the binding affinity of an antibody construct comprising an anti-CD28 binding domain that does not contain the one or more amino acid substitutions in one or more of the CDR sequences.

Embodiment 292. The antibody construct of embodiment 280, wherein the binding domain comprises a V_{H} sequence comprising or consisting of an amino acid sequence selected from the group consisting of SEQ ID NOs: 201, 203-208, and 210, and a V_{L} sequence comprising or consisting of an amino acid sequence selected from the group consisting of SEQ ID NOs: 200, 202, and 209.

Embodiment 293. An antibody construct, comprising a binding domain capable of binding Cldn 18.2, wherein the binding domain comprises a V_{H} sequence comprising a HCDR1 having the sequence SNPMI (SEQ ID NO: 333), a HCDR2 having the sequence IIDTDGSTYYADWAKG (SEQ ID NO: 334), and a HCDR3 having the sequence RLHGSSNGYYDDL (SEQ ID NO: 335), and a V_{L} sequence comprising a LCDR1 having the sequence QASQSIYSYLS (SEQ ID NO: 336), a LCDR2 having the sequence KASTLAS (SEQ ID NO: 337), and a LCDR3 having the sequence QQGYTVTNVDKNT (SEQ ID NO: 338).

Embodiment 294. The antibody construct of embodiment 293, wherein the binding domain comprises a V_{H} sequence comprising or consisting of an amino acid sequence having at least about 90%, 95%, 97%, 99%, or 100% sequence identity to the amino acid sequence set forth in SEQ ID NO: 127, and a V_{L} sequence comprising or consisting of an amino acid sequence having at least about 90%, 95%, 97%, 99%, or 100% sequence identity to the amino acid sequence set forth in SEQ ID NO: 128.

Embodiment 295. The antibody construct of any one of embodiments 293-294, wherein the binding domain comprises a V_{H} sequence comprising or consisting of the amino acid sequence set forth in SEQ ID NO: 127, and a V_{L} sequence comprising or consisting of the amino acid sequence set forth in SEQ ID NO: 128.

Embodiment 296. The antibody construct of any one of embodiments 280-295, further comprising one or more additional binding domains capable of binding one or more additional antigens.

Embodiment 297. An antibody construct, comprising: (i) a Fab domain comprising a heavy chain comprising a V_{H} sequence and a C_{H1} sequence and a light chain comprising a V_{L} sequence and C_{L} sequence, wherein the Fab domain is capable of binding CD3; (ii) a first scFv domain comprising a first V_{H} sequence and a first V_{L} sequence, wherein the first scFv domain is capable of binding CD28; (iii) a second scFv domain comprising a second V_{H} sequence and a second V_{L} sequence, wherein the second scFv domain is capable of binding Cldn18.2; and (iv) a dimeric Fc domain comprising a first Fc polypeptide and a second Fc polypeptide, wherein: a) the Fab domain is coupled via its C_{H1} sequence to the N-terminus of the first Fc polypeptide, b) the first scFv domain is coupled to the C-terminus of the C_{L} sequence of the Fab light chain, and c) the second scFv domain is coupled to the N-terminus of the second Fc polypeptide.

Embodiment 298. The antibody construct of embodiment 297, wherein the antibody construct does not reduce T cell viability by more than 5%, 3%, 1%, or by 0% compared to T cells treated with a negative control construct that does not contain a binding domains against Cldn 18.2, and wherein the antibody construct is incubated with the T cells for 48 hours.

Embodiment 299. The antibody construct of embodiment 297 or embodiment 298, wherein the antibody construct reduces T cell viability by about 1.5-fold to about 2-fold, by about 1.5-fold to about 3-fold, or by about 2-fold to about 3-fold less than an antibody construct in which the first scFv domain and the second scFv domain are independently coupled to either the N-terminus of the Fab heavy chain or the N-terminus of the second Fc polypeptide, and wherein the respective antibody construct is incubated with the T cells for 48 hours.

Embodiment 300. The antibody construct of any one of embodiments 297-299, wherein the antibody construct induces about 80-fold to about 2000-fold, about 100-fold to about 1000-fold, or about 100-fold to about 500-fold less cytokine in an assay comprising human CD3⁺ T cells only compared to an antibody construct in which the first scFv domain and the second scFv domain are independently coupled to either the N-terminus of the Fab heavy chain or the N-terminus of the second Fc polypeptide, and wherein the respective antibody construct is incubated with the T cells for 48 hours.

Embodiment 301. The antibody construct of any one of embodiments 297-300, wherein the antibody construct induces about 5-fold to about 900-fold, about 5-fold to about 500-fold, or about 5-fold to about 300-fold less cytokine in an assay comprising human PBMCs only compared to an antibody construct in which the first scFv domain and the second scFv domain are independently coupled to either the N-terminus of the Fab heavy chain or the N-terminus of the second Fc polypeptide, and wherein the respective antibody construct is incubated with the T cells for 48 hours.

Embodiment 302. The antibody construct of any one of embodiments 300-301, wherein the cytokine comprises one or more of IL-2, TNFα, IFNγ, and IL-6.

Embodiment 303. An antibody construct, comprising: (i) a Fab domain capable of binding a first antigen on a first cytotoxic effector cell; (ii) a first scFv domain and a second scFv domain, wherein one of the scFv domains is capable of binding a second antigen on a second cytotoxic effector cell and the other scFv domain is capable of binding a tumor-associated antigen (TAA) on a tumor cell, and (iii) an Fc domain comprising a first Fc polypeptide and a second Fc polypeptide, wherein: (a) the Fab domain is coupled to the N-terminus of the first Fc polypeptide, and (b) the first and second scFv domains are independently coupled to either (i) an N-terminus of the Fab domain, (ii) a C-terminus of the Fab domain, (iii) the C-terminus of one of the Fc polypeptides, or (iv) the N-terminus of the second Fc polypeptide, provided that when one of the scFv domains is coupled to the C-terminus of one of the Fc polypeptides, the first antigen is CD3 and the second antigen is CD28, or the first antigen is CD28 and the second antigen is CD3.

Embodiment 304. The antibody construct of any one of embodiment 303, wherein the first scFv domain and the second scFv domain are not coupled to each other in tandem.

Embodiment 305. The antibody construct of any one of embodiment 303 or embodiment 304, wherein the first antigen is CD28, and the second antigen is CD3.

Embodiment 306. The antibody construct of any one of embodiment 303 or embodiment 304, wherein the first antigen is CD3, and the second antigen is CD28.

Embodiment 307. The antibody construct of any one of embodiments 303-306, wherein the first scFv domain is coupled to an N-terminus of the Fab domain and the second scFv is coupled to the N-terminus of the second Fc polypeptide.

Embodiment 308. The antibody construct of embodiment 307, wherein the first scFv domain is coupled to the N-terminus of the V_{H} sequence of the heavy chain of the Fab domain.

Embodiment 309. The antibody construct of embodiment 307, wherein the first scFv domain is coupled to the N-terminus of the V_{L} sequence of the light chain of the Fab domain.

Embodiment 310. The antibody construct of embodiments 307 or 308, wherein the antibody construct comprises: a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, and (iii) the first Fc polypeptide; b) a second heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), and (ii) the second Fc polypeptide; and c) a light chain polypeptide comprising, from N-terminus to C-terminus: a Fab V_{L} sequence coupled to a Fab C_{L} sequence, wherein: the heavy chain Fab sequence and the light chain polypeptide associate to form the Fab domain, and the first Fc polypeptide and the second Fc polypeptide associate to form the Fc domain.

Embodiment 311. The antibody construct of any one of embodiments 303-306, wherein the first scFv domain is coupled to the C-terminus of the light chain C_{L} sequence of the Fab domain and the second scFv domain is coupled to the N-terminus of the second Fc polypeptide.

Embodiment 312. The antibody construct of embodiment 311, wherein the antibody construct comprises: a) a first heavy chain polypeptide comprising, from N-terminus to C-terminus: (i) a heavy chain Fab sequence comprising a Fab V_{H} sequence coupled to a Fab C_{H1} sequence, and (ii) the first Fc polypeptide; b) a second heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), and (ii) the second Fc polypeptide; and c) a light chain polypeptide comprising, from N-terminus to C-terminus: (i) a light chain Fab sequence comprising a Fab V_{L} sequence coupled to a Fab C_{L} sequence, and (ii) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), wherein: the heavy chain Fab sequence and the light chain Fab sequence associate to form the Fab domain, and the first Fc polypeptide and the second Fc polypeptide associate to form the Fc domain.

Embodiment 313. The antibody construct of any one of embodiments 303-306, wherein the first scFv domain is coupled to an N-terminus of the Fab domain and the second scFv domain is coupled to the C-terminus of one of the Fc polypeptides.

Embodiment 314. The antibody construct of embodiment 313, wherein the first scFv domain is coupled to the N-terminus of the V_{H} domain of the Fab domain.

Embodiment 315. The antibody construct of any one of embodiments 313-314, wherein the second scFv domain is coupled to the C-terminus of the first Fc polypeptide.

Embodiment 316. The antibody construct of any one of embodiments 313-314, wherein the second scFv domain is coupled to the C-terminus of the second Fc polypeptide.

Embodiment 317. The antibody construct of any one of embodiments 313-315, wherein the antibody construct comprises: a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, (iii) the first Fc polypeptide, and (iv) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}); b) a second heavy chain polypeptide comprising the second Fc polypeptide; and c) a light chain polypeptide comprising a Fab V_{L} sequence coupled to a Fab C_{L} sequence, wherein: the heavy chain Fab sequence and the light chain polypeptide associate to form the Fab domain, and the first Fc polypeptide and the second Fc polypeptide associate to form the Fc domain.

Embodiment 318. The antibody construct of any one of embodiments 313-314, or 316, wherein the antibody construct comprises: a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, and (iii) the first Fc polypeptide; b) a second heavy chain polypeptide comprising, from N-terminus to C-terminus: (i) the second Fc polypeptide, and (ii) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}); and c) a light chain polypeptide comprising a Fab V_{L} sequence coupled to a Fab C_{L} sequence, wherein: the heavy chain Fab sequence and the light chain polypeptide associate to form the Fab domain, and the first Fc polypeptide and the second Fc polypeptide associate to form the Fc domain.

Embodiment 319. The antibody construct of any one of embodiments 303-318, wherein the first scFv domain is capable of binding the TAA, and the second scFv domain is capable of binding the second antigen on the second cytotoxic effector cell.

Embodiment 320. The antibody construct of any one of embodiments 303-318, wherein the second scFv domain is capable of binding the TAA, and the first scFv domain is capable of binding the second antigen on the second cytotoxic effector cell.

Embodiment 321. The antibody construct of any one of embodiments 303, 311 or 312, comprising: (i) the Fab domain capable of binding CD3; (ii) the first scFv domain capable of binding CD28; (iii) the second scFv domain capable of binding Claudin 18.2 (Cldn18.2); and (iv) the Fc domain comprising the first Fc polypeptide and the second Fc polypeptide, wherein: a) the Fab domain is coupled via its C_{H1} sequence to the N-terminus of the first Fc polypeptide, b) the first scFv domain is coupled to the C-terminus of the C_{L} sequence of the Fab light chain, and c) the second scFv domain is coupled to the N-terminus of the second Fc polypeptide.

Embodiment 322. The antibody construct of embodiment 321, wherein the antibody construct does not reduce T cell viability by more than 5%, 3%, 1%, or by 0% compared to T cells treated with a negative control construct that does not contain a binding domain against Cldn18.2, and wherein the antibody construct is incubated with the T cells for 48 hours.

Embodiment 323. The antibody construct of embodiment 321 or embodiment 322, wherein the antibody construct reduces T cell viability by about 1.5-fold to about 2-fold, by about 1.5-fold to about 3-fold, or by about 2-fold to about 3-fold less than an antibody construct in which the first scFv domain and the second scFv domain are independently coupled to either the N-terminus of the Fab heavy chain or the N-terminus of the second Fc polypeptide, and wherein the respective antibody construct is incubated with the T cells for 48 hours.

Embodiment 324. The antibody construct of any one of embodiments 321-323, wherein the antibody construct induces about 80-fold to about 2000-fold, about 100-fold to about 1000-fold, or about 100-fold to about 500-fold less cytokine in an assay comprising human CD3⁺ T cells compared to an antibody construct in which the first scFv domain and the second scFv domain are independently coupled to either the N-terminus of the Fab heavy chain or the N-terminus of the second Fc polypeptide, and wherein the respective antibody construct is incubated with the T cells for 48 hours.

Embodiment 325. The antibody construct of any one of embodiments 321-324, wherein the antibody construct induces about 5-fold to about 900-fold, about 5-fold to about 500-fold, or about 5-fold to about 300-fold less cytokine in an assay comprising human PBMCs compared to an antibody construct in which the first scFv domain and the second scFv domain are independently coupled to either the N-terminus of the Fab heavy chain or the N-terminus of the second Fc polypeptide, and wherein the respective antibody construct is incubated with the T cells for 48 hours.

Embodiment 326. The antibody construct of any one of embodiments 324-325, wherein the cytokine comprises one or more of IL-2, TNFα, IFNγ, and IL-6.

Embodiment 327. The antibody construct of any one of embodiments 303-326, wherein the first scFv domain has the domain structure, from N- to C-terminus, of: V_{H}-V_{L}.

Embodiment 328. The antibody construct of any one of embodiments 303-326, wherein the first scFv domain has the domain structure, from N- to C-terminus, of: V_{L}-V_{H}.

Embodiment 329. The antibody construct of any one of embodiments 303-328, wherein the second scFv domain has the domain structure, from N- to C-terminus, of: V_{H}-V_{L}.

Embodiment 330. The antibody construct of any one of embodiments 303-328, wherein the second scFv domain has the domain structure, from N- to C-terminus, of: V_{L}-V_{H}.

Embodiment 331. The antibody construct of any one of embodiments 303-330, wherein the antibody construct comprises one or more linkers.

Embodiment 332. The antibody construct of embodiment 331, wherein the one or more linkers are peptide linkers that each comprise or consist of an amino acid sequence from 1 to about 50, from 2 to about 40, from 3 to about 30, or from 5 to about 25 consecutive amino acid residues in length.

Embodiment 333. The antibody construct of any one of embodiments 331-332, wherein the first scFv domain comprises a linker^{scFv1}.

Embodiment 334. The antibody construct of embodiment 333, wherein the linker^{scFv1} couples the N- or C-terminus of the V_{H} domain to the C- or N-terminus of the V_{L} domain, respectively, and comprises or consists of an amino acid sequence having about 80%, 90%, or 100% sequence identity to the amino acid sequence set forth in SEQ ID NO: 104.

Embodiment 335. The antibody construct of any one of embodiments 331-334, wherein the second scFv domain comprises a linker^{scFv2}.

Embodiment 336. The antibody construct of embodiment 335, wherein the linker^{scFv2} couples the N- or C-terminus of the V_{H} domain to the C- or N-terminus of the V_{L} domain, respectively, and comprises or consists of an amino acid sequence having about 80%, 90%, or 100% sequence identity to the amino acid sequence set forth in SEQ ID NO: 104.

Embodiment 337. The antibody construct of any one of embodiments 303-336, wherein the Fab domain that is capable of binding the first antigen on the first cytotoxic effector cell comprises a heavy chain constant domain (C_{H1}) comprising or consisting of an amino acid sequence having at least about 90%, 95%, 97%, 99%, or 100% sequence identity to the amino acid sequence set forth in SEQ ID NO: 107.

Embodiment 338. The antibody construct of any one of embodiments 303-337, wherein the antibody construct is capable of binding human CD28 with a dissociation constant (Kp) for CD28 of from about 10 nM to about 500 nM, from about 20 nM to about 600 nM, from about 20 nM to about 250 nM, from about 20 nM to about 150 nM, from about 20 nM to about 100 nM, or from about 20 nM to about 50 nM, as determined using SPR.

Embodiment 339. The antibody construct of any one of embodiments 303-338, wherein the antibody construct comprises an anti-CD28 V_{H} sequence comprising a HCDR1 having the sequence SX₁GVH (SEQ ID NO: 302), a HCDR2 having the sequence VIWX₂GGX₃TNFNSALMS (SEQ ID NO: 306), and a HCDR3 having the sequence DRAX₄GX₅YX₆X₇AMDY (SEQ ID NO: 312), and an anti-CD28 V_{L} sequence comprising a LCDR1 having the sequence RASESVEYYX₈TSLMQ (SEQ ID NO: 315), a LCDR2 having the sequence AASX₉ VX₁₀S (SEQ ID NO: 319), and a LCDR3 having the sequence QQSRKVPFT (SEQ ID NO: 320), and wherein X₁=Y or A; X₂=P or A; X₃=G or S; X₄=S or Y; X₅=N or A; X₆=L or N; X₇=S or Y; X₈=G or V; X₉=N or A; and X₁₀=E or D.

Embodiment 340. The antibody construct of any one of embodiments 303-339, wherein the antibody construct comprises an anti-CD28 V_{H} sequence having at least about 90%, 95%, 97%, 99%, or 100% sequence identity to the amino acid sequence set forth in SEQ ID NO: 106, and an anti-CD28 V_{L} sequence having at least about 90%, 95%, 97%, 99%, or 100% sequence identity to the amino acid sequence set forth in SEQ ID NO: 116.

Embodiment 341. The antibody construct of embodiment 340, wherein the anti-CD28 V_{H} sequence comprises or consists of an amino acid sequence selected from the group consisting of SEQ ID NOs: 201, 203-208, and 210, and the anti-CD28 V_{L} sequence comprises or consists of an amino acid sequence selected from the group consisting of SEQ ID NOs: 200, 202, and 209.

Embodiment 342. The antibody construct of any one of embodiments 303-341, wherein the antibody construct comprises an anti-CD28 V_{H} sequence comprising the HCDR1 having the sequence SYGVH (SEQ ID NO: 300), the HCDR2 having the sequence VIWPGGGTNFNSALMS (SEQ ID NO: 303), and the HCDR3 having the sequence DRAYGNYLYAMDY (SEQ ID NO: 307), and an anti-CD28 V_{L} sequence comprising the LCDR1 having the sequence RASESVEYYVTSLMQ (SEQ ID NO: 313), the LCDR2 having the sequence AASNVDS (SEQ ID NO: 316), and the LCDR3 having the sequence QQSRKVPFT (SEQ ID NO: 320).

Embodiment 343. The antibody construct of any one of embodiments 303-342, wherein the antibody construct is capable of binding human CD3 with a dissociation constant (K_{D}) for CD3 of from about 20 nM to about 200 nM, from about 30 nM to about 150 nM, from about 40 nM to about 100 nM, or from 50 nM to about 80 nM, as determined using SPR.

Embodiment 344. The antibody construct of any one of embodiments 303-343, wherein the antibody construct comprises an anti-CD3 V_{H} sequence comprising the HCDR1-3 sequences set forth in SEQ ID NOs: 321-323, respectively, and an anti-CD3 V_{L} sequence comprising the LCDR1-3 sequences set forth in SEQ ID NOs: 324-326, respectively.

Embodiment 345. The antibody construct of any one of embodiments 303-344, wherein the antibody construct comprises an anti-CD3 V_{H} sequence comprising a HCDR1 having the sequence GVTFNYYG (SEQ ID NO: 321), a HCDR2 having the sequence ITSSGGRI (SEQ ID NO: 322), and a HCDR3 having the sequence TLDGRDGWVAY (SEQ ID NO: 323), and an anti-CD3 V_{L} sequence comprising a LCDR1 having the sequence TGNIGSNY (SEQ ID NO: 324), a LCDR2 having the sequence RND (SEQ ID NO: 325), and a LCDR3 having the sequence QSYSSGFI (SEQ ID NO: 326).

Embodiment 346. The antibody construct of any one of embodiments 303-345, wherein the antibody construct comprises an anti-CD3 V_{H} sequence having at least about 90%, 95%, 97%, 99%, or 100% sequence identity to the amino acid sequence set forth in SEQ ID NO: 102, and an anti-CD3 V_{L} sequence having at least about 90%, 95%, 97%, 99%, or 100% sequence identity to the amino acid sequence set forth in SEQ ID NO: 103.

Embodiment 347. The antibody construct of any one of embodiments 303-346, wherein the first cytotoxic effector cell and the second cytotoxic effector cell are different cells.

Embodiment 348. The antibody construct of any one of embodiments 303-346, wherein the first cytotoxic effector cell and the second cytotoxic effector cell are the same cell.

Embodiment 349. The antibody construct of any one of embodiments 303-348, wherein the first and second antigens are on a T cell.

Embodiment 350. The antibody construct of any one of embodiments 303-349, wherein the TAA is Cldn18.2.

Embodiment 351. The antibody construct of embodiment 350, wherein the antibody construct comprises an anti-Cldn 18.2 V_{H} sequence comprising the HCDR1-3 sequences set forth in SEQ ID NOs: 333-335, respectively, and an anti-Cldn 18.2 V_{L} sequence comprising the LCDR1-3 sequences set forth in SEQ ID NOs: 336-338, respectively.

Embodiment 352. The antibody construct of embodiment 351, wherein the antibody construct comprises an anti-Cldn18.2 V_{H} sequence comprising a HCDR1 having the sequence SNPMI (SEQ ID NO: 333), a HCDR2 having the sequence IIDTDGSTYYADWAKG (SEQ ID NO: 334), and a HCDR3 having the sequence RLHGSSNGYYDDL (SEQ ID NO: 335), and an anti-Cldn18.2 V_{L} sequence comprising a LCDR1 having the sequence QASQSIYSYLS (SEQ ID NO: 336), a LCDR2 having the sequence KASTLAS (SEQ ID NO: 337), and a LCDR3 having the sequence QQGYTVTNVDKNT (SEQ ID NO: 338).

Embodiment 353. The antibody construct of any one of embodiments 350-352, wherein the anti-Cldn18.2 V_{H} sequence comprises or consists of an amino acid sequence having at least about 90%, 95%, 97%, 99%, or 100% sequence identity to the amino acid sequence set forth in SEQ ID NO: 127, and the anti-Cldn18.2 V_{L} sequence comprises or consists of an amino acid sequence having at least about 90%, 95%, 97%, 99%, or 100% sequence identity to the amino acid sequence set forth in SEQ ID NO: 128, and, optionally, wherein the antibody construct binds to human Cldn18.2 with an affinity from about 1 nM to about 80 nM, from about 10 nM to about 60 nM, from about 10 nM to about 50 nM, or from about 20 nM to about 50 nM, as determined by flow cytometry.

Embodiment 354. The antibody construct of any one of embodiments 303-353, wherein the first Fc polypeptide and the second Fc polypeptide of the Fc domain each comprise or consist of a CH2 sequence and a CH3 sequence.

Embodiment 355. The antibody construct of embodiment 354, wherein at least one of the CH2 sequences of the first and second Fc polypeptide is an IgG1 or IgG4 CH2 sequence and comprises one or more amino acid modifications when compared to an unmodified wildtype IgG1 or IgG4 CH2 sequence.

Embodiment 356. The antibody construct of embodiment 355, wherein both CH2 sequences of the first and second Fc polypeptide are IgG1 or IgG4 CH2 sequences and comprise the one or more amino acid modifications when compared to an unmodified wildtype IgG1 or IgG4 CH2 sequence.

Embodiment 357. The antibody construct of embodiment 356, wherein the one or more amino acid modifications to the CH2 sequences reduce or ablate interactions of the Fc domain with one or more Fc receptors, optionally, one or more Fcγ receptors.

Embodiment 358. The antibody construct of any one of embodiments 354-357, wherein the CH2 sequence of both the first and the second Fc polypeptide comprises or consists of an amino acid sequence having at least about 90%, 95%, 97%, 99%, or 100% sequence identity to the amino acid sequence set forth in SEQ ID NO: 109.

Embodiment 359. The antibody construct of any one of embodiments 354-358, wherein at least one of the CH3 sequences of the first and second Fc polypeptide is an IgG1 or IgG4 CH3 sequence and comprises one or more amino acid modifications when compared to an unmodified wildtype IgG1 or IgG4 CH3 sequence, and, optionally, wherein the first and second Fc polypeptides have different amino acid sequences and form a heterodimeric Fc domain.

Embodiment 360. The antibody construct of embodiment 359, wherein both CH3 sequences of the first and second Fc polypeptide are IgG1 or IgG4 CH3 sequences and comprise one or more amino acid modifications that promote preferential pairing of the first and second Fc polypeptide to form the heterodimeric Fc domain compared to the formation of a corresponding homodimeric Fc domain.

Embodiment 361. The antibody construct of any one of embodiments 359-360, wherein the CH3 sequence of one of the Fc polypeptides comprises a set of amino acid substitutions selected from the group consisting of: L351Y_F405A_Y407V, T350V_L351Y_F405A_Y407V and T350V_L351Y_S400E_F405A_Y407V, and the CH3 sequence of the other Fc polypeptide comprises a set of amino acid substitutions selected from the group consisting of: T366L_K392M_T394W, T366L_K392L_T394W, T350V_T366L_K392L_T394W, T350V_T366L_K392M_T394W and T350V_T366L_N390R_K392M_T394W, and wherein the numbering of amino acid residues in the Fc polypeptides is according to the EU numbering system.

Embodiment 362. The antibody construct of any one of embodiments 354-361, wherein the CH3 sequence of one Fc polypeptide comprises or consists of an amino acid sequence having at least about 90%, 95%, 97%, 99%, or 100% sequence identity to the amino acid sequence set forth in SEQ ID NO: 110.

Embodiment 363. The antibody construct of any one of embodiments 354-362, wherein the CH3 sequence of the other Fc polypeptide comprises or consists of an amino acid sequence having at least about 90%, 95%, 97%, 99%, or 100% sequence identity to the amino acid sequence set forth in SEQ ID NO: 114.

Embodiment 364. The antibody construct of any one of embodiments 303-363, wherein the antibody construct is trivalent and trispecific and binds each antigen monovalently.

Embodiment 365. The antibody construct of any one of embodiments 303-364, wherein the antibody construct has a melting temperature at Tm1, Tm2, and/or Tm3 that is within 10° C., 5° C., 2° C., or within 1° C. degree of that of a corresponding bivalent and monospecific IgG1 monoclonal antibody.

Embodiment 366. The antibody construct of any one of embodiments 303-365, wherein the antibody construct binds the cytotoxic effector cell that expresses CD3 and/or CD28 with an affinity that is about 2-fold, 5-fold, 10-fold, 20-fold, 50-fold, 100-fold, or about 200-fold higher than that of a corresponding bivalent and bispecific anti-CD3×TAA and/or anti-CD28×TAA antibody construct.

Embodiment 367. The antibody construct of any one of embodiments 303-366, wherein the antibody construct exhibits an IC₅₀ value from about 50 pM to about 0.01 pM, from about 25 pM to about 0.01 pM, from about from about 10 pM to about 0.05 pM, from about 10 pM to about 0.1 pM, from about 10 pM to about 1 pM, from about 5 pM to about 1 pM for killing TAA-expressing tumor cells that express at least about 100,000 TAA/cell by TDCC in the presence of the cytotoxic effector cell and using an E:T ratio of 2:1 and an incubation period of 72 hours.

Embodiment 368. The antibody construct of embodiment 367, wherein the antibody construct achieves a maximum killing of TAA-expressing tumor cells of at least about 60%, 65%, 70%, 75%, or 80%, 85%, or 90%, 100%, or from about 60% to about 100%, from about 70% to about 90%, or from about 75% to about 85%.

Embodiment 369. The antibody construct of any one of embodiments 303-368, wherein the antibody construct is capable of inducing the production of one or more cytokines by the cytotoxic effector cell ranging from about 300 μg/mL to about 9000 μg/mL, when TAA-expressing cells expressing at least about 100,000 TAA/cell are present and using an E:T ratio of 2:1 and an incubation period of 72 hours.

Embodiment 370. A pharmaceutical composition comprising the antibody construct of any one of embodiments 303-369, and a pharmaceutically acceptable carrier, excipient, diluent, or combination thereof.

Embodiment 371. A nucleic acid molecule or a set of nucleic acid molecules encoding one or more, two or more, or three or more polypeptide chains that form the antibody construct of any one of embodiments 303-369.

Embodiment 372. A vector or a set of vectors comprising the nucleic acid molecule or the set of nucleic acid molecules of embodiment 371.

Embodiment 373. A cell comprising the nucleic acid molecule or the set of nucleic acid molecules of embodiment 371, or the vector or set of vectors of embodiment 372.

Embodiment 374. A method of producing an antibody construct of any one of embodiments 303-369, the method comprising: (a) obtaining a host cell culture comprising at least one host cell comprising one or more nucleic acid molecules encoding one or more, two or more, or three or more polypeptide chains that form the antibody construct; and (b) recovering the antibody construct from the host cell culture.

Embodiment 375. The method of embodiment 374, further comprising, subsequent to step (b), purifying the antibody construct.

Embodiment 376. A method of eliciting an anti-tumor immune response in a cell population comprising immune cells and tumor cells, the method comprising contacting the cell population with an effective amount of the antibody construct of any one of embodiments 303-369, wherein the immune cells express the first and second antigen and the tumor cells express the TAA.

Embodiment 377. A method of inhibiting the proliferation of tumor cells, the method comprising contacting a cell population comprising the tumor cells and immune cells with an effective amount of the antibody construct of any one of embodiments 303-369, wherein the immune cells express the first and second antigen and the tumor cells express the TAA.

Embodiment 378. A method of killing tumor cells, the method comprising contacting a cell population comprising the tumor cells and immune cells with an effective amount of the antibody construct of any one of embodiments 303-369, wherein the immune cells express the first and second antigen and the tumor cells express the TAA.

Embodiment 379. The method of any one of embodiments 376-378, wherein the immune cells comprise T cells.

Embodiment 380. The method of any one of embodiments 376-379, wherein TAA is Cldn18.2.

Embodiment 381. The method of any one of embodiments 376-380, wherein the antibody construct binds CD3 and CD28 on either one T cell or two different T cells, and the TAA on a tumor cell.

Embodiment 382. The method of embodiment 381, wherein the binding of the antibody construct of the first and second antigen and the TAA forms a TCR-independent artificial immune synapse between the one or more immune cells and the tumor cell, thereby eliciting a cytotoxic immune response of the immune cell against the tumor cell.

Embodiment 383. The method of any one of embodiments 376-382, wherein the cell population is within a subject.

Embodiment 384. A method for treating a cancer in a subject in need thereof, the method comprising administering to the subject an antibody construct of any one of embodiments 303-369.

Embodiment 385. The method of embodiment 384, wherein a cytotoxic immune response against the cancer is elicited in the subject, thereby treating the cancer in the subject.

Embodiment 386. An antibody construct of any one of embodiments 303-369 for use in the treatment of cancer.

Embodiment 387. Use of an antibody construct of any one of embodiments 303-369 in the manufacture of a medicament for the treatment of cancer.

Embodiment 388. An antibody construct, comprising a binding domain capable of binding CD28, wherein the binding domain comprises a V_{H} sequence comprising a HCDR1 having the sequence SX₁GVH (SEQ ID NO: 302), a HCDR2 having the sequence VIWX₂GGX₃TNFNSALMS (SEQ ID NO: 306), and a HCDR3 having the sequence DRAX₄GX₅YX₆X₇AMDY (SEQ ID NO: 312), and a V_{L} sequence comprising a LCDR1 having the sequence RASESVEYYX₈TSLMQ (SEQ ID NO: 315), a LCDR2 having the sequence AASX₉VX₁₀S (SEQ ID NO: 319), and a LCDR3 having the sequence QQSRKVPFT (SEQ ID NO: 320), having one or more of the following amino acid substitutions at the positions as identified in the CDR sequences: X₁: Y to A, X₂: P to A, X₃: G to S, X₄: S to Y, X₅: N to A, X₆: L to N, X₇: S to Y, X₈: G to V, X₉: N to A, and/or X₁₀: E to D.

Embodiment 389. The antibody construct of embodiment 388, wherein the binding domain comprises a V_{H} sequence comprising or consisting of an amino acid sequence selected from the group consisting of SEQ ID NOs: 201, 203-208, and 210, and a V_{L} sequence comprising or consisting of an amino acid sequence selected from the group consisting of SEQ ID NOs: 200, 202, and 209.

Embodiment 390. An antibody construct, comprising a binding domain capable of binding Cldn18.2, wherein the binding domain comprises a V_{H} sequence comprising a HCDR1 having the sequence SNPMI (SEQ ID NO: 333), a HCDR2 having the sequence IIDTDGSTYYADWAKG (SEQ ID NO: 334), and a HCDR3 having the sequence RLHGSSNGYYDDL (SEQ ID NO: 335), and a V_{L} sequence comprising a LCDR1 having the sequence QASQSIYSYLS (SEQ ID NO: 336), a LCDR2 having the sequence KASTLAS (SEQ ID NO: 337), and a LCDR3 having the sequence QQGYTVTNVDKNT (SEQ ID NO: 338).

Embodiment 391. The antibody construct of embodiment 390, wherein the binding domain comprises a V_{H} sequence comprising or consisting of an amino acid sequence having at least about 90%, 95%, 97%, 99%, or 100% sequence identity to the amino acid sequence set forth in SEQ ID NO: 127, and a V_{L} sequence comprising or consisting of an amino acid sequence having at least about 90%, 95%, 97%, 99%, or 100% sequence identity to the amino acid sequence set forth in SEQ ID NO: 128.

Embodiment 392. The antibody construct of any one of embodiments 388-391, further comprising one or more additional binding domains capable of binding one or more additional antigens.

Embodiment 393. The antibody construct of any one of embodiments 1, 6, 7, 23-30, 147, 152, 153 and 169-176, wherein the first scFv domain and the second scFv domain are not coupled to each other in tandem.

### EXAMPLES

The following Examples are provided for illustrative purposes and are not intended to limit the scope of the invention in any way.

The practice of the present disclosure can employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Design and Preparation of Trivalent and Trispecific Anti-(MSLN×CD28×CD3) Antibody Constructs

Several trivalent and trispecific anti-(MSLN×CD28×CD3) antibody constructs as well as bispecific anti-(MSLN×CD3) and anti-(MSLN×CD28) control antibody constructs were produced as described below. The trivalent and trispecific antibody constructs and controls were prepared in different formats and geometries, as shown, e.g., in FIGS. 1A-1G. Such antibody constructs were prepared to examine the impact of antibody format, geometry and anti-CD28 and anti-CD3 paratope affinity on the potency of these T cell engager constructs, e.g., their capability of targeting and killing MSLN-expressing tumor cells.

### Design of Trivalent and Trispecific Antibody Constructs Targeting MSLN, CD28 and CD3

Trivalent and Trispecific antibody constructs that are monovalent for each antigen were prepared in a format in which the MSLN antigen binding domain was an scFv domain, the CD3 antigen binding domain was either an scFv domain or a Fab domain, and the CD28 antigen binding domain was either an scFv domain or a Fab domain.

Bispecific antibody constructs were prepared in a format in which MSLN antigen-binding domain was an scFv domain and the CD3 or CD28 antigen-binding domain was a Fab domain. These trispecific and bispecific control antibody constructs comprised a human IgG1 heterodimeric Fc domain which further comprised sets of CH3 domain amino acid substitutions promoting the formation of a heterodimeric Fc domain (see, e.g., TABLE 5). These sets of amino acid substitutions are referred to herein as (i) Het FcA having the amino acid substitutions T350V/L351Y/F405A/Y407V in the first Fc polypeptide chain (A) and (ii) Het FcB having the amino acid substitutions T350V/T366L/K392L/T394W in the second Fc polypeptide chain (B). The antibody construct variants in TABLE 6 are noted as having “FcKO” comprised the following CH2 amino acid substitutions which knock out, e.g., measurably reduce or ablate, FcγR binding: L234A, L235A and D265S. Amino acid residues in the Fc domain are identified according to the EU index.

The trivalent and trispecific antibody constructs against MSLN, CD28 and CD3 and bispecific control constructs were expressed and characterized as described in WO2015109131. Briefly, the genes encoding the antibody constructs' heavy and light chains were constructed via gene synthesis using codons optimized for human/mammalian expression. The anti-MSLN scFv domain sequence was generated from the V_{H} and V_{L} sequences of the mouse anti-MSLN antibody (see, e.g., Nat Biotechnol. 1999 June; 17 (6): 568-72) with additional humanizing mutations. The anti-CD3 scFv domain and Fab domain sequences were generated from the anti-CD3 monoclonal antibody C3E6 (see, e.g., U.S. Pat. Publ. No. 2019/0359712). The anti-CD28 scFv domain and Fab domain sequences were generated from the anti-CD28 monoclonal antibody huTN228 (see, e.g., Shiao et al., Transplantation. 2007; 83 (3): 304-13).

In the trispecific as well as bispecific antibody constructs of this disclosure containing an anti-CD3 Fab domain or an anti-CD28 Fab domain, the V_{H} and V_{L} sequences were coupled to human IgG1 C_{H1} and C_{L} sequences, respectively. In some trispecific antibody constructs of this disclosure, an anti-CD3 scFv domain was coupled to the N-terminus of the V_{H} domain of the anti-CD28 Fab domain or to the N- or C-termini of the first or second Fc polypeptide of the heterodimeric Fc domain. In other trispecific antibody constructs of this disclosure, an anti-CD28 scFv domain was coupled to the N-terminus of the V_{H} or V_{L} domain of the anti-CD3 Fab domain or to the C-terminus of the C_{L} domain of the anti-CD3 Fab domain. In various trivalent and trispecific antibody constructs of this disclosure, an anti-MSLN scFv domain was coupled to the N-terminus of the first or second Fc polypeptide of the heterodimeric Fc domain. In bispecific control antibody constructs containing an anti-CD3 Fab domain or an anti-CD28 Fab domain, the anti-CD3 or anti-CD28 V_{H} and V_{L} domain sequences were coupled to human IgG1 C_{H1} and C_{L} sequences, respectively. In cases of the bispecific control constructs, the scFv domains were coupled to one Fc polypeptide chain of the heterodimeric Fc domain and the V_{H}-C_{H1} domains of the Fab domain were fused to the second Fc polypeptide chain of the heterodimeric Fc domain.

Several heavy chain domain structures and formats that were used to generate certain antibody constructs of the present disclosure, e.g., the trivalent and trispecific as well as bispecific constructs, and/or other constructs for studying the impact of overall construct format and geometry on a construct's properties, are shown below in, e.g., TABLE 5, from N-terminus to C-terminus.

### Example 2: Design and Preparation of Trivalent and Trispecific Anti-(CLDN18.2×CD28×CD3) Antibody Constructs

Several trivalent and trispecific anti-(CLDN18.2 (Cldn18.2)×CD28×CD3) antibody constructs and bispecific anti-(CLDN18.2×CD3) and anti-(CLDN18.2×CD28) control antibody constructs were produced as described below. The antibody constructs and controls were prepared in different formats and geometries, as shown, e.g., in FIGS. 1A-1F. Such antibody constructs were prepared to examine the impact of antibody format, geometry and anti-CD28 and anti-CD3 paratope affinity on the potency of these T cell engager constructs, e.g., their capability of targeting CLDN18.2-expressing tumor cells.

### Design of Trivalent and Trispecific Antibody Constructs Targeting CLDN18.2, CD28 and CD3

Trivalent and trispecific antibody constructs were prepared in a format in which the CLDN18.2 antigen-binding arm was an scFv domain, the CD3 antigen-binding domain was either an scFv domain or a Fab domain, and the CD28 antigen-binding domain was either an scFv domain or a Fab domain.

Bispecific control antibody constructs were prepared in a format in which CLDN18.2 antigen-binding domain was an scFv domain and the CD3 or CD28 antigen-binding domain was a Fab domain.

These trispecific and bispecific antibody constructs comprised a human IgG1 heterodimeric Fc domain which further comprised sets of CH3 domain amino acid substitutions promoting the formation of a heterodimeric Fc domain (see, e.g., “Het-Fc” in TABLE 6). These sets of amino acid substitutions are referred to herein as (i) Het FcA having the amino acid substitutions T350V/L351Y/F405A/Y407V in the first Fc polypeptide chain (A) and (ii) Het FcB having the amino acid substitutions T350V/T366L/K392L/T394W in the second Fc polypeptide chain (B). These antibody construct variants in TABLE 6 herein noted as having “FcKO” comprised the following CH2 amino acid substitutions which knock out, e.g., measurably reduce or ablate, FcγR binding: L234A, L235A and D265S. Amino acid residues in the Fc domain are identified according to the EU index.

The trivalent and trispecific antibody constructs as well as the bispecific control constructs against CLDN18.2, CD28 and/or CD3 were designed, expressed, and characterized as described in WO2015109131. Briefly, the genes encoding the antibody constructs' heavy and light chains were constructed via gene synthesis using codons optimized for human/mammalian expression. The anti-CD3 scFv domain and Fab domain sequences were generated from the anti-CD3 monoclonal antibody C3E6 (see, e.g., U.S. Pat. Publ. No. 2019/0359712). The anti-CD28 scFv domain and Fab domain sequences were generated from the anti-CD28 monoclonal antibody huTN228 as described herein. The anti-CLDN18.2 scFv domain sequence was generated from the V_{H} and V_{L} sequences of the humanized rabbit anti-CLDN18.2 antibody (13B08, internal Ab). The anti-CLDN18.2 antibody was generated and humanized as described in EXAMPLE 3 of this disclosure, respectively.

In trivalent and trispecific antibody constructs as well as the bispecific control constructs of this disclosure containing an anti-CD3 Fab domain or an anti-CD28 Fab, the V_{H} and V_{L} sequences were fused to human IgG1 C_{H1} and C_{L} sequences, respectively. In some trispecific antibody constructs of this disclosure, anti-CD3 scFv domain or an anti-CD28 scFv domain were fused to the N- or C-termini of one of the Fc polypeptides of the heterodimeric Fc domain. In other trispecific antibody constructs of this disclosure, anti-CD3 scFv domain was fused to the N-terminus of the V_{H} domain of the anti-CD28 Fab domain or to the C-terminus of the C_{L} domain of the anti-CD28 Fab domain. In other trispecific antibody constructs of this disclosure, anti-CD28 scFv domain was fused to the N-terminus of the V_{H} domain of the anti-CD3 Fab domain or to the C-terminus of the C_{L} domain of the anti-CD3 Fab domain. In certain trispecific and bispecific antibody constructs of this disclosure, an anti-CLDN18.2 scFv domain was fused either to one of the heterodimeric Fc domains or to the N-terminus of the V_{H} domain of the anti-CD3 Fab domain or to the N-terminus of the V_{H} domain of the anti-CD28 Fab domain. In bispecific control antibody constructs containing an anti-CD3 Fab domain or an anti-CD28 Fab domain, the anti-CD3 or anti-CD28 V_{H} and V_{L} domain sequences were fused to human IgG1 C_{H1} and C_{L} sequences, respectively. In all embodiments, the scFv domains were fused to one chain of the heterodimeric Fc domain and the V_{H}-C_{H1} domains of the Fabs were fused to the second chain of the heterodimeric Fc domain.

Some heavy chain domain structures and formats that were used to generate certain antibody constructs of the present disclosure, e.g., some trivalent and trispecific constructs, bispecific control constructs, as well as other constructs for studying the impact of overall construct format and geometry, are shown below in TABLE 5, from N-terminus to C-terminus:

TABLE 5
Heavy Chain Domain Structures Used in Certain Antibody Constructs

| | Certain Antibody Constructs that can |
|---|---|
| Heavy Chain Domain Structure: | comprise the Chain Format: |
| V_{H}-C_{H1}-hinge-CH2-CH3 | v34915, v34916, v34919, v34927, v37634, |
| | v37639 |
| V_{H}-V_{L}-hinge-CH2-CH3 | v37633, v37634, v37635, v37637, v37638, |
| | v37639, v37640, v37642, v37658, v37660, |
| | v37662, v37663, v37665, v37667, v37668 |
| V_{L}-V_{H}-hinge-CH2-CH3 | v34913, v34914, v34915, v34916, v34917 |
| | v34918, v34919, v34927 |
| V_{H}-V_{L}-V_{H}-C_{H1}-hinge-CH2-CH3 | v34913, v34914, v37633, v37637, v37638, |
| | v37642, v37658, v37662, v37664, v37666, |
| | v37667 |
| V_{H}-C_{H1}-hinge-CH2-CH3-V_{L}-V_{H} | v34917, v34918, v37635, v37640, v37650, |
| | v37669 |
| V_{H}-V_{L}-VH-CH1-hinge-CH2-CH3-VL-VH | v37636, v37641 |
| CH2-CH3 | v37636, v37641 |

TABLE 6 below provides a description of certain trivalent and trispecific anti-(MSLN×CD28×CD3) and anti-(CLDN18.2×CD28×CD3) construct as well as bispecific control constructs that were prepared herein with varying valencies, formats and geometries. The respective numbers of antigen-targeting domains for each construct are indicated in the “Format” column. As described herein, the “1” indicates monovalent binding against a certain antigen, and a “2” indicates bivalent binding against a certain antigen.

TABLE 6
Description of Certain Antibody Constructs of the Present Disclosure

| Construct # | Format | Heavy Chain A | Heavy Chain B | Fc Description |
|---|---|---|---|---|
| 34913 | 1x1x1 | N-terminus C3E6 VH-VL | MSLN VL-VH scFv | Het Fc, FcKO |
| | | scFv, huTN228 Fab | | |
| 34914 | 1x1x1 | N-terminus huTN228 | MSLN VL-VH scFv | Het Fc, FcKO |
| | | VH-VL scFv, C3E6 Fab | | |
| 34915 | 1x1x1 | C3E6 Fab | MSLN VL-VH scFv | Het Fc, FcKO |
| 34916 | 1x1x1 | C3E6 Fab | MSLN VL-VH scFv | Het Fc, FcKO |
| 34917 | 1x1x1 | C3E6 Fab, C-terminus | MSLN VL-VH scFv | Het Fc, FcKO |
| | | huTN228 VL-VH scFv | | |
| 34918 | 1x1x1 | huTN228 Fab, C-terminus | MSLN VL-VH scFv | Het Fc, FcKO |
| | | C3E6 VL-VH scFv | | |
| 34919 | 1x1 | C3E6 Fab | MSLN VL-VH scFv | Het Fc, FcKO |
| 34927 | 1x1 | huTN228 Fab | MSLN VL-VH scFv | Het Fc, FcKO |
| 37633 | 1x1x1 | N-terminus huTN228 | 13B08 VH-VL scFv | Het Fc, FcKO |
| | | VH-VL scFv, C3E6 Fab | | |
| 37634 | 1x1x1 | C3E6 Fab | 13B08 VH-VL scFv | Het Fc, FcKO |
| 37635 | 1x1x1 | C3E6 Fab, C-terminus | 13B08 VH-VL scFv | Het Fc, FcKO |
| | | huTN228 VL-VH scFv | | |
| 37636 | 1x1x1 | N-terminus 13B08 VH- | No paratope | Het Fc, FcKO |
| | | VL scFv, C3E6 Fab, C- | | |
| | | terminus huTN228 VL- | | |
| | | VH scFv | | |
| 37637 | 1x1x1 | huTN228 VH-VL scFv | N-terminus 13B08 | Het Fc, FcKO |
| | | | VH-VL scFv, C3E6 | |
| | | | Fab | |
| 37638 | 1x1x1 | N-terminus C3E6 VH-VL | 13B08 VH-VL scFv | Het Fc, FcKO |
| | | scFv, huTN228 Fab | | |
| 37639 | 1x1x1 | huTN228 Fab | 13B08 VH-VL scFv | Het Fc, FcKO |
| 37640 | 1x1x1 | huTN228 Fab, C-terminus | 13B08 VH-VL scFv | Het Fc, FcKO |
| | | C3E6 VL-VH scFv | | |
| 37641 | 1x1x1 | N-terminus 13B08 VH- | No paratope | Het Fc, FcKO |
| | | VL scFv, huTN228 Fab, | | |
| | | C-terminus C3E6 VL-VH | | |
| | | scFv | | |
| 37642 | 1x1x1 | N-terminus 13B08 VH- | 13B08 VH-VL scFv | Het Fc, FcKO |
| | | VL scFv, huTN228 Fab | | |
| 37658 | 1x1x1 | N-terminus C3E6 VH-VL | 13B08 VH-VL scFv | Het Fc, FcKO |
| | | scFv, Palivizumab Fab | | |
| 37660 | 1x1x1 | Palivizumab Fab, C- | 13B08 VH-VL scFv | Het Fc, FcKO |
| | | terminus C3E6 VL-VH | | |
| | | scFv | | |
| 37662 | 1x1x1 | N-terminus 13B08 VH- | 13B08 VH-VL scFv | Het Fc, FcKO |
| | | VL scFv, Palivizumab | | |
| | | Fab | | |
| 37663 | 1x1 | C3E6 Fab | 13B08 VH-VL scFv | Het Fc, FcKO |
| 37664 | 1x1 | No paratope | N-terminus 13B08 | Het Fc, FcKO |
| | | | VH-VL scFv, C3E6 | |
| | | | Fab | |
| 37665 | 1x1 | huTN228 Fab | 13B08 VH-VL scFv | Het Fc, FcKO |
| 37666 | 1x1 | N-terminus 13B08 VH- | No paratope | Het Fc, FcKO |
| | | VL scFv, huTN228 Fab | | |
| 37667 | 2x1 | N-terminus C3E6 VH-VL | 13B08 VH-VL scFv | Het Fc, FcKO |
| | | scFv, C3E6 Fab | | |
| 37669 | 2x1 | C3E6 Fab, C-terminus | 13B08 VH-VL scFv | Het Fc, FcKO |
| | | C3E6 VL-VH scFv | | |
| 35923 | 1x1 | Anti-CLDN18.2 VH - VL | n/a | n/a |
| AMG-910 | | scFv- Anti-CD318.2 VH - | | |
| (Benchmark | | VL scFv | | |
| 22277 | 1x1 | Palivizumab Fab | Palivizumab Fab | Het Fc |
| 31926 | 1x1 | N-terminus anti- | Anti-hemagglutinin | Het Fc, FcKO |
| | | hemagglutinin VH-VL | VH-VL scFv | |
| | | scFv, C3E6 Fab | | |

By way of example, in TABLE 6, the nomenclature “1×1×1” indicates that the trispecific antibody construct is trivalent and trispecific (e.g., engaging each antigen monovalently) and comprised either one MSLN binding domain, one CD28 binding domain and one CD3 binding domain or one CLDN18.2 binding domain, one CD28 binding domain and one CD3 binding domain. Accordingly, “1×1” indicates that the bispecific control antibody construct is bivalent and comprised of either one MSLN or CLDN18.2 binding domains and one CD3 or CD28 binding domain, etc. Certain formats (e.g., 1×1×1, 1×1, 2×1, etc.) and geometries (e.g., relative positioning of binding domains) of certain antibody constructs described herein are also illustrated in, e.g., FIGS. 1A-1L.

### Production of Trivalent and Trispecific Anti-(MSLN×CD3×CD28) and Anti-(CLDN18.2×CD3×CD28) Antibody Constructs

Generally, the final gene products were sub-cloned into the mammalian expression vector pTT5 (NRC-BRI, Canada) and expressed in CHO cells (Durocher, Y., Perret, S. & Kamen, A. High-level and high-throughput recombinant protein production by transient transfection of suspension-growing CHO cells. Sec, e.g., Nucleic acids research 30, E9 (2002)).

The CHO cells were transfected in exponential growth phase (1.5 to 2 million cells/mL) with aqueous 1 mg/mL 25 kDa polyethylenimine (PEI, Polysciences) using an PEI: DNA ratio of 2.5:1 (see, e.g., Raymond C. et al. A simplified polyethylenimine-mediated transfection process for large-scale and high-throughput applications. Methods. 55 (1): 44-51 (2011)). In order to determine the optimal concentration range for forming heterodimers, the DNA was transfected in optimal DNA ratios of the heavy chain A (HC-A), heavy chain B (HC-B), and light chain(s) (LC) that allow for heterodimer formation (e.g., HC-A:HC-B:LC ratios=1:1:1 or 2:1:1 or 1:1.5:1 or 1:2:1.5 or 1:1:1.5 or 2:1:3). Transfected cells were harvested after 5-6 days with the culture medium collected after centrifugation at 4000 rpm and clarified using a 0.45 μm filter.

The clarified culture medium was loaded onto a MabSelect SuRe (GE Healthcare) protein-A column and washed with 10 column volumes of PBS buffer at pH 7.2-7.4. The antibody construct was eluted with 10 column volumes of 0.1 M citrate buffer at pH 3.6 with the pooled fractions containing the antibody construct neutralized with 1 M TRIS at pH 9 or 11. Antibody construct was then quantified based on A280 nm (NanoDrop™).

The antibody constructs were further purified by gel filtration chromatography using a Superdex 200 HiLoad 16/600 200 pg column (GE Healthcare) via an AKTA Express FPLC or AKTA Pure at a flowrate of 1 mL/min. PBS buffer at pH 7.4 or A5-NaCl (50 mM sodium acetate, 150 mM sodium chloride, pH 5.0) or H6-NaCl (50 mM histidine, 150 mM sodium chloride, pH 6.0) buffer was used at a flow-rate of 1 mL/min. Fractions of eluted antibody construct were collected based on absorbance at A280 nm and the fractions were assessed by non-reducing and reducing High Throughput Protein Express assay using Caliper LabChip GXII (Perkin Elmer, Waltham, MA). Procedures were carried out according to HT Protein Express LabChip User Guide version2 LabChip GXII User Manual, with the following modifications. Antibody construct samples, at either 2 μl or 5 μl (concentration range 5-2000 ng/μl), were added to separate wells in 96 well plates (BioRad, Hercules, CA) along with 7 μl of HT Protein Express Sample Buffer (Perkin Elmer #760328). Antibody construct samples were then denatured at 90° C. for 5 mins. The LabChip instrument was operated using the HT Protein Express Chip (Perkin Elmer, Waltham, MA) and the Ab-200 assay setting. Fractions corresponding to the purified antibody constructs were collected, buffer exchanged into A5Su (50 mM sodium acetate, 9% sucrose, pH 5.0) or H6Su (50 mM histidine, 9% sucrose, pH 6.0) using a Zeba Spin desalting column (Thermo Scientific), concentrated to ˜1 mg/mL, and stored at −80° C.

Endotoxin levels were determined by the LAL (limulus amebocyte lysate) assay using the Endosafe Portable Test System (PTS, Charles River, Wilmington, MA). Antibody constructs were qualified based on A280 nm absorbance (nanodrop) post protein-A and SEC purification. UPLC-SEC was performed using a Waters Acquity BEH200 SEC column (2.5 mL, 4.6×150 mm, stainless steel, 1.7 μm particles) (Waters LTD, Mississauga, ON) set to 30° C. or 25° C. and mounted on a Waters Acquity UPLC H-Class Bio system with a PDA detector. Run times consisted of 7 min and a total volume per injection of 2.8 μL or 5 μL with a running buffer of DPBS or DPBS with 0.02% Tween 20 pH 7.4- or 200 mM Potassium Phosphate with 200 mM Potassium Chloride pH 7.0 at 0.4 mL/min. Elution was monitored by UV absorbance in the range 210-500 nm, and chromatograms were extracted at 280 nm. Peak integration was performed using Empower 3 software.

The apparent purity and yield of the final antibody construct was estimated by UPLC-SEC and LC/MS as described in detail in, e.g., WO2015109131. All antibody constructs were expressed and purified to >90% heterodimer purity without contaminating homodimers.

TABLE 7 below describes the post purification yield for the production of certain antibody constructs described herein.

TABLE 7
Post Purification Yield and Purity for Certain Antibody Constructs

| Antibody | Yield | Purity | |
|---|---|---|---|
| Construct | (mg/L) | (%)* | Buffer |
| v34913 | 285 | 98.5 | 50 mM NaAc, 9% Sucrose, pH 5.0 |
| v34914 | 386 | 98.5 | 50 mM NaAc, 9% Sucrose, pH 5.0 |
| v34915 | 260 | 95.1 | 50 mM NaAc, 9% Sucrose, pH 5.0 |
| v34916 | 469 | 97.2 | 50 mM NaAc, 9% Sucrose, pH 5.0 |
| v34917 | 158 | 99.3 | 50 mM NaAc, 9% Sucrose, pH 5.0 |
| v34918 | 304 | 96.9 | 50 mM NaAc, 9% Sucrose, pH 5.0 |
| v34919 | 105 | 97.9 | 50 mM NaAc, 9% Sucrose, pH 5.0 |
| v34927 | 478 | 98.5 | 50 mM NaAc, 9% Sucrose, pH 5.0 |
| v37633 | 23 | 99.5 | 50 mM Histidine, 9% Sucrose, pH 6.0 |
| v37634 | 29 | 99.8 | 50 mM Histidine, 9% Sucrose, pH 6.0 |
| v37635 | 38 | 99.4 | 50 mM Histidine, 9% Sucrose, pH 6.0 |
| v37636 | 19 | 99.3 | 50 mM Histidine, 9% Sucrose, pH 6.0 |
| v37637 | 33 | 94.2 | 50 mM Histidine, 9% Sucrose, pH 6.0 |
| v37638 | 33 | 99.8 | 50 mM Histidine, 9% Sucrose, pH 6.0 |
| v37639 | 23 | 98.8 | 50 mM Histidine, 9% Sucrose, pH 6.0 |
| v37640 | 20 | 99.8 | 50 mM Histidine, 9% Sucrose, pH 6.0 |
| v37641 | 23 | 99.5 | 50 mM Histidine, 9% Sucrose, pH 6.0 |
| v37642 | 35 | 99.6 | 50 mM Histidine, 9% Sucrose, pH 6.0 |
| v37658 | 39 | 97.4 | 50 mM Histidine, 9% Sucrose, pH 6.0 |
| v37660 | 50 | 99.6 | 50 mM Histidine, 9% Sucrose, pH 6.0 |
| v37662 | 46 | 99.7 | 50 mM Histidine, 9% Sucrose, pH 6.0 |
| v37663 | 44 | 99.6 | 50 mM Histidine, 9% Sucrose, pH 6.0 |
| v35923 | 7 | 98.4 | 50 mM Histidine, 9% Sucrose, pH 6.0 |

*As determined by HPLC-SEC (size-exclusion chromatography HPLC)

### Example 3: Preparation of Anti-CLDN18.2 Antibodies

Antibodies that specifically bind CLDN18.2 were generated by immunizing rabbits with transiently transfected CHO cells expressing human CLDN18.2, as described below. These antibodies, or binding fragments thereof, can be useful for targeting human Cldn18.2, e.g., by using these generated anti-Cldn18.2 binding domains in the trivalent and trispecific antibody constructs of the present disclosure.

CHO-S cells (Invitrogen, Waltham, MA; Cat #R80007) were transiently transfected with a pTT5-based expression plasmid (National Research Council of Canada) encoding human CLDN18.2 according to manufacturer's instructions for the Neon Transfection System (Thermo Fisher Scientific, Waltham, MA). Two New Zealand White rabbits were subcutaneously immunized with transfected CHO cells over 63 days, after which blood was drawn and spleens harvested.

Anti-human CLDN18.2 antibody titers were determined by flow cytometry using HEK293-6E cells (National Research Council of Canada) expressing human CLDN18.2. Both rabbits mounted a significant response against human CLDN18.2.

Immunized rabbits were sacrificed, and the spleens harvested. Splenocytes for each rabbit were used for B cell enrichment and sorted on a FACSAria™ (Becton, Dickinson & Co., Franklin Lakes, NJ) into wells containing lysis buffer with a modified protocol based on the Selected Lymphocyte Antibody Method (SLAM) (Babcook et al., 1996, Proc Natl Acad Sci USA, 93 (15): 7843-7848).

Total RNA from wells containing a single B cell was used as template with SuperScript™ III (Thermo Fisher Scientific Corp., Waltham, MA) and oligo-dT20 (Integrated DNA Technologies, Inc., Coralville, IA) to transcribe cDNA from mRNA. Initial PCR of heavy and light chain antibody-coding sequences was performed using primers and methods modified from Babcook et al., 1996, Proc Natl Acad Sci USA, 93 (15): 7843-7848 and von Boehmer et al., 2016, Nat Protoc., 11 (10): 1908, with cDNA as the nucleic acid template. A subsequent PCR reaction was then performed on these unique sequences using V-segment family and J-segment family-specific primers and the resulting amplicons were cloned into pTT5-based expression plasmids (National Research Council of Canada). Unique heavy chain sequences and light chain sequences emerging from a single well sample were co-expressed in Expi293F cells (Thermo Fisher Scientific, Waltham, MA; Cat #A14527).

Cell supernatants containing secreted antibodies were assessed for human CLDN18.2 specificity by binding on HEK293-6E transfected with the same pTT5 based plasmid used to transfect CHO-S cells. The wells corresponding to human CLDN18.2-binding antibody containing supernatant were selected for sequencing.

Heavy and light chain PCR amplicons were sequenced using NGS-based Amplicon-EZ and analyzed for unique antibody-coding sequences. The following rabbit anti-human CLDN18.2 antibody V_{H} and V_{L} sequences was identified for the antibody referred to below as v35777:

TABLE 8
Rabbit V_{H} and V_{L} Sequences for Anti-Human
CLDN18.2 Antibody

| | | SEQ |
|---|---|---|
| | | ID |
| Description | Sequence | NO: |
| Rabbit V_{H} | QSLEESGGRLVTPGTPLTLTCTVSGIDLSSNPMI | 2 |
| | WVRQAPGKGLQYIGIIDTDGSTYYASWAKGRFTG | |
| | SKTSTTVDLKITSPTTEDTATYFCARRLHGSSNG | |
| | YYDDLWGQGTLVTISS | |
| Rabbit V_{L} | DGVMTQTPSSVSAAVGGTVTIKCQASQSIYSYLS | 3 |
| (kappa) | WYQQKPGQRPKLLIYKASTLASGVPSRFKGSGSG | |
| | TEFTLTISGVQCDDAATYYCQQGYTVTNVDKNTF | |
| | GGGTEVVVK | |

These rabbit V_{H} and V_{L} sequence was used to prepare a rabbit-human chimeric IgG1/kappa antibody construct, v35777, as follows. Coding sequences for antibody variable regions were cloned in frame into a human IgG1 expression vector or a human C kappa expression vector (based on the pTT5 vector). The human IgG1 constant region starts at alanine Kabat-114 and human C kappa constant region starts at arginine Kabat-108. The activities of the resultant recombinant chimeric antibody construct were confirmed in specificity binding assays.

### Creation of Humanized Candidate Sequences

The rabbit V_{H} and V_{L} sequences from chimeric antibody construct v35777 were aligned against human immunoglobulin germline sequences to select basis germline sequences for humanization. Human germline IGHV3-64*04 with IGHJ4*01 was selected for V_{H} humanization. Human germline IGKV1-39*01 with IGKJ4*01 was selected for V_{L} humanization. The CDR sequences by AbM definition from v35777 were swapped into the selected human germline frameworks to create a basis humanized construct. Several areas of the basis construct were identified for back mutation or deletion to the original rabbit parental sequence to minimize potential disruption to antigen binding. Five new candidate humanized V_{H} sequences, and four new candidate humanized V_{L} sequences were thus created.

### Screening of Humanized Candidate Sequences for Antigen Binding Activity

Recombinant human IgG1-based monoclonal antibodies containing each combination of the candidate V_{H} and V_{L} humanized sequences were expressed in Expi293F and the supernatants screened for binding to SNU-601 cells or HEK293-6E cells transfected with either human CLDN18.2 or human CLDN18.1 by FACS. All antibodies showed no detectable binding to HEK293-6E transfected with CLDN18.1. Five constructs represented by 4 humanized V_{H} and 2 humanized V_{L} sequences (TABLES 9 and 10) demonstrated binding activity similar to the parental v35777 construct (TABLE 13) and were selected for further analysis.

TABLE 9
Humanized V_{H} and V_{L} Domain Sequences

| | | SEQ |
|---|---|---|
| V_{H}/V_{L} | | ID |
| IDS | V_{H}/V_{L} Sequence | NO |
| H2 | QVQLVESGGGLVQPGGSLRLSCSVSGIDLSSNPMIWVRQ | 4 |
| | APGKGLEYIGIIDTDGSTYYADWAKGRFTISKDSSKNTVY | |
| | LQMNSLRAEDTAVYYCARRLHGSSNGYYDDLWGQGTL | |
| | VTVSS | |
| H3 | QVQLVESGGGLVQPGGSLRLSCSVSGIDLSSNPMIWVRQ | 5 |
| | APGKGLQYIGIIDTDGSTYYADWAKGRFTISKDSSKNTVY | |
| | LQMNSLRAEDTAVYYCARRLHGSSNGYYDDLWGQGTL | |
| | VTVSS | |
| H4 | QVQLVESGGGLVQPGGSLRLSCSVSGIDLSSNPMIWVRQ | 6 |
| | APGKGLEYIGIIDTDGSTYYADWAKGRFTISKDSTTVYLQ | |
| | MNSLRAEDTAVYYCARRLHGSSNGYYDDLWGQGTLVT | |
| | VSS | |
| H5 | QVQLVESGGGLVQPGGSLRLSCSVSGIDLSSNPMIWVRQ | 127 |
| | APGKGLQYIGIIDTDGSTYYADWAKGRFTISKDSTTVYLQ | |
| | INSPRAEDTAVYYCARRLHGSSNGYYDDLWGQGTLVTV | |
| | SS | |
| L2 | DGQMTQSPSSVSASVGDRVTITCQASQSIYSYLSWYQQK | 7 |
| | PGQRPKLLIYKASTLASGVPSRFSGSGSGTDFTLTISSVQ | |
| | PEDAATYYCQQGYTVTNVDKNTFGGGTKVEVK | |
| L4 | DGQMTQSPSSVSASVGDRVTITCQASQSIYSYLSWYQQK | 128 |
| | PGQRPKLLIYKASTLASGVPSRFSGSGSGTDFTLTISSVQ | |
| | PEDFATYYCQQGYTVTNVDKNTFGGGTKVEIK | |

TABLE 10
V_{H} and V_{L} Composition of Final Selected Constructs

| Construct | Humanized V_{H} and V_{L} composition |
|---|---|
| v37407 | H4-L4 |
| v37408 | H4-L2 |
| v37409 | H2-L4 |
| v37410 | H3-L4 |
| v37411 | H5-L4 |

The five selected humanized constructs (v37407, v37408, v37409, v37410, v37411) plus the parental rabbit chimeric construct (v35777) were expressed in 2.5 mL ExpiCHO cultures and purified by Protein A for further characterization. Production titers were significantly increased for the humanized variants compared to the parental chimeric. All humanized constructs expressed to high level and were easily purified by Protein A (mAb SelectSuRe) with high yield and purity (TABLE 11).

TABLE 11
Production of Parental Rabbit Chimeric and Humanized Constructs

| | | Culture | | | |
|---|---|---|---|---|---|
| | | volume | Titer | Yield | UPLC-SEC |
| Construct | Description | (mL) | (mg/L) | (mg) | % purity |
| v35777 | parental | 510 | 20.6 | 12.1 | 95.0 |
| | rabbit | | | | |
| | chimeric | | | | |
| v37407 | H4-L4 | 380 | 564.4 | 233.1 | >99 |
| v37408 | H4-L2 | 175 | 568.1 | 113.1 | >99 |
| v37409 | H2-L4 | 175 | 442.3 | 131.9 | >99 |
| v37410 | H3-L4 | 175 | 512.8 | 139.7 | >99 |
| v37411 | H5-L4 | 175 | 447.7 | 123.0 | >99 |

The purified rabbit chimeric and humanized constructs were assessed for binding to HEK293-6E cells transfected with human, cynomolgus monkey, or mouse CLDN18.2 orthologs (TABLE 12) by flow cytometry. All constructs bound each ortholog-transfected cell with similar single-digit nanomolar or stronger EC₅₀ and similar Bmax to the parental rabbit chimeric construct, suggesting broad cross-reactivity (TABLE 13). Constructs were also assessed for binding to HEK293-6E cells transfected with human CLDN18.1. All constructs were negative for CLDN18.1 binding activity, showing selectivity for the CLDN18.2 splice variant.

TABLE 12
CLDN18.2 Ortholog and CLDN18.1 Sequences

| Name | Sequence | SEQ ID NO |
|---|---|---|
| Human | MAVTACQGLGFVVSLIGIAGIIAATCMDQWSTQDLYNNPV | 8 |
| CLDN18.2 | TAVFNYQGLWRSCVRESSGFTECRGYFTLLGLPAMLQAV | |
| | RALMIVGIVLGAIGLLVSIFALKCIRIGSMEDSAKANMTLTS | |
| | GIMFIVSGLCAIAGVSVFANMLVTNFWMSTANMYTGMGG | |
| | MVQTVQTRYTFGAALFVGWVAGGLTLIGGVMMCIACRG | |
| | LAPEETNYKAVSYHASGHSVAYKPGGFKASTGFGSNTKN | |
| | KKIYDGGARTEDEVQSYPSKHDYV | |
| Cynomolgus | MAVTACQGLGFVVSLIGIAGIIAATCMDQWSTQDLYNNPV | 9 |
| monkey | TAVFNYQGLWRSCVRESSGFTECRGYFTLLGLPAMLQAV | |
| CLDN18 | RALMIVGIVLGAIGLLVSIFALKCIRIGSMEDSAKANMTLTS | |
| | GIMFIVSGLCAIAGVSVFANMLVTNFWMSTANMYTGMGG | |
| | MVQTVQTRYTFGAALFVGWVAGGLTLIGGVMMCIACRG | |
| | LAPEETNYKAVSYHASGHSVAYKPGGFKASTGFGSNTKN | |
| | KKTYDGGAHTEDELQSYPSKHDYV | |
| Mouse | MSVTACQGLGFVVSLIGFAGIIAATCMDQWSTQDLYNNPV | 10 |
| CLDN18 | TAVFNYQGLWRSCVRESSGFTECRGYFTLLGLPAMLQAV | |
| A2.1 | RALMIVGIVLGVIGILVSIFALKCIRIGSMDDSAKAKMTLTS | |
| | GILFIISGICAIIGVSVFANMLVTNFWMSTANMYSGMGGM | |
| | GGMVQTVQTRYTFGAALFVGWVAGGLTLIGGVMMCIAC | |
| | RGLTPDDSNFKAVSYHASGQNVAYRPGGFKASTGFGSNTR | |
| | NKKIYDGGARTEDDEQSHPTKYDYV | |
| Human | MSTTTCQVVAFLLSILGLAGCIAATGMDMWSTQDLYDNP | 11 |
| CLDN18.1 | VTSVFQYEGLWRSCVRQSSGFTECRPYFTILGLPAMLQAV | |
| | RALMIVGIVLGAIGLLVSIFALKCIRIGSMEDSAKANMTLTS | |
| | GIMFIVSGLCAIAGVSVFANMLVTNFWMSTANMYTGMGG | |
| | MVQTVQTRYTFGAALFVGWVAGGLTLIGGVMMCIACRG | |
| | LAPEETNYKAVSYHASGHSVAYKPGGFKASTGFGSNTKN | |
| | KKIYDGGARTEDEVQSYPSKHDYV | |

TABLE 13
FACS Binding Fits (4 parameter logistic) to HEK293-6E Transfected
with Human, Cyno, or Mouse CLDN18.2 Orthologs

| | | Human | | Cyno | | Mouse | |
|---|---|---|---|---|---|---|---|
| | | CLDN18.2 | | CLDN18 | | CLDN18 2.1 | |
| | | EC₅₀ | | EC₅₀ | | EC₅₀ | |
| Construct | Description | (nM) | Bmax | (nM) | Bmax | (nM) | Bmax |
| v35777 | parental rabbit | 1.29 | 2071 | 0.48 | 1658 | 0.87 | 1640 |
| | chimeric | | | | | | |
| v37407 | H4-L4 | 1.54 | 2042 | 0.24 | 1410 | 0.80 | 1543 |
| v37408 | H4-L2 | 1.87 | 2187 | 0.36 | 1520 | 2.65 | 1922 |
| v37409 | H2-L4 | 1.22 | 1962 | 0.35 | 1503 | 0.89 | 1527 |
| v37410 | H3-L4 | 0.97 | 1912 | 0.31 | 1497 | 1.02 | 1690 |
| v37411 | H5-L4 | 1.05 | 2166 | 0.37 | 1731 | 1.03 | 1793 |

### Example 4: Engineering of Anti-CD28 Paratope for Generating Variants with Varying Binding Affinity for CD28

This example demonstrates the engineering of the anti-CD28 paratope TN228 to modulate binding affinity for CD28 engagement.

A panel of constructs encompassing a wide range of affinities against CD28 was constructed by mutating residues in the V_{H} and V_{L} domains of the anti-CD28 antibody, specifically focusing on residues within the CDR domains by IMGT definition. A broad panel was created as one-arm Fab antibody constructs and a smaller panel was created “in-format”, i.e., in a trivalent and trispecific anti-CLDN18.2/anti-CD3/anti-CD28 antibody construct format in the same format as that of construct v37638 and v37642.

Positions were selected for mutation by comparing the huTN228 V_{H} and V_{L} sequences with the parental murine sequences and the closest murine germlines to form hypotheses about which sites may have been subject to affinity maturation-driven mutations. It was contemplated that CDR and adjacent framework regions that differ from the murine germline sequences may have been mutated due to affinity selection pressure. It was hypothesized that reversion of such sites in the humanized construct back to the murine germline parental sequence may modulate antigen binding affinity. Such positions, once identified, may also be swapped to other amino acid residues that may either increase or decrease affinity to differing extents, for example, by hypothetically strengthening or weakening electrostatic interactions or changing steric complementarity with the antigen. The extent of D-gene expansion within the CDR H3 loop may also suggest sites for affinity modulating mutations. Mutation sites may also be selected within CDRs, and adjacent framework regions based on 3D structure modeling to identify residues that are likely to be solvent exposed, not participating in intra-domain interactions and positioned to hypothetically contribute to antigen binding interactions. Regions thus identified may be mutated to an alternative amino acid residue to modulate antigen binding affinity. While the primary goal of the affinity modulation effort was to generate a panel of anti-CD28 paratopes with weaker affinity to varying degrees, such engineering may also uncover mutations that strengthen antigen binding affinity.

TABLE 14 below shows the varying binding affinities (K_{D}) for certain anti-CD28 engineered constructs in a one-arm Fab antibody (OAA) format to huCD28, with comparison to huTN228 parental (unmutated) construct in OAA format.

TABLE 14
Binding Data Against CD28 for Affinity Engineered
anti-CD28 Binding Domains Tested in a OAA Format

| | | | | | Fold-change |
|---|---|---|---|---|---|
| Sample | AA subst.* | kₐ (1/Ms) | k_{d} (1/s) | K_{D} (M) | from WT |
| v34891 (parental | — | 3.63E+04 | 3.19E−03 | 8.79E−08 | 1 |
| huTN228 OAA) | | | | | |
| v37841 | S1029A (V_{H}) | 5.11E+04 | 3.22E−03 | 6.34E−08 | 0.72 |
| v37843 | T1035A (V_{H}) | 4.71E+04 | 3.01E−03 | 6.40E−08 | 0.73 |
| v37844 | S1036A (V_{H}) | 5.08E+04 | 3.13E−03 | 6.16E−08 | 0.7 |
| v37845 | Y1037A (V_{H}) | 5.66E+04 | 1.39E−02 | 2.47E−07 | 2.81 |
| v37847 | P1058A (V_{H}) | 5.11E+04 | 6.26E−03 | 1.22E−07 | 1.39 |
| v37851 | N1066A (V_{H}) | 9.09E+04 | 3.70E−02 | 4.12E−07 | 4.69 |
| v37854 | Y1110A (V_{H}) | 4.32E+04 | 3.40E−03 | 7.90E−08 | 0.9 |
| v37855 | G1111A (V_{H}) | 4.64E+04 | 3.42E−03 | 7.37E−08 | 0.84 |
| v37856 | N1111AA (V_{H}) | 4.71E+04 | 2.54E−02 | 5.42E−07 | 6.16 |
| v37858 | L1112AA (V_{H}) | 6.47E+04 | 2.21E−02 | 3.44E−07 | 3.91 |
| v37868 | Y1034A (V_{L}) | 1.26E+05 | 6.01E−02 | 4.72E−07 | 5.37 |
| v37869 | V1035A (V_{L}) | 6.77E+04 | 2.77E−02 | 4.31E−07 | 4.9 |
| v37870 | T1036A (V_{L}) | 9.68E+04 | 2.58E−02 | 2.71E−07 | 3.08 |
| v37874 | N1066A (V_{L}) | 6.18E+04 | 3.21E−03 | 5.21E−08 | 0.59 |
| v37875 | D1068A (V_{L}) | 6.76E+04 | 3.15E−03 | 4.66E−08 | 0.53 |
| v37876 | S1069A (V_{L}) | 5.78E+04 | 3.26E−03 | 5.64E−08 | 0.64 |
| v37878 | K1109A (V_{L}) | 7.08E+04 | 1.17E−02 | 1.70E−07 | 1.93 |
| v37879 | V1114A (V_{L}) | 5.03E+04 | 1.74E−03 | 3.47E−08 | 0.39 |
| v37882 | S1036T (V_{H}) | 6.05E+04 | 3.11E−03 | 5.17E−08 | 0.59 |
| v37883 | N1111AQ (V_{H}) | 8.68E+04 | 2.90E−02 | 3.35E−07 | 3.81 |
| v37884 | L1112AI (V_{H}) | 7.60E+04 | 9.93E−03 | 1.33E−07 | 1.51 |
| v37888 | N1066Q (V_{L}) | 5.92E+04 | 3.19E−03 | 5.38E−08 | 0.61 |
| v37890 | A1024T (V_{H}) | 6.08E+04 | 3.49E−03 | 5.78E−08 | 0.66 |
| v37891 | L1053I (V_{H}) | 5.88E+04 | 2.80E−03 | 4.77E−08 | 0.54 |
| v37897 | V1087F (V_{H}) | 6.30E+04 | 4.87E−03 | 7.74E−08 | 0.88 |
| v37898 | M1039L_Q1040N (V_{L}) | 7.79E+04 | 5.73E−03 | 7.37E−08 | 0.84 |

*AA position numbering according to the IMGT numbering system.

### Example 5: Ability of Trivalent and Trispecific Antibody Constructs to Bind MSLN, CD3 and CD28 as Assessed by Surface Plasmon Resonance (SPR)

This example demonstrates the ability of the anti-MSLN, anti-CD3 and anti-CD28 paratopes when part of a trivalent and trispecific construct to bind to the respective targets as assessed by SPR. The changes in CD3 and CD28 binding properties in the trispecific (e.g., anti-(MSLN×CD3×CD28) and anti-(CLDN18.2×CD3×CD28)) antibody constructs based on their geometry and format is demonstrated as assessed by SPR.

### Binding of Trivalent and Trispecific Antibody Constructs to MSLN by SPR

All SPR binding experiments were carried out using a BiacoreT200 instrument at 25° C. with 10 mM HEPES, 150 mM NaCl, 3 mM EDTA, and 0.05% Tween 20 at pH 7.4. Recombinant MSLN his tag protein (R&D systems) was immobilized by amine coupling on CM5 sensor chip and purified bispecific antibody constructs to be tested was flowed on the sensor chip at 30 μL/min for 120 s, following a dissociation for 600 s. Resultant kₒₙ (also referred to as “kₐ”), k_{off} (also referred to as “k_{d}”) and K_{D} values were determined from fitting the sensorgrams using the 1:1 binding model with reported values as the mean of three independent runs.

### Binding of Trivalent and Trispecific Antibody Constructs to CD3 by SPR

All SPR binding experiments were carried out using a BiacoreT200 instrument at 25° C. with 10 mM HEPES, 150 mM NaCl, 3 mM EDTA, and 0.05% Tween 20 at pH 7.4. Recombinant human CD3 epsilon/delta heterodimer, his tag protein (Acro Biosystems) was covalently immobilized on CM5 sensor chips by amine coupling. Purified antibody constructs to be tested were indirectly captured on the sensor chip by binding to the recombinant CD3 protein when injected at 30 μL/min for 140-500 s following a dissociation for 600-1200 s. Resultant kₒₙ, k_{off} and K_{D} values were determined from fitting the sensorgrams using the 1:1 binding model with reported values as the mean of three independent runs.

### Binding of Trivalent and Trispecific Antibody Constructs to CD28 by SPR

All SPR binding experiments were carried out using a BiacoreT200 instrument at 25° C. with 10 mM HEPES, 150 mM NaCl, 3 mM EDTA, and 0.05% Tween 20 at pH 7.4. Recombinant human/cynomolgus/rhesus macaque CD28 mouse IgG2a Fc fusion protein (Acro Biosystems) was covalently immobilized on CM5 sensor chips by amine coupling. Purified antibody constructs to be tested were injected on the sensor chip at 30 μL/min for 140-500 s following a dissociation for 600-1200 s. Resultant kₒₙ, k_{off} and K_{D} values were determined from fitting the sensorgrams using the 1:1 binding model with reported values as the mean of three independent runs.

For the trivalent and trispecific construct v37634, an antihuman IgG Fc capture antibody was immobilized on the CM5 sensor ship and the trispecific construct was captured followed by injections of recombinant human/Cynomolgus/Rhesus macaque CD28 Protein, His Tag protein (Acro Biosystems).

### Results

TABLE 15 below shows the binding parameters, including the calculated K_{D} values, for certain tested trivalent and trispecific antibody constructs binding to MSLN.

TABLE 15
Binding of Certain Trivalent and Trispecific Constructs to MSLN

| | MSLN | | |
|---|---|---|---|
| Construct | kₐ (1/Ms) | k_{d} (1/s) | K_{D} (M) |
| v34913 | 3.40E+05 | 2.12E−03 | 6.49E−09 |
| v34914 | 3.74E+05 | 1.97E−03 | 5.39E−09 |
| v34916 | 3.88E+05 | 1.81E−03 | 4.78E−09 |
| v34917 | 4.11E+05 | 1.99E−03 | 4.96E−09 |
| v34918 | 2.94E+05 | 1.87E−03 | 7.08E−09 |
| v34919 | 4.58E+05 | 1.81E−03 | 4.16E−09 |
| v34927 | 2.70E+05 | 1.87E−03 | 6.86E−09 |

TABLE 16 shows the binding parameters, including the calculated Kp values, for certain tested trivalent and trispecific antibody constructs binding to CD3 and CD28. The impact of format and geometry of the binding properties of the anti-CD3 and anti-CD28 paratopes in all the trispecific and bispecific antibody constructs can be demonstrated by the binding parameters reported in TABLE 16.

TABLE 16
Binding of Certain Trivalent and Trispecific Constructs to CD3 and CD28

| | CD3 | | | CD28 | | |
|---|---|---|---|---|---|---|
| Construct | kₐ (1/Ms) | k_{d} (1/s) | K_{D} (M) | kₐ (1/Ms) | k_{d} (1/s) | K_{D} (M) |
| v34913 | 2.70E+05 | 1.12E−02 | 4.16E−08 | 4.74E+04 | 2.16E−03 | 4.58E−08 |
| v34914 | 2.37E+05 | 1.91E−02 | 8.11E−08 | 7.02E+04 | 8.49E−03 | 1.22E−07 |
| v34916 | 2.91E+05 | 1.04E−02 | 3.72E−08 | 1.30E+04 | 5.46E−04 | 5.65E−08 |
| v34917 | 4.44E+05 | 1.77E−02 | 4.08E−08 | 8.24E+03 | 2.09E−03 | 3.73E−07 |
| v34918 | 1.18E+05 | 1.35E−02 | 1.18E−07 | 5.27E+04 | 2.59E−03 | 5.00E−08 |
| v34919 | 2.67E+05 | 1.31E−02 | 5.04E−08 | n/a | n/a | n/a |
| v34927 | n/a | n/a | n/a | 5.09E+04 | 2.58E−03 | 5.07E−08 |
| v37633 | 1.30E+05 | 1.76E−02 | 1.35E−07 | 1.13E+05 | 6.20E−03 | 5.51E−08 |
| v37634* | 1.63E+05 | 1.23E−02 | 7.76E−08 | 7.23E+03 | 1.87E−03 | 2.58E−07 |
| v37635 | 1.57E+05 | 1.43E−02 | 9.25E−08 | 4.04E+04 | 1.07E−02 | 2.88E−07 |
| v37636 | 1.34E+05 | 2.10E−02 | 1.60E−07 | 3.04E+04 | 1.32E−02 | 4.34E−07 |
| v37637 | 1.21E+05 | 1.29E−02 | 1.06E−07 | 1.13E+05 | 6.92E−03 | 6.14E−08 |
| v37638 | 1.64E+05 | 1.23E−02 | 7.46E−08 | 9.55E+04 | 2.10E−03 | 2.22E−08 |
| v37639 | 2.92E+04 | 1.44E−02 | 4.92E−07 | 8.41E+04 | 2.01E−03 | 2.39E−08 |
| v37640 | 5.22E+04 | 1.55E−02 | 2.96E−07 | 7.78E+04 | 2.12E−03 | 2.73E−08 |
| v37641 | 6.08E+04 | 1.34E−02 | 2.25E−07 | 6.65E+04 | 2.06E−03 | 3.18E−08 |
| v37642 | 1.89E+05 | 1.17E−02 | 6.20E−08 | 9.82E+04 | 1.98E−03 | 2.02E−08 |
| v37663 | 2.06E+05 | 1.57E−02 | 7.64E−08 | n/a | n/a | n/a |
| v37664 | 1.80E+05 | 2.28E−02 | 1.27E−07 | n/a | n/a | n/a |
| v37665 | n/a | n/a | n/a | 8.94E+04 | 2.24E−03 | 2.51E−08 |
| v37666 | n/a | n/a | n/a | 6.73E+04 | 2.17E−03 | 3.24E−08 |
| v37667 | 1.28E+06 | 7.78E−03 | 5.93E−09 | n/a | n/a | n/a |
| v37669 | 5.89E+05 | 2.55E−03 | 4.38E−09 | n/a | n/a | n/a |
| v35923 | 8.31E+05 | 1.56E−03 | 1.90E−09 | n/a | n/a | n/a |

*Note
CD28 binding of construct v37634 was assessed by a different method; v37634 was captured on anti-human IgG Fc immobilized on CM5 sensor chip and CD28-his tag was flowed.

TABLE 17
Binding of In-Format Engineered Anti-CD28 Variants to huCD28

| | | | | | Fold-change |
|---|---|---|---|---|---|
| Construct | AA subst.* | kₐ (1/Ms) | k_{d} (1/s) | K_{D} (M) | from WT |
| v37638 | — | 9.55E+04 | 2.10E−03 | 2.22E−08 | 1.0 |
| v37683 | P1058A (V_{H}) | 9.58E+04 | 4.66E−03 | 4.86E−08 | 2.2 |
| v37684 | G1064S (V_{H}) | 8.06E+04 | 1.13E−02 | 1.43E−07 | 6.4 |
| v37685 | V1035G (V_{L}) | 6.79E+04 | 2.90E−02 | 6.31E−07 | 28.4 |
| v37686 | D1068E (V_{L}) | 8.93E+04 | 2.52E−03 | 2.81E−08 | 1.3 |
| v37687 | E1080K (V_{H}) | 1.01E+05 | 4.75E−03 | 4.70E−08 | 2.1 |
| v37688 | Y1110S (V_{H}) | 9.73E+04 | 2.53E−03 | 2.58E−08 | 1.2 |
| v37689 | N1111aA (V_{H}) | 8.96E+04 | 2.11E−02 | 2.39E−07 | 10.8 |
| v37690 | Y1112S (V_{H}) | n/a | n/a | n/a | n/a |
| v37691 | L1112aN (V_{H}) | 9.86E+04 | 1.07E−02 | 1.09E−07 | 4.9 |
| v37692 | Y1113S (V_{H}) | 3.22E+04 | 6.13E−02 | 2.11E−06 | 95.1 |
| v37693 | Y1037A (V_{H}) | 1.06E+05 | 9.68E−03 | 9.18E−08 | 4.1 |
| v37694 | Y1031A (V_{L}) | n/a | n/a | n/a | n/a |
| v37695 | N1066A (V_{L}) | 9.51E+04 | 2.66E−03 | 2.77E−08 | 1.3 |
| v37642 | — | 7.04E+04 | 2.86E−03 | 4.23E−08 | 1 |
| v38990 | Y1113S (V_{H}) | 4.61E+04 | 3.30E−02 | 9.51E−06 | 225 |
| v38991 | N1111aA (V_{H}) | 6.29E+04 | 1.45E−02 | 2.57E−07 | 6.1 |
| v38992 | G1064S (V_{H}) | 5.98E+04 | 7.38E−03 | 1.30E−07 | 3.1 |
| v38993 | L1112aN (V_{H}) | 5.90E+04 | 8.17E−03 | 1.44E−07 | 3.4 |

n/a indicates that no measurable binding signal was observed.
*AA position numbering according to the IMGT numbering system.

### Example 6: Thermal Stability of Anti-(CLDN18.2×CD3×CD28) Targeting Trivalent and Trispecific Antibody Constructs

The thermal stability of the engineered full-sized trispecific anti-CLDN18.2/anti-CD3/anti-CD28 antibody constructs, and bispecific anti-CLDN18.2/anti-CD3 as well as anti-CLDN18.2/anti-CD28 control constructs was assessed by differential scanning calorimetry (DSC).

All DSC experiments were carried out using a VP-Capillary DSC (Malvern Instruments Ltd) instrument. The proteins were diluted to 0.4 mg/mL with 0.4 mL loaded into the 96 well plates and measured with a scan rate of 1° C./min from 20 to 100° C. Data was analyzed using the Origin 7 software with the VP-Capillary DSC option with the H6Su (50 mM histidine, 9% sucrose, pH 6.0) buffer background subtracted.

TABLE 18A shows the maximum melting temperatures (Tm) for each of the peaks in the thermograms of the trispecific and bispecific antibody constructs. The term “scFv²” indicates that the construct comprises two scFv domains as binding domains, which are not coupled to one another in tandem.

TABLE 18A
Thermostability Data of Tested Antibody Constructs

| Antibody | Targeting | Tm1 | Tm2 | Tm3 |
|---|---|---|---|---|
| Construct | Domains | (° C.) | (° C.) | (° C.) |
| v37633 | scFv², Fab | 56.11 | 66.06 | 77.64 |
| v37634 | scFv², Fab | 79.30 | | |
| v37635 | scFv², Fab | 65.80 | 80.48 | |
| v37636 | scFv², Fab | 78.14 | | |
| v37637 | scFv², Fab | 65.27 | 78.51 | |
| v37638 | scFv², Fab | 66.32 | 88.83 | |
| v37639 | scFv², Fab | 88.91 | | |
| v37640 | scFv², Fab | 55.74 | 65.43 | 89.28 |
| v37641 | scFv², Fab | 65.89 | 89.61 | |
| v37642 | scFv², Fab | 67.07 | 89.16 | |
| v37663 | scFv, Fab | 56.13 | 66.50 | 81.18 |
| v37664 | scFv, Fab | 56.32 | 67.91 | 78.14 |
| v37665 | scFv, Fab | 55.95 | 66.45 | 88.80 |
| v37666 | scFv, Fab | 67.42 | 89.53 | |

The results shown in TABLE 18A demonstrate that the tested antibody constructs exhibited thermostability profiles comparable to those of conventional IgG1 antibodies.

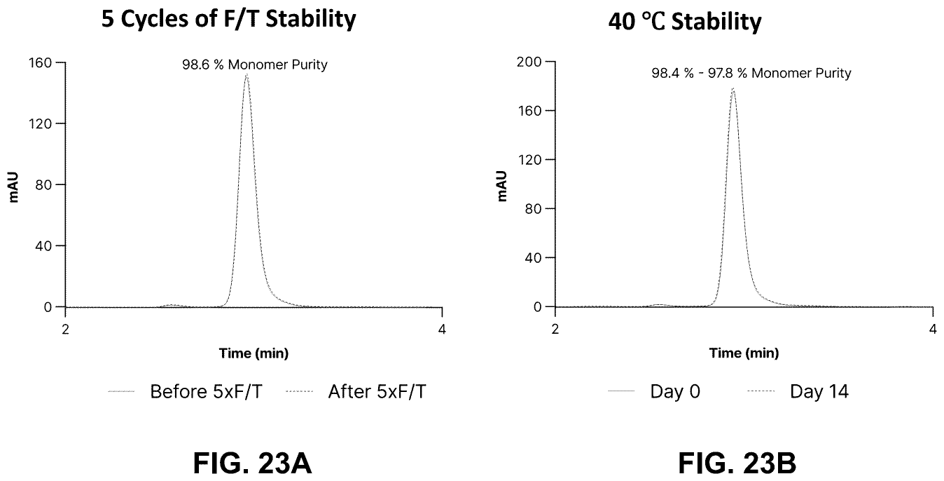

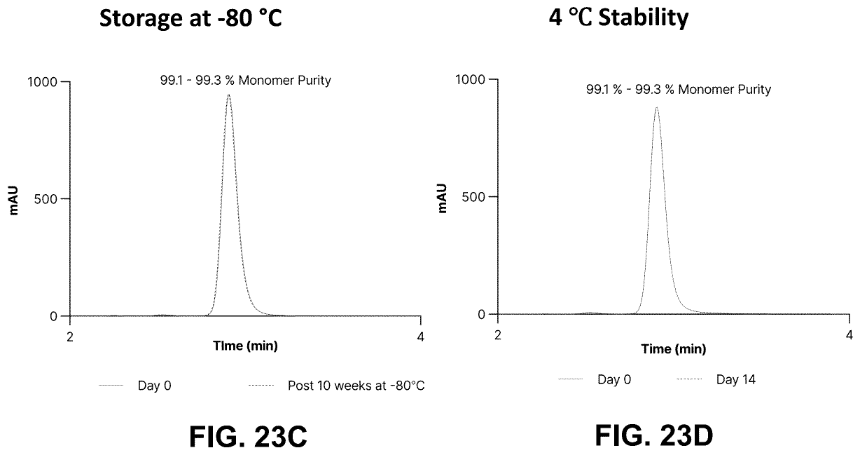

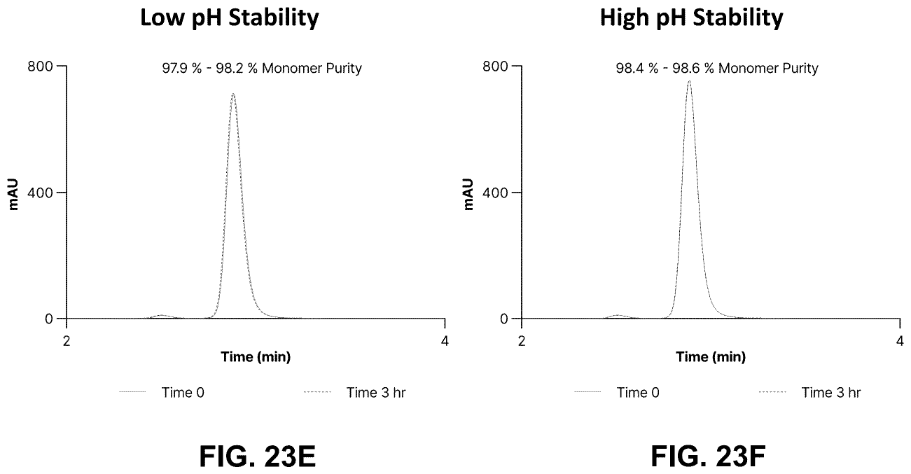

The trivalent and trispecific antibody construct v37634 was further tested for (i) its freeze-thaw stability, in which it showed no significant change in purity post 5 cycles freezing to −80° C. followed by thawing to 4° C. (FIG. 23A), and (ii) in an accelerated stress test in which the construct showed only minimal change in purity post 14 days incubation at 40° C. (FIG. 23B), using a construct concentration of 1 mg/mL.

The trivalent and trispecific antibody construct v37634 was further tested for long term storage at −80° C., in which the construct showed only minimal change in purity post 10 weeks storage at −80° C. (FIG. 23C), using a construct concentration of 5 mg/mL. Moreover, the trivalent and trispecific antibody construct v37634 was tested in an accelerated stress test for its stability, in which it showed minimal change in purity post 14 days incubation at 4° C. and 40° C., using a construct concentration of 5 mg/mL (FIG. 23D and FIG. 23B). Further, the trivalent and trispecific antibody construct v37634 was tested for its stability upon treatment with low pH (pH 3.5) and high pH (pH 9.0), in which the construct showed no significant change in purity post 3 hours (FIG. 23E and FIG. 23F). The purity of the trispecific antibody construct v37634 post each of the stress condition is compared in the TABLE 18B below.

TABLE 18B
Monomer Purity (%) of v37634 Construct Post Stress
Treatment Compared to No Treatment Condition

| | Monomer Purity (%) by |
|---|---|
| Construct v37634 | HPLC-SEC Analysis |
| No Treatment | 99.3 |
| 5 cycles freeze-thaw | 99.1 |
| 14 days incubation at 4° C. | 99.1 |
| 14 days incubation at 40° C. | 96.9 |
| 10 weeks at −80° C. | 99.1 |
| 3 hours at pH 3.5 | 97.9 |
| 3 hours at pH 9.0 | 98.4 |

### Example 7: Determination of MSLN Density on Cell Surface of Target Cell Lines

Target cell lines with differing levels of plasma membranous MSLN expression were used herein to assess, e.g., how trispecific construct format can impact T cell-mediating killing of target (e.g., tumor) cells. Therefore, plasma membranous MSLN expression was quantified on different cell lines.

The Antibody Binding Capacity on cancer cell lines expressing different levels of MSLN was assessed by flow cytometry using Quantum Simply Cellular anti-human IgG kit (Bangs Laboratories, Inc.). Briefly, Alexa Fluor 647-labelled anti-MSLN antibody, v18490, was used to stain beads and test samples according to the manufacturer's instructions and were run on the same day and at the same photomultiplier tube settings. To calculate the cell surface Antibody Binding Capacity values, the geometric means for the four Quantum Simply Cellular beads and test samples were uploaded to the QuickCal v2.3 Excel spreadsheet-based analysis template (Bangs Laboratories, Inc.).

Plasma membranous MSLN densities of cancer cell lines are presented in TABLE 19. OVCAR-3 cells showed the highest expression level of MSLN and OVTOKO cells expressed the lowest level of MSLN.

TABLE 19
MSLN Density on Certain Cancer Cell Lines Represented
as the Average Antibody Binding Capacity

| | Mesothelin Density |
|---|---|
| Cell line | (Antibody binding capacity) |
| OVCAR-3 | 786,926 |
| H292 | 152,986 |
| OVTOKO | 9,752 |

### Example 8: Ability of Anti-(MSLN×CD3×CD28) Trispecific Antibody Constructs to Bind to Native CD3 and CD28 as Assessed by Flow Cytometry

To determine the impact of the trispecific construct format on binding to CD3+CD28+ primary human T cells, the binding affinity to human T cells was measured by flow cytometry as described below.

The ability of trispecific anti-MSLN-CD3-CD28 constructs, as well as bispecific and benchmark controls, to bind to CD3+CD28+ primary human T cells was assessed via whole cell FACS binding analysis.

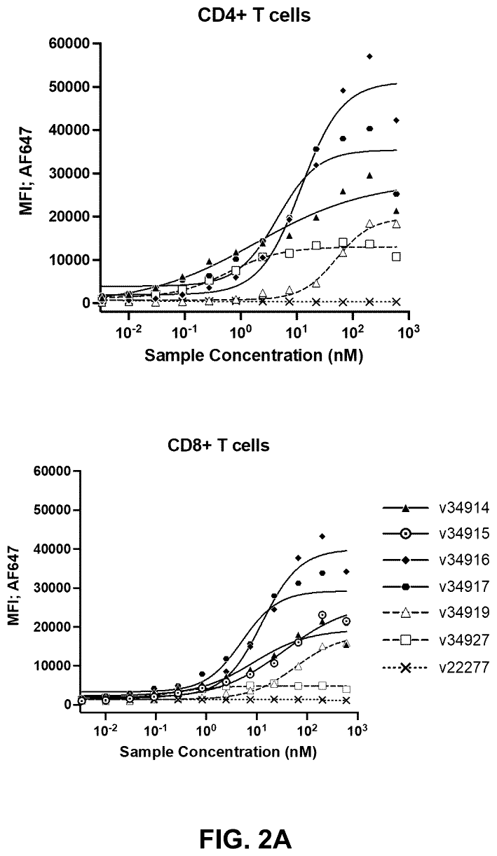

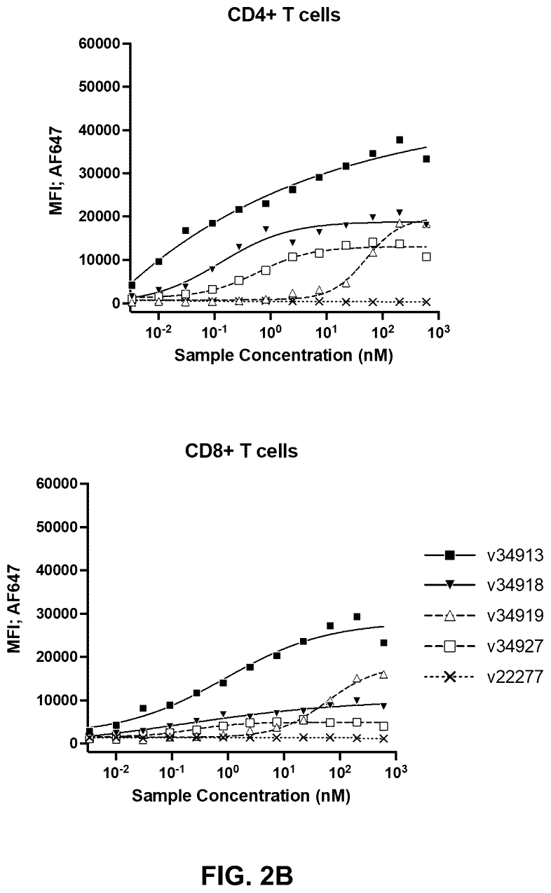

Briefly, primary human T cells (>80% viability) were resuspended to 1×10⁶ cells/mL in FACS buffer (PBS+2% FBS; Gibco) and seeded in a 96 well v-bottom plate (Sarstedt). Cells were mixed with antibody dilutions and incubated at 4° C. for one hour. Following incubation, cells were washed twice by adding 150 mL FACS buffer and centrifuging for 400×g for 3 minutes. The cell pellet was resuspended in 50 mL FACS buffer with Alexa Fluor 647-labeled goat anti-human IgG (Jackson Labs) and fluorophore-conjugated antibodies specific for CD4 and CD8. Cells were incubated for 1 hour at 4° C. Following incubation, cells were washed twice by adding 150 mL FACS buffer and centrifuging for 400×g for 3 minutes. Cells were resuspended in FACS buffer and acquired on the BD LSRFortessa Cell Analyzer. The apparent binding affinities and Bmax of the anti-MSLN-CD3-CD28 (1+1+1) trivalent and trispecific constructs and the bispecific controls to human T cells are shown in FIGS. 2A-2B.

### Ability of 1+1+1 Anti-(MSLN×CD3×CD28) Trivalent and Trispecific Antibody Constructs to Bind to Native CD3 and CD28 as Assessed by Flow Cytometry

The anti-CD3 bispecific construct, v34919, exhibited the weakest binding to CD4+ and CD8+ human T cells with a relative binding affinity around 50 nM. The relative binding affinity of the anti-CD28 bispecific constructs, v34927, was higher than that of v34919, measured at around 0.5 nM. The trivalent and trispecific constructs tested displayed a stronger binding to the human T cells, compared to both bispecific control constructs, v34919 and v34927, with two trispecific constructs, v34913 and v34918, exhibiting<1 nM binding.

This observed shift in binding affinity relative to the corresponding bispecific control constructs indicated bivalent binding of the trispecific constructs to T cells via their anti-CD3 and anti-CD28 binding domains.

### Example 9: Impact of Trispecific Construct Format on In Vitro Cytotoxicity Towards MSLN-Expressing Tumor Cell Lines in Co-Culture with Primary Human CD3+ CD28+ T Cells

To test the impact of a construct's format on its activity and preferential killing of tumor cells overexpressing MSLN, selected construct variants were tested in primary human T cell cultures with allogeneic tumor cell lines expressing MSLN.

Impact of Trispecific Construct Format on In Vitro Cytotoxicity Towards MSLN-Expressing Tumor Cell Lines in Co-culture with Primary Human CD3+ T Cells

Frozen human T cells were thawed at 37° C. and mixed with MSLN+H292 cells such that the ratio of T cells to allogeneic tumor cells was adjusted to an E:T ratio of 2:1. The mixtures were incubated together with the antibody constructs to be tested for 72 hours, after which Hoechst stain (vendor) was added to the cells. After a 30-minute incubation period, tumor cell viability was assessed through live tumor cell counts using the Operetta CLS High Content Analysis System (Perkin Elmer; HH16000020). Data are representative of 3 independent experiments with 1 donor. Percent cytotoxicity was calculated using the following formula:

$$\% cytotoxicity = ( 1 - \frac{n umber of tumor cells left in sample}{total number of tumor cells in untreated condition} ) \times 100$$

TABLE 20
Potency and Maximum Cytotoxicity Induced by Anti-MSLN
1 + 1 + 1 Trivalent and Trispecific Constructs
from Primary Human T Cells Co-cultured with H292 Target Cells

H292

| | IC₅₀ | Maximum |
|---|---|---|
| Construct | (pM) | Killing (%) |
| v34913 | 0.70 | 79.1 |
| v34914 | 19.07 | 76.6 |
| v34915 | Ambiguous | 57.3 |
| v34916 | 36.60 | 75.6 |
| v34917 | 46.68 | 70.0 |
| v34918 | 3.32 | 72.1 |
| v34919 | 262.20 | 66.1 |
| v34927 | Ambiguous | 7.7 |

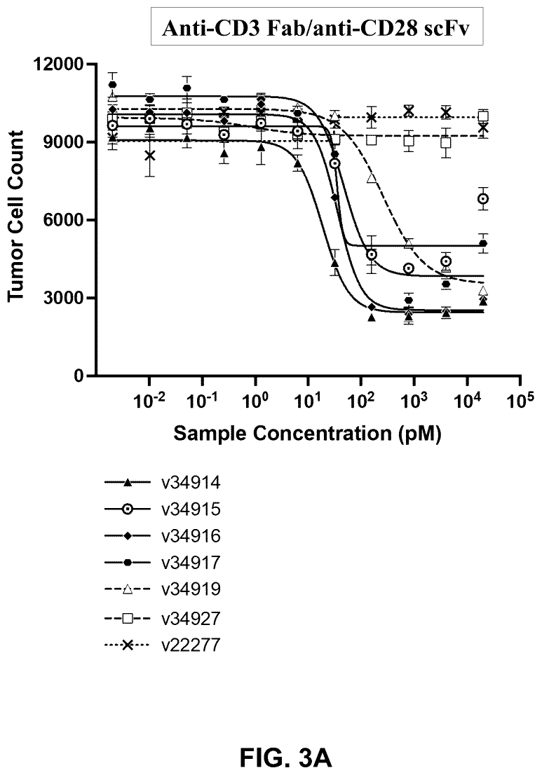

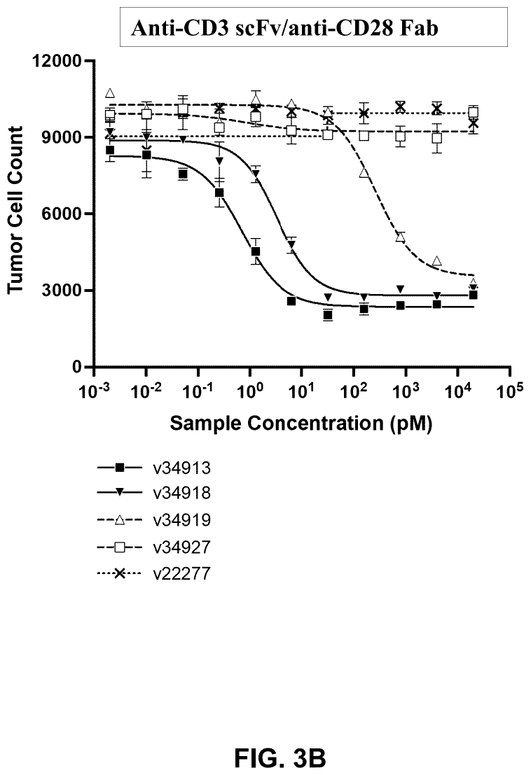

Cytotoxicity curves of primary human T cells in c-culture with MSLN+H292 cells following treatment with the tested anti-MSLN-CD3-CD28 (1+1+1) trivalent and trispecific antibody constructs or bispecific controls are shown in FIGS. 3A-3B.

The trivalent and trispecific constructs having formats comprising one CD3 Fab, one CD28 scFv, and one MSLN scFv exhibited a range of potencies against MSLN-overexpressing H292 allogeneic target cells. The most potent of the CD3 Fab-containing constructs, v34914, showed approximately 14-fold higher potency compared to the respective bivalent MSLN×CD3 bispecific control construct, v34919. The constructs v34916 and v34917 exhibited equivalent potencies, which were approximately 5-fold higher compared to v34919. The trivalent and trispecific constructs, v374914, v34916, and v34917 induced greater maximum cytotoxicity than the bivalent MSLN×CD3 bispecific control construct, 34919, with maximum cytotoxicity reaching between about 70-77% compared to the 66% reached by v34919.

The trivalent and trispecific constructs having formats comprising one CD28 Fab, one CD3 scFv, and one MSLN scFv exhibited the highest potencies (i.e., lowest IC₅₀ values) against MSLN-overexpressing H292 allogeneic target cells. The most potent of the CD28 Fab-containing constructs, v34913, showed approximately 375-fold higher potency than the corresponding MSLN×CD3 bispecific control construct, v34919. The construct v34918 exhibited an approximately 80-fold greater potency than v34919. Maximum cytotoxicity induced by constructs v34913 and v34918 reached 79.1 and 72.1%, respectively. Negative control, v22277, did not induce any measurable cytotoxicity of H292 cells.

These results demonstrate that the cytotoxicity toward allogeneic target tumor cells can be modulated through modification of a construct's format and/or geometry, with various formats exhibiting a range of potencies against H292 cells. Furthermore, these data show that a several hundred-fold increase in anti-tumor cytotoxicity can be achieved by adding a co-stimulatory anti-T cell antigen binding domain (e.g., anti-CD28) when compared to corresponding bispecific constructs that only contain one binding domain targeting an immune cell (e.g., T cell) antigen.

### Example 10: Impact of Trispecific Construct Format on Induction of Pro-Inflammatory Cytokine Release by Human CD3+ T Cells in Co-Culture with MSLN-Positive Tumor Cell Lines

This example demonstrates that the tested trispecific antibody constructs of the present disclosure activated T cells by simultaneously binding to tumor-associated antigen on cancer cells and both CD3e and CD28 on T cells, leading to cross-linking of CD3e and CD28. The resulting signalling cascade triggered T cell activation, leading to cytokine secretion. To assess the impact of a construct's formats on T cell activation, TNFα and IL-2 production was measured for various constructs with varying formats/geometries as described below.

Test articles (e.g., antibody constructs) were diluted in RPMI1640 (Gibco)+10% FBS (ThermoFisher) and added to appropriate wells of the 384-well black flat bottom assay plates (ThermoFisher, Watham, MA). Human CD3+ T cells were thawed and mixed with H292 (high MSLN density) cells at an E:T ratio of 2:1 then added to the plates. Plates were incubated for 72 hrs at 37° C. with 5% carbon dioxide. Post incubation, 30 μL/well of supernatant was transferred to non-binding 384-well plates (Greiner-Bio-One, Kremsmünster, Austria) and stored at −20° C.

TNFα and IL-2 were quantified using MSD (Mesoscale Discovery, Piscataway, NJ). The night before cytokine quantification, MSD plates were blocked and coated in capture antibodies according to the manufacturers' instructions. The following day, plates were washed in PBS-T and 5 μl of assay diluent was added to each plate. The supplied TNFα and IL-2 standard (calibrator 1) was titrated as per manufacturer's instructions. Supernatants were thawed at room temperature and 5 μL of samples or standards were transferred to MSD plates. Detection antibodies were prepared at appropriate dilutions and 10 μL was added to each sample and standard well in MSD plates. The plates were sealed with aluminum foil and incubated away from light at room temperature for two hours. Plates were washed 3× in PBS-T and 40 μL MSD Gold read buffer T was added to each well. Plates were read on the MESO SECTOR 6000 and cytokine concentration was determined using MSD software. Data from a standard curve and samples were used to perform a nonlinear curve-fit with x-interpolation to obtain TNFα and IL-2 concentrations in pg/mL. Data are representative of 1 independent experiments with 1 donor.

Potency and maximum cytokine production of TNFα and IL-2 by human CD3+ T cells in the presence of anti-MSLN-CD3-CD28 trispecific constructs are summarized in TABLE 21.

TABLE 21
Potency and Maximum Cytokine Production Induced
by Anti-MSLN Trispecific Constructs from Primary
Human T Cells Co-cultured with H292 Target Cells

| | IL-2 | | TNFα | |
|---|---|---|---|---|
| | | Maximum | | Maximum |
| Antibody | EC₅₀ | Cytokine | EC₅₀ | Cytokine |
| Construct | (pM) | (pg/mL) | (pM) | (pg/mL) |
| v34913 | 4.242 | 1810.0 | 17.22 | 128.2 |
| v34914 | 1014 | 3830.9 | ND | 196.4 |
| v34915 | 154.3 | 350.6 | 184.8 | 38.1 |
| v34916 | 508.9 | 2470.4 | 1143 | 109.1 |
| v34917 | 134.7 | 622.5 | 72.48 | 30.6 |
| v34918 | 134.2 | 3080.6 | 287.4 | 180.03 |
| v34919 | 226.6 | 15.7 | Ambiguous | 11.8 |
| v34927 | Ambiguous | 2.8 | Ambiguous | 2.1 |

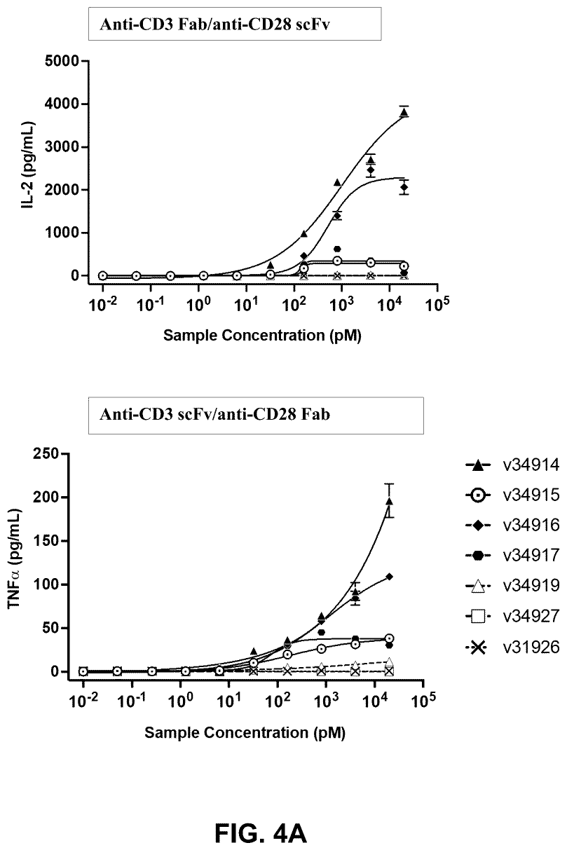

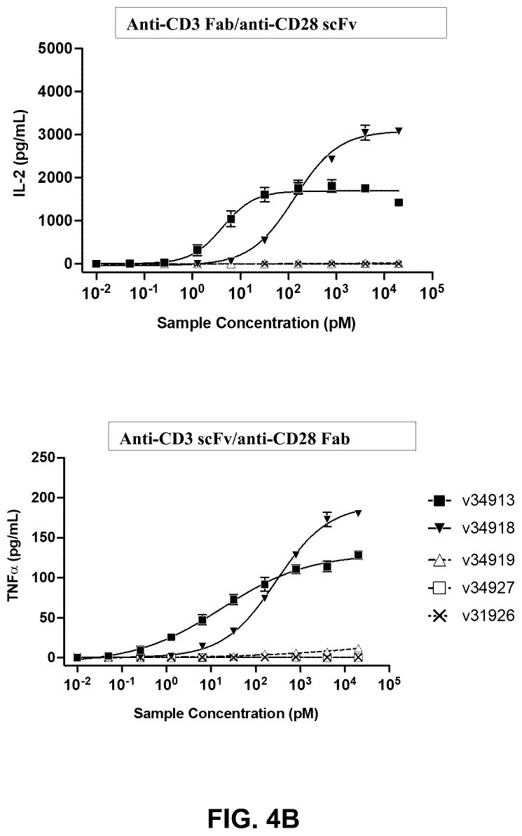

Human CD3+ T cell TNFα and IL-2 release after incubation in the presence of anti-(MSLN×CD3×CD28) trispecific constructs with different formats are summarized in FIGS. 4A-4B.

Of the trispecific constructs that comprised one anti-CD3 Fab domain, one anti-CD28 scFv domain, and one anti-MSLN scFv domain, the constructs v34915 and v34917 showed similar EC₅₀ values, with approximately 1.5-fold higher potency for IL-2 production compared to the bivalent anti-(MSLN×CD3) bispecific control, v34919. Maximum levels of IL-2 production induced by v34915 and v34917 reached 350.6 pg/mL and 622.5 pg/mL, respectively, which was approximately 20 to 40-fold higher than that induced by v34919. The constructs v34914 and v34916 exhibited reduced potency of IL-2 production compared to v34919, with an approximate 2-5-fold increase in their EC₅₀ values, respectively. Maximum cytokine production induced by v34914 and v34916 reached 3830.9 pg/mL and 2470.4 pg/mL, which was approximately 150 to 250-fold higher than that induced by v34919. The construct v34913 exhibited the highest potency in IL-2 production of the trispecific construct that comprised one anti-CD3 scFv domain, one anti-CD28 Fab domain, and one anti-MSLN scFv domain, which was 53-fold greater than that measured for the control v34919. The construct v34918 displayed an ECso of 134.2 pM, which was approximately 2-fold greater than that of v34919. The constructs v34913 and v34918 induced maximum cytokine levels of 1810.0 pg/mL and 3080.6 pg/mL, respectively, which was approximately 115 to 196-fold greater than that induced by v34919. The negative control, v22277, and the bivalent anti-(MSLN×CD28) bispecific control, v34927, did not induce any measurable IL-2 production.

Of the trispecific constructs that comprised one anti-CD3 Fab domain, one anti-CD28 scFv domain, and one anti-MSLN scFv domain, the construct v34917 exhibited the highest potency for TNFα production, followed by the constructs v34915 and v34916, with EC₅₀ values of 72.48 pM, 184.8 pM, and 1143 pM, respectively. TNFα production induced by construct v34914 did not reach plateau, and therefore the EC₅₀ value was not determined. Maximum TNFα induced by the constructs v34914 and v34916 reached 196.4 pg/mL and 109.1 pg/mL, respectively. Maximum TNFα induced by the constructs v34915 and v34917 reached 38.1 pg/mL and 30.6 pg/mL, respectively. Of the trispecific constructs that comprised one anti-CD3 scFv domain, one anti-CD28 Fab domain, and one anti-MSLN scFv domain, the construct v34913 exhibited the highest potency for TNFα production, followed by v34918, with EC₅₀ values of 17.22 pM, 287.4 pM, respectively. Maximum TNFα induced by the constructs v34913 and v34918 reached 128.2 pg/mL and 180.03 pg/mL, respectively. The negative control, v22277, and the bivalent anti-(MSLN×CD28) and anti-(MSLN×CD3) bispecific controls, v34927 and v34919, did not induce any measurable TNFα production.

These results show that induction of T cell mediated cytokine production can be significantly enhanced using antibody constructs comprising two T cell antigen binding domains (e.g., against CD and CD28) compared to conventional constructs targeting only one T cell antigen. Furthermore, cytokine production can be modulated through modification of a trispecific construct's format and/or geometry, with various construct formats/geometries exhibiting a range in potency and maximum cytokine production of IL-2 and TNFα.

### Example 11: MSLN-Targeting of Anti-(MSLN×CD3×CD28) Trispecific Antibody Constructs in In Vitro T-Cell Dependent Cytotoxicity Assays with Tumor Cells Expressing Varying Levels of MSLN

To determine whether certain trispecific antibody constructs exhibit tumor antigen dependency with respect to their in vitro cytotoxic activity, T-cell dependent cytotoxicity assays were conducted with tumor cells expressing high or low-levels of antigen.

Certain anti-MSLN trispecific constructs as well as benchmark and bispecific control constructs were diluted in RPMI1640 (Gibco)+10% FBS (ThermoFisher) and added to appropriate wells of the 384-well black flat bottom assay plates (ThermoFisher, Watham, MA). Primary human CD3+ T cells were thawed and mixed with H292 (152 986 MSLN/cell) or OVTOKO (9 752 MSLN/cell) target cells at an E:T ratio of 2:1. Plates were incubated for 72 hr at 37° C. and 5% carbon dioxide, after which Hoechst Stain (ThermoFisher) was added to the cells. After a 30-minute incubation period, tumor cell viability was assessed through live tumor cell counts using the Operetta CLS High Content Analysis System (PerkinElmer). Data are representative of 2 independent experiments with 1 donor.

Percent cell cytotoxicity was calculated using the following formula:

$$\% cytotoxicity = ( 1 - \frac{n umber of tumor cells left in sample}{total number of tumor cells in untreated condition} ) \times 100$$

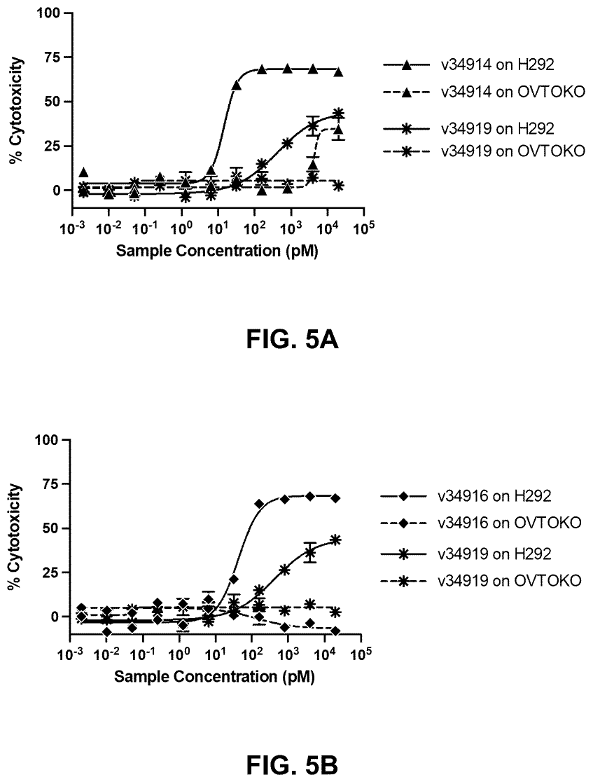

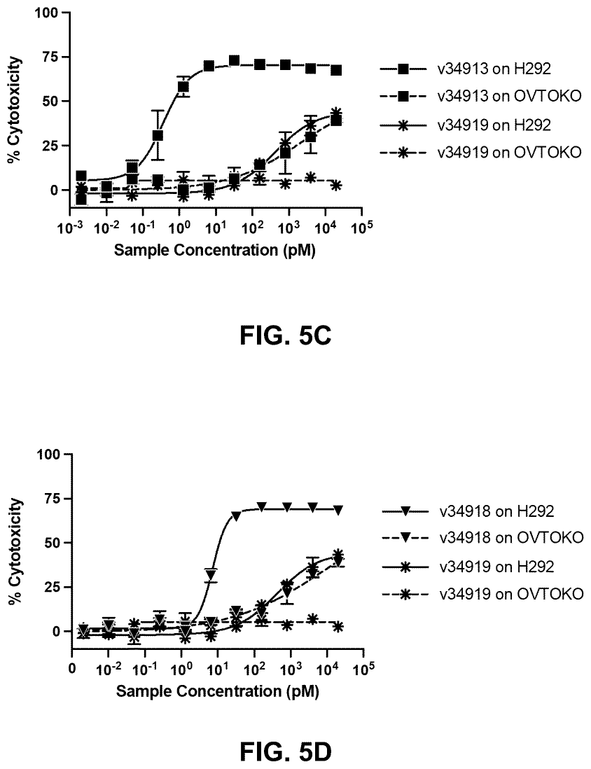

Concentration response curves for the tested antibody constructs are presented in FIGS. 5A-5D and IC₅₀ and maximum percent killing from the T cell-dependent cytotoxicity assay comparing the tested trispecific constructs and the bispecific/benchmark control, v34919, are presented in TABLE 22.

TABLE 22
Cytotoxic Activity of Anti-MSLN Trivalent and Trispecific
Constructs and Bispecific/Benchmarks Towards MSLN
High and Low/Negative Tumor Cells Grown in Culture
with Primary Human CD3+ T Cells

| | |
|---|---|
| H292 | OVTOKO |

| | IC₅₀ | Maximum | IC₅₀ | Maximum |
|---|---|---|---|---|
| Construct | (pM) | Killing (%) | (pM) | Killing (%) |
| v34913 | 0.363 | 73.1 | 2228 | 39.2 |
| v34914 | 14.67 | 68.9 | 4260 | 34.6 |
| v34916 | 42.86 | 68.1 | ND | 7.9 |
| v34917 | 36.19 | 67.9 | ND | 13.5 |
| v34918 | 7.214 | 70.0 | 8058 | 38.8 |
| v34919 | 420.7 | 43.4 | ND | 7.9 |

In sum, all trivalent and trispecific constructs tested exhibited measurable cytotoxicity against the MSLN+ cell line, H292, with maximum cytotoxicity reaching between 67-74%, whereas the MSLN×CD3 bispecific control construct, v34919, reached only 43.4%. The most potent (the IC₅₀ values were used as a measurement for potency in this study) trispecific construct, v34913, showed approximately 1200-fold higher potency as compared to the corresponding anti-CD3×MSLN bispecific control construct v34919. The constructs v34918 and v34914 exhibited similar potencies, which were approximately 30 to 60-fold higher than that measured for the control v34919. The constructs v34916 and v4917 exhibited similar potencies, which were approximately 10-fold higher than that measured for the control v34919.

Overall cytotoxicity induced by the trispecific constructs, v34913, v34914, v34917, and v34918, was reduced in MSLN^{low} cell lines (compared to the MSLN^{high} H292 cells), with maximum cytotoxicity reaching 39.2%, 34.6%, 13.5% and 38.8%, respectively. The benchmark control anti-(MSLN×CD3) bispecific construct, v34919, induced cytotoxicity toward MSLN^{low} OVTOKO cells reaching a maximum of 7.9%.

These results show that the trivalent and trispecific constructs not only exhibited antigen-dependent cytotoxic activity against tumor cells, with reduced cytotoxicity against MSLN^{low} OVTOKO cells, but also a significantly higher cytotoxic activity (e.g., at least 10-fold higher) than a corresponding control anti-(MSLN×CD3) bispecific construct (v34919).

### Example 12: Impact of Anti-CD28 Paratope of Antibody Constructs On Sustained In Vitro Cytotoxicity Towards MSLN-Positive Tumor Cell Lines in Co-Culture with Primary Human CD3+ T Cells

To test the impact of the CD28 paratope on the sustained in vitro activity and preferential killing of tumor cells overexpressing MSLN, selected trispecific construct variants were tested in primary human T cell cultures with allogeneic tumor cell lines expressing moderate levels MSLN.

Frozen human T cells were thawed at 37° C. and mixed with H292 (MSLN+) cells such that the ratio of T cells to allogeneic tumor cells was adjusted to an E:T ratio of 1:5, respectively. The mixtures were incubated together with the antibody constructs for 7 days, after which Hoechst stain (vendor) was added to the cells. After a 30-minute incubation period, tumor cell viability was assessed through live tumor cell counts using the Operetta CLS High Content Analysis System (Perkin Elmer; HH16000020). Data are representative of 2 independent experiments with 2 donors.

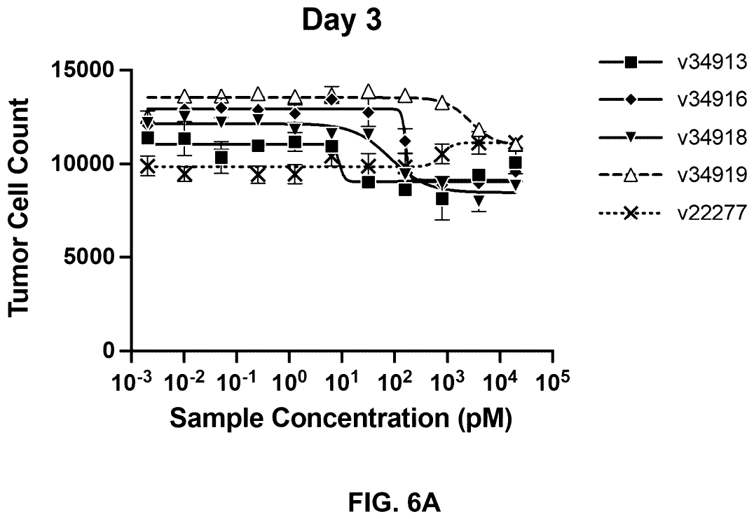

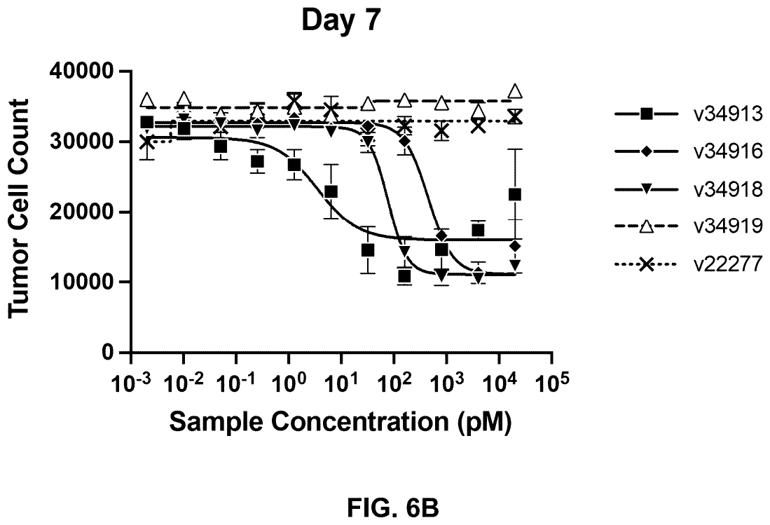

Concentration response curves for the anti-MSLN constructs are presented in FIGS. 6A-6B measured at days 3 (FIG. 6A) or day 7 (FIG. 6B) post-incubation, and IC₅₀ and maximum percent killing from the T cell-dependent cytotoxicity assay are presented in TABLE 23.

TABLE 23
Cytotoxic Activity of Anti-MSLN Trispecific Constructs and Bispecific
as well as Benchmark Constructs Towards MSLN⁺ H292 Tumor
Cells Grown in Culture with Primary Human CD3+ T Cells

| | Day 3 | | Day 7 | |
|---|---|---|---|---|
| | IC₅₀ | Maximum | IC₅₀ | Maximum |
| Construct | (pM) | Killing (%) | (pM) | Killing (%) |
| v34913 | 8.921 | 28.0 | 3.535 | 65.3 |
| v34916 | 83.11 | 19.5 | 452.3 | 66.8 |
| v34918 | 162.4 | 20.5 | 77.21 | 63.7 |
| v34919 | 2775 | 23.0 | Ambiguous | 0 |

After 7 days of co-culture, the trivalent and trispecific constructs v34913, v34916 and v34918, exhibited IC₅₀ values of 3.535 pM, 452.3 pM, and 77.2 pM, respectively, toward H292 cells, whereas no cytotoxic effect was determined for the bispecific control construct v34919. All trispecific constructs reached between about 63-67% maximum cytotoxicity of H292 target cells, whereas the control v34919 as well as the negative control v22277 did not induce any measurable cytotoxicity of H292 cells.

Taken together, these results demonstrate that the trivalent and trispecific anti-(MSLN×CD3×CD28) constructs can be superior in inducing sustained in vitro tumor cell-directed cytotoxicity toward target cells expressing moderate levels of MSLN when compared to corresponding bivalent anti-(MSLN×CD3) bispecific constructs. Furthermore, these data show that the trivalent and trispecific antibody constructs of the present disclosure are capable of inducing strong anti-tumor T cell responses over long incubation periods in long-term co-cultures (e.g., may be more reflective of conditions in solid tumors), and, particularly noteworthy, at low effector (e.g., T cell) to tumor cell ratio. This ability appears particularly striking when compared to the corresponding anti-(MSLN×CD3) bispecific construct, v34919, which, especially in the 7-day long-term co-culture, showed no significant anti-tumor activity.

### Example 13: Impact of Trispecific Construct Format on Proliferation of Human CD3+ T Cells in Co-Culture with MSLN-Positive Tumor Cell Lines

Activation of T cells through cross-linking of CD3c and CD28 via MSLN×CD3×CD28 trispecific antibodies can lead to T cell proliferation. Hence, the present example assessed the induction of target-dependent T cell proliferation by selected trivalent and trispecific anti-(MSLN×CD3×CD28) constructs and compared those to the bivalent anti-MSLN (scFv)/anti-CD3 (Fab) bispecific construct, v37663, as described below.

Antibody constructs to be tested were diluted in RPMI1640 (Gibco)+10% FBS (ThermoFisher Scientific)+1% Pen/Strep (Gibco) and titrated 1:5 in appropriate wells of the 96-well flat bottom assay plates (ThermoFisher Scientific). Human panT cells were thawed and incubated with 2 mM CellTrace CFSE staining solution (ThermoFisher Scientific) for 15 minutes at 37° C.+5% carbon dioxide. Following incubation with CellTrace CFSE staining solution or Cell Proliferation Dye cFluor 670, T cells were washed and mixed with OVCAR-3 (˜780 000 MSLN/cell) cells or H292 (˜150 000 MSLN/cell), cells at E:T ratios of 10:1 and 2:1, respectively. Plates were incubated at 37° C. with 5% carbon dioxide for 5 days, after which cells were transferred to the wells of a V-bottom 96 well plate and washed with 1×PBS (Gibco)+2% FBS (ThermoFisher Scientific). Cells were then incubated with a fluorophore-conjugated antibody specific for CD3, and a live/dead stain (Biolegend) and incubated at RT for 60 minutes. Following staining of cells, plates were washed three times with 1×PBS (Gibco)+2% FBS (ThermoFisher Scientific). CFSE, APC and BV421 signals of up to 100 000 events per well were measured by flow cytometry using BD LSRFortessa Cell Analyzer (BD Biosciences). Data analysis and T cell proliferation modeling was completed using FlowJo software (BD Biosciences). T cell counts were determined using event counts following gating on the CD3+, live cell population.

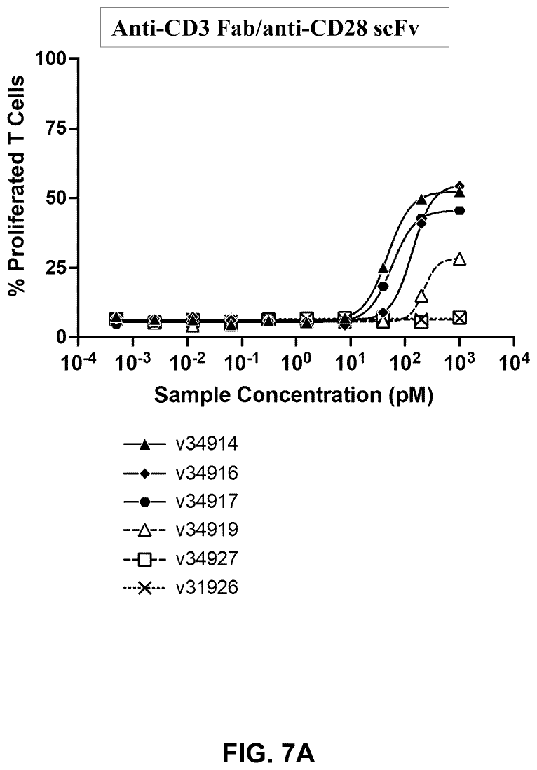

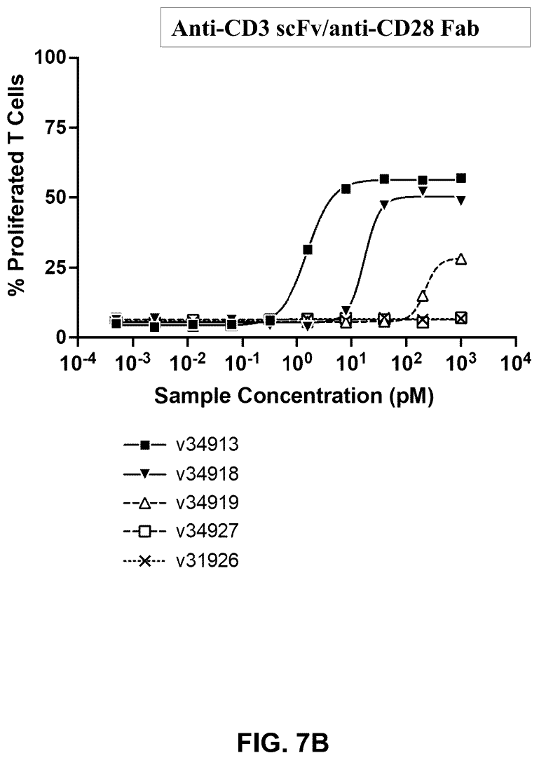

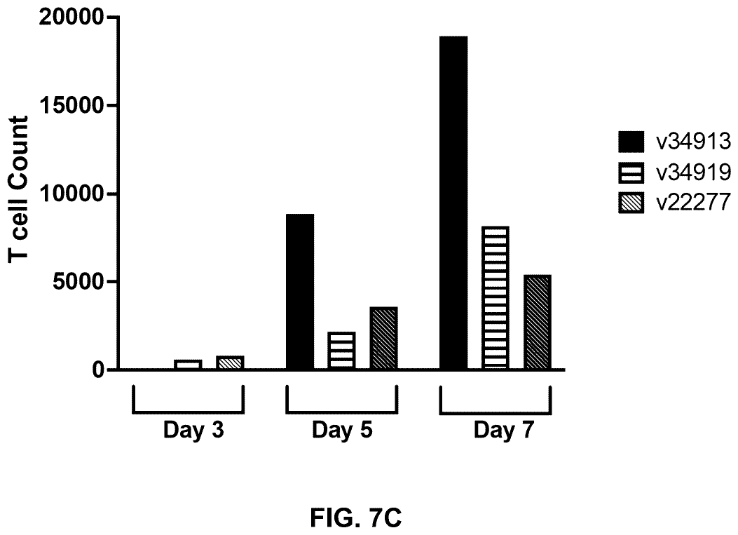

Proliferation induced by the tested trivalent and trispecific anti-(MSLN×CD3×CD28) constructs as well as for the anti-(MSLN×CD3) bispecific controls are presented in FIGS. 7A-7B.

The anti-(MSLN×CD28) bispecific control construct, v34927, and hemagglutinin anti-(HAxCD3) negative control, v31926, did not induce any T cell proliferation. The anti-(MSLN×CD3) bispecific control construct, v34919, exhibited limited proliferative activity with a calculated EC₅₀ of 221 pM and a maximum T cell proliferation of 28%. All of the tested trivalent and trispecific constructs had greater proliferative activity than v34919, with EC₅₀ values ranging from about 1.5 pM to about 189 pM, and maximum T cell proliferation ranging from about 46% to about 56%. The trivalent and trispecific construct that comprised one anti-CD28 Fab domain, one anti-CD3 scFv domain, and one anti-MSLN scFv domain, v34913 and v34918, were the most potent inducers of T cell proliferation with EC₅₀ values of approximately 1.5 pM and 17.5 pM, respectively, and maximum proliferation of 56% and 50%, respectively. This makes the constructs v34913 and v34918 a respective 147-fold and 13-fold more potent at inducing T cell proliferation than the control v34919. These two trispecific constructs also induced nearly 2-fold more T cell proliferation at the maximum concentration assessed.

The trivalent and trispecific constructs that contained one anti-CD3 Fab domain, one anti-CD28 scFv domain, and one anti-CLDN18.2 scFv domain, v34914, v34916, and v34917, were less potent inducers of T cell proliferation when compared to the trivalent and trispecific constructs v34913 and v34918, with EC₅₀ values of 48, 134, and 58 pM, respectively. Although these constructs were less potent, they induced a maximal T cell proliferation that was similar to those of v34913 and v34918, i.e., around 50%. These constructs were also still 1.6- to 4.6-fold more potent inducers of T cell proliferation than the bispecific control, v34919, and induced nearly 2-fold more T cell proliferation at the maximum concentration assessed.

T cell counts following 3, 5, and 7-days in co-culture with H292 cells are illustrated in FIG. 7C. The trivalent and trispecific anti-(CLDN18.2×CD3×CD28) construct, v34913, induced greater T cell proliferation than the bivalent bispecific following a 5-day and 7-day co-culture with H292 cells. Similarly, the trispecific construct, v34913, but not the bispecific control, v34919, induced cytotoxicity after a 7-day co-culture of T cells with H292 cells at a low E:T ratio.

These results show that induction of T cell proliferation can be enhanced for T cell engaging therapeutics by generating trispecific constructs that can bind CD3 and CD28 on T cells. These results also show that T cell proliferation can be further modulated through modification of the construct's format, with various formats exhibiting a range in potency and maximum T cell proliferation. In addition, FIG. 7C shows T cell proliferation data measured after 3 days, 5 days, and 7 days of co-culture for the most potent trivalent and trispecific construct, v34913, a corresponding bispecific anti-(MSLN×CD3) control construct, v34919, as well as the negative control v22277. These data further demonstrate the superior abilities of the trivalent and trispecific antibody constructs described herein to, e.g., induce T cell proliferation, when compared to corresponding bispecific constructs that only contain one T cell antigen binding domain.

### Example 14: Determination of Cldn18.2 Density on Cell Surface of Target Cell Lines

Target cell lines with differing levels of plasma membranous Cldn18.2 expression were used in this disclosure to assess how, e.g., trispecific construct format impacts T cell-mediating killing of target cells. Therefore, plasma membranous Cldn18.2 expression was quantified on different cell lines.

The Antibody Binding Capacity on cancer cell lines expressing different levels of Cldn 18.2 was assessed by flow cytometry using Quantum Simply Cellular anti-human IgG kit (Bangs Laboratories, Inc.). Briefly, Alexa Fluor 647-labelled anti-CLDN18.2 antibody, v35777, was used to stain beads and test samples according to the manufacturer's instructions and were run on the same day and at the same photomultiplier tube settings. To calculate the cell surface Antibody Binding Capacity values, the geometric means for the four Quantum Simply Cellular beads and test samples were uploaded to the QuickCal v2.3 Excel spreadsheet-based analysis template (Bangs Laboratories, Inc.).

Plasma membranous CLDN18.2 densities of several cancer cell lines are presented in TABLE 24. SNU 601 showed the highest expression level, followed by KATO III, and DAN G, with SKOV-3 showing the lowest expression of CLDN18.2.

TABLE 24
Cldn18.2 Density on Certain Cancer Cell Lines Represented
as the Average Antibody Binding Capacity

| | CLDN18.2 Density |
|---|---|
| Cell Line | (Antibody binding capacity) |
| SNU 601 | 276,125 |
| KATO III | 63,566 |
| DAN G | 33,164 |
| SKOV-3 | 12,951 |

### Example 15: Ability of Trispecific Antibody Constructs to Bind Native Cldn18.2 as Assessed by Flow Cytometry

To determine the impact of the trispecific construct format on the construct's ability to binding CLDN18.2, the binding affinity to CLDN18.2 was measured for certain constructs using flow cytometry as described below.

Specifically, the ability of certain trispecific anti-(CLDN18.2×CD3×CD28) antibody constructs as well as bispecific and benchmark controls to bind to CLDN18.2+ SNU 601 cells, was assessed via whole cell FACS binding analysis. To that end, cells (>80% viability) were resuspended to 1×10⁶ cells/mL in FACS buffer (PBS+2% FBS; Gibco) and seeded in a 96 well v-bottom plate (Sarstedt). Cells were mixed with antibody dilutions and incubated at 4° C. for one hour. Following incubation, cells were washed twice by adding 150 mL FACS buffer and centrifuging for 400×g for 3 minutes. The cell pellet was resuspended in 50 mL FACS buffer with Alexa Fluor 647-labeled goat anti-human IgG (Jackson Labs) and incubated for 1 hour at 4° C. Cells were washed twice by adding 150 mL FACS buffer and centrifuging for 400×g for 3 minutes. Cells were resuspended in FACS buffer and acquired on the BD LSRFortessa Cell Analyzer. The apparent binding affinities of the variants to MSLN are shown in TABLE 25.

TABLE 25
Whole Cell Binding of Engineered Trispecific
Constructs to Native CLDN18.2

SNU 601

| | Apparent | Bₘₐₓ (MFI; |
|---|---|---|
| | Affinity | anti-IgG |
| Construct | (EC₅₀; nM) | AF647) |
| v37633 | 40.8 | 8574.75 |
| v37634 | 27.2 | 6835.5 |
| v37635 | 30.1 | 6597.25 |
| v37638 | 47.5 | 9138.25 |
| v37640 | 26.4 | 9767.25 |
| v37642 | 24.1 | 9312.25 |
| v37663 | 31.3 | 9198.5 |
| v37411 | 8.9 | 11690 |
| v37675 | 37.1 | 9825.25 |
| v35923 | 244.6 | 7861.75 |

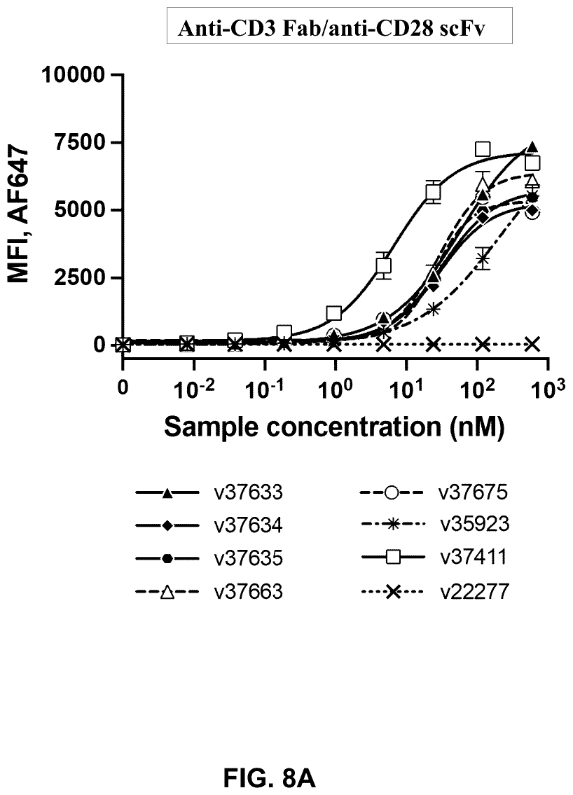

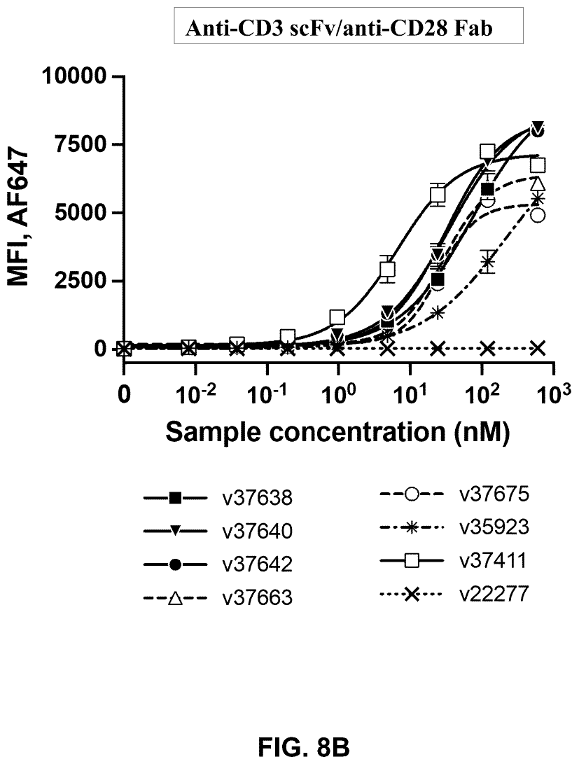

Binding curves for the tested anti-(CLDN18.2×CD3×CD28) trispecific constructs of varying geometry as well as those of the bispecific controls are shown in FIGS. 8A-8B.

All tested trivalent and trispecific anti-(CLDN18.2×CD3×CD28) constructs, the anti-(CLDN18.2×CD3) bispecific control, as well as the anti-CLDN18.2 scFv-OAA control exhibited similar binding to CLDN18.2+ SNU 601 cells, with apparent affinities in the range of about 25-50 nM. The anti-CLDN18.2 monoclonal antibody (mAb), v37411, exhibited the highest apparent affinity with an EC₅₀ of 8.9 nM. However, all of the tested trivalent and trispecific constructs showed an improved binding, with an approximately 5 to 10-fold lower EC₅₀ (i.e., higher binding) than the benchmark CLDN18.2×CD3 bispecific control, v35923.

The trispecific constructs, v37633, v37638, v37640, v37642, v37663, and the anti-CLDN18.2 scFv-OAA, v37675, exhibited similar maximal binding, with Bmax values of approximately 9,000. The trispecific constructs, v37634 and v37635, exhibited the lowest maximal binding, with Bmax values around 6,800 to 6,600, respectively. The anti-CLDN18.2 mAb, v37411, showed the highest maximal binding, with a Bmax value of 11,690. No binding was observed for the negative control, v22277.

Taken together, these results demonstrate that the trivalent and trispecific anti-(CLDN18.2×CD3×CD28) constructs were capable of binding native CLDN18.2 expressed on the surface of SNU 601 cells.

### Example 16: Ability of Cldn18.2×CD3×CD28 Trispecific Antibody Constructs to Bind to Native CD3 and CD28 as Assessed by Flow Cytometry

To determine the impact of the trispecific construct format on its ability to bind CD3+CD28+ primary human T cells, the binding affinity to human T cells was measured by flow cytometry as described below.

The ability of trispecific anti-(CLDN18.2×CD3×CD28) antibody constructs, as well as those of some bispecific and benchmark controls, to bind to CD3+CD28+ primary human T cells was assessed via whole cell FACS binding analysis. To that end, primary human T cells (>80% viability) were resuspended to 1×10⁶ cells/mL in FACS buffer (PBS+2% FBS; Gibco) and seeded in a 96 well v-bottom plate (Sarstedt). Cells were mixed with antibody dilutions and incubated at 4° C. for one hour. Following incubation, cells were washed twice by adding 150 mL FACS buffer and centrifuging for 400×g for 3 minutes. The cell pellet was resuspended in 50 mL FACS buffer with Alexa Fluor 647-labeled goat anti-human IgG (Jackson Labs) and fluorophore-conjugated antibodies specific for CD4 and CD8. Cells were incubated for 1 hour at 4° C. Following incubation, cells were washed twice by adding 150 mL FACS buffer and centrifuging for 400×g for 3 minutes. Cells were resuspended in FACS buffer and acquired on the BD LSRFortessa Cell Analyzer. Data are representative of 3 independent experiments with 3 donors.

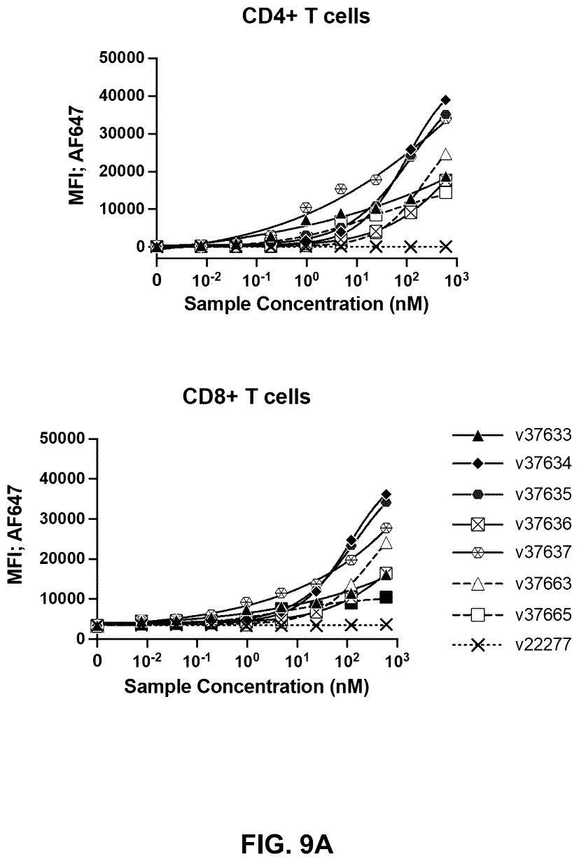

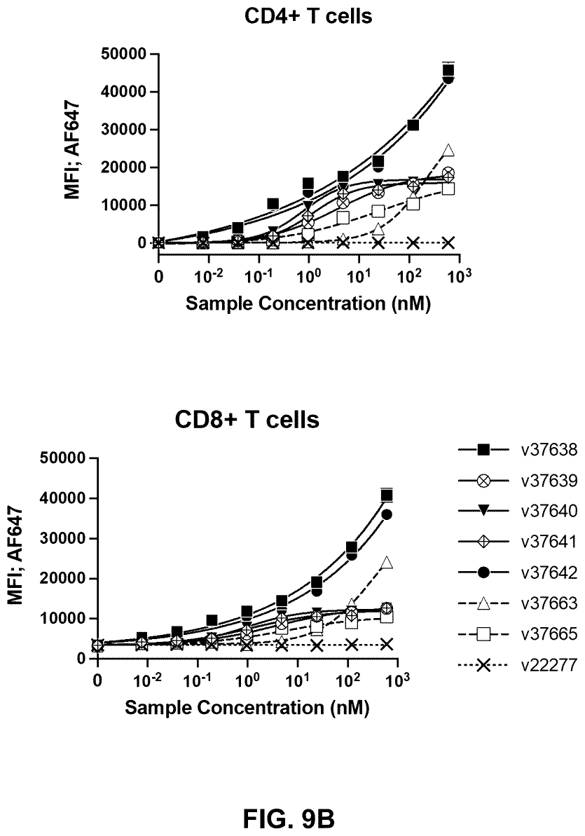

The binding curves of the tested constructs to CD3+CD28+ T cells are shown in FIGS. 9A-9B.

The anti-(Cldn18.2×CD3) bispecific control construct, v37663, exhibited the weakest binding to CD4+ and CD8+ human T cells with a relative binding affinity around 200 nM. The relative binding affinity of the anti-CD28 bispecific control, v37665, was higher than that of the CD3 bispecific, and measured around 25 nM. The tested trispecific constructs displayed stronger binding to the human T cells, compared to both bispecific controls, with two trispecific constructs, v37638 and v37642, exhibiting sub-1 nM binding. This increase in affinity relative to the bispecific controls indicates bivalent binding to both CD3 and CD28 on T cells of the trispecific constructs. No binding was observed with the negative control, v22277.

### Example 17: Impact of Trispecific Construct Format on In Vitro Cytotoxicity Towards Cldn18.2-Expressing Tumor Cell Lines in Co-Culture with Primary Human CD3+ T Cells

To test the impact of a construct's format and/or geometry on its anti-tumor cell activity and preferential killing of such tumor cells overexpressing CLDN18.2, selected constructs were tested in primary human T cell cultures with allogeneic tumor cell lines expressing CLDN18.2.

Frozen human T cells were thawed at 37° C. and mixed with CLDN18.2+ SNU 601-RFP cells such that the ratio of T cells to allogeneic tumor cells was adjusted to an E:T ratio of 2:1. The mixtures were incubated together with the antibody constructs for 72 hours, after which tumor cell viability was assessed through live tumor cell counts (RFP+) using the Operetta CLS High Content Analysis System (Perkin Elmer; HH16000020). Data are representative of 3 independent experiments with 2 donors.

Percent cytotoxicity was calculated using the following formula:

$$\% cytotoxicity = ( 1 - \frac{n umber of tumor cells left in sample}{total number of tumor cells in untreated condition} ) \times 100$$

TABLE 26
Potency and Maximum Cytotoxicity Induced by Anti-CLDN18.2
Trispecific Antibody Constructs from Primary Human
T Cells Co-cultured with SNU 601 Target Cells

SNU 601

| | IC₅₀ | Maximum |
|---|---|---|
| Construct | (pM) | Killing (%) |
| v37633 | — | 86.1 |
| v37634 | 0.032 | 88.0 |
| v37635 | 0.099 | 83.4 |
| v37636 | 6.082 | 81.5 |
| v37637 | 0.328 | 85.0 |
| v37638 | 0.006 | 84.8 |
| v37639 | 3.350 | 83.4 |
| v37640 | 0.126 | 87.8 |
| v37641 | 0.178 | 85.3 |
| v37642 | 0.001 | 87.0 |
| v37663 | 0.461 | 84.3 |
| v37665 | Ambiguous | 8.2 |

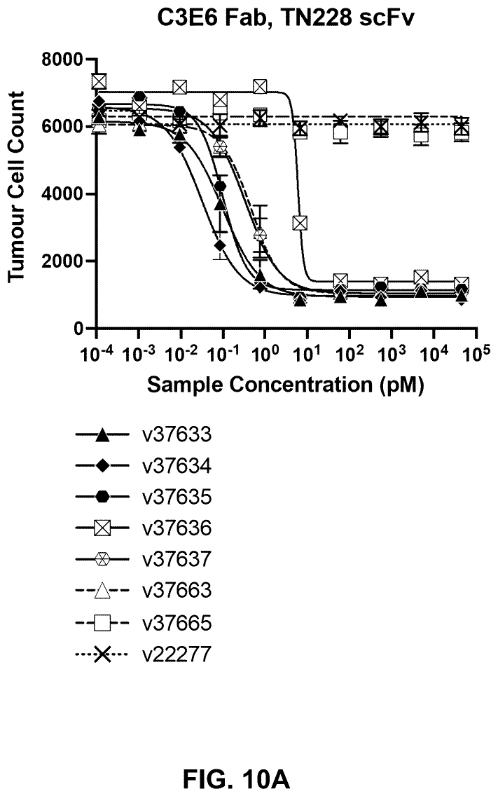

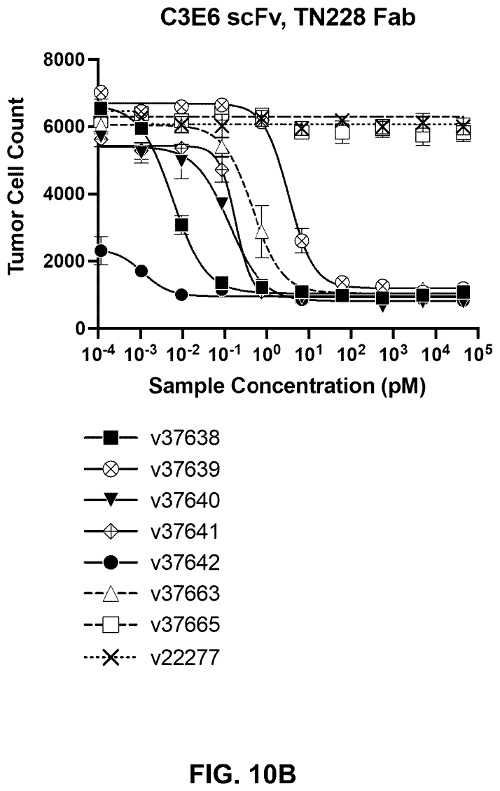

Concentration response cytotoxicity curves of primary human T cells in co-culture with CLDN18.2+ SNU 601 cells following treatment with tested anti-(CLDN18.2×CD3×CD28) trispecific constructs and bispecific controls are shown in FIGS. 10A-10B.

The trivalent and trispecific constructs that comprised one anti-CD3 Fab domain, one anti-CD28 scFv domain, and one anti-CLDN18.2 scFv domain exhibited a range of potencies against CLDN18.2-overexpressing SNU 601 allogeneic target cells. The most potent of the construct, v37634, showed approximately 15-fold higher potency compared to the respective bivalent anti-(CLDN18.2×CD3) bispecific control, v37663. The constructs v37633 and v37635 exhibited similar potencies, which were approximately 5-fold higher than v37663. The construct v37637 exhibited a similar potency when compared to v37663, with IC₅₀ values of 0.328 pM and 0.461 pM, respectively. The trivalent and trispecific constructs v37634, v37633, v37635, and v37637 all induced similar maximum cytotoxicities when compared to the bivalent anti-(CLDN18.2×CD3) bispecific control construct, with maximum cytotoxicities reaching between 83-88%. The construct v37636 exhibited a reduced cytotoxicity against SNU 601 cells compared to v37663, with an approximate 10-fold reduction in the IC₅₀ value and a maximum cytotoxicity of 81.5%.

The trivalent and trispecific constructs that comprised one anti-CD28 Fab domain, one anti-CD3 scFv domain, and one anti-CLDN18.2 scFv domain exhibited a range of potencies against CLDN18.2-overexpressing SNU 601 allogeneic target cells. The most potent of the anti-CD28 Fab-containing constructs, v37642, and the second most potent construct, v37638, showed approximately a 450-fold and 80-fold higher potency, respectively, when compared to the bivalent anti-(CLDN18.2×CD3) bispecific control construct, v37663. The constructs v37640 and v37641 exhibited equivalent potencies, which were approximately 4 to 2.5-fold higher, respectively, than that of the control construct v37663. The trivalent and trispecific constructs v37638, v37640, v37641, and v37642 all induced similar maximum cytotoxicities when compared to the bivalent anti-(CLDN18.2×CD3) bispecific control, with maximum cytotoxicity reaching between 84-88%. The construct v37639 exhibited reduced cytotoxicity against SNU 601 cells compared to v37663, with an approximate 10-fold reduction in the IC₅₀ value and a maximum cytotoxicity of 83.4%. Negative control, v22277, did not induce cytotoxicity of SNU 601 cells.

These results demonstrate that the cytotoxicity of the trivalent and trispecific constructs toward allogeneic target tumor cells can be modulated through modification of the construct's format and/or geometry, with various formats exhibiting a range of potencies against SNU 601 cells, and wherein the highest performing constructs demonstrated superior anti-tumor cytotoxicities when compared to bispecific controls that only contain a single binding domain against an immune cells antigen.

### Example 18: Impact of Trispecific Construct Format on Induction Of Pro-Inflammatory Cytokine Release by Human CD3+ T Cells in Co-Culture with CLDN18.2-Positive Tumor Cells

The trispecific antibody constructs described herein can activate T cells, e.g., by simultaneously binding a tumor-associated antigen (e.g., Cldn18.2) on cancer cells and both CD3c and CD28 on one or more T cell(s), which can lead to cross-linking of CD3e and CD28. The resulting signalling cascade can trigger T cell activation, leading to cytokine secretion. To assess the impact of trispecific construct formats and/or geometries on T cell activation, TNFα and IL-2 production was measured as described below.

Test articles (e.g., antibody constructs and controls) were diluted in RPMI1640 (Gibco)+10% FBS (ThermoFisher) and added to appropriate wells of the 384-well black flat bottom assay plates (ThermoFisher, Watham, MA). Human CD3+ T cells were thawed and mixed with SNU 601 (high CLDN18.2 density) cells at an E:T ratio of 2:1 then added to the plates. Plates were incubated for 72 hr at 37° C. with 5% carbon dioxide. Post incubation, 30 μL/well of supernatant was transferred to non-binding 384-well plates (Greiner-Bio-One, Kremsmünster, Austria) and stored at −20° C.

TNFα and IL-2 were quantified using MSD (Mesoscale Discovery, Piscataway, NJ). The night before cytokine quantification, MSD plates were blocked and coated in capture antibodies according to the manufacturers' instructions. The following day, plates were washed in PBS-T and 5 μl of assay diluent was added to each plate. The supplied TNFα and IL-2 standard (calibrator 1) was titrated as per manufacturer's instructions. Supernatants were thawed at room temperature and 5 μL of samples or standards were transferred to MSD plates. Detection antibodies were prepared at appropriate dilutions and 10 μL was added to each sample and standard well in MSD plates. The plates were sealed with aluminum foil and incubated away from light at room temperature for two hours. Plates were washed 3× in PBS-T and 40 μL MSD Gold read buffer T was added to each well. Plates were read on the MESO SECTOR 6000 and cytokine concentration was determined using MSD software. Data from a standard curve and samples were used to perform a nonlinear curve-fit with x-interpolation to obtain TNFα and IL-2 concentrations in pg/mL. Data are representative of 3 independent experiments with 2 donors.

Potency and maximum cytokine production of TNFα and IL-2 by human CD3+ T cells in the presence of the tested anti-(CLDN18.2×CD3×CD28) constructs are summarized in TABLE 27.

TABLE 27
Potency and Maximum Cytokine Production Induced
by Anti-CLDN18.2 Trispecific Constructs from Primary
Human T Cells Co-cultured with SNU 601 Cells

| | IL-2 | | TNFα | |
|---|---|---|---|---|
| | | Maximum | | Maximum |
| | | cytokine | | cytokine |
| | EC₅₀ | conc. | EC₅₀ | conc. |
| Construct | (pM) | (pg/mL) | (pM) | (pg/mL) |
| v37633 | ND | 9069.5 | 146.8 | 810.1 |
| v37634 | 67.3 | 2557.3 | 10.9 | 382.2 |
| v37635 | 102.5 | 3968.5 | 25.4 | 503.4 |
| v37636 | ND | 8172.0 | ND | 775.3 |
| v37637 | 500.8 | 4677.0 | 304.6 | 571.4 |
| v37638 | 1.2 | 3321.3 | 84.9 | 385.3 |
| v37639 | ND | 8813.0 | 640.5 | 426.8 |
| v37640 | 48.1 | 5743.4 | 35.9 | 574.9 |
| v37641 | 55.0 | 4982.6 | 41.9 | 577.3 |
| v37642 | 0.8 | 3633.0 | 1.0 | 422.1 |
| v37663 | 284.4 | 499.2 | 192.3 | 170.3 |
| v37665 | Ambiguous | 8.6 | Ambiguous | 0.5 |

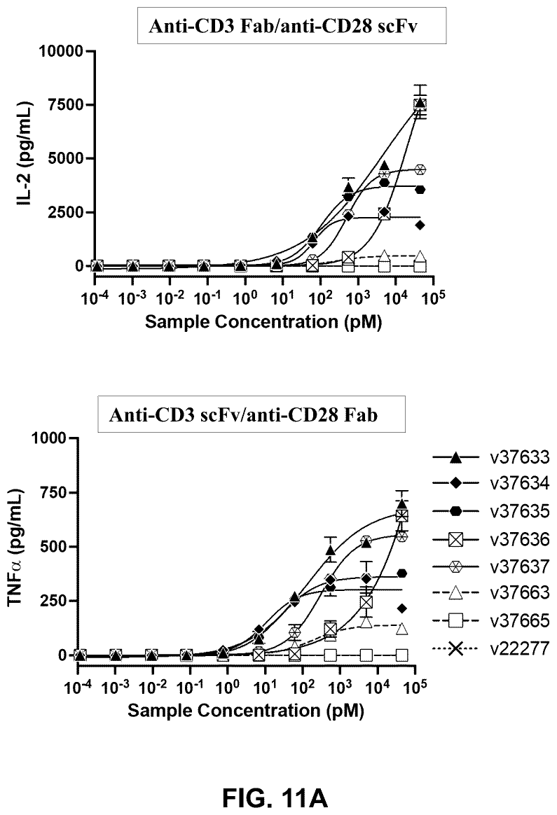

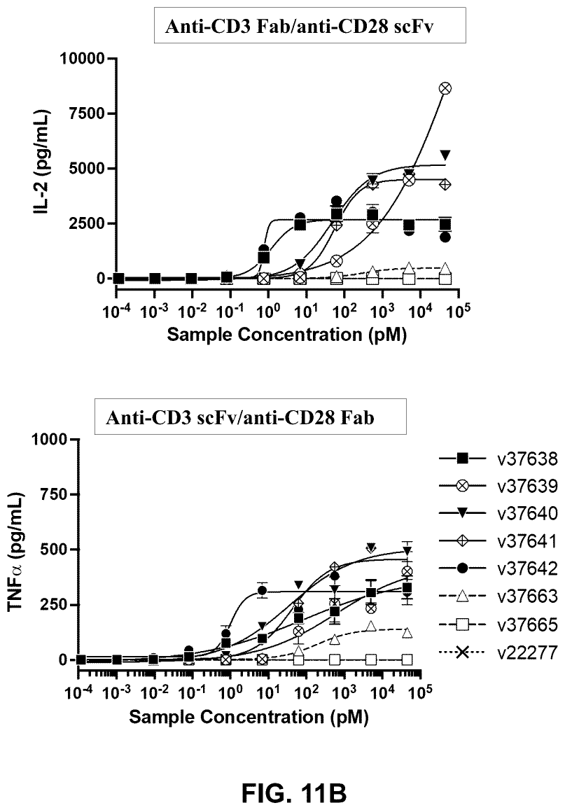

Human CD3+ T cell IL-2 (left) and TNFα (right) release after incubation in the presence of anti-(CLDN18.2×CD3×CD28) trispecific constructs with different formats are summarized in FIGS. 11A-11B.

The trivalent and trispecific constructs that comprised one anti-CD3 Fab domain, one anti-CD28 scFv domain, and one anti-CLDN18.2 scFv domain exhibited a range of potencies (e.g., EC₅₀ values) for T cell-mediated IL-2 production. The most potent and second most potent of these constructs, v37634 and v37635, respectively, showed approximately 3 to 4-fold higher potency for IL-2 production when compared to the bivalent anti-(CLDN18.2×CD3) bispecific control construct, v37663. Maximum levels of IL-2 production induced by the constructs v37634 and v37635 reached 2557.3 pg/mL and 3968.5 pg/mL, respectively, which was approximately 5 to 7-fold higher than IL-2 production induced by v37663. The construct v37637 exhibited a potency similar to v37663, with a less than 2-fold reduction in potency but resulted in an approximately 10-fold greater maximum level of IL-2 production. The potencies for the constructs v37633 and v37636 were not determined, as the curve did not reach plateau, but maximum IL-2 production reached 9096.5 pg/mL and 8172.0 pg/mL, respectively, which was approximately 15 to 20-fold greater than that induced by the control v37663.

The trivalent and trispecific constructs that contained one anti-CD3 Fab domain, one anti-CD28 scFv domain, and one anti-CLDN18.2 scFv domain exhibited a range of potencies for T cell mediated TNFα production. The most potent and second most potent constructs, v37634 and v37635, showed approximately an 8 to 15-fold higher potency for TNFα production compared to the bivalent anti-(CLDN18.2×CD3) bispecific control construct, v37663. Maximum levels of TNFα production induced by the constructs v37634 and v37635 reached 382.2 pg/mL and 503.4 pg/mL, respectively, which was approximately 2 to 4-fold higher than the TNFα production induced by the control v37663. The constructs v37633 and v37637 exhibited a similar potency to v37663 but induced an approximately 3 to 5-fold higher production of TNFα when compared to v37663. The potency for v37636 was not determined, as the concentration response curve did not reach plateau, but maximum TNFα production reached 775.3 pg/mL, which was approximately 5-fold higher than that induced by v37663.

The trivalent and trispecific constructs that contained one anti-CD3 scFv domain, one anti-CD28 Fab domain, and one anti-CLDN18.2 scFv domain exhibited a range of potencies for T cell-mediated IL-2 production. The most potent and second most potent constructs, v37642 and v37638, showed an approximately 250 to 350-fold higher potency for IL-2 production compared to the bivalent anti-(CLDN18.2×CD3) bispecific control, v37663. Maximum levels of IL-2 production induced by the constructs v37642 and v37638 reached 3633.0 pg/mL and 3321.3 pg/mL, respectively, which was approximately 6-fold higher than the IL-2 production induced by the control v37663. The constructs v37640 and v37641 exhibited similar potencies, with an approximately 5-fold increase in potency when compared to v37663, and maximum IL-2 production reached 5743.4 pg/mL and 4982.6 pg/mL, respectively, which was approximately 10-fold greater than that induced by v37663. The trivalent and trispecific constructs that comprised one anti-CD3 scFv domain, one anti-CD28 Fab domain, and one anti-CLDN18.2 scFv domain exhibited a range of potencies in T cell-mediated TNFα production. The most potent of these constructs, v37642, showed an approximately 200-fold higher potency for TNFα production compared to the bivalent anti-(CLDN18.2×CD3) bispecific control, v37663. Maximum levels of TNFα production induced by the construct v37642 reached 422.1 pg/mL, which was approximately 2-fold higher than TNFα production induced by the control v37663. The constructs v37640 and v37641 exhibited similar potencies, with an approximate 5-fold increase in potency compared to v37663, and maximum TNFα production reached 574.9 pg/mL and 577.3 pg/mL, respectively, which was approximately 3-fold greater than that induced by the control v37663. The construct v37638 exhibited an approximate 2-fold increase in potency compared to v37663, with maximum TNFα production reaching 385.3 pg/mL, approximately 2-fold greater than that induced by the control v37663. The construct v37639 showed an approximate 3-fold reduction in potency compared to v37663, with maximum TNFα production reaching 426.8 pg/mL, approximately 2.5-fold greater than that induced by the control v37663. Negative control, v22277, did not induce any measurable production of IL-2 or TNFα.

These results demonstrate that induction of cytokine production by immune cells (e.g., T cells) can be modulated through modification of a construct's format and/or geometry, with various formats exhibiting a range in potency and maximum cytokine production of IL-2 and TNFα.

### Example 19: Cldn18.2-Targeting of Anti-(Cldn18.2×CD3×CD28) Trispecific Constructs in In Vitro T-Cell Dependent Cytotoxicity Assays with Tumor Cells Expressing Varying Levels of CLDN18.2

To determine if certain trispecific antibody constructs exhibit tumor antigen dependency with respect to their in vitro cytotoxic activity, T-cell dependent cytotoxicity assays were conducted with tumor cells expressing different, e.g., high or low-levels, of antigen.

The selected anti-CLDN18.2 trispecific constructs as well as benchmark or bispecific control constructs were diluted in RPMI1640 (Gibco)+10% FBS (ThermoFisher) and added to appropriate wells of the 384-well black flat bottom assay plates (ThermoFisher, Watham, MA). Primary human CD3+ T cells were thawed and mixed SNU 601 (276,125 CLDN18.2/cell) or SKOV-3 (12,951 CLDN18.2/cell) target cells at an E:T ratio of 2:1. Plates were incubated for 72 hrs at 37° C. and 5% carbon dioxide, after which Hoechst Stain was added to the cells. After a 30-minute incubation period, tumor cell viability was assessed through live tumor cell counts using the Operetta CLS High Content Analysis System (PerkinElmer). Data are representative of 3 independent experiments with 2 donors.

Percent cytotoxicity was calculated using the following formula:

$$\% cytotoxicity = ( 1 - \frac{n umber of tumor cells left in sample}{total number of tumor cells in untreated condition} ) \times 100$$

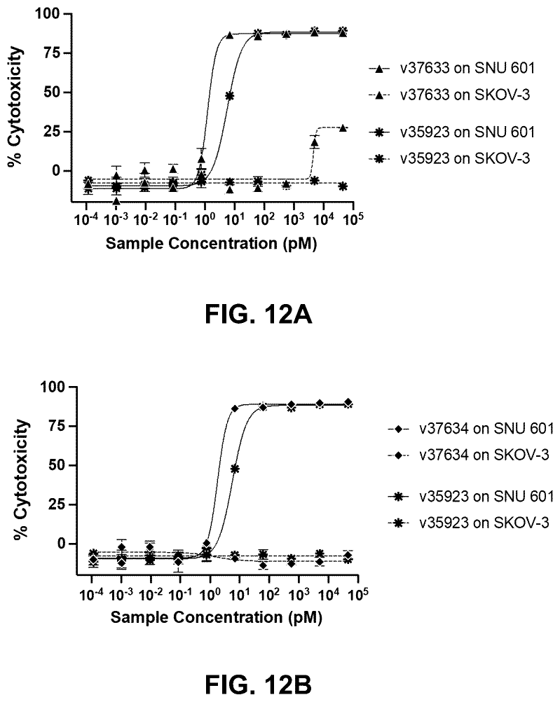

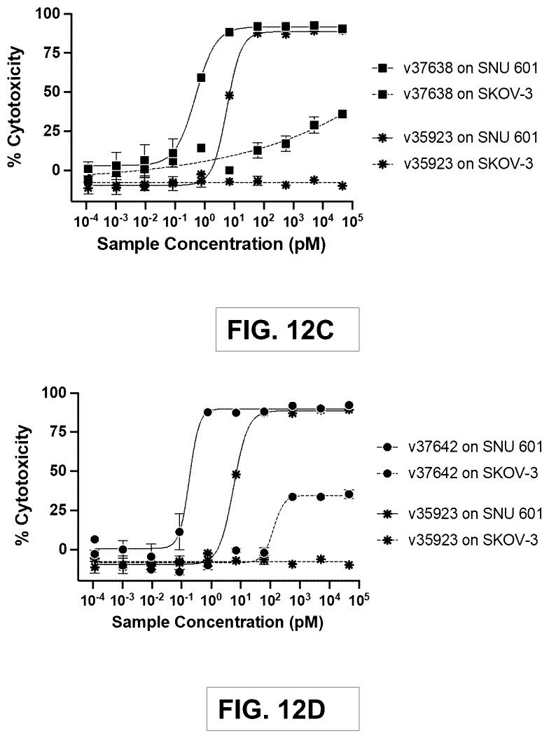

Concentration response curves for the anti-CLDN18.2 constructs are presented in FIGS. 12A-12D and IC₅₀ and maximum percent killing from the T cell-dependent cytotoxicity assay comparing the trispecific antibody constructs to the benchmark control, v35923, are presented in TABLE 28.

TABLE 28
Cytotoxic Activity of Anti-CLDN18.2 Trispecific Antibody
Constructs Towards CLDN18.2 high and low/negative
Tumor Cells Grown in Culture CD3+ T Cells

| | SNU 601 | | SKOV-3 | |
|---|---|---|---|---|
| | IC₅₀ | Maximum | IC₅₀ | Maximum |
| Construct | (pM) | Killing (%) | (pM) | Killing (%) |
| v37633 | 1.238 | 87.6 | ND | 27.67 |
| v37634 | 1.791 | 89.3 | Ambiguous | 0 |
| v37635 | 4.634 | 88.0 | ND | 21.3 |
| v37638 | 0.498 | 91.8 | ND | 36.1 |
| v37640 | 4.618 | 90.0 | ND | 34.4 |
| v37642 | 0.183 | 89.9 | 126.8 | 34.6 |
| v35923 | 5.652 | 88.7 | Ambiguous | 0 |

All trivalent and trispecific constructs tested were highly potent against the CLDN18.2+ cell line, SNU 601, with maximum cytotoxicity reaching between about 86-92%. The most potent construct, v37642, and the second most potent construct, v37638, showed an approximately 40-fold and 15-fold higher potency, respectively, when compared to the bivalent anti-(CLDN18.2×CD3) benchmark bispecific construct, v35923. The constructs v37633 and v37634 exhibited similar potencies, which were approximately 5-fold higher than v35923. The constructs v37640 and v37635 exhibited a similar potency to v35923, with ICs values of 4.618 pM and 4.634 pM, respectively.

The measured cytotoxicities induced by all tested trispecific constructs, except for v37634, were reduced in the CLDN18.2^{low} SKOV-3 cell line, with maximum cytotoxicity reaching between 21% to 36%. The construct v37642 exhibited an approximate 700-fold reduction in cytotoxic potency against the CLDN18.2^{low} SKOV-3 cells when compared to its potency against CLDN18.2^{high} SNU 601 cells. The benchmark anti-(CLDN18.2×CD3) bispecific control, v35923, however, induced no cytotoxicity towards CLDN18.2^{low} SKOV-3 cells.

These results demonstrate that the trivalent and trispecific constructs exhibit antigen-dependent cytotoxic activity, as shown first in the CLDN18.2^{high} cell line, SNU 601, followed by testing in CLDN18.2^{low} SKOV-3 cells and measuring a reduced cytotoxic activity.

### Example 20: Impact of the Presence of the Anti-CD28 Paratope in The Trispecific Constructs on Sustained In Vitro Cytotoxicity Towards CLDN18.2-Positive Tumor Cell Lines in Co-Culture with Primary Human CD3+ T Cells

To test the impact of the CD28 paratope used in the trivalent and trispecific constructs herein on their sustained in vitro activity and preferential killing of tumor cells overexpressing CLDN18.2, selected constructs were tested in primary human T cell cultures with allogeneic tumor cell lines expressing moderate levels CLDN18.2.

Frozen human T cells were thawed at 37° C. and mixed with CLDN18.2+ SNU 601-RFP, KATO III-RFP, or DAN G-RFP cells such that the ratio of T cells to allogeneic tumor cells was adjusted to an E:T ratio of 1:5. The mixtures were incubated together with the antibody constructs for 7 days, after which tumor cell viability was assessed through live tumor cell counts (RFP+) using the Operetta CLS High Content Analysis System (Perkin Elmer; HH16000020). Data are representative of 2 independent experiments with 2 donors.

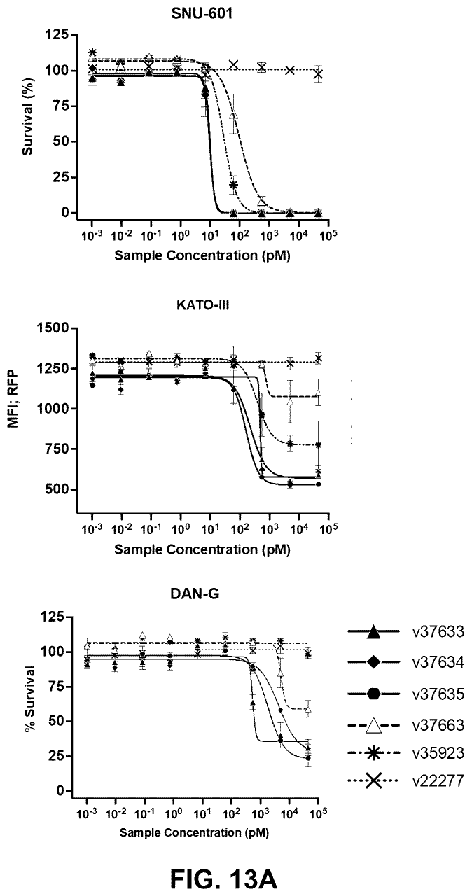

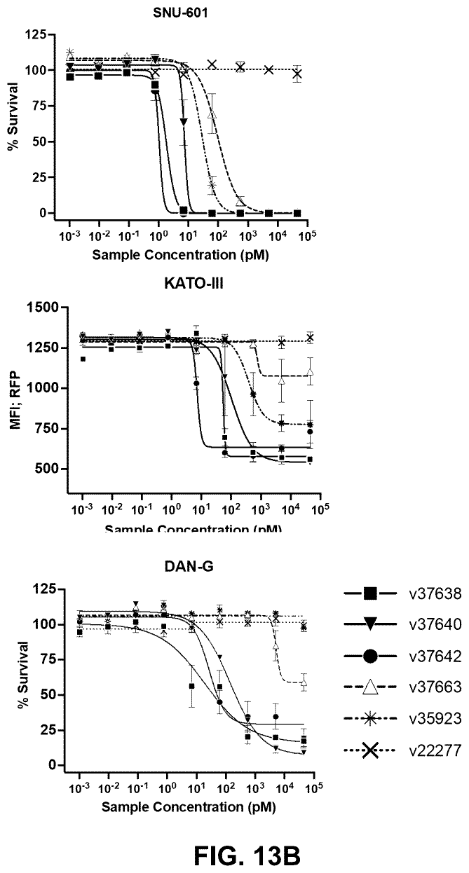

Concentration response curves for the anti-CLDN18.2 constructs are presented in FIGS. 13A-13B (left: SNU-601; middle: KATO-III; right: DAN-G) and IC₅₀ values and maximum percent killing from the T cell-dependent cytotoxicity assay are presented in TABLE 29.

TABLE 29
Cytotoxic Activity of Anti-CLDN18.2 Trispecific Constructs Towards
CLDN18.2^{med} Tumor Cells Grown in Culture with Primary Human CD3+ T Cells

| | SNU 601 | | KATO III | | DAN G | |
|---|---|---|---|---|---|---|
| | IC₅₀ | Maximum | IC₅₀ | Maximum | IC₅₀ | Maximum |
| Construct # | (pM) | Killing (%) | (pM) | Killing (%) | (pM) | Killing ( %) |
| v37633 | 10.1 | 100 | 221.9 | 89 | 546.1 | 69 |
| v37634 | 9.6 | 100 | 483.1 | 89 | 4316 | 69 |
| v37635 | 9.7 | 100 | 161.9 | 92 | 1844 | 76 |
| v37638 | 1.8 | 100 | 54.5 | 89 | 18.98 | 83 |
| v37640 | 7.3 | 100 | 106.4 | 90 | 141.7 | 91 |
| v37642 | 1.0 | 100 | 7.3 | 90 | 27.25 | 80 |
| v37663 | 96.2 | 99 | ND | 58 | 5170 | 41 |
| v35923 | 28.6 | 100 | 388.1 | 78 | Ambiguous | 2 |

The measured cytotoxic activities of the different constructs tested largely correlated with the level of CLDN18.2 expressed by the cell lines, with the highest potency observed toward SNU 601 cells (˜276,125 CLDN18.2/cell), and the lowest toward DAN G cells (˜33,164 CLDN18.2/cell). The most potent constructs, v37638 and v37642, exhibited approximately 10 to 100-fold higher potency toward SNU 601 cells when compared to the anti-(CLDN18.2×CD3) bispecific control constructs, v35923 and v37663, respectively. The constructs v37633, v37634, v37635, and v37640 exhibited similar potency toward SNU 601 cells, with an approximately 3 to 10-fold greater potency compared to those of the constructs v37663 and v35923, respectively. All tested trivalent and trispecific constructs induced 100% maximum cytotoxicity against SNU 601 target cells.

The most potent constructs, v37638 and v37642, exhibited an approximately 7 to 50-fold higher potency, respectively, toward KATO III cells when compared to the anti-(CLDN18.2×CD3) bispecific control, v35923. The constructs v37633, v37634, v37635, and v37640 exhibited an approximately 4-fold potency toward KATO III cells when compared of the potency of v35923. All trispecific constructs tested reached approximately 90% maximum cytotoxicity of KATO III target cells, whereas maximum cytotoxicity against KATO III induced by the bispecific controls, v37663 and v35923, reached 58% and 78%, respectively. Negative control, v22277, did not induce cytotoxicity of any cell line.

The most potent constructs, v37642 and v37638, exhibited an approximately 200 to 300-fold higher potency, respectively, toward DAN G cells than the anti-(CLDN18.2×CD3) bispecific control, v37663. The constructs v37640 and v37633 exhibited approximately 35 to 100-fold higher potency, respectively, toward DAN G cells when compared to v37663. The construct v37635 exhibited an approximately 10-fold higher potency toward DAN G cells when compared to v37663. The construct v37634 exhibited similar potency toward DAN G compared to v37663. All tested trispecific constructs reached between 70-90% maximum cytotoxicity against DAN G target cells, whereas maximum cytotoxicity against DAN G induced by the bispecific controls v37663 and v35923 reached 2% and 40%, respectively.

Taken together, these results suggest that the trivalent and trispecific anti-(CLDN18.2×CD3×CD28) constructs can be superior to the corresponding bivalent anti-(CLDN18.2×CD3) bispecific control constructs that only contain one immune cell antigen targeting domain at inducing a sustained in vitro cytotoxicity toward target cells expressing various levels of CLDN18.2.

### Example 21: Impact of Trivalent and Trispecific Construct Format on Proliferation of Human CD3+ T Cells in Co-Culture with Cldn-Positive Tumor Cell Lines

As further described herein, activation of T cells through cross-linking of CD3c and CD28 by the anti-(CLDN18.2×CD3×CD28) trivalent and trispecific constructs can lead to T cell proliferation. Hence, this example assessed the induction of target-dependent T cell proliferation by certain trivalent and trispecific constructs, a bivalent anti-(CLDN18.2×CD3) bispecific control, v37663, and the benchmark anti-(CLDN18.2×CD3) bispecific construct, v35923, as described below.

Test articles (e.g., antibody constructs, controls, etc.) were diluted in RPMI1640 (Gibco)+10% FBS (ThermoFisher Scientific)+1% Pen/Strep (Gibco) and titrated 1:5 in appropriate wells of 96-well flat bottom assay plates (ThermoFisher Scientific). Human panT cells were thawed and incubated with 2 mM CellTrace CFSE staining solution (ThermoFisher Scientific) for 15 minutes at 37° C., with 5% carbon dioxide. Following incubation with CellTrace CFSE staining solution, T cells were washed and mixed with SNU 601 (276,125 CLDN18.2/cell), cells at an E:T ratio of 10:1. Plates were incubated at 37° C., with 5% carbon dioxide, for 5 days, after which cells are transferred to the wells of a V-bottom 96 well plate and washed with 1×PBS (Gibco)+2% FBS (ThermoFisher Scientific). Cells were then stained with APC-conjugated anti-human CD3, OKT3, and Zombie Violet live/dead stain (Biolegend) and incubated at RT for 60 minutes. Following staining of cells, plates were washed three times with 1×PBS (Gibco)+2% FBS (ThermoFisher Scientific). CFSE, APC and BV421 signals of up to 100 000 events per well were measured by flow cytometry using the LSRFortessa Cell Analyzer (BD Biosciences). Data analysis and T cell proliferation modeling are completed using FlowJo software (BD Biosciences).

The trivalent and trispecific antibody constructs induced higher proliferation of T cells compared to corresponding bivalent and bispecific anti-(CLDN18.2×CD3) or anti-(CLDN18.2×CD28) construct that only contained a single T cell antigen binding domain.

### Example 22: Impact of Anti-Cd28 Paratope Used in Trispecific Constructs on Induction of Pro-Inflammatory Cytokine Release By Human CD3+ T Cells in Co-Culture with Cldn18.2-Positive Tumor Cells

To assess the impact of the presence of both anti-CD3 as well as the anti-CD28 paratopes in the trivalent and trispecific antibody constructs described herein as well as that of their formats on their ability to activate T cells, IFNγ, TNFα, and IL-2 production was measured as described below. These experiments compared the trispecific constructs of this disclosure to the bispecific control constructs, v37663 and v35923. In addition, bispecific control constructs in which the anti-CD28 Fab domain was replaced with an anti-RSV Fab domain were also included.

Test articles (e.g., antibody constructs, controls, etc.) were diluted in RPMI1640 (Gibco)+10% FBS (ThermoFisher) and added to appropriate wells of the 384-well black flat bottom assay plates (ThermoFisher, Watham, MA). Human CD3+ T cells were thawed and mixed with SNU 601 (high CLDN18.2 density) cells at an E:T ratio of 2:1 then added to the plates. Plates were incubated for 72 hrs at 37° C. with 5% carbon dioxide. Post incubation, 30 μL/well of supernatant was transferred to non-binding 384-well plates (Greiner-Bio-One, Kremsmünster, Austria) and stored at −20° C.

IFNγ, TNFα and IL-2 were quantified using MSD (Mesoscale Discovery, Piscataway, NJ). The night before cytokine quantification, MSD plates were blocked and coated in capture antibodies according to the manufacturers' instructions. The following day, plates were washed in PBS-T and 5 μl of assay diluent was added to each plate. The supplied IFNγ, TNFα, and IL-2 standard (calibrator 1) was titrated as per manufacturer's instructions. Supernatants were thawed at room temperature and 5 μL of samples or standards were transferred to MSD plates. Detection antibodies were prepared at appropriate dilutions and 10 μL was added to each sample and standard well in MSD plates. The plates were sealed with aluminum foil and incubated away from light at room temperature for two hours. Plates were washed 3× in PBS-T and 40 μL MSD Gold read buffer T was added to each well. Plates were read on the MESO SECTOR 6000 and cytokine concentration was determined using MSD software. Data from a standard curve and samples were used to perform a nonlinear curve-fit with x-interpolation to obtain IFNγ, TNFα and IL-2 concentrations in pg/mL. Data are representative of 1 independent experiments with 1 donors.

Potency and maximum cytokine production of IFNγ, TNFα and IL-2 by human CD3+ T cells in the presence of anti-(CLDN18.2×CD3×CD28) trispecific constructs and anti-CLDN18.2 bispecific controls are summarized in TABLE 30.

TABLE 30
Maximal Cytokine Production Induced by Anti-CLDN18.2
Trispecific Constructs from Primary Human T Cells
C o-cultured with SNU 601 Target Cells

| | Maximum | Maximum IL-2 | Maximum |
|---|---|---|---|
| Construct # | IFNγ (pg/mL) | (pg/mL) | TNFα (pg/mL) |
| v37633 | 4970.5 | 6918.9 | 653.6 |
| v37634 | 2962.7 | 1607.1 | 275.2 |
| v37635 | 4307.2 | 4129.8 | 513.5 |
| v37638 | 4070.5 | 2239.1 | 446.7 |
| v37640 | 4192.4 | 7497.7 | 780.7 |
| v37642 | 3160.2 | 3070.0 | 426.3 |
| v37658 | 716.4 | 34.0 | 40.2 |
| v37660 | 789.1 | 46.4 | 55.4 |
| v37662 | 249.9 | 13.7 | 17.6 |
| v37663 | 1958.8 | 199.9 | 120.6 |
| v37665 | 0.4 | 0.5 | 0.6 |
| v35923 | 2839.6 | 492.6 | 204.2 |

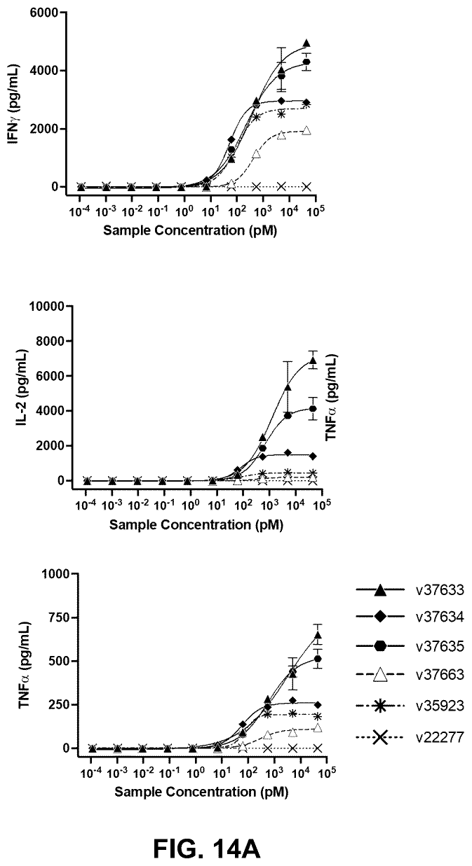

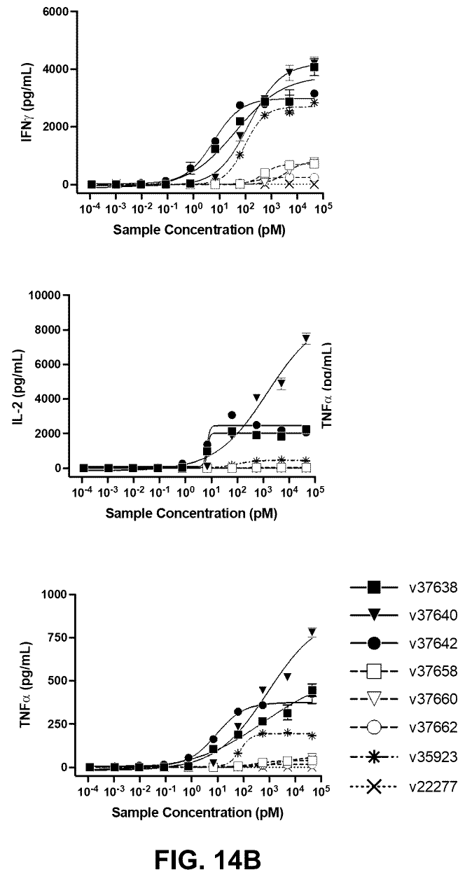

Human CD3+ T cell IFNγ, TNFα and IL-2 release after incubation in the presence of anti-(CLDN18.2×CD3×CD28) trispecific antibody constructs with different formats and anti-(CLDN18.2×CD3) bispecific controls are summarized in FIGS. 14A-14B (top left: IFNγ, top right: IL-2, bottom: TNFα).

IFNγ production induced by all tested trispecific constructs was similar to that induced by the benchmark bivalent anti-(CLDN18.2×CD3) bispecific construct, v35923, and was approximately 2-fold higher than that induced by v37663. IFNγ production induced by the trispecific constructs that comprised one anti-CD3 scFv domain, one anti-CD28 Fab domain, and one anti-CLDN18.2 scFv domain, v37638, v37640, and v37642, was approximately 5- to 10-fold greater than that induced by the trivalent, format-matched bispecific constructs, v37658, v37660, v37662, in which the anti-CD28 Fab domain was replaced with an anti-RSV Fab domain. Similar trends were observed for TNFα production of the tested trivalent and trispecific constructs.

The IL-2 production that was induced by the tested trivalent and trispecific antibody constructs was significantly higher than that produced by the corresponding bivalent anti-(CLDN18.2×CD3) constructs or the trivalent format-matched anti-(CLDN18.2×CD3×RSV) constructs. The tested trispecific constructs induced an approximately 10- to 40-fold increase in IL-2 production compared to the bivalent anti-(CLDN18.2×CD3) bispecific construct, v37663, and approximately 5- to 15-fold increase compared to the benchmark bivalent anti-(CLDN18.2×CD3) bispecific construct, v35923. IL-2 production induced by the trispecific construct that comprised one anti-CD3 scFv domain, one anti-CD28 Fab domain, and one anti-CLDN18.2 scFv domain, v37638, v37640, and v37642, was approximately 5- to 10-fold greater than that induced by the trivalent, format-matched bispecific constructs, v37658, v37660, v37662, in which the anti-CD28 Fab domain was replaced with an anti-RSV Fab domain.

These results demonstrate that induction of cytokine production can be significantly enhanced for T cell engaging therapeutics by using the trivalent and trispecific constructs of the present disclosure that can bind two antigens, e.g., CD3 and CD28, on T cells.

### Example 23: Impact of Anti-CD28 Paratope Affinity of Anti-(CLDN18.2×CD3×CD28) Trispecific Antibody Constructs to Bind to Native CD3 and CD28 as Assessed by Flow Cytometry

To determine the impact of reducing the affinity of the trispecific antibody toward CD28 on binding to CD3- and CD28-expressing cells, the binding affinity of selected constructs, including v37638, to CD3+CD28+ Jurkat Cells and primary human T cells was measured by flow cytometry as described below. Four additional constructs with the same format as v37638 but with reduced affinity to CD28, v37683, v37689, v37692, v37694 were included, as was a CLDN18.2×CD3 bispecific control, v37663.

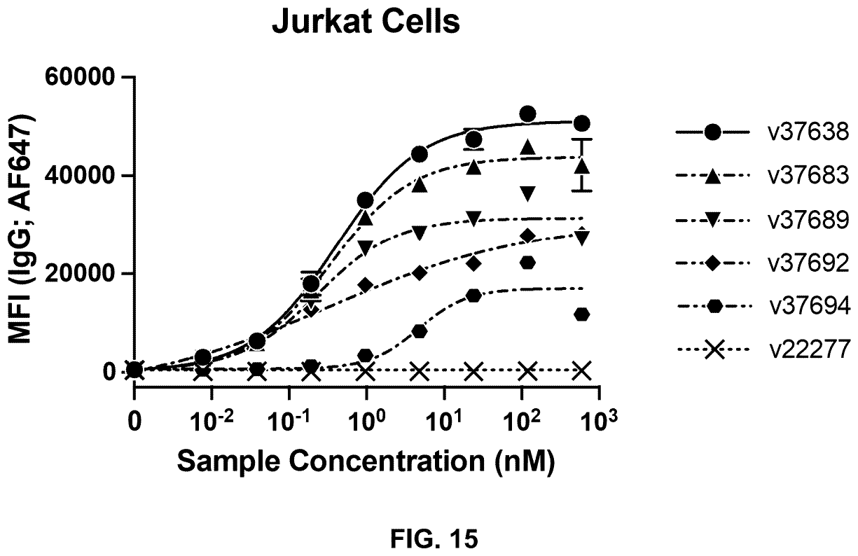

Primary human T cells and CD3+CD28+ Jurkat cells were incubated with either media or serial dilutions of the constructs for one hour at 4° C. Following incubation with the constructs, the cells were washed and stained with Alexa fluor 647-conjugated anti-IgG-Fc. The panT cells were also stained with fluorophore-conjugated antibodies specific for CD4 and CD8. The samples were acquired on a flow cytometer and analyzed using FlowJo version 10.1 software. The binding curves of some of the constructs are shown in FIG. 15 and apparent binding affinities are listed in TABLE 31.

Briefly, primary human T cells or Jurkat cells (>80% viability) were resuspended to 1×10⁶ cells/mL in FACS buffer (PBS+2% FBS; Gibco) and seeded in a 96 well v-bottom plate (Sarstedt). Cells were mixed with antibody dilutions and incubated at 4° C. for one hour. Following incubation, cells were washed twice by adding 150 mL FACS buffer and centrifuging for 400×g for 3 minutes. The cell pellet was resuspended in 50 mL FACS buffer with Alexa Fluor 647-labeled goat anti-human IgG (Jackson Labs) and fluorophore-conjugated antibodies specific for CD4 and CD8. Cells were incubated for 1 hour at 4° C. Following incubation, cells were washed twice by adding 150 mL FACS buffer and centrifuging for 400×g for 3 minutes. Cells were resuspended in FACS buffer and acquired on the BD LSRFortessa Cell Analyzer. The apparent binding affinities and Bmax of the variants are shown in TABLE 31.

TABLE 31
Whole Cell Binding of Trispecific Constructs with Reduced Affinity
for CD28 to Primary Human CD3+ T Cells and Jurkat Cells

| Construct # | Bₘₐₓ |
|---|---|
| v37638 | 52 260 |
| v37683 | 43 472 |
| v37684 | 35 541 |
| v37685 | 28 399 |
| v37686 | 50 274 |
| v37687 | 42 660 |
| v37688 | 51 732 |
| v37689 | 28 984 |
| v37690 | 21 548 |
| v37691 | 35 750 |
| v37692 | 26 851 |
| v37693 | 37 072 |
| v37694 | 14 921 |
| v37695 | 49 348 |

Apparent binding affinities (Bₘₐₓ) of tested constructs to CD3+CD28+ Jurkat cells appeared to positively correlate with the binding affinity for CD28, as measured by SPR. Taken together, these results show that certain mutations in the anti-CD28 Fab domain identified herein can specifically reduce the binding of trispecific constructs to CD3+CD28+ Jurkat cells. Negative control, v22277, did not exhibit any measurable binding to CD3+CD28+ Jurkat cells.

### Example 24: Impact of Anti-CD28 Paratope Affinity on Vitro Activity of Trispecific Constructs in T-Cell Dependent Cytotoxicity Assay With Tumor Cells Expressing Varying Levels of Cldn18.2

To assess the effect of reducing the affinity of the trispecific antibody construct toward CD28, selected constructs, including v37638, were tested in T cell-dependent cytotoxicity assays with tumor cell lines expressing varying levels of CLDN18.2. Four additional constructs with the same format as v37638 but with reduced affinity toward CD28, v37683, v37689, v37692, v37694 were included, as was a CLDN18.2×CD3 bispecific control, v37663.

Test articles (e.g., antibody constructs, controls, etc.) were diluted in RPMI1640 (Gibco)+10% FBS (ThermoFisher) and added to appropriate wells of the 384-well black flat bottom assay plates (ThermoFisher, Watham, MA). Human panT cells were thawed and mixed with SNU 601-RFP (high CLDN18.2 density) cells at an E:T ratios of 2:1. Plates were incubated for 168 hrs at 37° C. and 5% carbon dioxide, after which tumor cell viability was assessed through live tumor cell counts (RFP+) using the Operetta CLS High Content Analysis System (Perkin Elmer; HH16000020). Data are representative of 3 independent experiments with 3 donors.

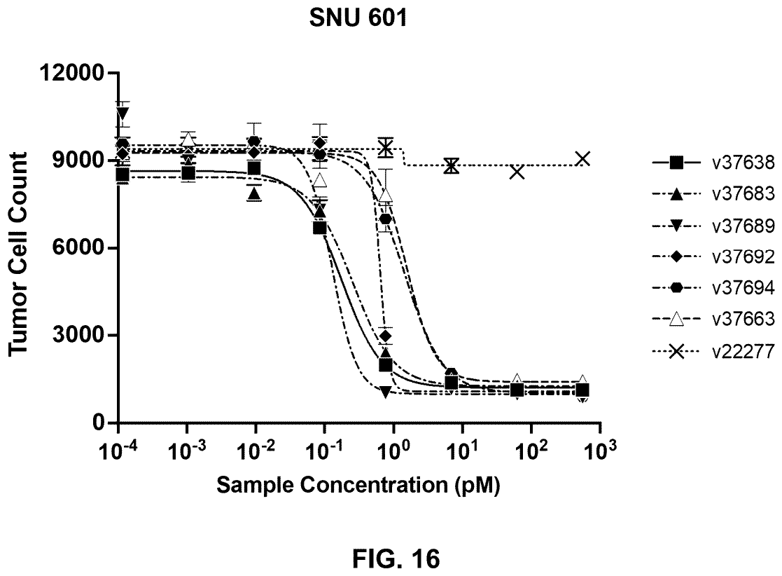

Concentration response curves for some of the anti-CLDN18.2 trispecific constructs with reduced affinity for CD28 are presented in FIG. 16 and IC₅₀ values and maximum percent killing from the T cell-dependent cytotoxicity assay are presented in TABLE 32.

TABLE 32
Cytotoxic Activity of Trispecific Constructs with
Reduced Affinity for CD28 Towards CDLN18.2 +
Tumor Cells Grown in Culture with Primary Human T Cells

SNU 601

| Construct # | IC₅₀ (pM) | Maximum Killing (%) |
|---|---|---|
| v37638 | 0.17 | 88.2 |
| v37683 | 0.25 | 88.6 |
| v37684 | 0.34 | 89.3 |
| v37685 | 0.81 | 88.7 |
| v37686 | 0.09 | 88.5 |
| v37687 | 0.10 | 88.5 |
| v37688 | 0.18 | 87.8 |
| v37689 | 0.12 | 93.4 |
| v37690 | 1.21 | 92.3 |
| v37691 | 0.08 | 91.0 |
| v37692 | 0.63 | 91.5 |
| v37693 | 0.13 | 95.3 |
| v37694 | 1.44 | 93.1 |
| v37695 | 0.04 | 89.1 |
| v37663 | 1.542 | 89.5 |

All tested trivalent and trispecific constructs that have mutations in the anti-CD28 Fab domain (mutations relative to the hTN228 wildtype V_{H} and/or V_{L} sequences) induced similar maximum cytotoxicity when compared to both (i) the trispecific constructs that comprise the wildtype TN228 paratope, e.g., construct v37638, and (ii) the bivalent and bispecific control construct, v37663, with maximum cytotoxicity reaching between 88-95%. Trispecific constructs that showed a 10-fold reduction or less in affinity for CD28 as measured by SPR maintained similar cytotoxic potency (i.e., IC₅₀ values) toward CLDN18.2+ SNU 601 cells as that of the trispecific construct with the wild type TN228 Fab domain, v37638. Trispecific constructs, v37685, and v37692, with an approximately 30 and 90-fold reduction in affinity, respectively, for CD28 by SPR exhibited a 3 to 5-fold reduction in cytotoxic potency toward CLDN18.2+ SNU 601 cells when compared to the trispecific construct with the wild type TN228 Fab domain, v37638. The trispecific constructs, v37690, and v37694, which did not exhibit measurable binding to CD28 by SPR still exhibited similar cytotoxic potency toward CLDN18.2+ SNU 601 cells when compared the bispecific construct, v37663. Negative control, v22277, did not induce cytotoxicity of SNU 601 cells.

Taken together, these results demonstrate that modifications to the anti-CD28 Fab domain that reduce the affinity of the construct for CD28 within 10-fold of wild type TN228, have minimal impact on the ability of the trispecific constructs to induce cytotoxicity toward CLDN18.2+ target cells. Reduction in affinity to CD28 greater than 10-fold compared to wild type TN228, still maintained quantifiable binding to CD28 by SPR, reduces cytotoxic potency toward CLDN+ target cells, but still maintained cytotoxic superiority to that of the corresponding bispecific control construct.

### Example 25: Impact of Anti-CD28 Paratope Affinity on Constructs' Ability to Induce Pro-Inflammatory Cytokine Release by Human CD3+ T Cells in Co-Culture with CLDN18.2+ Target Cells

To assess the effect of reducing the affinity of the trispecific antibody construct to CD28 on its ability to induce cytokine production, selected constructs, including v37638, were assessed for IL-2 and TNFα production by primary human CD3+ T cells in co-culture with CLDN18.2+ SNU 601 target cells. Four additional molecules with the same format as v37638 but with reduced affinity to CD28, v37683, v37689, v37692, v37694 were included, as was an anti-(CLDN18.2×CD3) bispecific control, v37663.

Test articles (e.g., antibody constructs, controls, etc.) were diluted in RPMI1640 (Gibco)+10% FBS (ThermoFisher) and added to appropriate wells of the 384-well black flat bottom assay plates (ThermoFisher, Watham, MA). Human CD3+ T cells were thawed and mixed with SNU 601 (high CLDN18.2 density) cells at an E:T ratio of 2:1 then added to the plates. Plates were incubated for 72 hrs at 37° C. with 5% carbon dioxide. Post incubation, 30 μL/well of supernatant was transferred to non-binding 384-well plates (Greiner-Bio-One, Kremsmünster, Austria) and stored at −20° C.

TNFα and IL-2 were quantified using MSD (Mesoscale Discovery, Piscataway, NJ). The night before cytokine quantification, MSD plates were blocked and coated in capture antibodies according to the manufacturers' instructions. The following day, plates were washed in PBS-T and 5 μl of assay diluent was added to each plate. The supplied TNFα, and IL-2 standard (calibrator 1) was titrated as per manufacturer's instructions. Supernatants were thawed at room temperature and 5 μL of samples or standards were transferred to MSD plates. Detection antibody constructs were prepared at appropriate dilutions and 10 μL was added to each sample and standard well in MSD plates. The plates were sealed with aluminum foil and incubated away from light at room temperature for two hours. Plates were washed 3× in PBS-T and 40 μL MSD Gold read buffer T was added to each well. Plates were read on the MESO SECTOR 6000 and cytokine concentration was determined using MSD software. Data from a standard curve and samples were used to perform a nonlinear curve-fit with x-interpolation to obtain TNFα and IL-2 concentrations in pg/mL. Data are representative of 3 independent experiments with 2 donors.

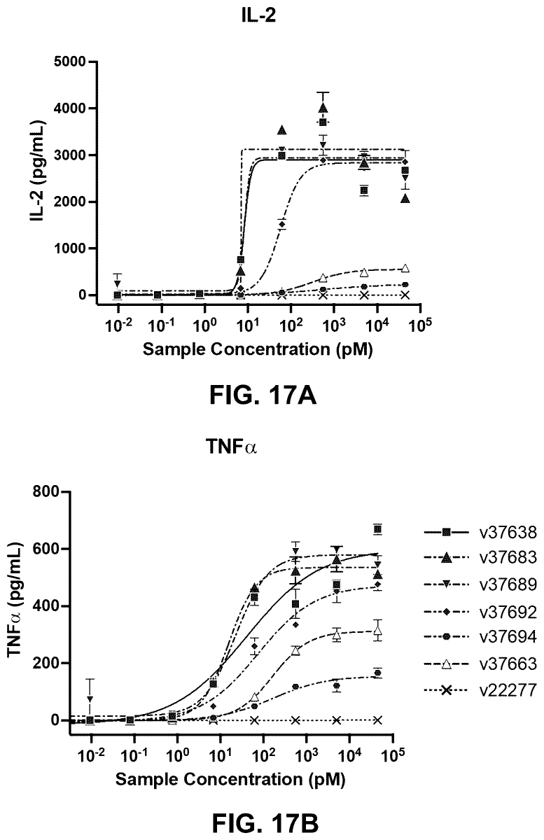

Potency and maximum cytokine production of TNFα and IL-2 by human CD3+ T cells in the presence of Cldn-expressing SNU601 cells and anti-(CLDN18.2×CD3×CD28) trispecific constructs or anti-CLDN18.2 bispecific controls are summarized in TABLE 33, as are maximum cytokine production values for experiments in which the trispecific constructs were incubated with isolated T cells only (i.e., no TAA-expressing tumor cells present). Concentration response curves for some of the tested constructs in co-cultures of T cells and SNU601 cells are illustrated in FIGS. 17A-17B.

TABLE 33
Potency and Maximal Cytokine Production Induced by Anti-CLDN18.2
Trispecific Constructs with Reduced Affinity to CD28 from Primary
Human T Cells Co-cultured with SNU 601 Target Cells

| | With SNU 601 Cells | | T cells Alone | |
|---|---|---|---|---|
| | IL-2 | TNFα | IL-2 | TNFα |
| | Maximum | Maximum | Maximum | Maximum |
| | cytokine | cytokine | cytokine | cytokine |
| Construct # | (pg/mL) | (pg/mL) | (pg/mL) | (pg/mL) |
| v37638 | 3706.7 | 669.5 | 1753.9 | 115.4 |
| v37683 | 4028.1 | 513.0 | 82.9 | 25.0 |
| v37684 | 3431.1 | 515.9 | 292.6 | 56.2 |
| v37685 | 3397.3 | 408.6 | 190.0 | 25.0 |
| v37686 | 4182.7 | 612.7 | 388.1 | 41.0 |
| v37687 | 4139.2 | 411.6 | 170.5 | 21.7 |
| v37688 | 3779.5 | 513.7 | 279.3 | 39.6 |
| v37689 | 3212.5 | 543.0 | 676.7 | 58.9 |
| v37690 | 2703.8 | 518.3 | 243.2 | 23.0 |
| v37691 | 3358.0 | 473.2 | 184.7 | 33.56 |
| v37692 | 2891.1 | 476.3 | 119.6 | 20.2 |
| v37693 | 3600.3 | 452.8 | 104.0 | 19.7 |
| v37694 | 226.4 | 166.0 | 29.9 | 12.9 |
| v37695 | 3473.7 | 403.5 | 28.9 | 13.0 |
| v37663 | 584.1 | 42.7 | 1.9 | 3.8 |

Maximum IL-2 and TNFα productions from T cells in co-culture with CLDN18.2+ SNU 601 cells induced by all constructs tested with affinity-reducing mutations in their anti-CD28 Fab domain, with the exception of construct v37694, were similar to that induced by the construct with the wild type anti-CD28 Fab, v37638. Maximum IL-2 and TNFα production induced by these trispecific constructs was on average 6-fold and 12-fold higher, respectively, than that induced by the corresponding anti-(CLDN18.2×CD3) bispecific, v37663. The construct v37694 did not induce greater maximum IL-2 production by T cells in co-culture with SNU-601 than that induced by control v37663. Furthermore, the maximum production of TNFα induced by v37694 was approximately 4-fold greater than that induced by v37663.

Target-independent (i.e., isolated T cells only in culture, no co-culture with TAA-expressing tumor cells) maximum IL-2 and TNFα productions from stimulated T cells induced by all tested constructs with affinity-reducing mutations in their anti-CD28 Fab domain was reduced compared to that induced by the construct with the wild type anti-CD28 Fab, v37638.

The construct v37695, which exhibited similar binding to CD28 as v37638, as measured by SPR, showed surprisingly an approximate 60-fold and 10-fold reduction in maximum non-specific IL-2 and TNFα production, respectively, compared to that induced by v37638, while exhibiting an approximate 15-fold and 3-fold increase in IL-2 and TNFα production, respectively, compared to that induced of v37663. The construct v37694, which did not show binding to CD28 by SPR, induced similar levels of non-specific IL-2 and TNFα production as v37695. Negative control, v22277, did not induce IL-2 or TNFα production in T cells. These data suggest that certain mutations in the anti-CD28 paratope region unexpectedly led to a reduction in non-specific activity while still providing for potent cytokine production.

Taken together, these results show that induction of nonspecific cytokine production can be modulated (e.g., reduced) by introducing specific amino acid substitutions in the anti-CD28 Fab domain, without significantly impacting the trispecific constructs' ability to induce cytokine production by T cells in co-culture with CLDN18.2+ SNU 601 cells. Furthermore, these results show that the trivalent and trispecific antibody constructs of the present disclosure can provide a strict tumor cell dependent cytotoxicity profile, with no significant activation of isolated T cells (i.e., no presence of tumor cells).

### Example 26: In Vivo Tumor Growth Inhibition Study Using Trivalent and Trispecific Constructs as Well as Corresponding Anti-(CLDN18.2×CD3) Bispecific Control Constructs

This example assessed the ability of certain trivalent and trispecific antibody constructs of the present disclosure to inhibit tumor growth and/or reduce tumor volume in the SNU620 gastric cancer model and compares their performance against corresponding bispecific control constructs.

### SNU620 Tumor Induction and PBMC Engraftment in NCG Mice for Antitumor Activity

NCG female mice (Jiangsu GemPharmatech Co, Ltd.) are inoculated subcutaneously in the right front flank region with SNU620 tumor cells (5×10⁶ cells) in 0.1 ml of PBS mixed with Matrigel (1:1) for tumor development. The date of tumor inoculation is denoted as day 0.

Animals with a single tumor of a volume of 100-120 mm³ are randomized and distributed in experimental groups according to the study protocol. Randomization is performed based on “Matched distribution” method (StudyDirector™ software, version 3.1.399.19). Immediately after randomization, animals are inoculated intravenously with PBMCs (5×10⁶ cells) from donor A or donor B.

24 hours after randomization, animals are treated intravenously as per the study design shown in TABLE 34. The test article administration and the animal numbers in each study group are also shown in TABLE 34.

After randomization, tumor volumes are measured twice per week in two dimensions using a caliper. The animals are checked daily for morbidity and mortality. During routine monitoring, the animals are checked for any effects of tumor growth and treatments on behavior such as mobility, food and water consumption, body weight gain/loss (body weights will be measured twice per week after randomization), eye/hair matting and any other abnormalities. Mortality and observed clinical signs are recorded for individual animals in detail. Animals are euthanized and necropsied when tumor volume reached a maximal tumor volume of 2 000 mm³ or when tumor exceeds 10% of normal body weight or when greater that 20% body weight loss, or after a maximum of 40 days post tumor injection.

TABLE 34
In Vivo Study Groups and Dosing Regiment

| | | | | Treatment |
|---|---|---|---|---|
| Study Group | Test Article | Dose (IV) | | Schedule |
| 1A (6 mice-donor A) | v37634 | 0.2 | mg/kg | Q7Dx4 |
| 1B (6 mice-donor B) | | | | |
| 2A (6 mice-donor A) | v37634 | 1 | mg/kg | Q7Dx4 |
| 2B (6 mice-donor B) | | | | |
| 3A (6 mice-donor A) | v37634 | 5 | mg/kg | Q7Dx4 |
| 3B (6 mice-donor B) | | | | |
| 4A (6 mice-donor A) | v37663 | 0.2 | mg/kg | Q7Dx4 |
| 4B (6 mice-donor B) | | | | |
| 5A (6 mice-donor A) | v37663 | 1 | mg/kg | Q7Dx4 |
| 5B (6 mice-donor B) | | | | |
| 6A (6 mice-donor A) | v37663 | 5 | mg/kg | Q7Dx4 |
| 6B (6 mice-donor B) | | | | |
| 7A (6 mice-donor A) | v37638 | 0.2 | mg/kg | Q7Dx4 |
| 7B (6 mice-donor B) | | | | |
| 8A (6 mice-donor A) | v37638 | 1 | mg/kg | Q7Dx4 |
| 8B (6 mice-donor B) | | | | |
| 9A (6 mice-donor A) | v37638 | 5 | mg/kg | Q7Dx4 |
| 9B (6 mice-donor B) | | | | |
| 10A (6 mice-donor A) | v37692 | 0.2 | mg/kg | Q7Dx4 |
| 10B (6 mice-donor B) | | | | |
| 11A (6 mice-donor A) | v37692 | 1 | mg/kg | Q7Dx4 |
| 11B (6 mice-donor B) | | | | |
| 12A (6 mice-donor A) | v37692 | 5 | mg/kg | Q7Dx4 |
| 12B (6 mice-donor B) | | | | |
| 13A (6 mice-donor A) | v37658 | 0.2 | mg/kg | Q7Dx4 |
| 13B (6 mice-donor B) | | | | |
| 14A (6 mice-donor A) | v37658 | 1 | mg/kg | Q7Dx4 |
| 14B (6 mice-donor B) | | | | |
| 15A (6 mice-donor A) | v37658 | 5 | mg/kg | Q7Dx4 |
| 15B (6 mice-donor B) | | | | |
| 16A (6 mice-donor A) | v35923 | 0.2 | mg/kg | Q7Dx4 |
| 16B (6 mice-donor B) | | | | |
| 17A (6 mice-donor A) | v35923 | 1 | mg/kg | Q7Dx4 |
| 17B (6 mice-donor B) | | | | |
| 18A (6 mice-donor A) | v22277 | 5 | mg/kg | Q7Dx4 |
| 18B (6 mice-donor B) | | | | |
| 19A (6 mice-No PBMC) | v38874 | 0 | mg/kg | Q7Dx4 |

19B (6 mice-No PBMC)

### Example 27: Impact of Trispecific Antibody Construct Format on Upregulation of BCL-XL by Human CD3+ T Cells in Co-Culture with CLDN18.2-Positive Tumor Cell Lines

Activation of T cells through cross-linking of CD3e and CD28 using the trivalent and trispecific anti-(CLDN18.2×CD3×CD28) antibody constructs can lead to upregulation of anti-apoptotic, pro-survival signals in T cells. Hence, this example aimed at assessing the target-dependent upregulation of Bcl-xL expression in activated T-cells using certain constructs of the present disclosure and compare those to corresponding bivalent anti-CLDN18.2 (scFv)/anti-CD3 (Fab) bispecific control constructs, as further described below.

To that end, antibody constructs were diluted to 20 nM in RPMI1640 (Gibco)+10% FBS (ThermoFisher Scientific)+1% Pen/Strep (Gibco) and plated in respective wells on a 96-well flat-bottom assay plate (ThermoFisher Scientific). Human CD3+ Pan T cells were thawed and mixed with SNU-601 (CLDN18.2-positive) tumor cell line at 2:1 E:T ratio and seeded into wells with 20 nM antibody constructs at 37° C. with 5% CO2 overnight. Cells were transferred to 96-well V-bottom plates (Sarstedt) and washed twice with cold 1×PBS (Gibco). Cells were then stained with 1:1000 Zombie Violet Dye (BioLegend) and washed 2 times with FACS buffer consisting of 1×PBS (Gibco)+2% FBS (ThermoFisher Scientific)+2.5 mM EDTA (Sigma Aldrich). Next, cells were stained with cocktail of anti-CD45, anti-CD4, and anti-CD8 or single stain (BioLegend) controls for 60 minutes and washed with FACS buffer. Then, cells were fixed and permeabilized for intracellular staining with 1× Fix/Perm concentrate (Invitrogen eBiosciences) diluted in fix/perm diluent (Invitrogen eBiosciences). Cells were incubated for 30 minutes away from light at room temperature (RT). After staining, cells were washed twice with 1× Perm buffer and stained with anti-Bcl-xL or isotype intracellular stains (CellSignaling) diluted in 1× perm buffer (Invitrogen eBiosciences) and incubated for 30 minutes at room temperature away from light. Finally, cells were washed twice with 1× perm buffer (Invitrogen eBiosciences), resuspended in FACS buffer, and events were captured on the BD LSRFortessa X-20 (BD Biosciences). Data were analyzed using FlowJo software (BD Biosciences) to generate values representing median fluorescence intensity (MFI).

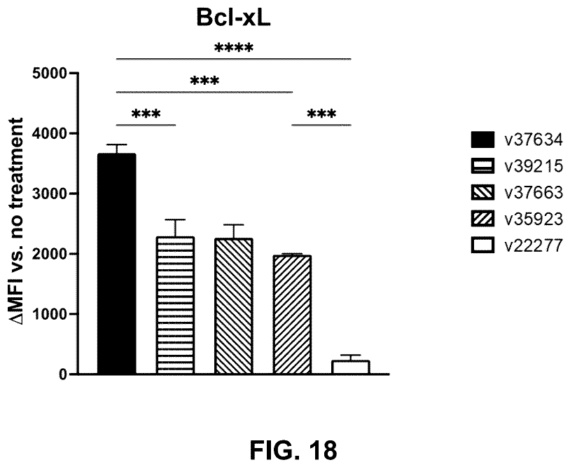

Upregulation of Bcl-XL expression in activated T-cells using the trivalent and trispecific antibody constructs of the present disclosure as well as bispecific controls are shown in FIG. 18. The negative control, v22277, showed no significant Bcl-xL upregulation. The anti-(CLDN18.2×CD3) bispecific constructs, v35923, v37663, and v39215, induced similar levels of Bcl-xL, with a change in median fluorescence intensity (MFI) of around 2000 compared to untreated cells. The trivalent and trispecific antibody construct v37634, induced the highest level of Bcl-xL expression, with a change in MFI of approximately 3500 compared to untreated cells.

Thus, the data further demonstrate that the trivalent and trispecific antibody constructs described herein can possess superior properties, e.g., induction of T cell survival, when compared to corresponding bispecific constructs that only contain one T cell antigen binding domain.

### Example 28: Impact of Trispecific Constructs on Proliferation of Human CD3+ T Cells in Co-Culture with Cldn18.2-Positive Tumor Cell Lines

Activation of T cells through cross-linking of CD3e and CD28 using the trivalent and trispecific anti-(CLDN18.2×CD3×CD28) antibody constructs can lead to T cell proliferation. Hence, this example aimed at assessing the target-dependent T cell proliferation using certain constructs of the present disclosure and compare those to a corresponding bivalent anti-CLDN18.2 (scFv)/anti-CD3 (Fab) bispecific control construct, v37663, as further described below.

Antibody constructs were diluted in RPMI1640 (Gibco)+10% FBS (ThermoFisher Scientific)+1% Pen/Strep (Gibco) to 200 pM (ThermoFisher Scientific). Human pan T cells were thawed and incubated with 5 M CellTrace CFSE staining solution (ThermoFisher Scientific) in PBS (Gibco)+1% FBS (ThermoFisher Scientific) for 10 minutes at room temperature. Following incubation with CellTrace CFSE staining solution, the T cells were incubated with an equal volume of FBS (ThermoFisher Scientific) at room temperature for 5 minutes, followed by 2 washes with RPMI1640 (Gibco)+10% FBS (ThermoFisher Scientific). CFSE-stained T cells were mixed with SNU-601 (276,125 CLDN18.2/cell) cells at an E:T ratio of 5:1 and treated with diluted test article. Plates were incubated at 37° C. with 5% carbon dioxide for 5-7 days, after which cells were transferred to the wells of a V-bottom 96 well plate and washed with 1×PBS (Gibco)+2% FBS (ThermoFisher Scientific). Cells were then incubated with a fluorophore-conjugated antibodies specific for CD45, CD4, and CD8 (BioLegend) and a live/dead stain (eBiosciences) and incubated at RT for 60 minutes. Following staining of cells, plates were washed three times with 1×PBS (Gibco)+2% FBS (ThermoFisher Scientific). CFSE signals of up to 100,000 events per well were measured by flow cytometry using BD LSRFortessa Cell Analyzer (BD Biosciences). Data analysis completed using FlowJo software (BD Biosciences). T cell counts were determined using event counts following gating on the CD45+, live cell population.

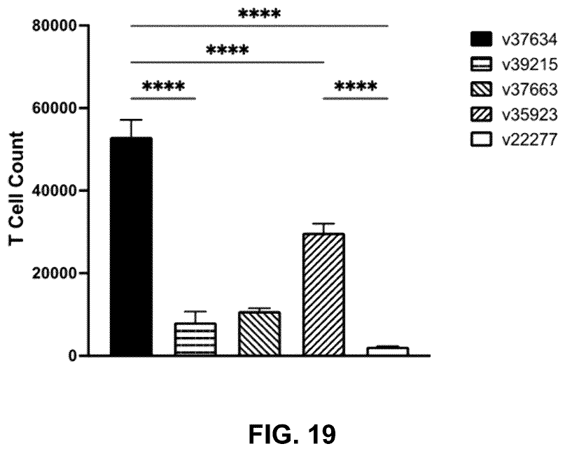

Proliferation induced by the tested trivalent and trispecific constructs as well as for the anti-(CLDN18.2×CD3) bispecific controls are shown in FIG. 19.

The negative control, v22277, did not induce any T cell proliferation. The anti-(CLDN18.2×CD28) bivalent and bispecific control construct, v34927, and the trivalent, bispecific control, v39215, induced minimal T cell proliferation, with a total event count between 8000 to 11000. The anti-(CLDN18.2×CD3) bispecific control construct, v35923, induced T cell proliferation, with the total event count reaching approximately 30 000 T cells. Finally, the trispecific and trivalent anti-(CLDN18.2×CD3×CD28) trispecific construct of the present disclosure, v37634, induced the highest level of T cell proliferation, resulting in a final event count of approximately 53 000 T cells.

These results show that induction of T cell proliferation can be enhanced for T cell engaging therapeutics by using the trispecific constructs binding CD3 and CD28 on T cells as disclosed herein.

### Example 29: In Vivo Tumor Growth Inhibition Using Trivalent and Trispecific Constructs of the Present Disclosure Compared to Corresponding Anti-(CLDN18.2×CD3) Bispecific Control Constructs

The ability of certain trivalent and trispecific antibody constructs of the present disclosure to inhibit tumor growth and/or reduce tumor volume in the SNU620 gastric cancer model was assessed and their performance was compared against that of corresponding bispecific control constructs.

### SNU620 Tumor Induction and PBMC Engraftment in NCG Mice for Antitumor Activity

NCG female mice (Jiangsu GemPharmatech Co, Ltd.) were inoculated subcutaneously in the right front flank region with SNU620 tumor cells (5×10⁶ cells) in 0.1 ml of PBS mixed with Matrigel (1:1) for tumor development. The date of tumor inoculation was denoted as day 0.

Animals with a single tumor of a volume of 100-120 mm³ were randomized and distributed in experimental groups according to the study protocol. Randomization was performed based on “Matched distribution” method (StudyDirector™ software, version 3.1.399.19). Immediately after randomization, animals were inoculated intravenously with PBMCs (5×10⁶ cells) from donor A or donor B. 24 hours after randomization, animals were treated intravenously as per the study design shown in TABLE 35 below. The test article administration and the animal numbers in each study group are also shown in TABLE 35.

TABLE 35
In Vivo Study Groups and Dosing Regiment

| | | | | Treatment |
|---|---|---|---|---|
| Study Group | Test Article | Dose (IV) | | Schedule |
| 1A (6 mice-donor A) | v37634 | 0.2 | mg/kg | Q7Dx4 |
| 1B (6 mice-donor B) | | | | |
| 2A (6 mice-donor A) | v37634 | 1 | mg/kg | Q7Dx4 |
| 2B (6 mice-donor B) | | | | |
| 3A (6 mice-donor A) | v37634 | 5 | mg/kg | Q7Dx4 |
| 3B (6 mice-donor B) | | | | |
| 4A (6 mice-donor A) | v37663 | 0.2 | mg/kg | Q7Dx4 |
| 4B (6 mice-donor B) | | | | |
| 5A (6 mice-donor A) | v37663 | 1 | mg/kg | Q7Dx4 |
| 5B (6 mice-donor B) | | | | |
| 6A (6 mice-donor A) | v37663 | 5 | mg/kg | Q7Dx4 |
| 6B (6 mice-donor B) | | | | |
| 7A (6 mice-donor A) | v37638 | 0.2 | mg/kg | Q7Dx4 |
| 7B (6 mice-donor B) | | | | |
| 8A (6 mice-donor A) | v37638 | 1 | mg/kg | Q7Dx4 |
| 8B (6 mice-donor B) | | | | |
| 9A (6 mice-donor A) | v37638 | 5 | mg/kg | Q7Dx4 |
| 9B (6 mice-donor B) | | | | |
| 10A (6 mice-donor A) | v37692 | 0.2 | mg/kg | Q7Dx4 |
| 10B (6 mice-donor B) | | | | |
| 11A (6 mice-donor A) | v37692 | 1 | mg/kg | Q7Dx4 |
| 11B (6 mice-donor B) | | | | |
| 12A (6 mice-donor A) | v37692 | 5 | mg/kg | Q7Dx4 |
| 12B (6 mice-donor B) | | | | |
| 13A (6 mice-donor A) | v37658 | 0.2 | mg/kg | Q7Dx4 |
| 13B (6 mice-donor B) | | | | |
| 14A (6 mice-donor A) | v37658 | 1 | mg/kg | Q7Dx4 |
| 14B (6 mice-donor B) | | | | |
| 15A (6 mice-donor A) | v37658 | 5 | mg/kg | Q7Dx4 |
| 15B (6 mice-donor B) | | | | |
| 16A (6 mice-donor A) | v35923 | 0.2 | mg/kg | Q7Dx4 |
| 16B (6 mice-donor B) | | | | |
| 17A (6 mice-donor A) | v35923 | 1 | mg/kg | Q7Dx4 |
| 17B (6 mice-donor B) | | | | |
| 18A (6 mice-donor A) | v22277 | 5 | mg/kg | Q7Dx4 |
| 18B (6 mice-donor B) | | | | |
| 19A (6 mice-No PBMC) | v38874 | 0 | mg/kg | Q7Dx4 |

19B (6 mice-No PBMC)

After randomization, tumor volumes were measured twice per week in two dimensions using a caliper. The animals were checked daily for morbidity and mortality. During routine monitoring, the animals were checked for any effects of tumor growth and treatments on behavior such as mobility, food and water consumption, body weight gain/loss (body weights were measured twice per week after randomization), eye/hair matting and any other abnormalities. Mortality and observed clinical signs were recorded for individual animals in detail. Animals were euthanized and necropsied when tumor volume reached a maximal tumor volume of 2 000 mm³ or when tumor exceeded 10% of normal body weight or when greater than 20% body weight loss, or after a maximum of 40 days post tumor injection. Tumor volume data were analyzed using a mixed-effect model.

### Example 30: In Vivo Tumor Growth Inhibition Study Using Trivalent and Trispecific Constructs as Well as Corresponding Anti-(CLDN18.2×CD3) Bispecific Control Constructs

The ability of certain trivalent and trispecific antibody constructs of the present disclosure to inhibit tumor growth and/or reduce tumor volume in the SNU620 gastric cancer model was assessed and their performance was compared against corresponding bispecific control constructs.

### SNU620 Tumor Induction and PBMC Engraftment in NCG Mice for Antitumor Activity

NCG female mice (Jiangsu GemPharmatech Co, Ltd.) were inoculated subcutaneously in the right front flank region with SNU620 tumor cells (5×10⁶ cells) in 0.1 ml of PBS mixed with Matrigel (1:1) for tumor development. The date of tumor inoculation was denoted as day 0.

Animals with a single tumor of a volume of 100-120 mm³ were randomized and distributed in experimental groups according to the study protocol. Randomization was performed based on “Matched distribution” method (StudyDirector™ software, version 3.1.399.19). Immediately after randomization, animals were inoculated intravenously with PBMCs (5×10⁶ cells) from donor X.

24 hours after randomization, animals were treated intravenously as per the study design shown in TABLE 36. The test article administration and the animal numbers in each study group are also shown in TABLE 36.

After randomization, tumor volumes were measured twice per week in two dimensions using a caliper. The animals were checked daily for morbidity and mortality. During routine monitoring, the animals were checked for any effects of tumor growth and treatments on behavior such as mobility, food and water consumption, body weight gain/loss (body weights were measured twice per week after randomization), eye/hair matting and any other abnormalities. Mortality and observed clinical signs were recorded for individual animals in detail. Animals were euthanized and necropsied when tumor volume reached a maximal tumor volume of 2 000 mm³ or when tumor exceeded 10% of normal body weight or when greater than 20% body weight loss, or after a maximum of 40 days post tumor injection. Tumor volume data were analyzed using a mixed-effect model.

TABLE 36
In Vivo Study Groups and Dosing Regiment

| | | | | Treatment |
|---|---|---|---|---|
| Study Group | Test Article | Dose (IV) | | Schedule |
| 1A (6 mice-donor X) | v37634 | 0.001 | mg/kg | Q7Dx4 |
| 2A (6 mice-donor X) | v37634 | 0.01 | mg/kg | Q7Dx4 |
| 3A (6 mice-donor X) | v37634 | 0.1 | mg/kg | Q7Dx4 |
| 4A (6 mice-donor X) | v38417 | 0.001 | mg/kg | Q7Dx4 |
| 5A (6 mice-donor X) | v38417 | 0.01 | mg/kg | Q7Dx4 |
| 6A (6 mice-donor X) | v38417 | 0.1 | mg/kg | Q7Dx4 |
| 7A (6 mice-donor X) | v35923 | 0.001 | mg/kg | Q7Dx4 |
| 8A (6 mice-donor X) | v35923 | 0.01 | mg/kg | Q7Dx4 |
| 9A (6 mice-donor X) | v35923 | 0.1 | mg/kg | Q7Dx4 |
| 10A (6 mice-donor X) | v22277 | 0.11 | mg/kg | Q7Dx4 |

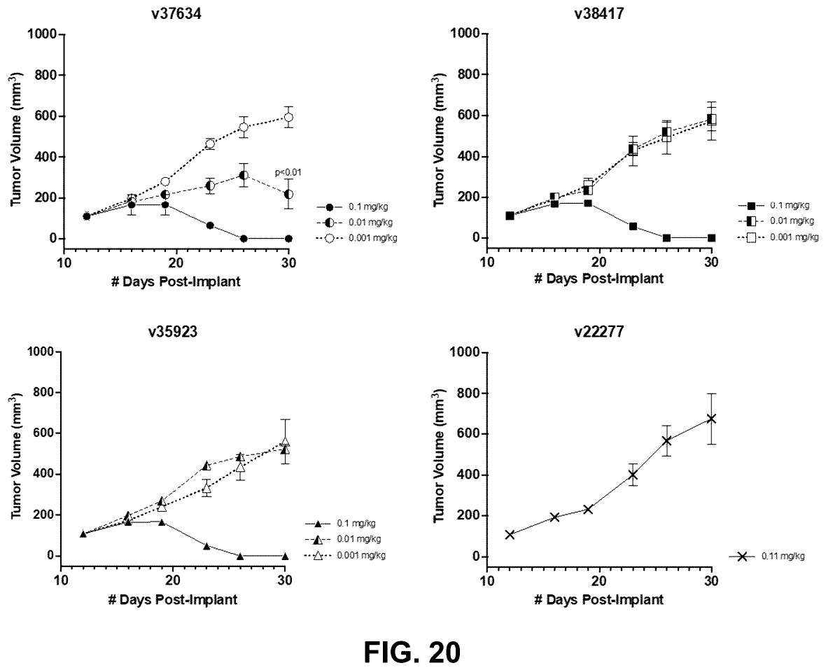

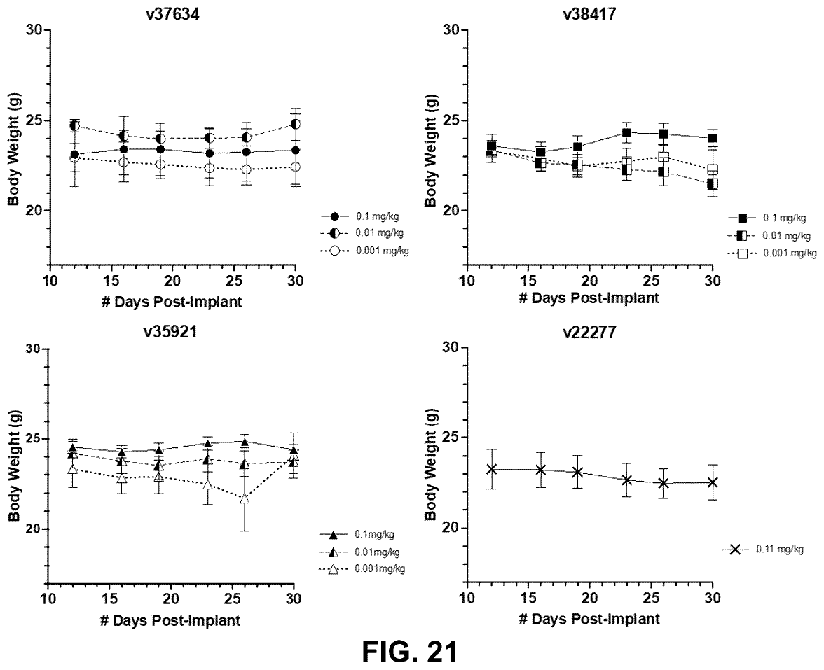

As shown in FIG. 20 and TABLE 37, tumor regression was observed in all donor X-engrafted mice treated with 0.1 mg/kg of either the bispecific constructs, v35923 or v38417, or the trispecific construct, v37634. Tumor growth rate inhibition was significantly higher in mice receiving 0.01 mg/kg of v37634 compared to mice receiving equivalent doses of either v35923 or v38417. Tumor outgrowth was observed in animals treated with the negative control (v22277). Body weights remained stable for all mice (FIG. 21).

TABLE 37
Tumor Growth Inhibition Constants
and Rate Inhibition for Donor X

| Test | | | Growth Constant (1/day; | | Growth Rate |
|---|---|---|---|---|---|
| Article | Dose | | 95% CI) | | Inhibition |
| v37634 | 0.1 | mg/kg | −0.2271 | (−0.2668, −0.1875) | 319.63% |
| | 0.01 | mg/kg | −0.1429 | (−0.1800, −0.1057) | 238.2% |
| | 0.001 | mg/kg | 0.0211 | (−0.0069, 0.0490) | 79.59% |
| v38417 | 0.1 | mg/kg | −0.2717 | (−0.3119, −0.2315) | 362.77% |
| | 0.01 | mg/kg | 0.0276 | (−0.0035, 0.0586) | 73.31% |
| | 0.001 | mg/kg | 0.0435 | (−0.0193, 0.0677) | 57.93% |
| v35923 | 0.1 | mg/kg | −0.2837 | (−0.3234, −0.2440) | 374.37% |
| | 0.01 | mg/kg | 0.0229 | (−0.0017, 0.0476) | 77.85% |
| | 0.001 | mg/kg | 0.0877 | (0.0748, 0.1006) | 15.18% |
| v22277 | 0.11 | mg/kg | 0.1034 | (0.0910, 0.1157) | 0% |

### Example 31: Impact of CD3 & CD28 Co-Engagement with a Trivalent Trispecific Antibody Compared to Combination Therapy Using Anti-(CD3×CLDN18.2) & Anti-(CD28×CLDN18.2) Bispecific Antibodies on In Vitro Cytotoxicity and T Cell Mediated Cytokine Production Towards CLDN18.2-Expressing Tumor Cell Lines in Co-Culture with Primary Human CD3+CD28+ T Cells

To interrogate the difference in cytotoxicity and induction of proinflammatory cytokine release induced by the trispecific anti-(CLDN18.2×CD3×CD28) antibody construct, v37634, in comparison to a combination of the bispecific anti-(CLDN18.2×CD3) and anti-(CLDN18.2×CD28) bispecific antibody constructs, v37663 and v37665 (i.e., v37663+v37665 vs. v37634 alone), respectively, selected constructs were tested in primary human T cell cultures with allogeneic tumor cell lines expressing CLDN18.2. Trispecific control constructs containing a paratope against the irrelevant antigen, RSV protein F (RSV-F), in place of the CD3, CD28, or CLDN18.2 paratopes, v37654, v39215, and v37644, respectively, were included as format controls for each binding arm of the tested trispecific antibody construct, v37634. A monoclonal anti-RSV protein F construct, v22277, was included as a negative control.

Frozen human CD3+ T cells were thawed at 37° C. and mixed with CLDN18.2-expressing SNU 601- and KATO III-RFP cells such that the ratio of T cells to allogeneic tumor cells was adjusted to effector-to-target (E:T) ratios of 2:1 and 1:5. The mixtures were incubated together with the constructs for 72 and 168 hours, respectively, after which tumor cell viability was assessed through live tumor cell counts (RFP+) using the Operetta CLS High Content Analysis System (Perkin Elmer; HH16000020).

Percent cytotoxicity was calculated using the following formula:

$$\% cytotoxicity = ( 1 - \frac{n umber of tumor cells left in sample}{total number of tumor cells in untreated condition} ) \times 100$$

Supernatant was collected from the 2:1 E:T experiment at the 72-hour timepoint and analyzed for IFNγ, TNFα, IL-2, and IL-6 by MSD (Mesoscale Discovery) using 4-plex U-plex MSD kits. The night before cytokine quantification, MSD plates were blocked and coated in capture constructs according to the manufacturer's instructions. The following day, plates were washed in PBS-T and 5 μl of assay diluent was added to each plate. The supplied IFNγ, TNFα, IL-2, and IL-6 standard (calibrator 1) was titrated as per manufacturer's instructions. Supernatants were thawed at room temperature and 5 μL of samples or standards were transferred to MSD plates. Detection constructs were prepared at appropriate dilutions and 10 μL was added to each sample and standard well in MSD plates. The plates were sealed with aluminum foil and incubated away from light at room temperature for two hours. Plates were washed 3× in PBS-T and 40 μL MSD Gold read buffer T was added to each well. Plates were read on the MESO SECTOR 6000 and cytokine concentration was determined using MSD software. Data from a standard curve and samples were used to perform a nonlinear curve-fit with x-interpolation to obtain IFNγ, TNFα, IL-6, and IL-2 concentrations in pg/mL.

TABLE 38
Potency and Maximum Mean Cytotoxicity (MMC) Induced
by Anti-CLDN18.2 Trivalent and Trispecific Antibody
Constructs, Bispecific Constructs alone, or a Combination
of Anti-(CLDN18.2 × CD3) and anti-(CLDN18.2 ×
CD28) Bispecific Constructs, from Primary Human T cells
co-cultured with SNU 601-RFP Target Cells

| | 2:1 E:T | | 1:5 E:T | |
|---|---|---|---|---|
| Construct # | IC₅₀ (pM) | MMC (%) | IC₅₀ (pM) | MMC (%) |
| v37634 | 1.01 | 98.3 | 14.94 | 99.7 |
| v39215 | 3.95 | 97.9 | 116.9 | 97.2 |
| v37644 | Ambiguous | 19.1 | Ambiguous | 14.3 |
| v37654 | Ambiguous | 11.4 | Ambiguous | 6.8 |
| v37663 | 2.47 | 98.6 | 33.17 | 98.6 |
| v37663 + v37665 | 13.89 | 99.2 | 19.44 | 99.8 |
| v37665 | Ambiguous | 18.2 | Ambiguous | 4.2 |
| v22277 | Ambiguous | 20.4 | Ambiguous | 5.4 |

TABLE 39
Potency and Maximum Mean Cytotoxicity (MMC) Induced by
Anti-CLDN18.2 Trivalent and Trispecific Antibody Constructs
or a Combination of Anti-(CLDN18.2 × CD3) and anti-
(CLDN18.2 × CD28) Bispecific Constructs from Primary
Human T cells co-cultured with KATO III-RFP Target Cells

| | 2:1 E:T | | 1:5 E:T | |
|---|---|---|---|---|
| Construct # | IC₅₀ (pM) | MMC (%) | IC₅₀ (pM) | MMC (%) |
| v37634 | 4.112 | 89.9 | 41.17 | 89.7 |
| v39215 | 50.65 | 84.7 | 1330 | 67.4 |
| v37644 | Ambiguous | 14.5 | Ambiguous | 9.5 |
| v37654 | Ambiguous | 26.7 | Ambiguous | 8.9 |
| v37663 | 21.38 | 81.1 | 722.3 | 59.5 |
| v37663 + v37665 | 19.97 | 87.0 | 36.94 | 88.5 |
| v37665 | Ambiguous | 9.1 | Ambiguous | −0.4 |
| v22277 | Ambiguous | 16.0 | Ambiguous | 23.3 |

Cytotoxic potency and maximum cytotoxicity values from T cells in co-culture with CLDN18.2-expressing SNU 601- and KATO III-RFP cells following treatment with a trivalent anti-(CLDN18.2×CD3×CD28) trispecific construct, combination therapy of anti-(CLDN18.2×CD3) and anti-(CLDN18.2×CD28) bispecific constructs, and bispecific and negative control constructs are shown in TABLE 38, and from T cells co-cultured with KATO III-RFP cells are shown in TABLE 39. Potency and maximum cytokine concentrations (as well as standard deviation, “SD”) of IFNγ, IL-2, TNFα, and IL-6 production by primary human T cells in co-culture with CLDN18.2-expressing SNU 601 cells following treatment with trivalent anti-(CLDN18.2×CD3×CD28) trispecific construct, combination therapy of anti-(CLDN18.2×CD3) and anti-(CLDN18.2×CD28) bispecific constructs, and bispecific and negative control constructs are shown in TABLE 40. Data from T cells in co-culture with SNU 601-RFP cells are representative of two independent donors from three independent experiments.

The trivalent and trispecific anti-(CLDN18.2×CD3×CD28) trispecific construct, v37634, exhibited the highest cytotoxic potency in high E:T T cell co-cultures with SNU 601- and KATO III-RFP cells, with IC₅₀ values of 1.01 and 4.112 pM, respectively, and reaching a maximum cytotoxicity of 98.3 and 89.9%, respectively. The bispecific anti-(CLDN18.2×CD3) and trispecific anti-(CLDN18.2×CD3×RSV-F) constructs, v37663 and v39215, respectively, induced approximately 2.5- to 4-fold reduced potency in high E:T T cell co-cultures with SNU 601-RFP cells compared to v37634, with IC₅₀ values of 2.47 and 3.95 pM, respectively, and reaching a maximum cytotoxicity of 98.6% and 97.9%, respectively. In high E:T KATO III-RFP co-cultures, v37663 and v39215 induced approximately 5- to 10-fold reduced potency compared to v37634, with IC₅₀ values of 21.38 and 50.65 pM, respectively and reaching 81.1% and 84.7% maximum cytotoxicity. Combination of anti-(CLDN18.2×CD3) and anti-(CLDN18.2×CD28) bispecific constructs, v37663+v37665, induced reduced cytotoxic potency in high E:T co-cultures with SNU 601- or KATO III-RFP cell lines compared to v37634, with IC₅₀ values of 13.89 and 19.97 pM, respectively, and reaching maximum cytotoxicity of 99.2% and 87.0%, respectively.

The construct v37634 alone and the combination v37663+v37665 induced similar cytotoxic potency in low E:T T cell co-cultures with SNU 601- and KATO III-RFP cells, with IC₅₀ values of 14.94 and 19.44 pM, respectively, in SNU 601-RFP co-cultures, and 41.17 and 36.94 pM, respectively, in KATO III-RFP co-cultures. Maximum cytotoxicity induced by v37634 and v37663+v37665 in SNU 601-RFP co-cultures reached 99.7% and 99.8%, respectively, and in KATO III-RFP co-cultures, reached 89.7% and 88.5%, respectively. The bispecific anti-(CLDN18.2×CD3) and trispecific format control anti-(CLDN18.2×CD3×RSV-F) constructs, v37663 and v39215, induced reduced potency in low E:T T cell co-cultures with SNU 601-RFP cells compared to v37634 and v37663+v37665, with IC₅₀ values of 33.17 and 116.9 pM, respectively, and reaching a maximum cytotoxicity of 98.6% and 97.2%, respectively. In low E:T KATO III-RFP co-cultures, v37663 and v39215 induced reduced potency compared to v37634, with IC₅₀ values of 722.3 and 1330 pM, respectively and reaching 59.5% and 67.4% maximum cytotoxicity.

Control constructs anti-(CLDN18.2×RSV-F×CD28), v37654, and anti-(RSV-F×CD3×CD28), v37644, and bispecific anti-(CLDN18.2×CD28), v37665, as well as the negative control construct, v22277, did not induce cytotoxicity against SNU 601-RFP or KATO III-RFP cell lines at either high or low E:T co-cultures.

TABLE 40
Maximum IFNγ, IL-2, TNFα, and IL-6 Production Induced by Anti-
CLDN18.2 Trivalent and Trispecific Antibody Constructs, Bispecific
Constructs alone, or a Combination of Anti-(CLDN18.2 × CD3)
and anti-(CLDN18.2 × CD28) Bispecific Constructs, from Primary
Human T cells co-cultured with SNU 601-RFP Target Cells

| | Maximum Mean Cytokine Concentration (pg/mL) | | | | | | | |
|---|---|---|---|---|---|---|---|---|
| Construct # | IFNγ | SD | IL-2 | SD | TNFα | SD | IL-6 | SD |
| v37634 | 33195.9 | 3441.1 | 3642.3 | 411.1 | 367.8 | 92.3 | 16.1 | 4.6 |
| v39215 | 14430.4 | 2136.3 | 113.5 | 20.2 | 87.1 | 13.0 | 10.6 | 0.8 |
| v37644 | 58.6 | 18.7 | 12.2 | 5.8 | 2.6 | 1.0 | 1.6 | 0.7 |
| v37654 | 5.3 | 1.3 | 1.1 | 0.4 | 1.2 | 0.7 | 3.9 | 5.6 |
| v37663 | 13624.5 | 1932.7 | 108.0 | 8.9 | 108.0 | 14.4 | 9.3 | 3.2 |
| v37663 + v37665 | 65422.1 | 10791.5 | 9116.4 | 1095.8 | 1037.3 | 253.7 | 19.7 | 7.5 |
| v37665 | 4.1 | 0.2 | 3.2 | 2.3 | 1.5 | 0.3 | 2.4 | 3.3 |
| v22277 | 8.6 | 7.4 | 1.8 | 1.1 | 1.2 | 0.7 | 2.0 | 0.8 |

The trivalent and trispecific anti-(CLDN18.2×CD3×CD28) antibody construct, v37634, induced IFNγ production that reached a maximum of 33195.9 pg/mL. IFNγ production induced by v39215 and v37663 was approximately 2-fold reduced compared to v37634, reaching a maximum of 14430.4 and 13624.5 pg/mL, respectively. The combination of bispecific anti-(CLDN18.2×CD3) and anti-(CLDN18.2×CD28) bispecific constructs, v37663+v37665, induced the highest level of IFNγ production, reaching a maximum of 65422.1 pg/mL, approximately 2-fold higher than that induced by v37634. v37634 induced IL-2 production that reached a maximum of 3642.3 pg/mL. IL-2 production induced by v39215 and v37663 was approximately 30-fold reduced compared to v37634, reaching a maximum of 113.5 and 108.0 pg/mL, respectively. The combination, v37663+v37665, induced the highest level of IL-2 production, reaching a maximum of 9116.4 pg/mL, approximately 2.5-fold higher than that induced by v37634. v37634 induced TNFα production that reached a maximum of 367.8 pg/mL. TNFα production induced by v39215 and v37663 was approximately 3.5- to 4-fold reduced compared to v37634, reaching a maximum of 87.1 and 108.0 pg/mL, respectively. The combination, v37663+v37665, induced the highest level of TNFα production, reaching a maximum of 1037.3 pg/mL, approximately 3-fold higher than that induced by v37634. v37634 and the combination, v37663+v37665, induced similar levels of IL-6 production that reached a maximum of 16.1 and 19.7 pg/mL, respectively. IL-6 production induced by v39215 and v37663 was approximately 1.5- to 2-fold reduced compared to v37634 and v37663+v37665, reaching a maximum of 10.6 and 9.3 pg/mL, respectively.

Control anti-(CLDN18.2×RSV-F×CD28) construct, v37654, and anti-(RSV-F×CD3×CD28) construct, v37644, the bispecific anti-(CLDN18.2×CD28) construct, v37665, as well as the negative control construct, v22277, did not induce a measurable production of IFNγ, IL-2, TNFα, or IL-6 following co-culture of human Pan T cells with SNU 601-RFP cell lines.

These results show that the trivalent and trispecific anti-(CLDN18.2×CD3×CD28) antibody construct, v37634, can induce non-inferior or similar cytotoxicity of target tumor cells but with a reduced induction of cytokine production by T cells when compared to a combination treatment of anti-(CLDN18.2×CD3) and anti-(CLDN18.2×CD28) bispecific constructs.

### Example 32: Impact of Anti-CD28 Paratope Affinity on Constructs' Ability to Induce Pro-Inflammatory Cytokine Release by Human CD3+ T Cells in Co-Culture with CLDN18.2+ Target Cells

To assess the effect of reducing the affinity of the trispecific antibody construct to CD28 on its ability to induce cytokine production, selected constructs, including v37642, were assessed for IL-2 and IFNγ production by primary human CD3+ T cells in co-culture with CLDN18.2+ SNU 601 target cells. Four additional molecules with the same format and geometry as v37642, but with reduced affinity to CD28, v38990, v38991, v38992, and v38993, were included, as was a trivalent anti-(CLDN18.2×CD3×RSV-F) control construct, v37662, a CLDN18.2×CD3 bispecific control construct, v35923, and a negative control construct, v22277.

Test articles (e.g., antibody constructs, controls, etc.) were titrated in RPMI1640 (Gibco)+10% FBS (ThermoFisher) and added to appropriate wells of the 384-well black flat bottom assay plates (ThermoFisher, Watham, MA). Human CD3+ T cells were thawed and resuspended as monocultures of CD3+ T cells or mixed with SNU 601 (high CLDN18.2 density) cells at an E:T ratio of 2:1 then added to the plates. Plates were incubated for 72 hrs at 37° C. with 5% carbon dioxide. Post incubation, 30 μL/well of supernatant was transferred to non-binding 384-well plates (Greiner-Bio-One, Kremsmünster, Austria) and stored at −20° C.

IFNγ and IL-2 were quantified using MSD (Mesoscale Discovery, Piscataway, NJ). The night before cytokine quantification, MSD plates were blocked and coated in capture constructs according to the manufacturers' instructions. The following day, plates were washed in PBS-T and 5 μl of assay diluent was added to each plate. The supplied and IL-2 standard (calibrator 1) and IFNγ standard (R&D Biosystems) was titrated as per manufacturer's instructions. Supernatants were thawed at room temperature and 5 μL of samples or standards were transferred to MSD plates. Detection antibody constructs were prepared at appropriate dilutions and 10 μL was added to each sample and standard well in MSD plates. The plates were sealed with aluminum foil and incubated away from light at room temperature for two hours. Plates were washed 3× in PBS-T and 40 μL MSD Gold read buffer T was added to each well. Plates were read on the MESO SECTOR 6000 and cytokine concentration was determined using MSD software. Data from a standard curve and samples were used to perform a nonlinear curve-fit with x-interpolation to obtain IFNγ and IL-2 concentrations in pg/mL.

Production of IFNγ and IL-2 by human CD3+ T cells in the presence of 45 nM of anti-(CLDN18.2×CD3×CD28) trispecific constructs and anti-(CLDN18.2×CD3) and anti-(CLDN18.2×CD28) bispecific controls are summarized in TABLE 41.

TABLE 41
Mean Cytokine Production Induced by Anti-CLDN18.2
Trivalent and Trispecific Antibody Constructs with
Reduced Affinity to CD28 from Primary Human T Cells
Co-cultured with SNU 601 Target Cells

| | T cells Alone | | | |
|---|---|---|---|---|
| | IL-2 | | IFNγ | |
| | Mean | | Mean | |
| | Cytokine | Standard | Cytokine | Standard |
| Construct # | (pg/mL) | Deviation | (pg/mL) | Deviation |
| v37642 | 2933.9 | 1010.9 | 857.4 | 493.0 |
| v37662 | 0.1 | 0.1 | 1.7 | 0.6 |
| v38990 | 1.8 | 2.2 | 0.9 | 0.1 |
| v38991 | 26.5 | 19.2 | 15.4 | 5.6 |
| v38992 | 1690.2 | 63.7 | 559.3 | 114.0 |
| v38993 | 1524.2 | 163.6 | 474.6 | 145.3 |
| v35923 | 1.3 | 0.2 | 1.0 | 0.5 |
| v22277 | 0.5 | 0.1 | 0.3 | 0.3 |

TABLE 42
Potency of Cytokine Production Induced by Anti-CLDN18.2
Trivalent and Trispecific Antibody Constructs with
Reduced Affinity to CD28 from Primary Human T Cells
Co-cultured with SNU 601 Target Cells

| | T cells + SNU 601 | | | |
|---|---|---|---|---|
| | IL-2 | | IFNγ | |
| | | Maximum | | Maximum |
| | EC₅₀ | Cytokine | EC₅₀ | Cytokine |
| Construct # | (pM) | (pg/mL) | (pM) | (pg/mL) |
| v37642 | 2.666 | 5065 | 3.669 | 3041 |
| v37662 | Ambiguous | 49.04 | 12.37 | 391.7 |
| v38990 | 1559 | 5244 | 603.7 | 3291 |
| v38991 | 22.24 | 9615 | 90.92 | 6146 |
| v38992 | 9.157 | 5556 | 23.02 | 3992 |
| v38993 | 28.69 | 8414 | 79.32 | 4449 |
| v35923 | 76.33 | 5643 | 26 | 1999 |
| v22277 | Ambiguous | 52.79 | Ambiguous | 51.57 |

Maximum IL-2 and IFNγ production from monocultures of T cells induced by the anti-(CLDN18.2×CD3×CD28) construct with the wild type anti-CD28 Fab, v37642, was 2933.9 and 857.4 pg/mL, respectively. IL-2 and IFNγ production induced by v38992 and 38993—with 3.1-fold and 3.4-fold reduction in CD28 affinity by SPR, respectively (see also, e.g., EXAMPLE 5)-induced approximately 1.5-fold to 2-fold reduced production if IL-2 and IFNγ compared to v37642, with maximum IL-2 production of 1690.2 and 1524.2 pg/mL, respectively, and maximum IFNγ production of 559.3 and 474.6 pg/mL, respectively. The trispecific construct, v38991—with 6.1-fold reduction in CD28 affinity by SPR-exhibited an approximate 100-fold reduction in IL-2 production, with maximum of 26.5 pg/mL induced, and an approximately 55-fold reduction in IFNγ production, with a maximum of 15.4 pg/mL induced. The trispecific construct, v38990, with a 225-fold reduction in CD28 by SPR, did not induce IL-2 or IFNγ production, relative to the negative control construct, v22277.

Maximum IL-2 and IFNγ produced by T cells in co-culture with SNU 601 cells induced by the trispecific constructs with affinity attenuated CD28, v38990, 38991, 38992, and 38993, remained within two-fold of that induced by the trispecific construct with a wild type anti-CD28 domain, v37642. Trispecific construct, v37642, exhibited the highest potency of IL-2 and IFNγ production by T cells co-cultured with SNU 601 target cells, with EC₅₀ value of 2.66 pM and 3.689 pM, respectively. The constructs v38992, 38993, and v38991—with 3.1-, and 3.4-, and 6.1-fold reduction in CD28 affinity by SPR, respectively (see also, e.g., EXAMPLE 5)—exhibited between 3-fold to 10-fold fold reduction in potency of IL-2 production compared to v37642, with EC₅₀ values of 9.175, 28.69, and 22.24 pM, respectively, and a 6-fold to 25-fold reduction in potency of IFNγ production, with EC₅₀ values of 23.02, 79.32, and 90.92 pM, respectively. The construct v38990—with 225-fold reduction in CD28 affinity by SPR—exhibited approximately a 586-fold reduction in potency of IL-2 production compared to v37642, with an EC₅₀ value of 1559 pM, and 165-fold reduction in potency of IFNγ production, with an EC₅₀ value of 603.7 pM.

Taken together, these results show that induction of nonspecific cytokine production can be modulated (e.g., reduced) by introducing specific amino acid substitutions in the anti-CD28 Fab domain, without significantly impacting the trispecific constructs' ability to induce cytokine production by T cells in co-culture with CLDN18.2+ SNU 601 cells. Furthermore, these results show that the trivalent and trispecific antibody constructs of the present disclosure can provide a strict tumor cell dependent cytotoxicity profile, with no significant activation of isolated T cells (i.e., no presence of tumor cells).

### Example 33: Impact of Format and Geometry of Immobilized Trivalent and Trispecific Antibody Constructs on Induction of Cytokine in an In Vitro Predictive Assay for Cytokine Release Syndrome

To interrogate the impact of the format and geometry of certain trivalent and trispecific antibody constructs on the induction of proinflammatory cytokine release in in vitro predictive assays of cytokine release syndrome, selected trivalent trispecific anti-(CLDN18.2×CD3×CD28) antibody constructs and control constructs were immobilized onto the surface of 96 well microtiter plates and tested in monocultures of primary human PBMCs. Monoclonal anti-CD3 (catalogue: MAB9929, R&D Systems) and anti-CD28, v36577, monoclonal antibodies, which have been shown to induce cytokine release syndrome in patients as well as a mitogen, PHA (Invitrogen), were included as positive controls. An anti-TNFα monoclonal antibody (catalogue: A2019, Universal Biologicals) was included as a negative control for cytokine release syndrome. Anti-RSV-F monoclonal antibodies with IgG4 (v36992) and IgG1 LALADS (v29982) backbones were included as negative isotype control constructs.

Antibody constructs were immobilized onto the surface of 96 well U bottom polypropylene plates by incubating 1 μg of antibody in 60 μL of PBS for 2 hours at room temperature. Plates were washed twice with 200 μL of PBS to remove any unbound antibody. Frozen human PBMCs were thawed at 37° C., washed in fresh cell culture media (RPMI+10% heat-inactivated human AB serum) and 200 μL of 200 000 cells were added to the wells of the plate containing the immobilized antibody constructs. PBMCs were incubated with immobilized antibody constructs for 48 hours, after which supernatant was harvested and analyzed for IFNγ, IL-2, and IL-6 by MSD (Mesoscale Discovery).

The night before cytokine quantification, MSD plates were blocked and coated in capture antibodies according to the manufacturer's instructions. The following day, plates were washed in PBS-T and 5 μl of assay diluent was added to each plate. The supplied IFNγ, IL-2, and IL-6 standard (calibrator 1) was titrated as per manufacturer's instructions. Supernatants were thawed at room temperature and 5 μL of samples or standards were transferred to MSD plates. Detection antibodies were prepared at appropriate dilutions and 10 μL was added to each sample and standard well in MSD plates. The plates were sealed with aluminum foil and incubated away from light at room temperature for two hours. Plates were washed 3× in PBS-T and 40 μL MSD Gold read buffer T was added to each well. Plates were read on the MESO SECTOR 6000 and cytokine concentration was determined using MSD software. Data from a standard curve and samples were used to perform a nonlinear curve-fit with x-interpolation to obtain IFNγ, IL-6, and IL-2 concentrations in pg/mL. Data are pooled from 2-5 independent experiments with 2-4 individual donors.

Production of IL-2, IFNγ and IL-6 by human PBMCs in the presence of the tested anti-(CLDN18.2×CD3×CD28) trivalent and trispecific antibody constructs and controls are summarized in TABLE 43.

TABLE 43
Mean Cytokine Production (and Standard Error of the Mean = SEM)
Induced by Immobilized Anti-(CLDN18.2 × CD3 × CD28) Trivalent
and Trispecific Antibody Constructs from Monocultures of Primary PBMCs

| | PBMCs | | | | | |
|---|---|---|---|---|---|---|
| | IL-2 | | IFNγ | | IL-6 | |
| | Mean | | Mean | | Mean | |
| Construct/ | Cytokine | | Cytokine | | Cytokine | |
| Catalogue # | (pg/mL) | SEM | (pg/mL) | SEM | (pg/mL) | SEM |
| v37634 | 86.6 | 14.9 | 3.39E+4 | 1.03E+4 | 245.0 | 58.4 |
| v39215 | 43.8 | 12.0 | 1.55E+4 | 3.92E+3 | 124.3 | 25.4 |
| v37654 | 7.1 | 1.2 | 380.4 | 147.4 | 14.6 | 4.8 |
| v37638 | 3.88E+4 | 2.83E+3 | 7.83E+5 | 3.45E+5 | 1302.1 | 819.5 |
| v39231 | 4.9 | 4.9 | 0.0 | 0.0 | 6.2 | 0.9 |
| v37658 | 75.5 | 9.8 | 1.95E+4 | 5.51E+3 | 212.7 | 66.3 |
| v37692 | 1.67E+4 | 4.01E+3 | 2.80E+5 | 4.71E+4 | 1107.0 | 246.8 |
| v39232 | 38.1 | 35.6 | 232.7 | 232.7 | 0.9 | 0.9 |
| v37642 | 5.25E+4 | 1.84E+4 | 5.51E+5 | 7.71E+4 | 941.5 | 454.0 |
| v37662 | 45.6 | 21.5 | 1.60E+4 | 8.39E+2 | 184.4 | 1.0 |
| v37665 | 21.2 | 14.3 | 625.7 | 625.0 | 156.9 | 139.8 |
| v37663 | 51.0 | 17.8 | 6.71E+4 | 4.28E+4 | 256.1 | 40.3 |
| v35923 | 41.6 | 12.8 | 1.59E+4 | 8.52E+3 | 121.8 | 25.0 |
| v36992 | 20.8 | 16.8 | 120.1 | 63.8 | 64.5 | 26.9 |
| v39982 | 14.7 | 6.3 | 175.9 | 142.9 | 231.0 | 217.2 |
| v36577 | 7.44E+3 | 1.49E+3 | 4.28E+4 | 7.80E+3 | 349.1 | 51.0 |
| A2019 | 10.6 | 3.5 | 0.5 | 0.5 | 207.9 | 170.0 |
| MAB9929 | 32.8 | 13.2 | 1.01E+4 | 7.79E+3 | 879.9 | 796.7 |
| PHA | 423.4 | 174.9 | 1.73E+5 | 4.69E+4 | 2.44E+4 | 8.73E+3 |

The trispecific anti-(CLDN18.2×CD3×CD28) antibody constructs, v37638, v37692, and v37642, induced higher levels of IL-2, IFNγ, and IL-6 than the positive control anti-CD28 construct, v36577. The trispecific anti-(CLDN18.2×CD3×CD28) construct, v37634, induced markedly lower IL-2 production compared to v36577, and an approximate 2- to 3-fold reduction in IL-6 production, relative to that induced by v36577 and MAB9929. IL-6 production by v37634 was similar to that induced by the negative CRS control, A2019.

Taken together, these data show that induction of cytokine in in vitro predictive assays of cytokine release syndrome can be modified by modulating the geometry and/or format of trispecific anti-(CLDN18.2×CD3×CD28) antibody constructs.

### Example 34: Impact of CD3 & CD28 Co-Engagement with Trivalent and Trispecific Antibody Constructs on T Cell Memory Induction Following Co-Culture of Human PBMCS with CLDN18.2-Expressing SNU 601 Target Cells

To interrogate the difference in cytotoxicity and induction of proinflammatory cytokine release induced by the trivalent and trispecific anti-(CLDN18.2×CD3×CD28) constructs, v37634, v37638, and v37642, in comparison to bispecific anti-(CLDN18.2×CD3) bispecific antibodies, v35923 and v37663, and a combination of bispecific anti-(CLDN18.2×CD3) and anti-(CLDN18.2×CD28) bispecific antibodies, v37663+v37665, selected constructs were tested in primary human PBMC cultures with SNU 601, an allogeneic tumor cell line expressing CLDN18.2. Trispecific control constructs containing a paratope against an irrelevant antigen, RSV protein F (RSV-F), in place of the CD3 or CD28 paratopes, v37654 and v39215, respectively, were included as format controls for each binding arm of the trispecific antibody construct, v37634. Trispecific control constructs containing a paratope against an irrelevant antigen, RSV protein F (RSV-F), in place of the CD28 paratope, v37658 and v37662, respectively, were included as format controls for each binding arm of the trispecific antibody constructs, v37638 and v37642, respectively. A monoclonal anti-RSV protein F antibody, v39982, was included as a negative control.

Antibody constructs were diluted to 5 nM in RPMI1640 (Gibco)+10% FBS (ThermoFisher Scientific)+1% Pen/Strep (Gibco) and plated in respective wells on a 96-well flat-bottom assay plate (ThermoFisher Scientific). Frozen human PBMCs were thawed at 37° C. and mixed with CLDN18.2-expressing SNU 601 cells such that the ratio of T cells to allogeneic tumor cells was adjusted to an effector-to-target (E:T) ratio of 1:1. The mixtures were incubated together with the antibody constructs for 5 days or 7 days, after which T cell memory subset analysis was performed by flow cytometry.

Cells were transferred to 96-well V-bottom plates (Sarstedt) and washed twice with cold FACS buffer (PBS+2% FBS+2.5 mM EDTA). Cells were stained with a cocktail of Zombie Violet Dye, anti-CD45, anti-CD4, and anti-CD8, anti-CCR7, and anti-CD45RO antibodies for 60 minutes and washed twice with FACS buffer. Following staining, events were captured on the LSRFortessa X-20 (BD Biosciences).

Data were analyzed using FlowJo software (BD Biosciences). Cells were gated by size and granularity using FSC-A and SSC-A gating. Singlet cell events were then selected by gating on SSC-A and SSC-H. Live cells were selected by gating on events that were negative for viability dye. Tumor cells were excluded from analysis by gating on events that stained positive for CD45. Cells were further separated into CD4+ and CD8+ T cells. CD4+ and CD8+ T cell memory subsets were determined as follows: Naïve T cells (T_{Naïve;} CCR7+CD45RO−), Central Memory T cells (T_{CM}; CCR7+CD45RO+), Effector Memory T cells (T_{EM}; CCR7−CD45RO+), and double negative T cells (T_{DN}; CCR7−CD45RO−).

Summary of memory subsets induced by tested trivalent and trispecific anti-(CLDN18.2×CD3×CD28) constructs, and anti-(CLDN18.2×CD3), and anti-(CLDN18.2×CD28) bispecific constructs are shown in TABLE 48.

TABLE 48
Memory T Cell Subset Induction

| | DN (CCR7− | | T_{Naïve} (CCR7 + | | T_{CM} (CCR7 + | | T_{EM} (CCR7− | |
|---|---|---|---|---|---|---|---|---|
| | CD45RO−) | | CD45RO−) | | CD45RO+) | | CD45RO+) | |
| Construct | Day 4 | Day 7 | Day 4 | Day 7 | Day 4 | Day 7 | Day 4 | Day 7 |

Mean CD4+ T cell Memory Subset

| | | | | | | | | |
|---|---|---|---|---|---|---|---|---|
| v35923 | 0.2 | 0.0 | 0.0 | 0.2 | 27.4 | 62.0 | 72.4 | 37.7 |
| v37663 | 0.0 | 0.0 | 0.0 | 0.1 | 27.1 | 65.6 | 72.9 | 34.4 |
| v37663 + v37665 | 0.2 | 0.3 | 0.1 | 0.4 | 35.7 | 46.2 | 64.0 | 53.1 |
| v37634 | 0.5 | 0.6 | 0.2 | 0.8 | 35.2 | 52.1 | 64.2 | 46.7 |
| v39215 | 0.0 | 0.0 | 0.0 | 0.2 | 29.5 | 65.6 | 70.4 | 34.3 |
| v37654 | 3.9 | 5.2 | 55.6 | 51.0 | 15.6 | 13.6 | 24.9 | 30.3 |
| v37638 | 0.5 | 0.2 | 0.2 | 0.6 | 29.9 | 73.4 | 69.5 | 25.9 |
| v37662 | 1.0 | 0.3 | 0.2 | 0.8 | 28.6 | 70.8 | 70.3 | 28.1 |
| v37665 | 4.4 | 4.5 | 53.8 | 49.5 | 15.0 | 15.7 | 26.9 | 30.3 |
| v39982 | 3.5 | 5.0 | 56.7 | 55.7 | 15.8 | 12.8 | 24.1 | 26.6 |
| No Treatment | 4.8 | 5.2 | 54.9 | 47.4 | 13.3 | 11.8 | 27.1 | 35.7 |

Mean CD8+ T cell Memory Subset

| | | | | | | | | |
|---|---|---|---|---|---|---|---|---|
| v35923 | 0.3 | 0.4 | 0.0 | 0.4 | 8.6 | 22.6 | 91.1 | 76.6 |
| v37663 | 0.1 | 0.2 | 0.0 | 0.1 | 7.7 | 18.2 | 92.1 | 81.5 |
| v37663 + v37665 | 0.4 | 0.6 | 0.1 | 0.2 | 11.9 | 15.2 | 87.7 | 84.1 |
| v37634 | 0.9 | 1.1 | 0.2 | 0.4 | 9.9 | 11.8 | 89.0 | 86.7 |
| v39215 | 0.2 | 0.2 | 0.0 | 0.2 | 9.2 | 18.0 | 90.6 | 81.7 |
| v37654 | 16.8 | 15.2 | 36.7 | 29.8 | 4.6 | 3.0 | 41.9 | 52.1 |
| v37638 | 0.7 | 0.6 | 0.1 | 0.3 | 8.3 | 19.1 | 90.9 | 80.1 |
| v37662 | 1.1 | 0.8 | 0.2 | 0.4 | 6.8 | 18.6 | 91.9 | 80.2 |
| v37665 | 16.9 | 15.6 | 33.6 | 31.8 | 3.5 | 2.8 | 46.1 | 49.9 |
| v39982 | 15.7 | 15.3 | 36.8 | 34.4 | 4.9 | 2.7 | 42.7 | 47.7 |
| No Treatment | 18.4 | 14.2 | 34.6 | 31.8 | 3.6 | 2.6 | 43.4 | 51.5 |

The trispecific anti-(CLDN18.2×CD3×CD28) constructs, v37638 and v37692, the bispecific anti-(CLDN18.2×CD3) constructs, v35923 and v37663, as well as trispecific anti-(CLDN18.2×CD3×RSV-F) construct, v39215, induced similar levels of T_{CM} cells, ranging from 27.1% to 29.9% and 62.0% to 73.4% of CD4+ cells after 5 days and 7 days of stimulation, respectively, and T_{CM} of CD8+ T cells ranged from 6.8% to 9.2% and 18.0% to 22.6% of CD8+ cells after 5 days and 7 days of stimulation, respectively. T_{EM} cell induction ranged from 69.5% to 72.9% and 25.9% to 37.7% of CD4+ cells after 5 days and 7 days of stimulation, respectively. T_{EM} of CD8+ T cells ranged from 90.6% to 92.1% and 80.1% to 81.7% of CD8+ cells after 5 days and 7 days of stimulation, respectively.

The trispecific anti-(CLDN18.2×CD3×CD28) construct, v37634, and the combination of the bispecific anti-(CDLN18.2×CD3) and anti-(CLDN18.2×CD28) constructs, v37663+v37665, similarly induced the highest percentage of T_{CM} (CCR7+CD45RO+) cells by day 5 post stimulation, with 35.2% and 35.7% of the total CD4+ population, respectively, and 9.9% and 11.9% of the CD8+ population, respectively. T_{EM} (CCR7−CD45RO+) induction by day 5 post stimulation by v37634 and v37663+v37665 was 64.2% and 64.0% of the total CD4+ population, respectively, and 89% and 87.7% of the CD8+ population, respectively. Compared to memory subset induction by anti-(CLDN18.2×CD3) bispecifics, v35923 and v37663, as well as the anti-(CLDN18.2×CD3×RSV-F) trispecific construct, v39215, v37634 as well as the combination of bispecific anti-CDLN18.2×CD3 and anti-(CLDN18.2×CD28) constructs, v37663+v37665, similarly induced the lowest percentage of T_{CM} (CCR7+CD45RO+) cells by day 7 post stimulation with 52.1% and 46.2% of the total CD4+ population, respectively, and 11.8% and 15.2% of the CD8+ population, respectively. T_{EM} (CCR7-CD45RO+) induction by day 7 post stimulation by v37634 and v37663+v37665 was 46.7% and 53.1% of the total CD4+ population, respectively, and 86.7% and 84.1% of the CD8+ population, respectively.

The trispecific anti-(CLDN18.2×RSV-F×CD28) format control, v37654, and the bispecific anti-(CLDN18.2×CD28) antibody, v37665, did not induce differentiation of CD4+ or CD8+ T cell memory phenotypes compared to the negative control, v39982.

Taken together, these data show that memory T cell subset induction by anti-(CLDN18.2×CD3×CD28) constructs can be modulated by using a trispecific format and/or geometry. Furthermore, the anti-(CLDN18.2×CD3×CD28) trispecific antibody construct, v37634, induced similar memory subsets compared to those induced by a combination of anti-(CLDN18.2×CD3) and anti-(CLDN18.2×CD28) bispecific antibodies, v37663+v37665, i.e., a single trivalent and trispecific antibody construct induced similar memory subsets compared to a combination of respective bispecific antibodies.

### Example 35: Impact of Format and Geometry of Trispecific Anti-(CLDN18.2×CD3×CD28) Antibody Constructs on Viability of Primary CD3+ T Cells

To determine the impact of the format and/or geometry of certain trivalent and trispecific anti-(CLDN18.2×CD3×CD28) antibody constructs described herein on the viability of human CD3+ T cells, selected antibody constructs were assessed in monocultures of primary human CD3+ T cells. Selected trispecific control constructs containing a paratope against an irrelevant antigen, RSV protein F (RSV-F), in place of the CD3, CD28, or CLDN18.2 paratopes, respectively, were included as format controls for each binding arm of the trispecific constructs, v37634, v37638, v37692 and v37642. Bispecific anti-(CLDN18.2×CD3) constructs, v37663 and v35923, as well as bispecific anti-(CLDN18.2×CD28) construct, v37665, were included as controls. Puromycin (Life Technologies) was included as a positive control for T cell death.

Constructs were diluted to 50 nM in RPMI1640 (Gibco)+10% FBS (ThermoFisher Scientific)+1% Pen/Strep (Gibco) and plated in respective wells on a 384-well flat, optical-bottom assay plate (ThermoFisher Scientific). Frozen human CD3+ T cells were thawed at 37° C. and combined with CellTox™ Green dye (Promega). CellTox™ Green measured changes in membrane integrity as a result of cell death—as cells lost viability, cells fluoresced green. CD3+ T cells with CellTox™ Green dye were added to diluted constructs and incubated at 37° C. with 5% CO₂ for 48 hours. After 48 hours, fluorescence was detected using the Operetta® CLS™ (PerkinElmer) and data were analyzed using the Operetta CLS High Content Analysis System (PerkinElmer).

MFI of fluorescence following treatment of primary CD3+ T cells with trispecific anti-(CLDN18.2×CD3×CD28) antibody constructs is summarized in TABLE 44 below.

TABLE 44
Impact of the Format and/or Geometry of Certain
Antibody Constructs on T Cell Viability

MFI, CellTox ™ Green

| Construct # | Mean | Standard Deviation |
|---|---|---|
| v37634 | 121.5 | 19.6 |
| v39215 | 142.3 | 6.6 |
| v37644 | 133.6 | 7.2 |
| v37654 | 141.5 | 1.8 |
| v37638 | 309.5 | 10.3 |
| v37658 | 133.0 | 5.0 |
| v39231 | 152.7 | 14.8 |
| v37692 | 234.8 | 2.5 |
| v39232 | 131.2 | 13.1 |
| v37642 | 363.9 | 23.7 |
| v37662 | 168.9 | 15.4 |
| v39250 | 158.5 | 2.6 |
| v39239 | 178.5 | 21.1 |
| v37665 | 159.9 | 3.8 |
| v37663 | 165.7 | 12.6 |
| v35923 | 141.6 | 7.3 |
| v22277 | 163.1 | 4.9 |
| Puromycin | 1045.2 | 114.0 |

CD3+ T cells following incubation with the trispecific anti-(CLDN18.2×CD3×CD28) antibody constructs, v37638, v37692, and v37642 displayed a decrease in cell viability compared to v22277, with an increase in MFI of CellTox™ Green from 163.1 to 309.5, 234.8, and 363.9, respectively. CD3+ T cells following incubation with the trispecific anti-(CLDN18.2×CD3×CD28) antibody construct, v37634, did not exhibit loss of viability relative to cells treated with the negative control, v22277. Treatment with control trispecific anti-(CLDN18.2×CD3×RSV-F) constructs, v39215, v37658, v37662, and v39250, trispecific anti-(CLDN18.2×RSV-F×CD28) constructs, 37654, v39231, v39232, and v39250, trispecific anti-(RSV-F×CD3×CD28) construct, v37644, bispecific anti-(CLDN18.2×CD3) constructs, v37665 and v35923, and anti-(CLDN18.2×CD28) construct, v37665, did not result in a decrease in T cell viability relative to the negative control, v22277. Treatment with puromycin resulted in the greatest decrease in T cell death relative to the negative control, v22277, with an MFI of 1045.2.

These results demonstrate that the format and geometry of a trivalent and trispecific anti-(CLDN18.2×CD3×CD28) antibody constructs can impact the construct's ability to negatively affect the viability of monocultures of human primary CD3+ T cells. Specifically, the antibody construct v37634 showed no increased (and even decreased) T cell cytotoxicity compared to a negative control construct, the anti-RSV protein F construct v22277. The trivalent and trispecific constructs v37638 and v37642, however—which contain the same antigen binding domains but in a different spatial organization compared to v27642—did show a significant increase in T cell cytotoxicity, demonstrating the fact that a construct's format and geometry alone can significantly impact its in vitro and/or in vivo properties.

### Example 36: Impact of Anti-CD28 Paratope Affinity on In Vitro Activity of Trivalent and Trispecific Antibody Constructs in T Cell Dependent Cytotoxicity Assay with CLDN18.2+ SNU-601 Tumor Cells

To assess the impact of reducing the affinity of certain anti-(CLDN18.2×CD3×CD28) trispecific antibody constructs toward CD28, selected constructs with the same format as v37642 but with reduced binding affinity towards CD28 were tested in T cell-dependent cytotoxicity assays with CLDN18.2+ tumor cell line SNU-601. Additionally, anti-(CLDN18.2×CD3) and anti-(CLDN18.2×CD28) bispecific control constructs, v37663 and v37665, respectively, were included. A trispecific construct, v37662, containing a paratope against an irrelevant antigen, RSV protein F (RSV-F), in place of the anti-CD28 paratope in the same format as v37642, was included as a format control. A monoclonal anti-RSV protein F antibody, v22277, was included as a negative control.

Test articles (e.g., antibody constructs, controls, etc.) were diluted in RPMI1640 (Gibco)+10% FBS (ThermoFisher) and added to appropriate wells of the 384-well black flat bottom assay plates (ThermoFisher, Watham, MA). Human panT cells were thawed and mixed with SNU 601-RFP (high CLDN18.2 density) cells at E:T ratios of 2:1 and 1:5 and plates were incubated for 72 hrs and 168 hrs, respectively, at 37° C. and 5% carbon dioxide, after which tumor cell viability was assessed through live tumor cell counts (RFP+) using the Operetta CLS High Content Analysis System (Perkin Elmer; HH16000020). Data are representative of 3 independent experiments with 2 donors.

Calculated IC₅₀ values and maximum mean cytotoxicity from the T cell-dependent cytotoxicity assay are presented in TABLE 45.

Percent cytotoxicity was calculated using the following formula:

$$\% cytotoxicity = ( 1 - \frac{n umber of tumor cells left in sample}{total number of tumor cells in untreated condition} ) \times 100$$

TABLE 45
Cytotoxic Activity of Trispecific Constructs with
Reduced Affinity for CD28 Towards CDLN18.2 +
Tumor Cells Grown in Culture with Primary Human T Cells

| | 2:1 E:T | | 1:5 E:T | |
|---|---|---|---|---|
| | | Maximum Mean | | Maximum Mean |
| Construct | IC₅₀ | Cytotoxicity | IC₅₀ | Cytotoxicity |
| # | (pM) | (%) | (pM) | (%) |
| v37642 | 0.166 | 98.74 | 2.295 | 96.94 |
| v37662 | 17.92 | 96.92 | Am- | 4.01 |
| | | | biguous | |
| v38991 | 0.8377 | 98.24 | 12.71 | 95.57 |
| v38990 | 3.579 | 97.79 | 145.3 | 95.4 |
| v38994 | 0.1584 | 98.03 | 2.42 | 94.09 |
| v37663 | 13.23 | 97.69 | 206.7 | 92.95 |
| v37665 | Am- | −9.28 | Am- | −4.68 |
| | biguous | | biguous | |
| v22277 | Am- | 8.47 | Am- | 0.72 |
| | biguous | | biguous | |

All tested trivalent and trispecific constructs that have mutations in the anti-CD28 Fab domain (mutations relative to the hTN228 wildtype V_{H} and/or V_{L} sequences) induced similar maximum cytotoxicity when compared to both (i) the trispecific constructs that comprise the wildtype TN228 paratope, e.g., construct v37642, and (ii) the bivalent and bispecific control construct, v37663, with maximum cytotoxicity reaching between 93-99% for both of the E:T ratios tested. The trivalent and trispecific construct v37642 exhibited the highest potency in a CLDN18.2+ SNU-601 co-culture at both 2:1 and 1:5 E:T ratios, with IC₅₀ values of 0.166 pM and 2.295 pM, respectively. At 2:1 E:T ratio, the trivalent and trispecific constructs with mutations in the anti-CD28 paratope as well as the CD28 KO anti-(CD3×RSV-F×CLDN18.2) control construct exhibited similar maximum cytotoxicity relative to the trispecific construct with the wild-type anti-CD28, v37642. At 1:5 E:T ratio, while the trivalent and trispecific constructs bearing mutations in the anti-CD28 paratope, v38990, v38991 and v38994, showed similar maximum cytotoxicity, the CD28 KO control, v37662, displayed a 24-fold reduction in cytotoxicity. The trivalent and trispecific construct, v38990, having a 225-fold reduction in binding affinity compared to anti-CD28 WT, as determined by SPR, showed a 22-fold and 60-fold reduced cytotoxic potency for the 2:1 and 1:5 E:T ratios, respectively, as compared to v37642 (containing the WT (unmodified) anti-CD28 domain). The construct v38991 which exhibits a 6.1-fold reduced binding affinity to CD28 as shown by SPR, showed a 5-fold reduction in cytotoxic potency towards CLDN18.2+ SNU-601 cells, relative to the wild-type trivalent and trispecific construct, v37642, for both E:T ratios. The trivalent and trispecific construct, v38994, displayed comparable potency to the wild-type trivalent and trispecific construct v37642 at both E:T ratios. The negative control, v22277, did not induce cytotoxicity of SNU 601 cells.

Taken together, these results demonstrate that mutations in the anti-CD28 paratope that reduce the binding affinity of the trivalent and trispecific antibody constructs can also impact the cytotoxic potency of these constructs in a co-culture of CLDN18.2+ SNU-601 target cells and human CD3+ T cells. This reduction in cytotoxic potency became more distinct in the lower E:T condition experiments of 1:5 ratio for, e.g., v38990 (225-fold binding affinity reduction determined by SPR) but was maintained for v38991 (6-fold binding affinity reduction determined by SPR). All of the trispecific constructs tested that contained mutations in the CD28 paratope exhibited higher cytotoxic potency than the corresponding bispecific controls (anti-(CD3×CLDN18.2) and anti-(CD28×CLDN18.2)).

### Example 37: Impact of the Format and Geometry of Trispecific Anti-(CLDN18.2×CD3×CD28) Constructs on Target-Independent Cytokine Production by Primary Human Immune Cells

To assess the impact of the format and geometry of certain trivalent and trispecific anti-(CLDN18.2×CD3×CD28) constructs on target-independent activation of primary human immune cells, selected constructs were assessed for their ability to stimulate cytokine production following incubation with human PBMCs and CD3+ T cells.

Test articles (e.g., antibody constructs, controls, etc.) were diluted to 45 nM, titrated 1:9 in RPMI1640 (Gibco)+10% FBS (ThermoFisher), and added to appropriate wells of the 384-well black flat bottom assay plates (ThermoFisher, Watham, MA). Human PBMCs or CD3+ T cells were thawed and resuspended as monocultures of cells and added to the plates. Plates were incubated for 72 hrs at 37° C. with 5% carbon dioxide. Post incubation, 30 μL/well of supernatant was transferred to non-binding 384-well plates (Greiner-Bio-One, Kremsmünster, Austria) and stored at −20° C.

IFNγ, TNFα, and IL-2 were quantified using MSD (Mesoscale Discovery, Piscataway, NJ). The night before cytokine quantification, MSD plates were blocked and coated in capture antibodies according to the manufacturers' instructions. The following day, plates were washed in PBS-T and 5 μl of assay diluent was added to each plate. The supplied and IL-2 standard (calibrator 1) and IFNγ standard (R&D Biosystems) was titrated as per manufacturer's instructions. Supernatants were thawed at room temperature and 5 μL of samples or standards were transferred to MSD plates. Detection antibody constructs were prepared at appropriate dilutions and 10 μL was added to each sample and standard well in MSD plates. The plates were sealed with aluminum foil and incubated away from light at room temperature for two hours. Plates were washed 3× in PBS-T and 40 μL MSD Gold read buffer T was added to each well. Plates were read on the MESO SECTOR 6000 and cytokine concentration was determined using MSD software. Data from a standard curve and samples were used to perform a nonlinear curve-fit with x-interpolation to obtain IFNγ, TNFα, and IL-2 concentrations in pg/mL.

Maximum cytokine production of IFNγ, TNFα and IL-2 by human CD3+ T cells in the presence of anti-(CLDN18.2×CD3×CD28) trispecific constructs and anti-(CLDN18.2×CD3) and anti-(CLDN18.2×CD28) bispecific control constructs are summarized in TABLE 46. Maximum cytokine production of IFNγ, TNFα and IL-2 by human PBMCs in the presence of anti-(CLDN18.2×CD3×CD28) trispecific constructs and anti-(CLDN18.2×CD3) and anti-(CLDN18.2×CD28) bispecific control constructs are summarized in TABLE 47.

TABLE 46
Mean Maximum Cytokine Production Induced by Anti-CLDN18.2 Trispecific
Constructs from Monocultures of Primary Human CD3+ T Cells

| | CD3+ T Cells | | | | | |
|---|---|---|---|---|---|---|
| | IL-2 | | IFNγ | | TNFα | |
| | Mean | | Mean | | Mean | |
| | Cytokine | Standard | Cytokine | Standard | Cytokine | Standard |
| Construct # | (pg/mL) | Deviation | (pg/mL) | Deviation | (pg/mL) | Deviation |
| v37633 | 2141.1 | 1514.3 | 430.0 | 316.7 | 149.5 | 100.4 |
| v37634 | 4.2 | 4.0 | 0.5 | 0.5 | 2.4 | 1.0 |
| v37635 | 1559.4 | 404.4 | 1079.0 | 392.6 | 340.3 | 98.4 |
| v37638 | 936.0 | 422.4 | 448.0 | 248.5 | 198.7 | 77.6 |
| v37640 | 2392.5 | 1214.7 | 808.8 | 500.6 | 280.9 | 122.4 |
| v37642 | 1458.2 | 935.9 | 1040.7 | 698.8 | 291.0 | 186.9 |
| v37663 | 0.8 | 0.2 | 9.9 | 9.0 | 0.7 | 0.4 |
| v37665 | 0.0 | 0.0 | 0.6 | 0.2 | 0.2 | 0.1 |
| v35923 | 0.2 | 0.3 | 0.2 | 0.6 | 0.2 | 0.2 |
| v22277 | 0.3 | 10.3 | 0.2 | 0.5 | 0.3 | 0.2 |

TABLE 47
Mean Maximum Cytokine Production Induced by Anti-CLDN18.2 Trispecific
Constructs from Monocultures of Primary Human PBMCs

| | PBMCs | | | | | |
|---|---|---|---|---|---|---|
| | IL-2 | | IFNγ | | TNFα | |
| | Mean | | Mean | | Mean | |
| | Cytokine | Standard | Cytokine | Standard | Cytokine | Standard |
| Construct # | (pg/mL) | Deviation | (pg/mL) | Deviation | (pg/mL) | Deviation |
| v37634 | 7.6 | 9.3 | 17.2 | 12.9 | 2.1 | 1.4 |
| v37638 | 116.6 | 96.0 | 823.0 | 713.6 | 23.4 | 20.8 |
| v37642 | 3545.3 | 369.3 | 20049.8 | 5800.2 | 866.8 | 30.9 |
| v37663 | 5.8 | 8.0 | 17.9 | 28.9 | 2.0 | 3.2 |
| v37665 | 2.5 | 0.9 | 3.5 | 2.5 | 0.2 | 0.2 |
| v35923 | 1.1 | 0.6 | 3.0 | 2.8 | 0.3 | 0.4 |
| v22277 | 1.8 | 0.9 | 2.0 | 1.7 | 0.4 | 0.4 |

The trivalent and trispecific anti-(CLDN18.2×CD3×CD28) constructs, v37633, v37635, v37638, v37640, and v37642, induced nonspecific cytokine production by monocultures of T cells, ranging from approximately 1000-2500 μg/mL of IL-2, 400-1100 μg/mL of IFNγ, and 150-350 pg/mL of TNFα. Surprisingly, the trivalent and trispecific anti-(CLDN18.2×CD3×CD28) construct, v37634, which has a different format than the other WT trispecific constructs v37638 and v37642, induced less than 10 μg/mL of IL-2, IFNγ, or TNFα; similar values for measured for the bispecific anti-(CLDN18.2×CD3) control constructs, v37663 and 35923, the bispecific anti-(CLDN18.2×CD28) control construct, v37665, and the negative control, v22277.

The trivalent and trispecific anti-(CLDN18.2×CD3×CD28) constructs, v37638 and v37642, induced nonspecific cytokine production by monocultures of PBMCs, ranging from approximately 100-3500 μg/mL of IL-2, 800-20000 μg/mL of IFNγ, and 23-900 μg/mL of TNFα. The trivalent and trispecific anti-(CLDN18.2×CD3×CD28) construct, v37634, which has a different format than the other WT trispecific constructs v37638 and v37642, induced less than 20 μg/mL of IL-2, IFNγ, or TNFα; similar values for measured for the bispecific anti-(CLDN18.2×CD3) control constructs, v37663 and 35923, the bispecific anti-(CLDN18.2×CD28) control construct, v37665, and the negative control, v22277.

Taken together, these results show that induction of nonspecific cytokine production can be modulated (e.g., reduced) through modification of an antibody construct's format and/or geometry.

The description, for purposes of explanation, has been described with reference to specific implementations. However, the illustrative discussions above are not intended to be exhaustive or to limit the implementations to the precise forms disclosed. Many modifications and variations are possible in view of the embodiments described herein. The implementations were chosen and described in order to best explain the principles and their practical applications, to thereby enable others skilled in the art to best utilize the implementations and various implementations with various modifications as are suited to the particular use contemplated.

All publications, patents, and patent applications mentioned in this specification are herein incorporated by reference in their entireties to the same extent as if each individual publication, patent, or patent application was specifically and individually indicated to be incorporated by reference. In the event of a conflict between a term herein and a term in an incorporated reference, the term herein controls.

### SEQUENCE TABLE

Selected embodiments and amino acid sequences of antibody constructs and portions or fragments thereof (e.g., chain portions, scFv, Fab or Fc domains, linker, etc.) that are disclosed and described herein are shown in the sequence table below. Unless otherwise defined herein, antibody construct polypeptide chains labeled with “H” refer to an Ig heavy chain, and polypeptide chains labeled with “L” refer to an Ig light chain.

| | | | | |
|---|---|---|---|---|
| Variant | | Type/ | SEQ | |
| # | Description | Target | ID NO | Sequence |

v34913-CD3xCD28xMSLN Trispecific Antibody Construct

| | | | | |
|---|---|---|---|---|
| 26149 | H | CD3/ | 100 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSYGVHWIRQPPGKGLEWLGVIWPGGGTNENS |
| | | | | ALMSRLTISEDTSKNQVSLKLSSVTAADTAVYYCA |
| | | | | RDRAYGNYLYAMDYWGQGTLVTVSSASTKGPSV |
| | | | | FPLAPSSKSTSGGTAALGCLVKDYFPEPVTVSWNS |
| | | | | GALTSGVHTFPAVLQSSGLYSLSSVVTVPSSSLGTQ |
| | | | | TYICNVNHKPSNTKVDKKVEPKSCDKTHTCPPCPA |
| | | | | PEAAGGPSVFLFPPKPKDTLMISRTPEVTCVVVSVS |
| | | | | HEDPEVKFNWYVDGVEVHNAKTKPREEQYNSTYR |
| | | | | VVSVLTVLHQDWLNGKEYKCKVSNKALPAPIEKTI |
| | | | | SKAKGQPREPQVYVYPPSRDELTKNQVSLTCLVKG |
| | | | | FYPSDIAVEWESNGQPENNYKTTPPVLDSDGSFAL |
| | | | | VSKLTVDKSRWQQGNVFSCSVMHEALHNHYTQK |
| | | | | SLSLSPG |
| | scFv | CD3 | 101 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VL |
| | V_{H} | CD3 | 102 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSS |
| | V_{L} | CD3 | 103 | NFMLTQPHSVSESPGKTVTISCKRNTGNIGSNYVN |
| | | | | WYQQHEGSSPTTIIYRNDKRPDGVSDRFSGSIDRSS |
| | | | | KSASLTISNLKTEDEADYFCQSYSSGFIFGGGTKLT |
| | | | | VL |
| | Linker^{scFv} | | 104 | GGGGSGGGGSGGGGSGGGGS |
| | Linker^{scFv-Fab} | | 105 | GGGGSG |
| | V_{H} | CD28 | 106 | QVQLQESGPGLVKPSETLSLTCAVSGFSLTSYGVH |
| | | | | WIRQPPGKGLEWLGVIWPGGGTNFNSALMSRLTIS |
| | | | | EDTSKNQVSLKLSSVTAADTAVYYCARDRAYGNY |
| | | | | LYAMDYWGQGTLVTVSS |
| | C_{H1} | | 107 | ASTKGPSVFPLAPSSKSTSGGTAALGCLVKDYFPEP |
| | | | | VTVSWNSGALTSGVHTFPAVLQSSGLYSLSSVVTV |
| | | | | PSSSLGTQTYICNVNHKPSNTKVDKKV |
| | Linker^{Fab-Fc} | Hinge | 108 | EPKSCDKTHTCPPCP |
| | CH2 | Het-Fc | 109 | APEAAGGPSVFLFPPKPKDTLMISRTPEVTCVVVSV |
| | | | | SHEDPEVKFNWYVDGVEVHNAKTKPREEQYNSTY |
| | | | | RVVSVLTVLHQDWLNGKEYKCKVSNKALPAPIEK |
| | | | | TISKAK |
| | CH3 | Het-Fc | 110 | GQPREPQVYVYPPSRDELTKNQVSLTCLVKGFYPS |
| | | | | DIAVEWESNGQPENNYKTTPPVLDSDGSFALVSKL |
| | | | | TVDKSRWQQGNVFSCSVMHEALHNHYTQKSLSLS |
| | | | | PG |
| 26161 | H | MSLN | 153 | EIVLTQSPATLSLSPGERVTMSCSASSSVSYMHWY |
| | | | | QQKPGQAPRLWIYDTSKLASGVPARFSGSGSGTDY |
| | | | | TLTISSVEPEDNAVYYCQQWSGHPLTFGQGTKVEI |
| | | | | KGGGGSGGGGSGGGGSGGGGSQVQLVQSGAEVK |
| | | | | KPGASVKISCKASGYSFTGYTMNWVRQAPGQGLE |
| | | | | WIGLITPYSGASSYAQKFQGRATLTVDKSTSTAYM |
| | | | | ELSSLRSEDTAVYYCARGGYDGRGFDYWGQGTNV |
| | | | | TVSSAAEPKSSDKTHTCPPCPAPEAAGGPSVFLFPP |
| | | | | KPKDTLMISRTPEVTCVVVSVSHEDPEVKFNWYVD |
| | | | | GVEVHNAKTKPREEQYNSTYRVVSVLTVLHQDWL |
| | | | | NGKEYKCKVSNKALPAPIEKTISKAKGQPREPQVY |
| | | | | VLPPSRDELTKNQVSLLCLVKGFYPSDIAVEWESN |
| | | | | GQPENNYLTWPPVLDSDGSFFLYSKLTVDKSRWQ |
| | | | | QGNVFSCSVMHEALHNHYTQKSLSLSPG |
| | scFv | MSLN | 2 | EIVLTQSPATLSLSPGERVTMSCSASSSVSYMHWY |
| | | | | QQKPGQAPRLWIYDTSKLASGVPARFSGSGSGTDY |
| | | | | TLTISSVEPEDNAVYYCQQWSGHPLTFGQGTKVEI |
| | | | | KGGGGSGGGGSGGGGSGGGGSQVQLVQSGAEVK |
| | | | | KPGASVKISCKASGYSFTGYTMNWVRQAPGQGLE |
| | | | | WIGLITPYSGASSYAQKFQGRATLTVDKSTSTAYM |
| | | | | ELSSLRSEDTAVYYCARGGYDGRGFDYWGQGTNV |
| | | | | TVSS |
| | Linker^{scFv-Fc} | Hinge | 111 | AAEPKSSDKTHTCPPCP |
| | Linker^{scFv-Fc} | Hinge | 112 | EPKSSDKTHTCPPCP |
| | CH2 | Het-Fc | 109 | APEAAGGPSVFLFPPKPKDTLMISRTPEVTCVVVSV |
| | | | | SHEDPEVKFNWYVDGVEVHNAKTKPREEQYNSTY |
| | | | | RVVSVLTVLHQDWLNGKEYKCKVSNKALPAPIEK |
| | | | | TISKAK |
| | CH3 | Het-Fc | 114 | GQPREPQVYVLPPSRDELTKNQVSLLCLVKGFYPS |
| | | | | DIAVEWESNGQPENNYLTWPPVLDSDGSFFLYSKL |
| | | | | TVDKSRWQQGNVFSCSVMHEALHNHYTQKSLSLS |
| | | | | PG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |
| | V_{L} | CD28 | 116 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |
| | C_{L} | | 117 | RTVAAPSVFIFPPSDEQLKSGTASVVCLLNNFYPRE |
| | | | | AKVQWKVDNALQSGNSQESVTEQDSKDSTYSLSS |
| | | | | TLTLSKADYEKHKVYACEVTHQGLSSPVTKSFNRG |
| | | | | EC |

v34914-CD28xCD3xMSLN Trispecific Antibody Construct

| | | | | |
|---|---|---|---|---|
| 26150 | H | CD28/ | 118 | QVQLQESGPGLVKPSETLSLTCAVSGFSLTSYGVH |
| | | CD3 | | WIRQPPGKGLEWLGVIWPGGGTNFNSALMSRLTIS |
| | | | | EDTSKNQVSLKLSSVTAADTAVYYCARDRAYGNY |
| | | | | LYAMDYWGQGTLVTVSSGGGGSGGGGSGGGGSG |
| | | | | GGGSDIQMTQSPSSLSASVGDRVTITCRASESVEYY |
| | | | | VTSLMQWYQQKPGKAPKLLIYAASNVDSGVPSRF |
| | | | | SGSGSGTDFTLTISSLQPEDIATYYCQQSRKVPFTFG |
| | | | | GGTKVEIKGGGGSGEVQLVESGGGLVQPGGSLRLS |
| | | | | CAASGVTFNYYGMSWIRQAPGKGLEWVASITSSG |
| | | | | GRIYYPDSVKGRFTISRENTQKTLYLQMNSLRAED |
| | | | | TAVYYCTLDGRDGWVAYWGQGTLVTVSSASTKG |
| | | | | PSVFPLAPSSKSTSGGTAALGCLVKDYFPEPVTVSW |
| | | | | NSGALTSGVHTFPAVLQSSGLYSLSSVVTVPSSSLG |
| | | | | TQTYICNVNHKPSNTKVDKKVEPKSCDKTHTCPPC |
| | | | | PAPEAAGGPSVFLFPPKPKDTLMISRTPEVTCVVVS |
| | | | | VSHEDPEVKFNWYVDGVEVHNAKTKPREEQYNST |
| | | | | YRVVSVLTVLHQDWLNGKEYKCKVSNKALPAPIE |
| | | | | KTISKAKGQPREPQVYVYPPSRDELTKNQVSLTCL |
| | | | | VKGFYPSDIAVEWESNGQPENNYKTTPPVLDSDGS |
| | | | | FALVSKLTVDKSRWQQGNVFSCSVMHEALHNHYT |
| | | | | QKSLSLSPG |
| | scFv | CD28 | 119 | QVQLQESGPGLVKPSETLSLTCAVSGFSLTSYGVH |
| | | | | WIRQPPGKGLEWLGVIWPGGGTNFNSALMSRLTIS |
| | | | | EDTSKNQVSLKLSSVTAADTAVYYCARDRAYGNY |
| | | | | LYAMDYWGQGTLVTVSSGGGGSGGGGSGGGGSG |
| | | | | GGGSDIQMTQSPSSLSASVGDRVTITCRASESVEYY |
| | | | | VTSLMQWYQQKPGKAPKLLIYAASNVDSGVPSRF |
| | | | | SGSGSGTDFTLTISSLQPEDIATYYCQQSRKVPFTFG |
| | | | | GGTKVEIK |
| | V_{H} | | 106 | QVQLQESGPGLVKPSETLSLTCAVSGFSLTSYGVH |
| | | | | WIRQPPGKGLEWLGVIWPGGGTNFNSALMSRLTIS |
| | | | | EDTSKNQVSLKLSSVTAADTAVYYCARDRAYGNY |
| | | | | LYAMDYWGQGTLVTVSS |
| | Linker^{scFv} | | 104 | GGGGSGGGGSGGGGSGGGGS |
| | V_{L} | | 116 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |
| | Linker^{scFv-Fab} | | 105 | GGGGSG |
| | Fab | CD3 | 154 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSASTKGPSVFPLAPSSKSTSG |
| | | | | GTAALGCLVKDYFPEPVTVSWNSGALTSGVHTFPA |
| | | | | VLQSSGLYSLSSVVTVPSSSLGTQTYICNVNHKPSN |
| | | | | TKVDKKV |
| | V_{H} | CD3 | 102 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSS |
| | C_{H1} | | 107 | ASTKGPSVFPLAPSSKSTSGGTAALGCLVKDYFPEP |
| | | | | VTVSWNSGALTSGVHTFPAVLQSSGLYSLSSVVTV |
| | | | | PSSSLGTQTYICNVNHKPSNTKVDKKV |
| | Linker^{Fab-Fc} | Hinge | 108 | EPKSCDKTHTCPPCP |
| | CH2 | Het-Fc | 109 | APEAAGGPSVFLFPPKPKDTLMISRTPEVTCVVVSV |
| | | | | SHEDPEVKFNWYVDGVEVHNAKTKPREEQYNSTY |
| | | | | RVVSVLTVLHQDWLNGKEYKCKVSNKALPAPIEK |
| | | | | TISKAK |
| | CH3 | Het-Fc | 110 | GQPREPQVYVYPPSRDELTKNQVSLTCLVKGFYPS |
| | | | | DIAVEWESNGQPENNYKTTPPVLDSDGSFALVSKL |
| | | | | TVDKSRWQQGNVFSCSVMHEALHNHYTQKSLSLS |
| | | | | PG |
| 26161 | H | MSLN | 153 | EIVLTQSPATLSLSPGERVTMSCSASSSVSYMHWY |
| | | | | QQKPGQAPRLWIYDTSKLASGVPARFSGSGSGTDY |
| | | | | TLTISSVEPEDNAVYYCQQWSGHPLTFGQGTKVEI |
| | | | | KGGGGSGGGGSGGGGSGGGGSQVQLVQSGAEVK |
| | | | | KPGASVKISCKASGYSFTGYTMNWVRQAPGQGLE |
| | | | | WIGLITPYSGASSYAQKFQGRATLTVDKSTSTAYM |
| | | | | ELSSLRSEDTAVYYCARGGYDGRGFDYWGQGTNV |
| | | | | TVSSAAEPKSSDKTHTCPPCPAPEAAGGPSVFLFPP |
| | | | | KPKDTLMISRTPEVTCVVVSVSHEDPEVKFNWYVD |
| | | | | GVEVHNAKTKPREEQYNSTYRVVSVLTVLHQDWL |
| | | | | NGKEYKCKVSNKALPAPIEKTISKAKGQPREPQVY |
| | | | | VLPPSRDELTKNQVSLLCLVKGFYPSDIAVEWESN |
| | | | | GQPENNYLTWPPVLDSDGSFFLYSKLTVDKSRWQ |
| | | | | QGNVFSCSVMHEALHNHYTQKSLSLSPG |
| 16412 | L | CD3 | 120 | NFMLTQPHSVSESPGKTVTISCKRNTGNIGSNYVN |
| | | | | WYQQHEGSSPTTIIYRNDKRPDGVSDRFSGSIDRSS |
| | | | | KSASLTISNLKTEDEADYFCQSYSSGFIFGGGTKLT |
| | | | | VLGQPKAAPSVTLFPPSSEELQANKATLVCLISDFY |
| | | | | PGAVTVAWKADSSPVKAGVETTTPSKQSNNKYAA |
| | | | | SSYLSLTPEQWKSHRSYSCQVTHEGSTVEKTVAPT |
| | | | | ECS |
| | V_{L} | CD3 | 103 | NFMLTQPHSVSESPGKTVTISCKRNTGNIGSNYVN |
| | | | | WYQQHEGSSPTTIIYRNDKRPDGVSDRFSGSIDRSS |
| | | | | KSASLTISNLKTEDEADYFCQSYSSGFIFGGGTKLT |
| | | | | VL |
| | C_{L} | | 121 | GQPKAAPSVTLFPPSSEELQANKATLVCLISDFYPG |
| | | | | AVTVAWKADSSPVKAGVETTTPSKQSNNKYAASS |
| | | | | YLSLTPEQWKSHRSYSCQVTHEGSTVEKTVAPTEC |
| | | | | S |

v34916-CD3xCD28xMSLN Trispecific Antibody Construct

| | | | | |
|---|---|---|---|---|
| 25094 | H | CD3 | 122 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSASTKGPSVFPLAPSSKSTSG |
| | | | | GTAALGCLVKDYFPEPVTVSWNSGALTSGVHTFPA |
| | | | | VLQSSGLYSLSSVVTVPSSSLGTQTYICNVNHKPSN |
| | | | | TKVDKKVEPKSCDKTHTCPPCPAPEAAGGPSVFLF |
| | | | | PPKPKDTLMISRTPEVTCVVVSVSHEDPEVKFNWY |
| | | | | VDGVEVHNAKTKPREEQYNSTYRVVSVLTVLHQD |
| | | | | WLNGKEYKCKVSNKALPAPIEKTISKAKGQPREPQ |
| | | | | VYVYPPSRDELTKNQVSLTCLVKGFYPSDIAVEWE |
| | | | | SNGQPENNYKTTPPVLDSDGSFALVSKLTVDKSRW |
| | | | | QQGNVFSCSVMHEALHNHYTQKSLSLSPG |
| | V_{H} | CD3 | 102 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSS |
| | C_{H1} | | 107 | ASTKGPSVFPLAPSSKSTSGGTAALGCLVKDYFPEP |
| | | | | VTVSWNSGALTSGVHTFPAVLQSSGLYSLSSVVTV |
| | | | | PSSSLGTQTYICNVNHKPSNTKVDKKV |
| | Linker^{Fab-Fc} | Hinge | 108 | EPKSCDKTHTCPPCP |
| | CH2 | Het-Fc | 109 | APEAAGGPSVFLFPPKPKDTLMISRTPEVTCVVVSV |
| | | | | SHEDPEVKFNWYVDGVEVHNAKTKPREEQYNSTY |
| | | | | RVVSVLTVLHQDWLNGKEYKCKVSNKALPAPIEK |
| | | | | TISKAK |
| | CH3 | Het-Fc | 110 | GQPREPQVYVYPPSRDELTKNQVSLTCLVKGFYPS |
| | | | | DIAVEWESNGQPENNYKTTPPVLDSDGSFALVSKL |
| | | | | TVDKSRWQQGNVFSCSVMHEALHNHYTQKSLSLS |
| | | | | PG |
| 26161 | H | MSLN | 153 | EIVLTQSPATLSLSPGERVTMSCSASSSVSYMHWY |
| | | | | QQKPGQAPRLWIYDTSKLASGVPARFSGSGSGTDY |
| | | | | TLTISSVEPEDNAVYYCQQWSGHPLTFGQGTKVEI |
| | | | | KGGGGSGGGGSGGGGSGGGGSQVQLVQSGAEVK |
| | | | | KPGASVKISCKASGYSFTGYTMNWVRQAPGQGLE |
| | | | | WIGLITPYSGASSYAQKFQGRATLTVDKSTSTAYM |
| | | | | ELSSLRSEDTAVYYCARGGYDGRGFDYWGQGTNV |
| | | | | TVSSAAEPKSSDKTHTCPPCPAPEAAGGPSVFLFPP |
| | | | | KPKDTLMISRTPEVTCVVVSVSHEDPEVKFNWYVD |
| | | | | GVEVHNAKTKPREEQYNSTYRVVSVLTVLHQDWL |
| | | | | NGKEYKCKVSNKALPAPIEKTISKAKGQPREPQVY |
| | | | | VLPPSRDELTKNQVSLLCLVKGFYPSDIAVEWESN |
| | | | | GQPENNYLTWPPVLDSDGSFFLYSKLTVDKSRWQ |
| | | | | QGNVFSCSVMHEALHNHYTQKSLSLSPG |
| 26152 | L | CD3/ | 123 | NFMLTQPHSVSESPGKTVTISCKRNTGNIGSNYVN |
| | | CD28 | | WYQQHEGSSPTTIIYRNDKRPDGVSDRFSGSIDRSS |
| | | | | KSASLTISNLKTEDEADYFCQSYSSGFIFGGGTKLT |
| | | | | VLGQPKAAPSVTLFPPSSEELQANKATLVCLISDFY |
| | | | | PGAVTVAWKADSSPVKAGVETTTPSKQSNNKYAA |
| | | | | SSYLSLTPEQWKSHRSYSCQVTHEGSTVEKTVAPT |
| | | | | ECSGGGGSGDIQMTQSPSSLSASVGDRVTITCRASE |
| | | | | SVEYYVTSLMQWYQQKPGKAPKLLIYAASNVDSG |
| | | | | VPSRFSGSGSGTDFTLTISSLQPEDIATYYCQQSRKV |
| | | | | PFTFGGGTKVEIKGGGGSGGGGSGGGGSGGGGSQ |
| | | | | VQLQESGPGLVKPSETLSLTCAVSGFSLTSYGVHWI |
| | | | | RQPPGKGLEWLGVIWPGGGTNFNSALMSRLTISED |
| | | | | TSKNQVSLKLSSVTAADTAVYYCARDRAYGNYLY |
| | | | | AMDYWGQGTLVTVSS |
| | V_{L} | CD3 | 103 | NFMLTQPHSVSESPGKTVTISCKRNTGNIGSNYVN |
| | | | | WYQQHEGSSPTTIIYRNDKRPDGVSDRESGSIDRSS |
| | | | | KSASLTISNLKTEDEADYFCQSYSSGFIFGGGTKLT |
| | | | | VL |
| | C_{L} | | 121 | GQPKAAPSVTLFPPSSEELQANKATLVCLISDFYPG |
| | | | | AVTVAWKADSSPVKAGVETTTPSKQSNNKYAASS |
| | | | | YLSLTPEQWKSHRSYSCQVTHEGSTVEKTVAPTEC |
| | | | | S |
| | Linker^{Fab-scFv} | | 105 | GGGGSG |
| | V_{L} | CD28 | 116 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |
| | Linker^{scFv} | | 104 | GGGGSGGGGSGGGGSGGGGS |
| | V_{H} | CD28 | 106 | QVQLQESGPGLVKPSETLSLTCAVSGFSLTSYGVH |
| | | | | WIRQPPGKGLEWLGVIWPGGGTNFNSALMSRLTIS |
| | | | | EDTSKNQVSLKLSSVTAADTAVYYCARDRAYGNY |
| | | | | LYAMDYWGQGTLVTVSS |

v34918-CD28xCD3xMSLN Trispecific Antibody Construct

| | | | | |
|---|---|---|---|---|
| 26154 | H | CD28/ | 124 | QVQLQESGPGLVKPSETLSLTCAVSGFSLTSYGVH |
| | | CD3 | | WIRQPPGKGLEWLGVIWPGGGTNFNSALMSRLTIS |
| | | | | EDTSKNQVSLKLSSVTAADTAVYYCARDRAYGNY |
| | | | | LYAMDYWGQGTLVTVSSASTKGPSVFPLAPSSKST |
| | | | | SGGTAALGCLVKDYFPEPVTVSWNSGALTSGVHTF |
| | | | | PAVLQSSGLYSLSSVVTVPSSSLGTQTYICNVNHKP |
| | | | | SNTKVDKKVEPKSCDKTHTCPPCPAPEAAGGPSVF |
| | | | | LFPPKPKDTLMISRTPEVTCVVVSVSHEDPEVKFN |
| | | | | WYVDGVEVHNAKTKPREEQYNSTYRVVSVLTVL |
| | | | | HQDWLNGKEYKCKVSNKALPAPIEKTISKAKGQPR |
| | | | | EPQVYVYPPSRDELTKNQVSLTCLVKGFYPSDIAV |
| | | | | EWESNGQPENNYKTTPPVLDSDGSFALVSKLTVDK |
| | | | | SRWQQGNVFSCSVMHEALHNHYTQKSLSLSPGGG |
| | | | | GGSGNFMLTQPHSVSESPGKTVTISCKRNTGNIGSN |
| | | | | YVNWYQQHEGSSPTTIIYRNDKRPDGVSDRFSGSID |
| | | | | RSSKSASLTISNLKTEDEADYFCQSYSSGFIFGGGTK |
| | | | | LTVLGGGGSGGGGSGGGGSGGGGSEVQLVESGGG |
| | | | | LVQPGGSLRLSCAASGVTFNYYGMSWIRQAPGKG |
| | | | | LEWVASITSSGGRIYYPDSVKGRFTISRENTQKTLY |
| | | | | LQMNSLRAEDTAVYYCTLDGRDGWVAYWGQGTL |
| | | | | VTVSS |
| | V_{H} | CD28 | 106 | QVQLQESGPGLVKPSETLSLTCAVSGFSLTSYGVH |
| | | | | WIRQPPGKGLEWLGVIWPGGGTNFNSALMSRLTIS |
| | | | | EDTSKNQVSLKLSSVTAADTAVYYCARDRAYGNY |
| | | | | LYAMDYWGQGTLVTVSS |
| | C_{H1} | | 107 | ASTKGPSVFPLAPSSKSTSGGTAALGCLVKDYFPEP |
| | | | | VTVSWNSGALTSGVHTFPAVLQSSGLYSLSSVVTV |
| | | | | PSSSLGTQTYICNVNHKPSNTKVDKKV |
| | Linker^{Fab-Fc} | Hinge | 108 | EPKSCDKTHTCPPCP |
| | CH2 | Het-Fc | 109 | APEAAGGPSVFLFPPKPKDTLMISRTPEVTCVVVSV |
| | | | | SHEDPEVKFNWYVDGVEVHNAKTKPREEQYNSTY |
| | | | | RVVSVLTVLHQDWLNGKEYKCKVSNKALPAPIEK |
| | | | | TISKAK |
| | CH3 | Het-Fc | 110 | GQPREPQVYVYPPSRDELTKNQVSLTCLVKGFYPS |
| | | | | DIAVEWESNGQPENNYKTTPPVLDSDGSFALVSKL |
| | | | | TVDKSRWQQGNVFSCSVMHEALHNHYTQKSLSLS |
| | | | | PG |
| | Linker^{Fc-scFv} | | 105 | GGGGSG |
| | V_{L} | CD3 | 103 | NFMLTQPHSVSESPGKTVTISCKRNTGNIGSNYVN |
| | | | | WYQQHEGSSPTTIIYRNDKRPDGVSDRFSGSIDRSS |
| | | | | KSASLTISNLKTEDEADYFCQSYSSGFIFGGGTKLT |
| | | | | VL |
| | Linker^{scFv} | | 104 | GGGGSGGGGSGGGGSGGGGS |
| | V_{H} | CD3 | 102 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSS |
| 26161 | H | MSLN | 153 | EIVLTQSPATLSLSPGERVTMSCSASSSVSYMHWY |
| | | | | QQKPGQAPRLWIYDTSKLASGVPARFSGSGSGTDY |
| | | | | TLTISSVEPEDNAVYYCQQWSGHPLTFGQGTKVEI |
| | | | | KGGGGSGGGGSGGGGSGGGGSQVQLVQSGAEVK |
| | | | | KPGASVKISCKASGYSFTGYTMNWVRQAPGQGLE |
| | | | | WIGLITPYSGASSYAQKFQGRATLTVDKSTSTAYM |
| | | | | ELSSLRSEDTAVYYCARGGYDGRGFDYWGQGTNV |
| | | | | TVSSAAEPKSSDKTHTCPPCPAPEAAGGPSVFLFPP |
| | | | | KPKDTLMISRTPEVTCVVVSVSHEDPEVKFNWYVD |
| | | | | GVEVHNAKTKPREEQYNSTYRVVSVLTVLHQDWL |
| | | | | NGKEYKCKVSNKALPAPIEKTISKAKGQPREPQVY |
| | | | | VLPPSRDELTKNQVSLLCLVKGFYPSDIAVEWESN |
| | | | | GQPENNYLTWPPVLDSDGSFFLYSKLTVDKSRWQ |
| | | | | QGNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v34917-CD28xCD3xMSLN Trispecific Antibody Construct

| | | | | |
|---|---|---|---|---|
| 26153 | H | CD3/ | 129 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSASTKGPSVFPLAPSSKSTSG |
| | | | | GTAALGCLVKDYFPEPVTVSWNSGALTSGVHTFPA |
| | | | | VLQSSGLYSLSSVVTVPSSSLGTQTYICNVNHKPSN |
| | | | | TKVDKKVEPKSCDKTHTCPPCPAPEAAGGPSVFLF |
| | | | | PPKPKDTLMISRTPEVTCVVVSVSHEDPEVKFNWY |
| | | | | VDGVEVHNAKTKPREEQYNSTYRVVSVLTVLHQD |
| | | | | WLNGKEYKCKVSNKALPAPIEKTISKAKGQPREPQ |
| | | | | VYVYPPSRDELTKNQVSLTCLVKGFYPSDIAVEWE |
| | | | | SNGQPENNYKTTPPVLDSDGSFALVSKLTVDKSRW |
| | | | | QQGNVFSCSVMHEALHNHYTQKSLSLSPGGGGGS |
| | | | | GDIQMTQSPSSLSASVGDRVTITCRASESVEYYVTS |
| | | | | LMQWYQQKPGKAPKLLIYAASNVDSGVPSRFSGS |
| | | | | GSGTDFTLTISSLQPEDIATYYCQQSRKVPFTFGGG |
| | | | | TKVEIKGGGGSGGGGSGGGGSGGGGSQVQLQESG |
| | | | | PGLVKPSETLSLTCAVSGESLTSYGVHWIRQPPGKG |
| | | | | LEWLGVIWPGGGTNFNSALMSRLTISEDTSKNQVS |
| | | | | LKLSSVTAADTAVYYCARDRAYGNYLYAMDYWG |
| | | | | QGTLVTVSS |
| 26161 | H | MSLN | 153 | EIVLTQSPATLSLSPGERVTMSCSASSSVSYMHWY |
| | | | | QQKPGQAPRLWIYDTSKLASGVPARFSGSGSGTDY |
| | | | | TLTISSVEPEDNAVYYCQQWSGHPLTFGQGTKVEI |
| | | | | KGGGGSGGGGSGGGGSGGGGSQVQLVQSGAEVK |
| | | | | KPGASVKISCKASGYSFTGYTMNWVRQAPGQGLE |
| | | | | WIGLITPYSGASSYAQKFQGRATLTVDKSTSTAYM |
| | | | | ELSSLRSEDTAVYYCARGGYDGRGFDYWGQGTNV |
| | | | | TVSSAAEPKSSDKTHTCPPCPAPEAAGGPSVFLFPP |
| | | | | KPKDTLMISRTPEVTCVVVSVSHEDPEVKFNWYVD |
| | | | | GVEVHNAKTKPREEQYNSTYRVVSVLTVLHQDWL |
| | | | | NGKEYKCKVSNKALPAPIEKTISKAKGQPREPQVY |
| | | | | VLPPSRDELTKNQVSLLCLVKGFYPSDIAVEWESN |
| | | | | GQPENNYLTWPPVLDSDGSFFLYSKLTVDKSRWQ |
| | | | | QGNVFSCSVMHEALHNHYTQKSLSLSPG |
| 16412 | L | CD3 | 120 | NFMLTQPHSVSESPGKTVTISCKRNTGNIGSNYVN |
| | | | | WYQQHEGSSPTTIIYRNDKRPDGVSDRFSGSIDRSS |
| | | | | KSASLTISNLKTEDEADYFCQSYSSGFIFGGGTKLT |
| | | | | VLGQPKAAPSVTLFPPSSEELQANKATLVCLISDFY |
| | | | | PGAVTVAWKADSSPVKAGVETTTPSKQSNNKYAA |
| | | | | SSYLSLTPEQWKSHRSYSCQVTHEGSTVEKTVAPT |
| | | | | ECS |

v37633-CD28xCD3xCldn18.2 Trispecific Antibody Construct

| | | | | |
|---|---|---|---|---|
| 26150 | H | CD28/ | 118 | QVQLQESGPGLVKPSETLSLTCAVSGFSLTSYGVH |
| | | CD3 | | WIRQPPGKGLEWLGVIWPGGGTNFNSALMSRLTIS |
| | | | | EDTSKNQVSLKLSSVTAADTAVYYCARDRAYGNY |
| | | | | LYAMDYWGQGTLVTVSSGGGGSGGGGSGGGGSG |
| | | | | GGGSDIQMTQSPSSLSASVGDRVTITCRASESVEYY |
| | | | | VTSLMQWYQQKPGKAPKLLIYAASNVDSGVPSRF |
| | | | | SGSGSGTDFTLTISSLQPEDIATYYCQQSRKVPFTFG |
| | | | | GGTKVEIKGGGGSGEVQLVESGGGLVQPGGSLRLS |
| | | | | CAASGVTFNYYGMSWIRQAPGKGLEWVASITSSG |
| | | | | GRIYYPDSVKGRFTISRENTQKTLYLQMNSLRAED |
| | | | | TAVYYCTLDGRDGWVAYWGQGTLVTVSSASTKG |
| | | | | PSVFPLAPSSKSTSGGTAALGCLVKDYFPEPVTVSW |
| | | | | NSGALTSGVHTFPAVLQSSGLYSLSSVVTVPSSSLG |
| | | | | TQTYICNVNHKPSNTKVDKKVEPKSCDKTHTCPPC |
| | | | | PAPEAAGGPSVFLFPPKPKDTLMISRTPEVTCVVVS |
| | | | | VSHEDPEVKFNWYVDGVEVHNAKTKPREEQYNST |
| | | | | YRVVSVLTVLHQDWLNGKEYKCKVSNKALPAPIE |
| | | | | KTISKAKGQPREPQVYVYPPSRDELTKNQVSLTCL |
| | | | | VKGFYPSDIAVEWESNGQPENNYKTTPPVLDSDGS |
| | | | | FALVSKLTVDKSRWQQGNVFSCSVMHEALHNHYT |
| | | | | QKSLSLSPG |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| | | scFv | 126 | QVQLVESGGGLVQPGGSLRLSCSVSGIDLSSNPMI |
| | | | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIK |
| | | VH | 127 | QVQLVESGGGLVQPGGSLRLSCSVSGIDLSSNPMI |
| | | 13b08_H5 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSS |
| | | VL | 128 | DGQMTQSPSSVSASVGDRVTITCQASQSIYSYLSW |
| | | 13b08_L4 | | YQQKPGQRPKLLIYKASTLASGVPSRFSGSGSGTDF |
| | | | | TLTISSVQPEDFATYYCQQGYTVTNVDKNTFGGGT |
| | | | | KVEIK |
| | Linker | (G₄S)₄ | 104 | GGGGSGGGGSGGGGSGGGGS |
| | | Hinge | 111 | AAEPKSSDKTHTCPPCP |
| | | Hinge | 112 | EPKSSDKTHTCPPCP |
| | CH2 | Het-Fc | 109 | APEAAGGPSVFLFPPKPKDTLMISRTPEVTCVVVSV |
| | | | | SHEDPEVKFNWYVDGVEVHNAKTKPREEQYNSTY |
| | | | | RVVSVLTVLHQDWLNGKEYKCKVSNKALPAPIEK |
| | | | | TISKAK |
| | CH3 | Het-Fc | 114 | GQPREPQVYVLPPSRDELTKNQVSLLCLVKGFYPS |
| | | | | DIAVEWESNGQPENNYLTWPPVLDSDGSFFLYSKL |
| | | | | TVDKSRWQQGNVFSCSVMHEALHNHYTQKSLSLS |
| | | | | PG |
| 16412 | L | CD3 | 120 | NFMLTQPHSVSESPGKTVTISCKRNTGNIGSNYVN |
| | | | | WYQQHEGSSPTTIIYRNDKRPDGVSDRFSGSIDRSS |
| | | | | KSASLTISNLKTEDEADYFCQSYSSGFIFGGGTKLT |
| | | | | VLGQPKAAPSVTLFPPSSEELQANKATLVCLISDFY |
| | | | | PGAVTVAWKADSSPVKAGVETTTPSKQSNNKYAA |
| | | | | SSYLSLTPEQWKSHRSYSCQVTHEGSTVEKTVAPT |
| | | | | ECS |

v37634-CD3xCD28xCldn18.2 Trispecific Antibody Construct

| | | | | |
|---|---|---|---|---|
| 25094 | H | CD3 | 122 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSASTKGPSVFPLAPSSKSTSG |
| | | | | GTAALGCLVKDYFPEPVTVSWNSGALTSGVHTFPA |
| | | | | VLQSSGLYSLSSVVTVPSSSLGTQTYICNVNHKPSN |
| | | | | TKVDKKVEPKSCDKTHTCPPCPAPEAAGGPSVFLF |
| | | | | PPKPKDTLMISRTPEVTCVVVSVSHEDPEVKFNWY |
| | | | | VDGVEVHNAKTKPREEQYNSTYRVVSVLTVLHQD |
| | | | | WLNGKEYKCKVSNKALPAPIEKTISKAKGQPREPQ |
| | | | | VYVYPPSRDELTKNQVSLTCLVKGFYPSDIAVEWE |
| | | | | SNGQPENNYKTTPPVLDSDGSFALVSKLTVDKSRW |
| | | | | QQGNVFSCSVMHEALHNHYTQKSLSLSPG |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 26152 | L | CD3/ | 123 | NFMLTQPHSVSESPGKTVTISCKRNTGNIGSNYVN |
| | | CD28 | | WYQQHEGSSPTTIIYRNDKRPDGVSDRFSGSIDRSS |
| | | | | KSASLTISNLKTEDEADYFCQSYSSGFIFGGGTKLT |
| | | | | VLGQPKAAPSVTLFPPSSEELQANKATLVCLISDFY |
| | | | | PGAVTVAWKADSSPVKAGVETTTPSKQSNNKYAA |
| | | | | SSYLSLTPEQWKSHRSYSCQVTHEGSTVEKTVAPT |
| | | | | ECSGGGGSGDIQMTQSPSSLSASVGDRVTITCRASE |
| | | | | SVEYYVTSLMQWYQQKPGKAPKLLIYAASNVDSG |
| | | | | VPSRFSGSGSGTDFTLTISSLQPEDIATYYCQQSRKV |
| | | | | PFTFGGGTKVEIKGGGGSGGGGSGGGGSGGGGSQ |
| | | | | VQLQESGPGLVKPSETLSLTCAVSGFSLTSYGVHWI |
| | | | | RQPPGKGLEWLGVIWPGGGTNFNSALMSRLTISED |
| | | | | TSKNQVSLKLSSVTAADTAVYYCARDRAYGNYLY |
| | | | | AMDYWGQGTLVTVSS |

v37635-CD3xCD28xCldn18.2 Trispecific Antibody Construct

| | | | | |
|---|---|---|---|---|
| 26153 | H | CD3/ | 129 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSASTKGPSVFPLAPSSKSTSG |
| | | | | GTAALGCLVKDYFPEPVTVSWNSGALTSGVHTFPA |
| | | | | VLQSSGLYSLSSVVTVPSSSLGTQTYICNVNHKPSN |
| | | | | TKVDKKVEPKSCDKTHTCPPCPAPEAAGGPSVFLF |
| | | | | PPKPKDTLMISRTPEVTCVVVSVSHEDPEVKFNWY |
| | | | | VDGVEVHNAKTKPREEQYNSTYRVVSVLTVLHQD |
| | | | | WLNGKEYKCKVSNKALPAPIEKTISKAKGQPREPQ |
| | | | | VYVYPPSRDELTKNQVSLTCLVKGFYPSDIAVEWE |
| | | | | SNGQPENNYKTTPPVLDSDGSFALVSKLTVDKSRW |
| | | | | QQGNVFSCSVMHEALHNHYTQKSLSLSPGGGGGS |
| | | | | GDIQMTQSPSSLSASVGDRVTITCRASESVEYYVTS |
| | | | | LMQWYQQKPGKAPKLLIYAASNVDSGVPSRESGS |
| | | | | GSGTDFTLTISSLQPEDIATYYCQQSRKVPFTFGGG |
| | | | | TKVEIKGGGGSGGGGSGGGGSGGGGSQVQLQESG |
| | | | | PGLVKPSETLSLTCAVSGFSLTSYGVHWIRQPPGKG |
| | | | | LEWLGVIWPGGGTNFNSALMSRLTISEDTSKNQVS |
| | | | | LKLSSVTAADTAVYYCARDRAYGNYLYAMDYWG |
| | | | | QGTLVTVSS |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 16412 | L | CD3 | 120 | NFMLTQPHSVSESPGKTVTISCKRNTGNIGSNYVN |
| | | | | WYQQHEGSSPTTIIYRNDKRPDGVSDRFSGSIDRSS |
| | | | | KSASLTISNLKTEDEADYFCQSYSSGFIFGGGTKLT |
| | | | | VLGQPKAAPSVTLFPPSSEELQANKATLVCLISDFY |
| | | | | PGAVTVAWKADSSPVKAGVETTTPSKQSNNKYAA |
| | | | | SSYLSLTPEQWKSHRSYSCQVTHEGSTVEKTVAPT |
| | | | | ECS |

v37636-Cldn18.2xCD3xCD28 Trispecific Antibody Construct

| | | | | |
|---|---|---|---|---|
| 28378 | H | | 130 | QVQLVESGGGLVQPGGSLRLSCSVSGIDLSSNPMI |
| | | | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKGGGGSGEVQLVESGGGLVQPGGSLRLSCAA |
| | | | | SGVTFNYYGMSWIRQAPGKGLEWVASITSSGGRIY |
| | | | | YPDSVKGRFTISRENTQKTLYLQMNSLRAEDTAVY |
| | | | | YCTLDGRDGWVAYWGQGTLVTVSSASTKGPSVFP |
| | | | | LAPSSKSTSGGTAALGCLVKDYFPEPVTVSWNSGA |
| | | | | LTSGVHTFPAVLQSSGLYSLSSVVTVPSSSLGTQTYI |
| | | | | CNVNHKPSNTKVDKKVEPKSCDKTHTCPPCPAPEA |
| | | | | AGGPSVFLFPPKPKDTLMISRTPEVTCVVVSVSHED |
| | | | | PEVKFNWYVDGVEVHNAKTKPREEQYNSTYRVVS |
| | | | | VLTVLHQDWLNGKEYKCKVSNKALPAPIEKTISKA |
| | | | | KGQPREPQVYVYPPSRDELTKNQVSLTCLVKGFYP |
| | | | | SDIAVEWESNGQPENNYKTTPPVLDSDGSFALVSK |
| | | | | LTVDKSRWQQGNVFSCSVMHEALHNHYTQKSLSL |
| | | | | SPGGGGGSGDIQMTQSPSSLSASVGDRVTITCRASE |
| | | | | SVEYYVTSLMQWYQQKPGKAPKLLIYAASNVDSG |
| | | | | VPSRFSGSGSGTDFTLTISSLQPEDIATYYCQQSRKV |
| | | | | PFTFGGGTKVEIKGGGGSGGGGSGGGGSGGGGSQ |
| | | | | VQLQESGPGLVKPSETLSLTCAVSGFSLTSYGVHWI |
| | | | | RQPPGKGLEWLGVIWPGGGTNFNSALMSRLTISED |
| | | | | TSKNQVSLKLSSVTAADTAVYYCARDRAYGNYLY |
| | | | | AMDYWGQGTLVTVSS |
| | V_{H} | Cldn18.2 | 127 | QVQLVESGGGLVQPGGSLRLSCSVSGIDLSSNPMI |
| | | | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSS |
| | Linker^{scFv} | | 104 | GGGGSGGGGSGGGGSGGGGS |
| | V_{L} | Cldn18.2 | 128 | DGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIK |
| | Linker^{scFv-Fab} | | 105 | GGGGSG |
| | V_{H} | CD3 | 102 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSS |
| | C_{H1} | | 107 | ASTKGPSVFPLAPSSKSTSGGTAALGCLVKDYFPEP |
| | | | | VTVSWNSGALTSGVHTFPAVLQSSGLYSLSSVVTV |
| | | | | PSSSLGTQTYICNVNHKPSNTKVDKKV |
| | Linker^{Fab-Fc} | Hinge | 108 | EPKSCDKTHTCPPCP |
| | CH2 | Het-Fc | 109 | APEAAGGPSVFLFPPKPKDTLMISRTPEVTCVVVSV |
| | | | | SHEDPEVKFNWYVDGVEVHNAKTKPREEQYNSTY |
| | | | | RVVSVLTVLHQDWLNGKEYKCKVSNKALPAPIEK |
| | | | | TISKAK |
| | CH3 | Het-Fc | 110 | GQPREPQVYVYPPSRDELTKNQVSLTCLVKGFYPS |
| | | | | DIAVEWESNGQPENNYKTTPPVLDSDGSFALVSKL |
| | | | | TVDKSRWQQGNVFSCSVMHEALHNHYTQKSLSLS |
| | | | | PG |
| | Linker^{Fc-scFv} | | 105 | GGGGSG |
| | V_{L} | CD28 | 116 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |
| | Linker^{scFv} | | 104 | GGGGSGGGGSGGGGSGGGGS |
| | V_{H} | CD28 | 106 | QVQLQESGPGLVKPSETLSLTCAVSGFSLTSYGVH |
| | | | | WIRQPPGKGLEWLGVIWPGGGTNFNSALMSRLTIS |
| | | | | EDTSKNQVSLKLSSVTAADTAVYYCARDRAYGNY |
| | | | | LYAMDYWGQGTLVTVSS |
| 12153 | H | | 131 | EPKSSDKTHTCPPCPAPEAAGGPSVFLFPPKPKDTL |
| | | | | MISRTPEVTCVVVSVSHEDPEVKFNWYVDGVEVH |
| | | | | NAKTKPREEQYNSTYRVVSVLTVLHQDWLNGKEY |
| | | | | KCKVSNKALPAPIEKTISKAKGQPREPQVYVLPPSR |
| | | | | DELTKNQVSLLCLVKGFYPSDIAVEWESNGQPENN |
| | | | | YLTWPPVLDSDGSFFLYSKLTVDKSRWQQGNVFS |
| | | | | CSVMHEALHNHYTQKSLSLSPG |
| | Linker | Hinge | 112 | EPKSSDKTHTCPPCP |
| | CH2 | Het-Fc | 109 | APEAAGGPSVFLFPPKPKDTLMISRTPEVTCVVVSV |
| | | | | SHEDPEVKFNWYVDGVEVHNAKTKPREEQYNSTY |
| | | | | RVVSVLTVLHQDWLNGKEYKCKVSNKALPAPIEK |
| | | | | TISKAK |
| | CH3 | Het-Fc | 114 | GQPREPQVYVLPPSRDELTKNQVSLLCLVKGFYPS |
| | | | | DIAVEWESNGQPENNYLTWPPVLDSDGSFFLYSKL |
| | | | | TVDKSRWQQGNVFSCSVMHEALHNHYTQKSLSLS |
| | | | | PG |
| 16412 | L | CD3 | 120 | NFMLTQPHSVSESPGKTVTISCKRNTGNIGSNYVN |
| | | | | WYQQHEGSSPTTIIYRNDKRPDGVSDRFSGSIDRSS |
| | | | | KSASLTISNLKTEDEADYFCQSYSSGFIFGGGTKLT |
| | | | | VLGQPKAAPSVTLFPPSSEELQANKATLVCLISDFY |
| | | | | PGAVTVAWKADSSPVKAGVETTTPSKQSNNKYAA |
| | | | | SSYLSLTPEQWKSHRSYSCQVTHEGSTVEKTVAPT |
| | | | | ECS |

v37637-Cldn18.2xCD3xCD28 Trispecific Antibody Construct

| | | | | |
|---|---|---|---|---|
| 26121 | H | CD28 | 132 | QVQLQESGPGLVKPSETLSLTCAVSGFSLTSYGVH |
| | | | | WIRQPPGKGLEWLGVIWPGGGTNFNSALMSRLTIS |
| | | | | EDTSKNQVSLKLSSVTAADTAVYYCARDRAYGNY |
| | | | | LYAMDYWGQGTLVTVSSGGGGSGGGGSGGGGSG |
| | | | | GGGSDIQMTQSPSSLSASVGDRVTITCRASESVEYY |
| | | | | VTSLMQWYQQKPGKAPKLLIYAASNVDSGVPSRF |
| | | | | SGSGSGTDFTLTISSLQPEDIATYYCQQSRKVPFTFG |
| | | | | GGTKVEIKAAEPKSSDKTHTCPPCPAPEAAGGPSVF |
| | | | | LFPPKPKDTLMISRTPEVTCVVVSVSHEDPEVKEN |
| | | | | WYVDGVEVHNAKTKPREEQYNSTYRVVSVLTVL |
| | | | | HQDWLNGKEYKCKVSNKALPAPIEKTISKAKGQPR |
| | | | | EPQVYVYPPSRDELTKNQVSLTCLVKGFYPSDIAV |
| | | | | EWESNGQPENNYKTTPPVLDSDGSFALVSKLTVDK |
| | | | | SRWQQGNVFSCSVMHEALHNHYTQKSLSLSPG |
| | V_{H} | CD28 | 106 | QVQLQESGPGLVKPSETLSLTCAVSGFSLTSYGVH |
| | | | | WIRQPPGKGLEWLGVIWPGGGTNFNSALMSRLTIS |
| | | | | EDTSKNQVSLKLSSVTAADTAVYYCARDRAYGNY |
| | | | | LYAMDYWGQGTLVTVSS |
| | Linker^{scFv} | | 104 | GGGGSGGGGSGGGGSGGGGS |
| | V_{L} | | 116 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |
| | Linker | Hinge | 111 | AAEPKSSDKTHTCPPCP |
| | CH2 | Het-Fc | 109 | APEAAGGPSVFLFPPKPKDTLMISRTPEVTCVVVSV |
| | | | | SHEDPEVKFNWYVDGVEVHNAKTKPREEQYNSTY |
| | | | | RVVSVLTVLHQDWLNGKEYKCKVSNKALPAPIEK |
| | | | | TISKAK |
| | CH3 | Het-Fc | 110 | GQPREPQVYVYPPSRDELTKNQVSLTCLVKGFYPS |
| | | | | DIAVEWESNGQPENNYKTTPPVLDSDGSFALVSKL |
| | | | | TVDKSRWQQGNVFSCSVMHEALHNHYTQKSLSLS |
| | | | | PG |
| 28415 | H | Cldn18.2/ | 133 | QVQLVESGGGLVQPGGSLRLSCSVSGIDLSSNPMI |
| | | CD3 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKGGGGSGEVQLVESGGGLVQPGGSLRLSCAA |
| | | | | SGVTFNYYGMSWIRQAPGKGLEWVASITSSGGRIY |
| | | | | YPDSVKGRFTISRENTQKTLYLQMNSLRAEDTAVY |
| | | | | YCTLDGRDGWVAYWGQGTLVTVSSASTKGPSVFP |
| | | | | LAPSSKSTSGGTAALGCLVKDYFPEPVTVSWNSGA |
| | | | | LTSGVHTFPAVLQSSGLYSLSSVVTVPSSSLGTQTYI |
| | | | | CNVNHKPSNTKVDKKVEPKSCDKTHTCPPCPAPEA |
| | | | | AGGPSVFLFPPKPKDTLMISRTPEVTCVVVSVSHED |
| | | | | PEVKFNWYVDGVEVHNAKTKPREEQYNSTYRVVS |
| | | | | VLTVLHQDWLNGKEYKCKVSNKALPAPIEKTISKA |
| | | | | KGQPREPQVYVLPPSRDELTKNQVSLLCLVKGFYP |
| | | | | SDIAVEWESNGQPENNYLTWPPVLDSDGSFFLYSK |
| | | | | LTVDKSRWQQGNVFSCSVMHEALHNHYTQKSLSL |
| | | | | SPG |
| | V_{H} | Cldn18.2 | 127 | QVQLVESGGGLVQPGGSLRLSCSVSGIDLSSNPMI |
| | | | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSS |
| | Linker^{scFv} | | 104 | GGGGSGGGGSGGGGSGGGGS |
| | V_{L} | Cldn18.2 | 128 | DGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIK |
| | Linker^{scFv-Fab} | | 105 | GGGGSG |
| | V_{H} | CD3 | 102 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSS |
| | C_{H1} | | 107 | ASTKGPSVFPLAPSSKSTSGGTAALGCLVKDYFPEP |
| | | | | VTVSWNSGALTSGVHTFPAVLQSSGLYSLSSVVTV |
| | | | | PSSSLGTQTYICNVNHKPSNTKVDKKV |
| | Linker^{Fab-Fc} | Hinge | 108 | EPKSCDKTHTCPPCP |
| | CH2 | Het-Fc | 109 | APEAAGGPSVFLFPPKPKDTLMISRTPEVTCVVVSV |
| | | | | SHEDPEVKFNWYVDGVEVHNAKTKPREEQYNSTY |
| | | | | RVVSVLTVLHQDWLNGKEYKCKVSNKALPAPIEK |
| | | | | TISKAK |
| | CH3 | Het-Fc | 114 | GQPREPQVYVLPPSRDELTKNQVSLLCLVKGFYPS |
| | | | | DIAVEWESNGQPENNYLTWPPVLDSDGSFFLYSKL |
| | | | | TVDKSRWQQGNVFSCSVMHEALHNHYTQKSLSLS |
| | | | | PG |
| 16412 | L | CD3 | 120 | NFMLTQPHSVSESPGKTVTISCKRNTGNIGSNYVN |
| | | | | WYQQHEGSSPTTIIYRNDKRPDGVSDRFSGSIDRSS |
| | | | | KSASLTISNLKTEDEADYFCQSYSSGFIFGGGTKLT |
| | | | | VLGQPKAAPSVTLFPPSSEELQANKATLVCLISDFY |
| | | | | PGAVTVAWKADSSPVKAGVETTTPSKQSNNKYAA |
| | | | | SSYLSLTPEQWKSHRSYSCQVTHEGSTVEKTVAPT |
| | | | | ECS |

v37638-Cldn18.2xCD3xCD28 Trispecific Antibody Construct

| | | | | |
|---|---|---|---|---|
| 26149 | H | CD3/ | 100 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSYGVHWIRQPPGKGLEWLGVIWPGGGTNENS |
| | | | | ALMSRLTISEDTSKNQVSLKLSSVTAADTAVYYCA |
| | | | | RDRAYGNYLYAMDYWGQGTLVTVSSASTKGPSV |
| | | | | FPLAPSSKSTSGGTAALGCLVKDYFPEPVTVSWNS |
| | | | | GALTSGVHTFPAVLQSSGLYSLSSVVTVPSSSLGTQ |
| | | | | TYICNVNHKPSNTKVDKKVEPKSCDKTHTCPPCPA |
| | | | | PEAAGGPSVFLFPPKPKDTLMISRTPEVTCVVVSVS |
| | | | | HEDPEVKFNWYVDGVEVHNAKTKPREEQYNSTYR |
| | | | | VVSVLTVLHQDWLNGKEYKCKVSNKALPAPIEKTI |
| | | | | SKAKGQPREPQVYVYPPSRDELTKNQVSLTCLVKG |
| | | | | FYPSDIAVEWESNGQPENNYKTTPPVLDSDGSFAL |
| | | | | VSKLTVDKSRWQQGNVFSCSVMHEALHNHYTQK |
| | | | | SLSLSPG |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v37639-Cldn18.2xCD28xCD3 Trispecific Antibody Construct

| | | | | |
|---|---|---|---|---|
| 26119 | H | CD28 | 134 | QVQLQESGPGLVKPSETLSLTCAVSGFSLTSYGVH |
| | | | | WIRQPPGKGLEWLGVIWPGGGTNFNSALMSRLTIS |
| | | | | EDTSKNQVSLKLSSVTAADTAVYYCARDRAYGNY |
| | | | | LYAMDYWGQGTLVTVSSASTKGPSVFPLAPSSKST |
| | | | | SGGTAALGCLVKDYFPEPVTVSWNSGALTSGVHTF |
| | | | | PAVLQSSGLYSLSSVVTVPSSSLGTQTYICNVNHKP |
| | | | | SNTKVDKKVEPKSCDKTHTCPPCPAPEAAGGPSVF |
| | | | | LFPPKPKDTLMISRTPEVTCVVVSVSHEDPEVKEN |
| | | | | WYVDGVEVHNAKTKPREEQYNSTYRVVSVLTVL |
| | | | | HQDWLNGKEYKCKVSNKALPAPIEKTISKAKGQPR |
| | | | | EPQVYVYPPSRDELTKNQVSLTCLVKGFYPSDIAV |
| | | | | EWESNGQPENNYKTTPPVLDSDGSFALVSKLTVDK |
| | | | | SRWQQGNVFSCSVMHEALHNHYTQKSLSLSPG |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 27550 | L | CD3/ | 135 | DIQMTQSPSSLSASVGDRVTITCRASESVEYYVTSL |
| | | CD28 | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGECGGGGSGNFMLTQPHSVSESPGKTVTISC |
| | | | | KRNTGNIGSNYVNWYQQHEGSSPTTIIYRNDKRPD |
| | | | | GVSDRFSGSIDRSSKSASLTISNLKTEDEADYFCQS |
| | | | | YSSGFIFGGGTKLTVLGGGGSGGGGSGGGGSGGG |
| | | | | GSEVQLVESGGGLVQPGGSLRLSCAASGVTFNYYG |
| | | | | MSWIRQAPGKGLEWVASITSSGGRIYYPDSVKGRF |
| | | | | TISRENTQKTLYLQMNSLRAEDTAVYYCTLDGRD |
| | | | | GWVAYWGQGTLVTVSS |

v37640-Cldn18.2xCD28xCD3 Trispecific Antibody Construct

| | | | | |
|---|---|---|---|---|
| 26154 | H | CD28/ | 124 | QVQLQESGPGLVKPSETLSLTCAVSGFSLTSYGVH |
| | | CD3 | | WIRQPPGKGLEWLGVIWPGGGTNFNSALMSRLTIS |
| | | | | EDTSKNQVSLKLSSVTAADTAVYYCARDRAYGNY |
| | | | | LYAMDYWGQGTLVTVSSASTKGPSVFPLAPSSKST |
| | | | | SGGTAALGCLVKDYFPEPVTVSWNSGALTSGVHTF |
| | | | | PAVLQSSGLYSLSSVVTVPSSSLGTQTYICNVNHKP |
| | | | | SNTKVDKKVEPKSCDKTHTCPPCPAPEAAGGPSVF |
| | | | | LFPPKPKDTLMISRTPEVTCVVVSVSHEDPEVKEN |
| | | | | WYVDGVEVHNAKTKPREEQYNSTYRVVSVLTVL |
| | | | | HQDWLNGKEYKCKVSNKALPAPIEKTISKAKGQPR |
| | | | | EPQVYVYPPSRDELTKNQVSLTCLVKGFYPSDIAV |
| | | | | EWESNGQPENNYKTTPPVLDSDGSFALVSKLTVDK |
| | | | | SRWQQGNVFSCSVMHEALHNHYTQKSLSLSPGGG |
| | | | | GGSGNFMLTQPHSVSESPGKTVTISCKRNTGNIGSN |
| | | | | YVNWYQQHEGSSPTTIIYRNDKRPDGVSDRFSGSID |
| | | | | RSSKSASLTISNLKTEDEADYFCQSYSSGFIFGGGTK |
| | | | | LTVLGGGGSGGGGSGGGGSGGGGSEVQLVESGGG |
| | | | | LVQPGGSLRLSCAASGVTFNYYGMSWIRQAPGKG |
| | | | | LEWVASITSSGGRIYYPDSVKGRFTISRENTQKTLY |
| | | | | LQMNSLRAEDTAVYYCTLDGRDGWVAYWGQGTL |
| | | | | VTVSS |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v37641-Cldn18.2xCD28xCD3 Trispecific Antibody Construct

| | | | | |
|---|---|---|---|---|
| 28381 | H | Cldn18.2 | 136 | QVQLVESGGGLVQPGGSLRLSCSVSGIDLSSNPMI |
| | | CD28 CD3 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKGGGGSGQVQLQESGPGLVKPSETLSLTCAV |
| | | | | SGFSLTSYGVHWIRQPPGKGLEWLGVIWPGGGTNF |
| | | | | NSALMSRLTISEDTSKNQVSLKLSSVTAADTAVYY |
| | | | | CARDRAYGNYLYAMDYWGQGTLVTVSSASTKGP |
| | | | | SVFPLAPSSKSTSGGTAALGCLVKDYFPEPVTVSW |
| | | | | NSGALTSGVHTFPAVLQSSGLYSLSSVVTVPSSSLG |
| | | | | TQTYICNVNHKPSNTKVDKKVEPKSCDKTHTCPPC |
| | | | | PAPEAAGGPSVFLFPPKPKDTLMISRTPEVTCVVVS |
| | | | | VSHEDPEVKFNWYVDGVEVHNAKTKPREEQYNST |
| | | | | YRVVSVLTVLHQDWLNGKEYKCKVSNKALPAPIE |
| | | | | KTISKAKGQPREPQVYVYPPSRDELTKNQVSLTCL |
| | | | | VKGFYPSDIAVEWESNGQPENNYKTTPPVLDSDGS |
| | | | | FALVSKLTVDKSRWQQGNVFSCSVMHEALHNHYT |
| | | | | QKSLSLSPGGGGGSGNFMLTQPHSVSESPGKTVTIS |
| | | | | CKRNTGNIGSNYVNWYQQHEGSSPTTIIYRNDKRP |
| | | | | DGVSDRFSGSIDRSSKSASLTISNLKTEDEADYFCQ |
| | | | | SYSSGFIFGGGTKLTVLGGGGSGGGGSGGGGSGGG |
| | | | | GSEVQLVESGGGLVQPGGSLRLSCAASGVTFNYYG |
| | | | | MSWIRQAPGKGLEWVASITSSGGRIYYPDSVKGRF |
| | | | | TISRENTQKTLYLQMNSLRAEDTAVYYCTLDGRD |
| | | | | GWVAYWGQGTLVTVSS |
| 12153 | H | Fc | 131 | EPKSSDKTHTCPPCPAPEAAGGPSVFLFPPKPKDTL |
| | | | | MISRTPEVTCVVVSVSHEDPEVKFNWYVDGVEVH |
| | | | | NAKTKPREEQYNSTYRVVSVLTVLHQDWLNGKEY |
| | | | | KCKVSNKALPAPIEKTISKAKGQPREPQVYVLPPSR |
| | | | | DELTKNQVSLLCLVKGFYPSDIAVEWESNGQPENN |
| | | | | YLTWPPVLDSDGSFFLYSKLTVDKSRWQQGNVFS |
| | | | | CSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v37642-Cldn18.2xCD28xCD3 Trispecific Antibody Construct

| | | | | |
|---|---|---|---|---|
| 28382 | H | Cldn18.2/ | 137 | QVQLVESGGGLVQPGGSLRLSCSVSGIDLSSNPMI |
| | | CD28 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKGGGGSGQVQLQESGPGLVKPSETLSLTCAV |
| | | | | SGFSLTSYGVHWIRQPPGKGLEWLGVIWPGGGTNF |
| | | | | NSALMSRLTISEDTSKNQVSLKLSSVTAADTAVYY |
| | | | | CARDRAYGNYLYAMDYWGQGTLVTVSSASTKGP |
| | | | | SVFPLAPSSKSTSGGTAALGCLVKDYFPEPVTVSW |
| | | | | NSGALTSGVHTFPAVLQSSGLYSLSSVVTVPSSSLG |
| | | | | TQTYICNVNHKPSNTKVDKKVEPKSCDKTHTCPPC |
| | | | | PAPEAAGGPSVFLFPPKPKDTLMISRTPEVTCVVVS |
| | | | | VSHEDPEVKFNWYVDGVEVHNAKTKPREEQYNST |
| | | | | YRVVSVLTVLHQDWLNGKEYKCKVSNKALPAPIE |
| | | | | KTISKAKGQPREPQVYVYPPSRDELTKNQVSLTCL |
| | | | | VKGFYPSDIAVEWESNGQPENNYKTTPPVLDSDGS |
| | | | | FALVSKLTVDKSRWQQGNVFSCSVMHEALHNHYT |
| | | | | QKSLSLSPG |
| 28383 | H | CD3 | 138 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLAAEPKSSDKTHTCPPCPAPEAAGGPSVFLFPPKP |
| | | | | KDTLMISRTPEVTCVVVSVSHEDPEVKFNWYVDG |
| | | | | VEVHNAKTKPREEQYNSTYRVVSVLTVLHQDWLN |
| | | | | GKEYKCKVSNKALPAPIEKTISKAKGQPREPQVYV |
| | | | | LPPSRDELTKNQVSLLCLVKGFYPSDIAVEWESNG |
| | | | | QPENNYLTWPPVLDSDGSFFLYSKLTVDKSRWQQ |
| | | | | GNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v37683-Cldn18.2xCD28(mut.)xCD3 Trispecific Antibody Construct

| | | | | |
|---|---|---|---|---|
| 28242 | H | CD3/ | 139 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSYGVHWIRQPPGKGLEWLGVIWAGGGTNFNS |
| | | | | ALMSRLTISEDTSKNQVSLKLSSVTAADTAVYYCA |
| | | | | RDRAYGNYLYAMDYWGQGTLVTVSSASTKGPSV |
| | | | | FPLAPSSKSTSGGTAALGCLVKDYFPEPVTVSWNS |
| | | | | GALTSGVHTFPAVLQSSGLYSLSSVVTVPSSSLGTQ |
| | | | | TYICNVNHKPSNTKVDKKVEPKSCDKTHTCPPCPA |
| | | | | PEAAGGPSVFLFPPKPKDTLMISRTPEVTCVVVSVS |
| | | | | HEDPEVKFNWYVDGVEVHNAKTKPREEQYNSTYR |
| | | | | VVSVLTVLHQDWLNGKEYKCKVSNKALPAPIEKTI |
| | | | | SKAKGQPREPQVYVYPPSRDELTKNQVSLTCLVKG |
| | | | | FYPSDIAVEWESNGQPENNYKTTPPVLDSDGSFAL |
| | | | | VSKLTVDKSRWQQGNVFSCSVMHEALHNHYTQK |
| | | | | SLSLSPG |
| | scFv | CD3 | 101 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRESGSIDRS |
| | | | | SKSASLTISNLKTEDEADYFCQSYSSGFIFGGGTKLT |
| | | | | VL |
| | V_{H} | CD28 | 204 | QVQLQESGPGLVKPSETLSLTCAVSGFSLTSYGVH |
| | (P1058A) | | | WIRQPPGKGLEWLGVIWAGGGTNFNSALMSRLTIS |
| | | | | EDTSKNQVSLKLSSVTAADTAVYYCARDRAYGNY |
| | | | | LYAMDYWGQGTLVTVSS |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v37684-Cldn18.2xCD28(mut.)xCD3 Trispecific Antibody Construct

| | | | | |
|---|---|---|---|---|
| 28245 | H | CD3/ | 140 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSYGVHWIRQPPGKGLEWLGVIWPGGSTNFNSA |
| | | | | LMSRLTISEDTSKNQVSLKLSSVTAADTAVYYCAR |
| | | | | DRAYGNYLYAMDYWGQGTLVTVSSASTKGPSVFP |
| | | | | LAPSSKSTSGGTAALGCLVKDYFPEPVTVSWNSGA |
| | | | | LTSGVHTFPAVLQSSGLYSLSSVVTVPSSSLGTQTYI |
| | | | | CNVNHKPSNTKVDKKVEPKSCDKTHTCPPCPAPEA |
| | | | | AGGPSVFLFPPKPKDTLMISRTPEVTCVVVSVSHED |
| | | | | PEVKFNWYVDGVEVHNAKTKPREEQYNSTYRVVS |
| | | | | VLTVLHQDWLNGKEYKCKVSNKALPAPIEKTISKA |
| | | | | KGQPREPQVYVYPPSRDELTKNQVSLTCLVKGFYP |
| | | | | SDIAVEWESNGQPENNYKTTPPVLDSDGSFALVSK |
| | | | | LTVDKSRWQQGNVFSCSVMHEALHNHYTQKSLSL |
| | | | | SPG |
| | V_{H} | CD28 | 207 | QVQLQESGPGLVKPSETLSLTCAVSGFSLTSYGVH |
| | (G1064S) | | | WIRQPPGKGLEWLGVIWPGGSTNFNSALMSRLTIS |
| | | | | EDTSKNQVSLKLSSVTAADTAVYYCARDRAYGNY |
| | | | | LYAMDYWGQGTLVTVSS |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v37685-Cldn18.2xCD28(mut.)xCD3 Trispecific Antibody Construct

| | | | | |
|---|---|---|---|---|
| 26149 | H | CD3/ | 100 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRESGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSYGVHWIRQPPGKGLEWLGVIWPGGGTNFNS |
| | | | | ALMSRLTISEDTSKNQVSLKLSSVTAADTAVYYCA |
| | | | | RDRAYGNYLYAMDYWGQGTLVTVSSASTKGPSV |
| | | | | FPLAPSSKSTSGGTAALGCLVKDYFPEPVTVSWNS |
| | | | | GALTSGVHTFPAVLQSSGLYSLSSVVTVPSSSLGTQ |
| | | | | TYICNVNHKPSNTKVDKKVEPKSCDKTHTCPPCPA |
| | | | | PEAAGGPSVFLFPPKPKDTLMISRTPEVTCVVVSVS |
| | | | | HEDPEVKFNWYVDGVEVHNAKTKPREEQYNSTYR |
| | | | | VVSVLTVLHQDWLNGKEYKCKVSNKALPAPIEKTI |
| | | | | SKAKGQPREPQVYVYPPSRDELTKNQVSLTCLVKG |
| | | | | FYPSDIAVEWESNGQPENNYKTTPPVLDSDGSFAL |
| | | | | VSKLTVDKSRWQQGNVFSCSVMHEALHNHYTQK |
| | | | | SLSLSPG |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 28426 | L | CD28 | 141 | DIQMTQSPSSLSASVGDRVTITCRASESVEYYGTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |
| | V_{L} | CD28 | 209 | DIQMTQSPSSLSASVGDRVTITCRASESVEYYGTSL |
| | (V1035G) | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |

v37686-Cldn18.2xCD28(mut.)xCD3 Trispecific Antibody Construct

| | | | | |
|---|---|---|---|---|
| 26149 | H | CD3/ | 100 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRESGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSYGVHWIRQPPGKGLEWLGVIWPGGGTNENS |
| | | | | ALMSRLTISEDTSKNQVSLKLSSVTAADTAVYYCA |
| | | | | RDRAYGNYLYAMDYWGQGTLVTVSSASTKGPSV |
| | | | | FPLAPSSKSTSGGTAALGCLVKDYFPEPVTVSWNS |
| | | | | GALTSGVHTFPAVLQSSGLYSLSSVVTVPSSSLGTQ |
| | | | | TYICNVNHKPSNTKVDKKVEPKSCDKTHTCPPCPA |
| | | | | PEAAGGPSVFLFPPKPKDTLMISRTPEVTCVVVSVS |
| | | | | HEDPEVKFNWYVDGVEVHNAKTKPREEQYNSTYR |
| | | | | VVSVLTVLHQDWLNGKEYKCKVSNKALPAPIEKTI |
| | | | | SKAKGQPREPQVYVYPPSRDELTKNQVSLTCLVKG |
| | | | | FYPSDIAVEWESNGQPENNYKTTPPVLDSDGSFAL |
| | | | | VSKLTVDKSRWQQGNVFSCSVMHEALHNHYTQK |
| | | | | SLSLSPG |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 28427 | L | CD28 | 142 | DIQMTQSPSSLSASVGDRVTITCRASESVEYYVTSL |
| | | | | MQWYQQKPGKAPKLLIYAASNVESGVPSRFSGSGS |
| | | | | GTDFTLTISSLQPEDIATYYCQQSRKVPFTFGGGTK |
| | | | | VEIKRTVAAPSVFIFPPSDEQLKSGTASVVCLLNNF |
| | | | | YPREAKVQWKVDNALQSGNSQESVTEQDSKDSTY |
| | | | | SLSSTLTLSKADYEKHKVYACEVTHQGLSSPVTKS |
| | | | | FNRGEC |
| | V_{L} | CD28 | 200 | DIQMTQSPSSLSASVGDRVTITCRASESVEYYVTSL |
| | (D1068E) | | | MQWYQQKPGKAPKLLIYAASNVESGVPSRFSGSGS |
| | | | | GTDFTLTISSLQPEDIATYYCQQSRKVPFTFGGGTK |
| | | | | VEIK |

v37687-Cldn18.2xCD28(mut.)xCD3 Trispecific Antibody Construct

| | | | | |
|---|---|---|---|---|
| 28428 | H | CD3/ | 143 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRESGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSYGVHWIRQPPGKGLEWLGVIWPGGGTNFNS |
| | | | | ALMSRLTISKDTSKNQVSLKLSSVTAADTAVYYCA |
| | | | | RDRAYGNYLYAMDYWGQGTLVTVSSASTKGPSV |
| | | | | FPLAPSSKSTSGGTAALGCLVKDYFPEPVTVSWNS |
| | | | | GALTSGVHTFPAVLQSSGLYSLSSVVTVPSSSLGTQ |
| | | | | TYICNVNHKPSNTKVDKKVEPKSCDKTHTCPPCPA |
| | | | | PEAAGGPSVFLFPPKPKDTLMISRTPEVTCVVVSVS |
| | | | | HEDPEVKFNWYVDGVEVHNAKTKPREEQYNSTYR |
| | | | | VVSVLTVLHQDWLNGKEYKCKVSNKALPAPIEKTI |
| | | | | SKAKGQPREPQVYVYPPSRDELTKNQVSLTCLVKG |
| | | | | FYPSDIAVEWESNGQPENNYKTTPPVLDSDGSFAL |
| | | | | VSKLTVDKSRWQQGNVFSCSVMHEALHNHYTQK |
| | | | | SLSLSPG |
| | V_{H} | CD28 | 203 | QVQLQESGPGLVKPSETLSLTCAVSGFSLTSYGVH |
| | (E1080K) | | | WIRQPPGKGLEWLGVIWPGGGTNFNSALMSRLTIS |
| | | | | KDTSKNQVSLKLSSVTAADTAVYYCARDRAYGNY |
| | | | | LYAMDYWGQGTLVTVSS |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v37688-Cldn18.2xCD28(mut.)xCD3 Trispecific Antibody Construct

| | | | | |
|---|---|---|---|---|
| 28429 | H | CD3/ | 144 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSYGVHWIRQPPGKGLEWLGVIWPGGGTNENS |
| | | | | ALMSRLTISEDTSKNQVSLKLSSVTAADTAVYYCA |
| | | | | RDRASGNYLYAMDYWGQGTLVTVSSASTKGPSVF |
| | | | | PLAPSSKSTSGGTAALGCLVKDYFPEPVTVSWNSG |
| | | | | ALTSGVHTFPAVLQSSGLYSLSSVVTVPSSSLGTQT |
| | | | | YICNVNHKPSNTKVDKKVEPKSCDKTHTCPPCPAP |
| | | | | EAAGGPSVFLFPPKPKDTLMISRTPEVTCVVVSVSH |
| | | | | EDPEVKFNWYVDGVEVHNAKTKPREEQYNSTYRV |
| | | | | VSVLTVLHQDWLNGKEYKCKVSNKALPAPIEKTIS |
| | | | | KAKGQPREPQVYVYPPSRDELTKNQVSLTCLVKGF |
| | | | | YPSDIAVEWESNGQPENNYKTTPPVLDSDGSFALV |
| | | | | SKLTVDKSRWQQGNVFSCSVMHEALHNHYTQKSL |
| | | | | SLSPG |
| | V_{H} | CD28 | 201 | QVQLQESGPGLVKPSETLSLTCAVSGFSLTSYGVH |
| | (Y1110S) | | | WIRQPPGKGLEWLGVIWPGGGTNFNSALMSRLTIS |
| | | | | EDTSKNQVSLKLSSVTAADTAVYYCARDRASGNY |
| | | | | LYAMDYWGQGTLVTVSS |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v37689-Cldn18.2xCD28(mut.)xCD3 Trispecific Antibody Construct

| | | | | |
|---|---|---|---|---|
| 28430 | H | CD3/ | 145 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSYGVHWIRQPPGKGLEWLGVIWPGGGTNENS |
| | | | | ALMSRLTISEDTSKNQVSLKLSSVTAADTAVYYCA |
| | | | | RDRAYGAYLYAMDYWGQGTLVTVSSASTKGPSV |
| | | | | FPLAPSSKSTSGGTAALGCLVKDYFPEPVTVSWNS |
| | | | | GALTSGVHTFPAVLQSSGLYSLSSVVTVPSSSLGTQ |
| | | | | TYICNVNHKPSNTKVDKKVEPKSCDKTHTCPPCPA |
| | | | | PEAAGGPSVFLFPPKPKDTLMISRTPEVTCVVVSVS |
| | | | | HEDPEVKFNWYVDGVEVHNAKTKPREEQYNSTYR |
| | | | | VVSVLTVLHQDWLNGKEYKCKVSNKALPAPIEKTI |
| | | | | SKAKGQPREPQVYVYPPSRDELTKNQVSLTCLVKG |
| | | | | FYPSDIAVEWESNGQPENNYKTTPPVLDSDGSFAL |
| | | | | VSKLTVDKSRWQQGNVFSCSVMHEALHNHYTQK |
| | | | | SLSLSPG |
| | V_{H} | CD28 | 208 | QVQLQESGPGLVKPSETLSLTCAVSGFSLTSYGVH |
| | (N1111aA) | | | WIRQPPGKGLEWLGVIWPGGGTNFNSALMSRLTIS |
| | | | | EDTSKNQVSLKLSSVTAADTAVYYCARDRAYGAY |
| | | | | LYAMDYWGQGTLVTVSS |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v37690-Cldn18.2xCD28(mut.)xCD3 Trispecific Antibody Construct

| | | | | |
|---|---|---|---|---|
| 28431 | H | CD3/ | 146 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSYGVHWIRQPPGKGLEWLGVIWPGGGTNENS |
| | | | | ALMSRLTISEDTSKNQVSLKLSSVTAADTAVYYCA |
| | | | | RDRAYGNSLYAMDYWGQGTLVTVSSASTKGPSVF |
| | | | | PLAPSSKSTSGGTAALGCLVKDYFPEPVTVSWNSG |
| | | | | ALTSGVHTFPAVLQSSGLYSLSSVVTVPSSSLGTQT |
| | | | | YICNVNHKPSNTKVDKKVEPKSCDKTHTCPPCPAP |
| | | | | EAAGGPSVFLFPPKPKDTLMISRTPEVTCVVVSVSH |
| | | | | EDPEVKFNWYVDGVEVHNAKTKPREEQYNSTYRV |
| | | | | VSVLTVLHQDWLNGKEYKCKVSNKALPAPIEKTIS |
| | | | | KAKGQPREPQVYVYPPSRDELTKNQVSLTCLVKGF |
| | | | | YPSDIAVEWESNGQPENNYKTTPPVLDSDGSFALV |
| | | | | SKLTVDKSRWQQGNVFSCSVMHEALHNHYTQKSL |
| | | | | SLSPG |
| | V_{H} | CD28 | 113 | QVQLQESGPGLVKPSETLSLTCAVSGFSLTSYGVH |
| | (Y1112S) | | | WIRQPPGKGLEWLGVIWPGGGTNFNSALMSRLTIS |
| | | | | EDTSKNQVSLKLSSVTAADTAVYYCARDRAYGNS |
| | | | | LYAMDYWGQGTLVTVSS |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v37691-Cldn18.2xCD28(mut.)xCD3 Trispecific Antibody Construct

| | | | | |
|---|---|---|---|---|
| 28432 | H | CD3/ | 147 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSYGVHWIRQPPGKGLEWLGVIWPGGGTNFNS |
| | | | | ALMSRLTISEDTSKNQVSLKLSSVTAADTAVYYCA |
| | | | | RDRAYGNYNYAMDYWGQGTLVTVSSASTKGPSV |
| | | | | FPLAPSSKSTSGGTAALGCLVKDYFPEPVTVSWNS |
| | | | | GALTSGVHTFPAVLQSSGLYSLSSVVTVPSSSLGTQ |
| | | | | TYICNVNHKPSNTKVDKKVEPKSCDKTHTCPPCPA |
| | | | | PEAAGGPSVFLFPPKPKDTLMISRTPEVTCVVVSVS |
| | | | | HEDPEVKFNWYVDGVEVHNAKTKPREEQYNSTYR |
| | | | | VVSVLTVLHQDWLNGKEYKCKVSNKALPAPIEKTI |
| | | | | SKAKGQPREPQVYVYPPSRDELTKNQVSLTCLVKG |
| | | | | FYPSDIAVEWESNGQPENNYKTTPPVLDSDGSFAL |
| | | | | VSKLTVDKSRWQQGNVFSCSVMHEALHNHYTQK |
| | | | | SLSLSPG |
| | V_{H} | | 206 | QVQLQESGPGLVKPSETLSLTCAVSGFSLTSYGVH |
| | (L1112aN) | | | WIRQPPGKGLEWLGVIWPGGGTNFNSALMSRLTIS |
| | | | | EDTSKNQVSLKLSSVTAADTAVYYCARDRAYGNY |
| | | | | NYAMDYWGQGTLVTVSS |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v37692-Cldn18.2xCD28(mut.)xCD3 Trispecific Antibody Construct

| | | | | |
|---|---|---|---|---|
| 28433 | H | CD3/ | 148 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSYGVHWIRQPPGKGLEWLGVIWPGGGTNFNS |
| | | | | ALMSRLTISEDTSKNQVSLKLSSVTAADTAVYYCA |
| | | | | RDRAYGNYLSAMDYWGQGTLVTVSSASTKGPSVF |
| | | | | PLAPSSKSTSGGTAALGCLVKDYFPEPVTVSWNSG |
| | | | | ALTSGVHTFPAVLQSSGLYSLSSVVTVPSSSLGTQT |
| | | | | YICNVNHKPSNTKVDKKVEPKSCDKTHTCPPCPAP |
| | | | | EAAGGPSVFLFPPKPKDTLMISRTPEVTCVVVSVSH |
| | | | | EDPEVKFNWYVDGVEVHNAKTKPREEQYNSTYRV |
| | | | | VSVLTVLHQDWLNGKEYKCKVSNKALPAPIEKTIS |
| | | | | KAKGQPREPQVYVYPPSRDELTKNQVSLTCLVKGF |
| | | | | YPSDIAVEWESNGQPENNYKTTPPVLDSDGSFALV |
| | | | | SKLTVDKSRWQQGNVFSCSVMHEALHNHYTQKSL |
| | | | | SLSPG |
| | V_{H} | | 210 | QVQLQESGPGLVKPSETLSLTCAVSGFSLTSYGVH |
| | (Y1113S) | | | WIRQPPGKGLEWLGVIWPGGGTNFNSALMSRLTIS |
| | | | | EDTSKNQVSLKLSSVTAADTAVYYCARDRAYGNY |
| | | | | LSAMDYWGQGTLVTVSS |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v37693-Cldn18.2xCD28(mut.)xCD3 Trispecific Antibody Construct

| | | | | |
|---|---|---|---|---|
| 28434 | H | CD3/ | 149 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSAGVHWIRQPPGKGLEWLGVIWPGGGTNFNS |
| | | | | ALMSRLTISEDTSKNQVSLKLSSVTAADTAVYYCA |
| | | | | RDRAYGNYLYAMDYWGQGTLVTVSSASTKGPSV |
| | | | | FPLAPSSKSTSGGTAALGCLVKDYFPEPVTVSWNS |
| | | | | GALTSGVHTFPAVLQSSGLYSLSSVVTVPSSSLGTQ |
| | | | | TYICNVNHKPSNTKVDKKVEPKSCDKTHTCPPCPA |
| | | | | PEAAGGPSVFLFPPKPKDTLMISRTPEVTCVVVSVS |
| | | | | HEDPEVKFNWYVDGVEVHNAKTKPREEQYNSTYR |
| | | | | VVSVLTVLHQDWLNGKEYKCKVSNKALPAPIEKTI |
| | | | | SKAKGQPREPQVYVYPPSRDELTKNQVSLTCLVKG |
| | | | | FYPSDIAVEWESNGQPENNYKTTPPVLDSDGSFAL |
| | | | | VSKLTVDKSRWQQGNVFSCSVMHEALHNHYTQK |
| | | | | SLSLSPG |
| | V_{H} | | 205 | QVQLQESGPGLVKPSETLSLTCAVSGFSLTSAGVH |
| | (Y1037A) | | | WIRQPPGKGLEWLGVIWPGGGTNFNSALMSRLTIS |
| | | | | EDTSKNQVSLKLSSVTAADTAVYYCARDRAYGNY |
| | | | | LYAMDYWGQGTLVTVSS |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v37694-Cldn18.2xCD28(mut.)xCD3 Trispecific Antibody Construct

| | | | | |
|---|---|---|---|---|
| 26149 | H | CD3/ | 100 | EVQLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSYGVHWIRQPPGKGLEWLGVIWPGGGTNFNS |
| | | | | ALMSRLTISEDTSKNQVSLKLSSVTAADTAVYYCA |
| | | | | RDRAYGNYLYAMDYWGQGTLVTVSSASTKGPSV |
| | | | | FPLAPSSKSTSGGTAALGCLVKDYFPEPVTVSWNS |
| | | | | GALTSGVHTFPAVLQSSGLYSLSSVVTVPSSSLGTQ |
| | | | | TYICNVNHKPSNTKVDKKVEPKSCDKTHTCPPCPA |
| | | | | PEAAGGPSVFLFPPKPKDTLMISRTPEVTCVVVSVS |
| | | | | HEDPEVKFNWYVDGVEVHNAKTKPREEQYNSTYR |
| | | | | VVSVLTVLHQDWLNGKEYKCKVSNKALPAPIEKTI |
| | | | | SKAKGQPREPQVYVYPPSRDELTKNQVSLTCLVKG |
| | | | | FYPSDIAVEWESNGQPENNYKTTPPVLDSDGSFAL |
| | | | | VSKLTVDKSRWQQGNVFSCSVMHEALHNHYTQK |
| | | | | SLSLSPG |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 28435 | L | CD28 | 150 | DIQMTQSPSSLSASVGDRVTITCRASESVEAYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |
| | V_{L} | CD28 | 151 | DIQMTQSPSSLSASVGDRVTITCRASESVEAYVTSL |
| | (Y1031A) | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |

v37695-Cldn18.2xCD28(mut.)xCD3 Trispecific Antibody Construct

| | | | | |
|---|---|---|---|---|
| 26149 | H | CD3/ | 100 | EVOLVESGGGLVQPGGSLRLSCAASGVTFNYYGM |
| | | CD28 | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLGGGGSGQVQLQESGPGLVKPSETLSLTCAVSGF |
| | | | | SLTSYGVHWIRQPPGKGLEWLGVIWPGGGTNFNS |
| | | | | ALMSRLTISEDTSKNQVSLKLSSVTAADTAVYYCA |
| | | | | RDRAYGNYLYAMDYWGQGTLVTVSSASTKGPSV |
| | | | | FPLAPSSKSTSGGTAALGCLVKDYFPEPVTVSWNS |
| | | | | GALTSGVHTFPAVLQSSGLYSLSSVVTVPSSSLGTQ |
| | | | | TYICNVNHKPSNTKVDKKVEPKSCDKTHTCPPCPA |
| | | | | PEAAGGPSVFLFPPKPKDTLMISRTPEVTCVVVSVS |
| | | | | HEDPEVKFNWYVDGVEVHNAKTKPREEQYNSTYR |
| | | | | VVSVLTVLHQDWLNGKEYKCKVSNKALPAPIEKTI |
| | | | | SKAKGQPREPQVYVYPPSRDELTKNQVSLTCLVKG |
| | | | | FYPSDIAVEWESNGQPENNYKTTPPVLDSDGSFAL |
| | | | | VSKLTVDKSRWQQGNVFSCSVMHEALHNHYTQK |
| | | | | SLSLSPG |
| 28373 | H | CLDN | 125 | QVQLVESGGGLVQPGGSLRLSCSVSGIDLSSNPMI |
| | | 18.2 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKAAEPKSSDKTHTCPPCPAPEAAGGPSVFLFP |
| | | | | PKPKDTLMISRTPEVTCVVVSVSHEDPEVKFNWYV |
| | | | | DGVEVHNAKTKPREEQYNSTYRVVSVLTVLHQD |
| | | | | WLNGKEYKCKVSNKALPAPIEKTISKAKGQPREPQ |
| | | | | VYVLPPSRDELTKNQVSLLCLVKGFYPSDIAVEWE |
| | | | | SNGQPENNYLTWPPVLDSDGSFFLYSKLTVDKSRW |
| | | | | QQGNVFSCSVMHEALHNHYTQKSLSLSPG |
| 28436 | L | CD28 | 152 | DIQMTQSPSSLSASVGDRVTITCRASESVEYYVTSL |
| | | | | MQWYQQKPGKAPKLLIYAASAVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |
| | V_{L} | | 202 | DIQMTQSPSSLSASVGDRVTITCRASESVEYYVTSL |
| | (N1066A) | | | MQWYQQKPGKAPKLLIYAASAVDSGVPSRFSGSG |
| | | | | SGTDFTLTISSLQPEDIATYYCQQSRKVPFTFGGGT |
| | | | | KVEIK |

v38990-Cldn18.2xCD28(mut.)xCD3 Trispecific Antibody Construct

| | | | | |
|---|---|---|---|---|
| 29584 | H | Cldn18.2/ | 211 | QVQLVESGGGLVQPGGSLRLSCSVSGIDLSSNPMI |
| | | CD28 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKGGGGSGQVQLQESGPGLVKPSETLSLTCAV |
| | | | | SGFSLTSYGVHWIRQPPGKGLEWLGVIWPGGGTNF |
| | | | | NSALMSRLTISEDTSKNQVSLKLSSVTAADTAVYY |
| | | | | CARDRAYGNYLSAMDYWGQGTLVTVSSASTKGPS |
| | | | | VFPLAPSSKSTSGGTAALGCLVKDYFPEPVTVSWN |
| | | | | SGALTSGVHTFPAVLQSSGLYSLSSVVTVPSSSLGT |
| | | | | QTYICNVNHKPSNTKVDKKVEPKSCDKTHTCPPCP |
| | | | | APEAAGGPSVFLFPPKPKDTLMISRTPEVTCVVVSV |
| | | | | SHEDPEVKFNWYVDGVEVHNAKTKPREEQYNSTY |
| | | | | RVVSVLTVLHQDWLNGKEYKCKVSNKALPAPIEK |
| | | | | TISKAKGQPREPQVYVYPPSRDELTKNQVSLTCLV |
| | | | | KGFYPSDIAVEWESNGQPENNYKTTPPVLDSDGSF |
| | | | | ALVSKLTVDKSRWQQGNVFSCSVMHEALHNHYT |
| | | | | QKSLSLSPG |
| 28383 | H | CD3 | 138 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLAAEPKSSDKTHTCPPCPAPEAAGGPSVFLFPPKP |
| | | | | KDTLMISRTPEVTCVVVSVSHEDPEVKFNWYVDG |
| | | | | VEVHNAKTKPREEQYNSTYRVVSVLTVLHQDWLN |
| | | | | GKEYKCKVSNKALPAPIEKTISKAKGQPREPQVYV |
| | | | | LPPSRDELTKNQVSLLCLVKGFYPSDIAVEWESNG |
| | | | | QPENNYLTWPPVLDSDGSFFLYSKLTVDKSRWQQ |
| | | | | GNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v38991-Cldn18.2xCD28(mut.)xCD3 Trispecific Antibody Construct

| | | | | |
|---|---|---|---|---|
| 29583 | H | Cldn18.2/ | 212 | QVQLVESGGGLVQPGGSLRLSCSVSGIDLSSNPMI |
| | | CD28 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKGGGGSGQVQLQESGPGLVKPSETLSLTCAV |
| | | | | SGFSLTSYGVHWIRQPPGKGLEWLGVIWPGGGTNF |
| | | | | NSALMSRLTISEDTSKNQVSLKLSSVTAADTAVYY |
| | | | | CARDRAYGAYLYAMDYWGQGTLVTVSSASTKGP |
| | | | | SVFPLAPSSKSTSGGTAALGCLVKDYFPEPVTVSW |
| | | | | NSGALTSGVHTFPAVLQSSGLYSLSSVVTVPSSSLG |
| | | | | TQTYICNVNHKPSNTKVDKKVEPKSCDKTHTCPPC |
| | | | | PAPEAAGGPSVFLFPPKPKDTLMISRTPEVTCVVVS |
| | | | | VSHEDPEVKFNWYVDGVEVHNAKTKPREEQYNST |
| | | | | YRVVSVLTVLHQDWLNGKEYKCKVSNKALPAPIE |
| | | | | KTISKAKGQPREPQVYVYPPSRDELTKNQVSLTCL |
| | | | | VKGFYPSDIAVEWESNGQPENNYKTTPPVLDSDGS |
| | | | | FALVSKLTVDKSRWQQGNVFSCSVMHEALHNHYT |
| | | | | QKSLSLSPG |
| 28383 | H | CD3 | 138 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLAAEPKSSDKTHTCPPCPAPEAAGGPSVFLFPPKP |
| | | | | KDTLMISRTPEVTCVVVSVSHEDPEVKFNWYVDG |
| | | | | VEVHNAKTKPREEQYNSTYRVVSVLTVLHQDWLN |
| | | | | GKEYKCKVSNKALPAPIEKTISKAKGQPREPQVYV |
| | | | | LPPSRDELTKNQVSLLCLVKGFYPSDIAVEWESNG |
| | | | | QPENNYLTWPPVLDSDGSFFLYSKLTVDKSRWQQ |
| | | | | GNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v38992-Cldn18.2xCD28(mut.)xCD3 Trispecific Antibody Construct

| | | | | |
|---|---|---|---|---|
| 29603 | H | Cldn18.2/ | 213 | QVQLVESGGGLVQPGGSLRLSCSVSGIDLSSNPMI |
| | | CD28 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKGGGGSGQVQLQESGPGLVKPSETLSLTCAV |
| | | | | SGFSLTSYGVHWIRQPPGKGLEWLGVIWPGGSTNF |
| | | | | NSALMSRLTISEDTSKNQVSLKLSSVTAADTAVYY |
| | | | | CARDRAYGNYLYAMDYWGQGTLVTVSSASTKGP |
| | | | | SVFPLAPSSKSTSGGTAALGCLVKDYFPEPVTVSW |
| | | | | NSGALTSGVHTFPAVLQSSGLYSLSSVVTVPSSSLG |
| | | | | TQTYICNVNHKPSNTKVDKKVEPKSCDKTHTCPPC |
| | | | | PAPEAAGGPSVFLFPPKPKDTLMISRTPEVTCVVVS |
| | | | | VSHEDPEVKFNWYVDGVEVHNAKTKPREEQYNST |
| | | | | YRVVSVLTVLHQDWLNGKEYKCKVSNKALPAPIE |
| | | | | KTISKAKGQPREPQVYVYPPSRDELTKNQVSLTCL |
| | | | | VKGFYPSDIAVEWESNGQPENNYKTTPPVLDSDGS |
| | | | | FALVSKLTVDKSRWQQGNVFSCSVMHEALHNHYT |
| | | | | QKSLSLSPG |
| 28383 | H | CD3 | 138 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLAAEPKSSDKTHTCPPCPAPEAAGGPSVFLFPPKP |
| | | | | KDTLMISRTPEVTCVVVSVSHEDPEVKFNWYVDG |
| | | | | VEVHNAKTKPREEQYNSTYRVVSVLTVLHQDWLN |
| | | | | GKEYKCKVSNKALPAPIEKTISKAKGQPREPQVYV |
| | | | | LPPSRDELTKNQVSLLCLVKGFYPSDIAVEWESNG |
| | | | | QPENNYLTWPPVLDSDGSFFLYSKLTVDKSRWQQ |
| | | | | GNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

v38993-Cldn18.2xCD28(mut.)xCD3 Trispecific Antibody Construct

| | | | | |
|---|---|---|---|---|
| 29604 | H | Cldn18.2/ | 214 | QVQLVESGGGLVQPGGSLRLSCSVSGIDLSSNPMI |
| | | CD28 | | WVRQAPGKGLQYIGIIDTDGSTYYADWAKGRFTIS |
| | | | | KDSTTVYLQINSPRAEDTAVYYCARRLHGSSNGYY |
| | | | | DDLWGQGTLVTVSSGGGGSGGGGSGGGGSGGGG |
| | | | | SDGQMTQSPSSVSASVGDRVTITCQASQSIYSYLSW |
| | | | | YQQKPGQRPKLLIYKASTLASGVPSRFSGSGSGTDF |
| | | | | TLTISSVQPEDFATYYCQQGYTVTNVDKNTFGGGT |
| | | | | KVEIKGGGGSGQVQLQESGPGLVKPSETLSLTCAV |
| | | | | SGFSLTSYGVHWIRQPPGKGLEWLGVIWPGGGTNF |
| | | | | NSALMSRLTISEDTSKNQVSLKLSSVTAADTAVYY |
| | | | | CARDRAYGNYNYAMDYWGQGTLVTVSSASTKGP |
| | | | | SVFPLAPSSKSTSGGTAALGCLVKDYFPEPVTVSW |
| | | | | NSGALTSGVHTFPAVLQSSGLYSLSSVVTVPSSSLG |
| | | | | TQTYICNVNHKPSNTKVDKKVEPKSCDKTHTCPPC |
| | | | | PAPEAAGGPSVFLFPPKPKDTLMISRTPEVTCVVVS |
| | | | | VSHEDPEVKFNWYVDGVEVHNAKTKPREEQYNST |
| | | | | YRVVSVLTVLHQDWLNGKEYKCKVSNKALPAPIE |
| | | | | KTISKAKGQPREPQVYVYPPSRDELTKNQVSLTCL |
| | | | | VKGFYPSDIAVEWESNGQPENNYKTTPPVLDSDGS |
| | | | | FALVSKLTVDKSRWQQGNVFSCSVMHEALHNHYT |
| | | | | QKSLSLSPG |
| 28383 | H | CD3 | 138 | EVQLVESGGGLVQPGGSLRLSCAASGVTFNYYGM |
| | | | | SWIRQAPGKGLEWVASITSSGGRIYYPDSVKGRFTI |
| | | | | SRENTQKTLYLQMNSLRAEDTAVYYCTLDGRDG |
| | | | | WVAYWGQGTLVTVSSGGGGSGGGGSGGGGSGGG |
| | | | | GSNFMLTQPHSVSESPGKTVTISCKRNTGNIGSNYV |
| | | | | NWYQQHEGSSPTTIIYRNDKRPDGVSDRFSGSIDRS |
| | | | | SKSASLTISNLKTEDEADYFCQSYSSGFIFGGGTKLT |
| | | | | VLAAEPKSSDKTHTCPPCPAPEAAGGPSVFLFPPKP |
| | | | | KDTLMISRTPEVTCVVVSVSHEDPEVKFNWYVDG |
| | | | | VEVHNAKTKPREEQYNSTYRVVSVLTVLHQDWLN |
| | | | | GKEYKCKVSNKALPAPIEKTISKAKGQPREPQVYV |
| | | | | LPPSRDELTKNQVSLLCLVKGFYPSDIAVEWESNG |
| | | | | QPENNYLTWPPVLDSDGSFFLYSKLTVDKSRWQQ |
| | | | | GNVFSCSVMHEALHNHYTQKSLSLSPG |
| 26120 | L | CD28 | 115 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIKRTVAAPSVFIFPPSDEQLKSGTASVVCLLNN |
| | | | | FYPREAKVQWKVDNALQSGNSQESVTEQDSKDST |
| | | | | YSLSSTLTLSKADYEKHKVYACEVTHQGLSSPVTK |
| | | | | SFNRGEC |

Affinity modified anti-CD28 binding domains derived from hu_TN228
(Mutation Positions According to the IMGT Numbering Scheme)

| | | | | |
|---|---|---|---|---|
| Hu_ | V_{H} | CD28 | 106 | QVQLQESGPGLVKPSETLSLTCAVSGESLTSYGVH |
| TN228_ | | | | WIRQPPGKGLEWLGVIWPGGGTNFNSALMSRLTIS |
| WT | | | | EDTSKNQVSLKLSSVTAADTAVYYCARDRAYGNY |
| | | | | LYAMDYWGQGTLVTVSS |
| | V_{L} | | 116 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |
| Mod. | V_{H} | | 106 | QVQLQESGPGLVKPSETLSLTCAVSGFSLTSYGVH |
| Hu_ | | | | WIRQPPGKGLEWLGVIWPGGGTNFNSALMSRLTIS |
| TN228 | | | | EDTSKNQVSLKLSSVTAADTAVYYCARDRAYGNY |
| | | | | LYAMDYWGQGTLVTVSS |
| | V_{L} | | 200 | DIQMTQSPSSLSASVGDRVTITCRASESVEYYVTSL |
| | (D1068E) | | | MQWYQQKPGKAPKLLIYAASNVESGVPSRFSGSGS |
| | | | | GTDFTLTISSLQPEDIATYYCQQSRKVPFTFGGGTK |
| | | | | VEIK |
| Mod. | V_{H} | | 201 | QVQLQESGPGLVKPSETLSLTCAVSGFSLTSYGVH |
| Hu_ | (Y1110S) | | | WIRQPPGKGLEWLGVIWPGGGTNFNSALMSRLTIS |
| TN228 | | | | EDTSKNQVSLKLSSVTAADTAVYYCARDRASGNY |
| | | | | LYAMDYWGQGTLVTVSS |
| | V_{L} | | 116 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |
| Mod. | V_{H} | | 106 | QVQLQESGPGLVKPSETLSLTCAVSGFSLTSYGVH |
| Hu_ | | | | WIRQPPGKGLEWLGVIWPGGGTNFNSALMSRLTIS |
| TN228 | | | | EDTSKNQVSLKLSSVTAADTAVYYCARDRAYGNY |
| | | | | LYAMDYWGQGTLVTVSS |
| | V_{L} | | 202 | DIQMTQSPSSLSASVGDRVTITCRASESVEYYVTSL |
| | (N1066A) | | | MQWYQQKPGKAPKLLIYAASAVDSGVPSRFSGSG |
| | | | | SGTDFTLTISSLQPEDIATYYCQQSRKVPFTFGGGT |
| | | | | KVEIK |
| Mod. | V_{H} | | 203 | QVQLQESGPGLVKPSETLSLTCAVSGFSLTSYGVH |
| Hu_ | (E1080K) | | | WIRQPPGKGLEWLGVIWPGGGTNFNSALMSRLTIS |
| TN228 | | | | KDTSKNQVSLKLSSVTAADTAVYYCARDRAYGNY |
| | | | | LYAMDYWGQGTLVTVSS |
| | V_{L} | | 116 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |
| Mod. | V_{H} | | 204 | QVQLQESGPGLVKPSETLSLTCAVSGFSLTSYGVH |
| Hu_ | (P1058A) | | | WIRQPPGKGLEWLGVIWAGGGTNFNSALMSRLTIS |
| TN228 | | | | EDTSKNQVSLKLSSVTAADTAVYYCARDRAYGNY |
| | | | | LYAMDYWGQGTLVTVSS |
| | V_{L} | | 116 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |
| Mod. | V_{H} | | 205 | QVQLQESGPGLVKPSETLSLTCAVSGFSLTSAGVH |
| Hu_ | (Y1037A) | | | WIRQPPGKGLEWLGVIWPGGGTNFNSALMSRLTIS |
| TN228 | | | | EDTSKNQVSLKLSSVTAADTAVYYCARDRAYGNY |
| | | | | LYAMDYWGQGTLVTVSS |
| | V_{L} | | 116 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |
| Mod. | V_{H} | | 206 | QVQLQESGPGLVKPSETLSLTCAVSGFSLTSYGVH |
| Hu_ | (L1112aN) | | | WIRQPPGKGLEWLGVIWPGGGTNFNSALMSRLTIS |
| TN228 | | | | EDTSKNQVSLKLSSVTAADTAVYYCARDRAYGNY |
| | | | | NYAMDYWGQGTLVTVSS |
| | V_{L} | | 116 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |
| Mod. | V_{H} | | 207 | QVQLQESGPGLVKPSETLSLTCAVSGFSLTSYGVH |
| Hu_ | (G1064S) | | | WIRQPPGKGLEWLGVIWPGGSTNFNSALMSRLTIS |
| TN228 | | | | EDTSKNQVSLKLSSVTAADTAVYYCARDRAYGNY |
| | | | | LYAMDYWGQGTLVTVSS |
| | V_{L} | | 116 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |
| Mod. | V_{H} | | 208 | QVQLQESGPGLVKPSETLSLTCAVSGFSLTSYGVH |
| Hu_ | (N1111aA) | | | WIRQPPGKGLEWLGVIWPGGGTNFNSALMSRLTIS |
| TN228 | | | | EDTSKNQVSLKLSSVTAADTAVYYCARDRAYGAY |
| | | | | LYAMDYWGQGTLVTVSS |
| | V_{L} | | 116 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |
| Mod. | V_{H} | | 106 | QVQLQESGPGLVKPSETLSLTCAVSGFSLTSYGVH |
| Hu_ | | | | WIRQPPGKGLEWLGVIWPGGGTNFNSALMSRLTIS |
| TN228 | | | | EDTSKNQVSLKLSSVTAADTAVYYCARDRAYGNY |
| | | | | LYAMDYWGQGTLVTVSS |
| | V_{L} | | 209 | DIQMTQSPSSLSASVGDRVTITCRASESVEYYGTSL |
| | (V1035G) | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |
| Mod. | V_{H} | | 210 | QVQLQESGPGLVKPSETLSLTCAVSGFSLTSYGVH |
| Hu_ | (Y1113S) | | | WIRQPPGKGLEWLGVIWPGGGTNFNSALMSRLTIS |
| TN228 | | | | EDTSKNQVSLKLSSVTAADTAVYYCARDRAYGNY |
| | | | | LSAMDYWGQGTLVTVSS |
| | V_{L} | | 116 | DIQMTQSPSSLSASVGDRVTITCRASESVEYYVTSL |
| | | | | MQWYQQKPGKAPKLLIYAASNVDSGVPSRFSGSG |
| | | | | SGTDFTLTISSLQPEDIATYYCQQSRKVPFTFGGGT |
| | | | | KVEIK |

CDRs (According to the Kabat Numbering Scheme, unless stated otherwise);
cons. = consensus sequence

| | | | | |
|---|---|---|---|---|
| WT | HCDR1 | CD28 | 300 | SYGVH |
| | HCDR1 | CD28 | 301 | SAGVH |
| Cons. | HCDR1 | CD28 | 302 | SX₁GVH, X₁ = Y, A (“,” is equivalent to |
| | | | | “or”) |
| WT | HCDR2 | CD28 | 303 | VIWPGGGTNFNSALMS |
| | HCDR2 | CD28 | 304 | VIWAGGGTNFNSALMS |
| | HCDR2 | CD28 | 305 | VIWPGGSTNFNSALMS |
| Cons. | HCDR2 | CD28 | 306 | VIWX₂GGX₃TNFNSALMS, X₂ = P, A; X₃ = G, S |
| WT | HCDR3 | CD28 | 307 | DRAYGNYLYAMDY |
| | HCDR3 | CD28 | 308 | DRASGNYLYAMDY |
| | HCDR3 | CD28 | 309 | DRAYGAYLYAMDY |
| | HCDR3 | CD28 | 310 | DRAYGNYNYAMDY |
| | HCDR3 | CD28 | 311 | DRAYGNYLSAMDY |
| Cons. | HCDR3 | CD28 | 312 | DRAX₄GX₅YX₆X₇AMDY, X₄ = S, Y; X₅ = N, A; |
| | | | | X₆ = L, N; X₇ = S, Y |
| WT | LCDR1 | CD28 | 313 | RASESVEYYVTSLMQ |
| V1035G | LCDR1 | CD28 | 314 | RASESVEYYGTSLMQ |
| Cons. | LCDR1 | CD28 | 315 | RASESVEYYX₈TSLMQ, X₈ = G, V |
| WT | LCDR2 | CD28 | 316 | AASNVDS |
| D1068E | LCDR2 | CD28 | 317 | AASNVES |
| N1066A | LCDR2 | CD28 | 318 | AASAVDS |
| Cons. | LCDR2 | CD28 | 319 | AASX₉VX₁₀S, X₉ = N, A; X₁₀ = E, D |
| WT | LCDR3 | CD28 | 320 | QQSRKVPFT |
| | HCDR1 | CD3 | 321 | GVTFNYYG (IMGT) |
| | HCDR2 | CD3 | 322 | ITSSGGRI (IMGT) |
| | HCDR3 | CD3 | 323 | TLDGRDGWVAY (IMGT) |
| | LCDR1 | CD3 | 324 | TGNIGSNY (IMGT) |
| | LCDR2 | CD3 | 325 | RND (IMGT) |
| | LCDR3 | CD3 | 326 | QSYSSGFI (IMGT) |
| | HCDR1 | MSLN | 327 | GYTMN |
| | HCDR2 | MSLN | 328 | LITPYSGASSYAQKFQG |
| | HCDR3 | MSLN | 329 | GGYDGRGFDY |
| | LCDR1 | MSLN | 330 | SASSSVSYMH |
| | LCDR2 | MSLN | 331 | DTSKLAS |
| | LCDR3 | MSLN | 332 | QQWSGHPLT |
| | HCDR1 | Cldn18.2 | 333 | SNPMI |
| | HCDR2 | Cldn18.2 | 334 | IIDTDGSTYYADWAKG |
| | HCDR3 | Cldn18.2 | 335 | RLHGSSNGYYDDL |
| | LCDR1 | Cldn18.2 | 336 | QASQSIYSYLS |
| | LCDR2 | Cldn18.2 | 337 | KASTLAS |
| | LCDR3 | Cldn18.2 | 338 | QQGYTVTNVDKNT |

Additional Linkers

| | | |
|---|---|---|
| Linker | 339 | EAAAK)ₙ, n = 1, 2, 3, 4 or 5 |
| Linker | 340 | EAAAK |
| Linker | 341 | EAAAKEAAAK |
| Linker | 342 | PPP |
| Linker | 343 | PPPP |
| Linker | 344 | GPPPG |
| Linker | 345 | GGPPPGG |
| Linker | 346 | GPPPPG |
| Linker | 347 | GGPPPPGG |
| Linker | 348 | (GnS)ₘ, n, m can independently be 1, 2, 3, |
| | | 4 or 5 |
| Linker | 349 | (G₃S)ₙ(G₄S)ₗ, n = 1, 2, 3, 4 or 5 |
| Linker | 350 | (G₃S)ₗ(G₄S)ₙ, n = 1, 2, 3, 4 or 5 |
| Linker | 351 | (G3S)ₙ(G₄S)ₙ, n = 1, 2, 3, 4 or 5 |
| Linker | 352 | (GₘS)ₙ-GG, m, n are independently integers |
| | | from 0 to 20 |
| Linker | 353 | (SGₙ)ₘ, m, n are independently integers |
| | | from 0 to 20 but not 0 at the same time |
| Linker | 354 | (SEGₙ)ₘ, m, n are independently integers |
| | | from 0 to 20 but not 0 at the same time |

What is claimed is:

1. An antibody construct, comprising:
(i) a Fab domain capable of binding a first antigen on a first cytotoxic effector cell;
(ii) a first scFv domain and a second scFv domain, wherein one of the scFv domains is capable of binding a second antigen on a second cytotoxic effector cell and the other scFv domain is capable of binding a tumor-associated antigen (TAA) on a tumor cell, and
(iii) an Fc domain comprising a first Fc polypeptide and a second Fc polypeptide, wherein:
(a) the Fab domain is coupled to the N-terminus of the first Fc polypeptide, and
(b) the first and second scFv domains are independently coupled to either (i) an N-terminus of the Fab domain, (ii) a C-terminus of the Fab domain, (iii) the C-terminus of one of the Fc polypeptides, or (iv) the N-terminus of the second Fc polypeptide,
provided that when one of the scFv domains is coupled to the C-terminus of one of the Fc polypeptides, the first antigen is CD3 and the second antigen is CD28, or the first antigen is CD28 and the second antigen is CD3.

2. The antibody construct of any one of claim 1, wherein the first scFv domain and the second scFv domain are not coupled to each other in tandem.

3. The antibody construct of any one of claim 1 or claim 2, wherein the first antigen is CD28, and the second antigen is CD3.

4. The antibody construct of any one of claim 1 or claim 2, wherein the first antigen is CD3, and the second antigen is CD28.

5. The antibody construct of any one of claims 1-4, wherein the first scFv domain is coupled to an N-terminus of the Fab domain and the second scFv is coupled to the N-terminus of the second Fc polypeptide.

6. The antibody construct of claim 5, wherein the first scFv domain is coupled to the N-terminus of the V_{H} sequence of the heavy chain of the Fab domain.

7. The antibody construct of claim 5, wherein the first scFv domain is coupled to the N-terminus of the V_{L} sequence of the light chain of the Fab domain.

8. The antibody construct of claim 5 or 6, wherein the antibody construct comprises:
a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, and (iii) the first Fc polypeptide;
b) a second heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), and (ii) the second Fc polypeptide; and
c) a light chain polypeptide comprising, from N-terminus to C-terminus: a Fab V_{L} sequence coupled to a Fab C_{L} sequence,
wherein:
the heavy chain Fab sequence and the light chain polypeptide associate to form the Fab domain, and
the first Fc polypeptide and the second Fc polypeptide associate to form the Fc domain.

9. The antibody construct of any one of claims 1-4, wherein the first scFv domain is coupled to the C-terminus of the light chain C_{L} sequence of the Fab domain and the second scFv domain is coupled to the N-terminus of the second Fc polypeptide.

10. The antibody construct of claim 9, wherein the antibody construct comprises:
a) a first heavy chain polypeptide comprising, from N-terminus to C-terminus: (i) a heavy chain Fab sequence comprising a Fab V_{H} sequence coupled to a Fab C_{H1} sequence, and (ii) the first Fc polypeptide;
b) a second heavy chain polypeptide comprising, from N-terminus to C-terminus: (i) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}), and (ii) the second Fc polypeptide; and
c) a light chain polypeptide comprising, from N-terminus to C-terminus: (i) a light chain Fab sequence comprising a Fab V_{L} sequence coupled to a Fab C_{L} sequence, and (ii) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}),
wherein:
the heavy chain Fab sequence and the light chain Fab sequence associate to form the Fab domain, and
the first Fc polypeptide and the second Fc polypeptide associate to form the Fc domain.

11. The antibody construct of any one of claims 1-4, wherein the first scFv domain is coupled to an N-terminus of the Fab domain and the second scFv domain is coupled to the C-terminus of one of the Fc polypeptides.

12. The antibody construct of claim 11, wherein the first scFv domain is coupled to the N-terminus of the V_{H} domain of the Fab domain.

13. The antibody construct of any one of claims 11-12, wherein the second scFv domain is coupled to the C-terminus of the first Fc polypeptide.

14. The antibody construct of any one of claims 11-12, wherein the second scFv domain is coupled to the C-terminus of the second Fc polypeptide.

15. The antibody construct of any one of claims 11-13, wherein the antibody construct comprises:
a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, (iii) the first Fc polypeptide, and (iv) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H});
b) a second heavy chain polypeptide comprising the second Fc polypeptide; and
c) a light chain polypeptide comprising a Fab V_{L} sequence coupled to a Fab C_{L} sequence,
wherein:
the heavy chain Fab sequence and the light chain polypeptide associate to form the Fab domain, and
the first Fc polypeptide and the second Fc polypeptide associate to form the Fc domain.

16. The antibody construct of any one of claim 11-12, or 14, wherein the antibody construct comprises:
a) a first heavy chain polypeptide comprising, from N-terminus to C-terminus: (i) the first scFv domain comprising either a first scFv V_{H} sequence coupled to a first scFv V_{L} sequence (V_{H}-V_{L}), or a first scFv V_{L} sequence coupled to a first scFv V_{H} sequence (V_{L}-V_{H}), (ii) a heavy chain Fab sequence comprising a Fab V_{H} sequence coupled to a Fab C_{H1} sequence, and (iii) the first Fc polypeptide;
b) a second heavy chain polypeptide comprising, from N-terminus to C-terminus: (i) the second Fc polypeptide, and (ii) the second scFv domain comprising either a second scFv V_{H} sequence coupled to a second scFv V_{L} sequence (V_{H}-V_{L}), or a second scFv V_{L} sequence coupled to a second scFv V_{H} sequence (V_{L}-V_{H}); and
c) a light chain polypeptide comprising a Fab V_{L} sequence coupled to a Fab C_{L} sequence,
wherein:
the heavy chain Fab sequence and the light chain polypeptide associate to form the Fab domain, and
the first Fc polypeptide and the second Fc polypeptide associate to form the Fc domain.

17. The antibody construct of any one of claims 1-16, wherein the first scFv domain is capable of binding the TAA, and the second scFv domain is capable of binding the second antigen on the second cytotoxic effector cell.

18. The antibody construct of any one of claims 1-16, wherein the second scFv domain is capable of binding the TAA, and the first scFv domain is capable of binding the second antigen on the second cytotoxic effector cell.

19. The antibody construct of any one of claim 1, 9 or 10, comprising:
(i) the Fab domain capable of binding CD3;
(ii) the first scFv domain capable of binding CD28;
(iii) the second scFv domain capable of binding Claudin18.2 (Cldn18.2); and
(iv) the Fc domain comprising the first Fc polypeptide and the second Fc polypeptide,
wherein:
a) the Fab domain is coupled via its C_{H1} sequence to the N-terminus of the first Fc polypeptide,
b) the first scFv domain is coupled to the C-terminus of the C_{L} sequence of the Fab light chain, and
c) the second scFv domain is coupled to the N-terminus of the second Fc polypeptide.

20. The antibody construct of claim 19, wherein the antibody construct does not reduce T cell viability by more than 5%, 3%, 1%, or by 0% compared to T cells treated with a negative control construct that does not contain a binding domain against Cldn 18.2, and wherein the antibody construct is incubated with the T cells for 48 hours.

21. The antibody construct of claim 19 or claim 20, wherein the antibody construct reduces T cell viability by about 1.5-fold to about 2-fold, by about 1.5-fold to about 3-fold, or by about 2-fold to about 3-fold less than an antibody construct in which the first scFv domain and the second scFv domain are independently coupled to either the N-terminus of the Fab heavy chain or the N-terminus of the second Fc polypeptide, and wherein the respective antibody construct is incubated with the T cells for 48 hours.

22. The antibody construct of any one of claims 19-21, wherein the antibody construct induces about 80-fold to about 2000-fold, about 100-fold to about 1000-fold, or about 100-fold to about 500-fold less cytokine in an assay comprising human CD3⁺ T cells compared to an antibody construct in which the first scFv domain and the second scFv domain are independently coupled to either the N-terminus of the Fab heavy chain or the N-terminus of the second Fc polypeptide, and wherein the respective antibody construct is incubated with the T cells for 48 hours.

23. The antibody construct of any one of claims 19-22, wherein the antibody construct induces about 5-fold to about 900-fold, about 5-fold to about 500-fold, or about 5-fold to about 300-fold less cytokine in an assay comprising human PBMCs compared to an antibody construct in which the first scFv domain and the second scFv domain are independently coupled to either the N-terminus of the Fab heavy chain or the N-terminus of the second Fc polypeptide, and wherein the respective antibody construct is incubated with the T cells for 48 hours.

24. The antibody construct of any one of claims 22-23, wherein the cytokine comprises one or more of IL-2, TNFα, IFNγ, and IL-6.

25. The antibody construct of any one of claims 1-24, wherein the first scFv domain has the domain structure, from N- to C-terminus, of: V_{H}-V_{L}.

26. The antibody construct of any one of claims 1-24, wherein the first scFv domain has the domain structure, from N- to C-terminus, of: V_{L}-V_{H}.

27. The antibody construct of any one of claims 1-26, wherein the second scFv domain has the domain structure, from N- to C-terminus, of: V_{H}-V_{L}.

28. The antibody construct of any one of claims 1-26, wherein the second scFv domain has the domain structure, from N- to C-terminus, of: V_{L}-V_{H}.

29. The antibody construct of any one of claims 1-28, wherein the antibody construct comprises one or more linkers.

30. The antibody construct of claim 29, wherein the one or more linkers are peptide linkers that each comprise or consist of an amino acid sequence from 1 to about 50, from 2 to about 40, from 3 to about 30, or from 5 to about 25 consecutive amino acid residues in length.

31. The antibody construct of any one of claims 29-30, wherein the first scFv domain comprises a linker^{scFv1}.

32. The antibody construct of claim 31, wherein the linker^{scFv1} couples the N- or C-terminus of the V_{H} domain to the C- or N-terminus of the V_{L} domain, respectively, and comprises or consists of an amino acid sequence having about 80%, 90%, or 100% sequence identity to the amino acid sequence set forth in SEQ ID NO: 104.

33. The antibody construct of any one of claims 29-32, wherein the second scFv domain comprises a linker^{scFv2}.

34. The antibody construct of claim 33, wherein the linker^{scFv2} couples the N- or C-terminus of the V_{H} domain to the C- or N-terminus of the V_{L} domain, respectively, and comprises or consists of an amino acid sequence having about 80%, 90%, or 100% sequence identity to the amino acid sequence set forth in SEQ ID NO: 104.

35. The antibody construct of any one of claims 1-34, wherein the Fab domain that is capable of binding the first antigen on the first cytotoxic effector cell comprises a heavy chain constant domain (C_{H1}) comprising or consisting of an amino acid sequence having at least about 90%, 95%, 97%, 99%, or 100% sequence identity to the amino acid sequence set forth in SEQ ID NO: 107.

36. The antibody construct of any one of claims 1-35, wherein the antibody construct is capable of binding human CD28 with a dissociation constant (K_{D}) for CD28 of from about 10 nM to about 500 nM, from about 20 nM to about 600 nM, from about 20 nM to about 250 nM, from about 20 nM to about 150 nM, from about 20 nM to about 100 nM, or from about 20 nM to about 50 nM, as determined using SPR.

37. The antibody construct of any one of claims 1-36, wherein the antibody construct comprises an anti-CD28 V_{H} sequence comprising a HCDR1 having the sequence SX₁GVH (SEQ ID NO: 302), a HCDR2 having the sequence VIWX₂GGX₃TNFNSALMS (SEQ ID NO: 306), and a HCDR3 having the sequence DRAX₄GX₅YX₆X₇AMDY (SEQ ID NO: 312), and an anti-CD28 V_{L} sequence comprising a LCDR1 having the sequence RASESVEYYX₈TSLMQ (SEQ ID NO: 315), a LCDR2 having the sequence AASX₉VX₁₀S (SEQ ID NO: 319), and a LCDR3 having the sequence QQSRKVPFT (SEQ ID NO: 320), and wherein X₁=Y or A; X₂=P or A; X₃=G or S; X₄=S or Y; X₅=N or A; X₆=L or N; X₇=S or Y; X₈=G or V; X₉=N or A; and X₁₀=E or D.

38. The antibody construct of any one of claims 1-37, wherein the antibody construct comprises an anti-CD28 V_{H} sequence having at least about 90%, 95%, 97%, 99%, or 100% sequence identity to the amino acid sequence set forth in SEQ ID NO: 106, and an anti-CD28 V_{L} sequence having at least about 90%, 95%, 97%, 99%, or 100% sequence identity to the amino acid sequence set forth in SEQ ID NO: 116.

39. The antibody construct of claim 38, wherein the anti-CD28 V_{H} sequence comprises or consists of an amino acid sequence selected from the group consisting of SEQ ID NOS: 201, 203-208, and 210, and the anti-CD28 V_{L} sequence comprises or consists of an amino acid sequence selected from the group consisting of SEQ ID NOs: 200, 202, and 209.

40. The antibody construct of any one of claims 1-39, wherein the antibody construct comprises an anti-CD28 V_{H} sequence comprising the HCDR1 having the sequence SYGVH (SEQ ID NO: 300), the HCDR2 having the sequence VIWPGGGTNFNSALMS (SEQ ID NO: 303), and the HCDR3 having the sequence DRAYGNYLYAMDY (SEQ ID NO: 307), and an anti-CD28 V_{L} sequence comprising the LCDR1 having the sequence RASESVEYYVTSLMQ (SEQ ID NO: 313), the LCDR2 having the sequence AASNVDS (SEQ ID NO: 316), and the LCDR3 having the sequence QQSRKVPFT (SEQ ID NO: 320).

41. The antibody construct of any one of claims 1-40, wherein the antibody construct is capable of binding human CD3 with a dissociation constant (K_{D}) for CD3 of from about 20 nM to about 200 nM, from about 30 nM to about 150 nM, from about 40 nM to about 100 nM, or from 50 nM to about 80 nM, as determined using SPR.

42. The antibody construct of any one of claims 1-41, wherein the antibody construct comprises an anti-CD3 V_{H} sequence comprising the HCDR1-3 sequences set forth in SEQ ID NOs: 321-323, respectively, and an anti-CD3 V_{L} sequence comprising the LCDR1-3 sequences set forth in SEQ ID NOs: 324-326, respectively.

43. The antibody construct of any one of claims 1-42, wherein the antibody construct comprises an anti-CD3 V_{H} sequence comprising a HCDR1 having the sequence GVTFNYYG (SEQ ID NO: 321), a HCDR2 having the sequence ITSSGGRI (SEQ ID NO: 322), and a HCDR3 having the sequence TLDGRDGWVAY (SEQ ID NO: 323), and an anti-CD3 V_{L} sequence comprising a LCDR1 having the sequence TGNIGSNY (SEQ ID NO: 324), a LCDR2 having the sequence RND (SEQ ID NO: 325), and a LCDR3 having the sequence QSYSSGFI (SEQ ID NO: 326).

44. The antibody construct of any one of claims 1-43, wherein the antibody construct comprises an anti-CD3 V_{H} sequence having at least about 90%, 95%, 97%, 99%, or 100% sequence identity to the amino acid sequence set forth in SEQ ID NO: 102, and an anti-CD3 V_{L} sequence having at least about 90%, 95%, 97%, 99%, or 100% sequence identity to the amino acid sequence set forth in SEQ ID NO: 103.

45. The antibody construct of any one of claims 1-44, wherein the first cytotoxic effector cell and the second cytotoxic effector cell are different cells.

46. The antibody construct of any one of claims 1-44, wherein the first cytotoxic effector cell and the second cytotoxic effector cell are the same cell.

47. The antibody construct of any one of claims 1-46, wherein the first and second antigens are on a T cell.

48. The antibody construct of any one of claims 1-47, wherein the TAA is Cldn18.2.

49. The antibody construct of claim 48, wherein the antibody construct comprises an anti-Cldn 18.2 V_{H} sequence comprising the HCDR1-3 sequences set forth in SEQ ID NOs: 333-335, respectively, and an anti-Cldn18.2 V_{L} sequence comprising the LCDR1-3 sequences set forth in SEQ ID NOs: 336-338, respectively.

50. The antibody construct of claim 49, wherein the antibody construct comprises an anti-Cldn18.2 V_{H} sequence comprising a HCDR1 having the sequence SNPMI (SEQ ID NO: 333), a HCDR2 having the sequence IIDTDGSTYYADWAKG (SEQ ID NO: 334), and a HCDR3 having the sequence RLHGSSNGYYDDL (SEQ ID NO: 335), and an anti-Cldn18.2 V_{L} sequence comprising a LCDR1 having the sequence QASQSIYSYLS (SEQ ID NO: 336), a LCDR2 having the sequence KASTLAS (SEQ ID NO: 337), and a LCDR3 having the sequence QQGYTVTNVDKNT (SEQ ID NO: 338).

51. The antibody construct of any one of claims 48-50, wherein the anti-Cldn 18.2 V_{H} sequence comprises or consists of an amino acid sequence having at least about 90%, 95%, 97%, 99%, or 100% sequence identity to the amino acid sequence set forth in SEQ ID NO: 127, and the anti-Cldn 18.2 V_{L} sequence comprises or consists of an amino acid sequence having at least about 90%, 95%, 97%, 99%, or 100% sequence identity to the amino acid sequence set forth in SEQ ID NO: 128, and, optionally, wherein the antibody construct binds to human Cldn18.2 with an affinity from about 1 nM to about 80 nM, from about 10 nM to about 60 nM, from about 10 nM to about 50 nM, or from about 20 nM to about 50 nM, as determined by flow cytometry.

52. The antibody construct of any one of claims 1-51, wherein the first Fc polypeptide and the second Fc polypeptide of the Fc domain each comprise or consist of a CH2 sequence and a CH3 sequence.

53. The antibody construct of claim 52, wherein at least one of the CH2 sequences of the first and second Fc polypeptide is an IgG1 or IgG4 CH2 sequence and comprises one or more amino acid modifications when compared to an unmodified wildtype IgG1 or IgG4 CH2 sequence.

54. The antibody construct of claim 53, wherein both CH2 sequences of the first and second Fc polypeptide are IgG1 or IgG4 CH2 sequences and comprise the one or more amino acid modifications when compared to an unmodified wildtype IgG1 or IgG4 CH2 sequence.

55. The antibody construct of claim 53-54, wherein the one or more amino acid modifications to the CH2 sequences reduce or ablate interactions of the Fc domain with one or more Fc receptors, optionally, one or more Fcγ receptors.

56. The antibody construct of any one of claims 52-55, wherein the CH2 sequence of both the first and the second Fc polypeptide comprises or consists of an amino acid sequence having at least about 90%, 95%, 97%, 99%, or 100% sequence identity to the amino acid sequence set forth in SEQ ID NO: 109.

57. The antibody construct of any one of claims 52-56, wherein at least one of the CH3 sequences of the first and second Fc polypeptide is an IgG1 or IgG4 CH3 sequence and comprises one or more amino acid modifications when compared to an unmodified wildtype IgG1 or IgG4 CH3 sequence, and, optionally, wherein the first and second Fc polypeptides have different amino acid sequences and form a heterodimeric Fc domain.

58. The antibody construct of claim 57, wherein both CH3 sequences of the first and second Fc polypeptide are IgG1 or IgG4 CH3 sequences and comprise one or more amino acid modifications that promote preferential pairing of the first and second Fc polypeptide to form the heterodimeric Fc domain compared to the formation of a corresponding homodimeric Fc domain.

59. The antibody construct of any one of claims 57-58, wherein the CH3 sequence of one of the Fc polypeptides comprises a set of amino acid substitutions selected from the group consisting of: L351Y_F405A_Y407V, T350V_L351Y_F405A_Y407V and T350V_L351Y_S400E_F405A_Y407V, and the CH3 sequence of the other Fc polypeptide comprises a set of amino acid substitutions selected from the group consisting of: T366L_K392M_T394W, T366L_K392L_T394W, T350V_T366L_K392L_T394W, T350V_T366L_K392M_T394W and T350V_T366L_N390R_K392M_T394W, and wherein the numbering of amino acid residues in the Fc polypeptides is according to the EU numbering system.

60. The antibody construct of any one of claims 52-59, wherein the CH3 sequence of one Fc polypeptide comprises or consists of an amino acid sequence having at least about 90%, 95%, 97%, 99%, or 100% sequence identity to the amino acid sequence set forth in SEQ ID NO: 110.

61. The antibody construct of any one of claims 52-60, wherein the CH3 sequence of the other Fc polypeptide comprises or consists of an amino acid sequence having at least about 90%, 95%, 97%, 99%, or 100% sequence identity to the amino acid sequence set forth in SEQ ID NO: 114.

62. The antibody construct of any one of claims 1-61, wherein the antibody construct is trivalent and trispecific and binds each antigen monovalently.

63. The antibody construct of any one of claims 1-62, wherein the antibody construct has a melting temperature at Tm1, Tm2, and/or Tm3 that is within 10° C., 5° C., 2° C., or within 1° C. degree of that of a corresponding bivalent and monospecific IgG1 monoclonal antibody.

64. The antibody construct of any one of claims 1-63, wherein the antibody construct binds the cytotoxic effector cell that expresses CD3 and/or CD28 with an affinity that is about 2-fold, 5-fold, 10-fold, 20-fold, 50-fold, 100-fold, or about 200-fold higher than that of a corresponding bivalent and bispecific anti-CD3×TAA and/or anti-CD28×TAA antibody construct.

65. The antibody construct of any one of claims 1-64, wherein the antibody construct exhibits an IC₅₀ value from about 50 pM to about 0.01 pM, from about 25 pM to about 0.01 pM, from about from about 10 pM to about 0.05 pM, from about 10 pM to about 0.1 pM, from about 10 pM to about 1 pM, from about 5 pM to about 1 pM for killing TAA-expressing tumor cells that express at least about 100,000 TAA/cell by TDCC in the presence of the cytotoxic effector cell and using an E:T ratio of 2:1 and an incubation period of 72 hours.

66. The antibody construct of claim 65, wherein the antibody construct achieves a maximum killing of TAA-expressing tumor cells of at least about 60%, 65%, 70%, 75%, or 80%, 85%, or 90%, 100%, or from about 60% to about 100%, from about 70% to about 90%, or from about 75% to about 85%.

67. The antibody construct of any one of claims 1-66, wherein the antibody construct is capable of inducing the production of one or more cytokines by the cytotoxic effector cell ranging from about 300 μg/mL to about 9000 μg/mL, when TAA-expressing cells expressing at least about 100,000 TAA/cell are present and using an E:T ratio of 2:1 and an incubation period of 72 hours.

68. A pharmaceutical composition comprising the antibody construct of any one of claims 1-67, and a pharmaceutically acceptable carrier, excipient, diluent, or combination thereof.

69. A nucleic acid molecule or a set of nucleic acid molecules encoding one or more, two or more, or three or more polypeptide chains that form the antibody construct of any one of claims 1-67.

70. A vector or a set of vectors comprising the nucleic acid molecule or the set of nucleic acid molecules of claim 69.

71. A cell comprising the nucleic acid molecule or the set of nucleic acid molecules of claim 69, or the vector or set of vectors of claim 70.

72. A method of producing an antibody construct of any one of claims 1-67, the method comprising:
(a) obtaining a host cell culture comprising at least one host cell comprising one or more nucleic acid molecules encoding one or more, two or more, or three or more polypeptide chains that form the antibody construct; and
(b) recovering the antibody construct from the host cell culture.

73. The method of claim 72, further comprising, subsequent to step (b), purifying the antibody construct.

74. A method of eliciting an anti-tumor immune response in a cell population comprising immune cells and tumor cells, the method comprising contacting the cell population with an effective amount of the antibody construct of any one of claims 1-67, wherein the immune cells express the first and second antigen and the tumor cells express the TAA.

75. A method of inhibiting the proliferation of tumor cells, the method comprising contacting a cell population comprising the tumor cells and immune cells with an effective amount of the antibody construct of any one of claims 1-67, wherein the immune cells express the first and second antigen and the tumor cells express the TAA.

76. A method of killing tumor cells, the method comprising contacting a cell population comprising the tumor cells and immune cells with an effective amount of the antibody construct of any one of claims 1-67, wherein the immune cells express the first and second antigen and the tumor cells express the TAA.

77. The method of any one of claims 74-76, wherein the immune cells comprise T cells.

78. The method of any one of claims 74-77, wherein TAA is Cldn18.2.

79. The method of any one of claims 74-78, wherein the antibody construct binds CD3 and CD28 on either one T cell or two different T cells, and the TAA on a tumor cell.

80. The method of claim 79, wherein the binding of the antibody construct of the first and second antigen and the TAA forms a TCR-independent artificial immune synapse between the one or more immune cells and the tumor cell, thereby eliciting a cytotoxic immune response of the immune cell against the tumor cell.

81. The method of any one of claims 74-80, wherein the cell population is within a subject.

82. A method for treating a cancer in a subject in need thereof, the method comprising administering to the subject an antibody construct of any one of claims 1-67.

83. The method of claim 82, wherein a cytotoxic immune response against the cancer is elicited in the subject, thereby treating the cancer in the subject.

84. An antibody construct of any one of claims 1-67 for use in the treatment of cancer.

85. Use of an antibody construct of any one of claims 1-67 in the manufacture of a medicament for the treatment of cancer.

86. An antibody construct, comprising a binding domain capable of binding CD28, wherein the binding domain comprises a V_{H} sequence comprising a HCDR1 having the sequence SX₁GVH (SEQ ID NO: 302), a HCDR2 having the sequence VIWX₂GGX₃TNFNSALMS (SEQ ID NO: 306), and a HCDR3 having the sequence DRAX₄GX₅YX₆X₇AMDY (SEQ ID NO: 312), and a V_{L} sequence comprising a LCDR1 having the sequence RASESVEYYX₈TSLMQ (SEQ ID NO: 315), a LCDR2 having the sequence AASX₉ VX₁₀S (SEQ ID NO: 319), and a LCDR3 having the sequence QQSRKVPFT (SEQ ID NO: 320), having one or more of the following amino acid substitutions at the positions as identified in the CDR sequences: X₁: Y to A, X₂: P to A, X₃: G to S, X₄: S to Y, X₅: N to A, X₆: L to N, X₇: S to Y, X₈: G to V, X₉: N to A, and/or X₁₀: E to D.

87. The antibody construct of claim 86, wherein the binding domain comprises a V_{H} sequence comprising or consisting of an amino acid sequence selected from the group consisting of SEQ ID NOs: 201, 203-208, and 210, and a V_{L} sequence comprising or consisting of an amino acid sequence selected from the group consisting of SEQ ID NOs: 200, 202, and 209.

88. An antibody construct, comprising a binding domain capable of binding Cldn18.2, wherein the binding domain comprises a V_{H} sequence comprising a HCDR1 having the sequence SNPMI (SEQ ID NO: 333), a HCDR2 having the sequence IIDTDGSTYYADWAKG (SEQ ID NO: 334), and a HCDR3 having the sequence RLHGSSNGYYDDL (SEQ ID NO: 335), and a V_{L} sequence comprising a LCDR1 having the sequence QASQSIYSYLS (SEQ ID NO: 336), a LCDR2 having the sequence KASTLAS (SEQ ID NO: 337), and a LCDR3 having the sequence QQGYTVTNVDKNT (SEQ ID NO: 338).

89. The antibody construct of claim 88, wherein the binding domain comprises a V_{H} sequence comprising or consisting of an amino acid sequence having at least about 90%, 95%, 97%, 99%, or 100% sequence identity to the amino acid sequence set forth in SEQ ID NO: 127, and a V_{L} sequence comprising or consisting of an amino acid sequence having at least about 90%, 95%, 97%, 99%, or 100% sequence identity to the amino acid sequence set forth in SEQ ID NO: 128.

90. The antibody construct of any one of claims 86-89, further comprising one or more additional binding domains capable of binding one or more additional antigens.


SEQUENCE LISTING (355 sequences)

SEQ ID NO: 1 - protein, 217 aa, Homo sapiens
  source 1..217: note = Human IgG1 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 245 aa, synthetic construct
  source 1..245: note = scFv MSLN
EIVLTQSPATLSLSPGERVTMSCSASSSVSYMHWYQQKPGQAPRLWIYDTSKLASGVPARFSGSGSGTDYTLTISSVEPEDNAVYYCQQWSGHPLTFGQGTKVEIKGGGGSGGGGSGGGGSGGGGSQVQLVQSGAEVKKPGASVKISCKASGYSFTGYTMNWVRQAPGQGLEWIGLITPYSGASSYAQKFQGRATLTVDKSTSTAYMELSSLRSEDTAVYYCARGGYDGRGFDYWGQGTNVTVSS

SEQ ID NO: 3-99 - skipped (97 entries)

SEQ ID NO: 100 - protein, 703 aa, synthetic construct
  source 1..703: note = v26149 H CD3/CD28
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGGGGSGQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 101 - protein, 247 aa, synthetic construct
  source 1..247: note = scFv CD3
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVL

SEQ ID NO: 102 - protein, 118 aa, synthetic construct
  source 1..118: note = VH CD3
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSS

SEQ ID NO: 103 - protein, 109 aa, synthetic construct
  source 1..109: note = VL CD3
NFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVL

SEQ ID NO: 104 - protein, 20 aa, synthetic construct
  source 1..20: note = Linker scFv
GGGGSGGGGSGGGGSGGGGS

SEQ ID NO: 105 - protein, 6 aa, synthetic construct
  source 1..6: note = Linker scFv-Fab
GGGGSG

SEQ ID NO: 106 - protein, 121 aa, synthetic construct
  source 1..121: note = VH CD28
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSS

SEQ ID NO: 107 - protein, 98 aa, synthetic construct
  source 1..98: note = CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 108 - protein, 15 aa, synthetic construct
  source 1..15: note = Linker Fab-Fc Hinge
EPKSCDKTHTCPPCP

SEQ ID NO: 109 - protein, 110 aa, synthetic construct
  source 1..110: note = CH2 Het-Fc
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK

SEQ ID NO: 110 - protein, 106 aa, synthetic construct
  source 1..106: note = CH3 Het-Fc
GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 111 - protein, 17 aa, synthetic construct
  source 1..17: note = Linker scFv-Fc Hinge
AAEPKSSDKTHTCPPCP

SEQ ID NO: 112 - protein, 15 aa, synthetic construct
  source 1..15: note = Linker scFv-Fc Hinge
EPKSSDKTHTCPPCP

SEQ ID NO: 113 - protein, 121 aa, synthetic construct
  source 1..121: note = VH (Y1112S)
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNSLYAMDYWGQGTLVTVSS

SEQ ID NO: 114 - protein, 106 aa, synthetic construct
  source 1..106: note = CH3 Het-Fc
GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 115 - protein, 218 aa, synthetic construct
  source 1..218: note = v26120 CD28
DIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 111 aa, synthetic construct
  source 1..111: note = VL CD28
DIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIK

SEQ ID NO: 117 - protein, 107 aa, synthetic construct [sequence omitted]

SEQ ID NO: 118 - protein, 705 aa, synthetic construct
  source 1..705: note = v26150 H CD28/CD3
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSSGGGGSGGGGSGGGGSGGGGSDIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIKGGGGSGEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 119 - protein, 252 aa, synthetic construct
  source 1..252: note = scFv CD28
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSSGGGGSGGGGSGGGGSGGGGSDIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIK

SEQ ID NO: 120 - protein, 215 aa, synthetic construct
  source 1..215: note = v16412 L CD3
NFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 121 - protein, 106 aa, synthetic construct
  source 1..106: note = CL
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 122 - protein, 447 aa, synthetic construct
  source 1..447: note = v25094 H CD3
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 123 - protein, 473 aa, synthetic construct
  source 1..473: note = v26152 L CD3/CD28
NFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGDIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIKGGGGSGGGGSGGGGSGGGGSQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSS

SEQ ID NO: 124 - protein, 703 aa, synthetic construct
  source 1..703: note = v26154 H CD28/CD3
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGSGNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGGGGSGGGGSGGGGSGGGGSEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSS

SEQ ID NO: 125 - protein, 483 aa, synthetic construct
  source 1..483: note = v28373 H CLDN 18.2
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 126 - protein, 250 aa, synthetic construct
  source 1..250: note = scFv
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIK

SEQ ID NO: 127 - protein, 119 aa, synthetic construct
  source 1..119: note = VH 13b08_L4
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSS

SEQ ID NO: 128 - protein, 111 aa, synthetic construct
  source 1..111: note = VL CLDN 18.2
DGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIK

SEQ ID NO: 129 - protein, 705 aa, synthetic construct
  source 1..705: note = v26153 H CD3/CD28
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGSGDIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIKGGGGSGGGGSGGGGSGGGGSQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSS

SEQ ID NO: 130 - protein, 961 aa, synthetic construct
  source 1..961: note = v28378 H
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKGGGGSGEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGSGDIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIKGGGGSGGGGSGGGGSGGGGSQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSS

SEQ ID NO: 131 - protein, 231 aa, synthetic construct
  source 1..231: note = v12153 H
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 132 - protein, 485 aa, synthetic construct
  source 1..485: note = v26121 H CD28
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSSGGGGSGGGGSGGGGSGGGGSDIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 133 - protein, 703 aa, synthetic construct
  source 1..703: note = v28415 H CLDN 18.2/CD3
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKGGGGSGEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 134 - protein, 450 aa, synthetic construct
  source 1..450: note = v26119 H CD28
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 135 - protein, 471 aa, synthetic construct
  source 1..471: note = v27550 L CD3/CD28
DIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGGGGSGGGGSGGGGSGGGGSEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSS

SEQ ID NO: 136 - protein, 959 aa, synthetic construct
  source 1..959: note = v28381 H CLDN 18.2/CD28/CD3
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKGGGGSGQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGSGNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGGGGSGGGGSGGGGSGGGGSEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSS

SEQ ID NO: 137 - protein, 706 aa, synthetic construct
  source 1..706: note = v28382 H CLDN 18.2/CD28
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKGGGGSGQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 138 - protein, 480 aa, synthetic construct
  source 1..480: note = v28383 H CD3
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 139 - protein, 703 aa, synthetic construct
  source 1..703: note = v28242 H CD3/CD28
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGGGGSGQVQLQESGPGLVKPSETLSLTCAVSGFSLTSYGVHWIRQPPGKGLEWLGVIWAGGGTNFNSALMSRLTISEDTSKNQVSLKLSSVTAADTAVYYCARDRAYGNYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 140 - protein, 703 aa, synthetic construct
  source 1..703: note = v28245 H CD3/CD28
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGGGGSGQVQLQESGPGLVKPSETLSLTCAVSGFSLTSYGVHWIRQPPGKGLEWLGVIWPGGSTNFNSALMSRLTISEDTSKNQVSLKLSSVTAADTAVYYCARDRAYGNYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 141 - protein, 218 aa, synthetic construct
  source 1..218: note = v28426 L CD28
DIQMTQSPSSLSASVGDRVTITCRASESVEYYGTSLMQWYQQKPGKAPKLLIYAASNVDSGVPSRFSGSGSGTDFTLTISSLQPEDIATYYCQQSRKVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 218 aa, synthetic construct
  source 1..218: note = v28427 L CD28
DIQMTQSPSSLSASVGDRVTITCRASESVEYYVTSLMQWYQQKPGKAPKLLIYAASNVESGVPSRFSGSGSGTDFTLTISSLQPEDIATYYCQQSRKVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 703 aa, synthetic construct
  source 1..703: note = v28428 H CD3/CD28
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGGGGSGQVQLQESGPGLVKPSETLSLTCAVSGFSLTSYGVHWIRQPPGKGLEWLGVIWPGGGTNFNSALMSRLTISKDTSKNQVSLKLSSVTAADTAVYYCARDRAYGNYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 144 - protein, 703 aa, synthetic construct
  source 1..703: note = v28429 H CD3/CD28
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGGGGSGQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRASGNYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 145 - protein, 703 aa, synthetic construct
  source 1..703: note = CD3/CD28
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGGGGSGQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGAYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 146 - protein, 703 aa, synthetic construct
  source 1..703: note = v28431 H CD3/CD28
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGGGGSGQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNSLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 147 - protein, 703 aa, synthetic construct
  source 1..703: note = v28432 H CD3/CD28
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGGGGSGQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 148 - protein, 703 aa, synthetic construct
  source 1..703: note = v28433 H CD3/CD28
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGGGGSGQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 149 - protein, 703 aa, synthetic construct
  source 1..703: note = v28434 H CD3/CD38
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGGGGSGQVQLQESGPGLVKPSETLSLTCAVSGFSLTSAGVHWIRQPPGKGLEWLGVIWPGGGTNFNSALMSRLTISEDTSKNQVSLKLSSVTAADTAVYYCARDRAYGNYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 150 - protein, 218 aa, synthetic construct
  source 1..218: note = v28435 L CD28
DIQMTQSPSSLSASVGDRVTITCRASESVEAYVTSLMQWYQQKPGKAPKLLIYAASNVDSGVPSRFSGSGSGTDFTLTISSLQPEDIATYYCQQSRKVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 111 aa, synthetic construct
  source 1..111: note = VL (Y1031A) CD28
DIQMTQSPSSLSASVGDRVTITCRASESVEAYVTSLMQWYQQKPGKAPKLLIYAASNVDSGVPSRFSGSGSGTDFTLTISSLQPEDIATYYCQQSRKVPFTFGGGTKVEIK

SEQ ID NO: 152 - protein, 218 aa, synthetic construct
  source 1..218: note = v28436 L CD28
DIQMTQSPSSLSASVGDRVTITCRASESVEYYVTSLMQWYQQKPGKAPKLLIYAASAVDSGVPSRFSGSGSGTDFTLTISSLQPEDIATYYCQQSRKVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 478 aa, synthetic construct
  source 1..478: note = v26161 H MSLN
EIVLTQSPATLSLSPGERVTMSCSASSSVSYMHWYQQKPGQAPRLWIYDTSKLASGVPARFSGSGSGTDYTLTISSVEPEDNAVYYCQQWSGHPLTFGQGTKVEIKGGGGSGGGGSGGGGSGGGGSQVQLVQSGAEVKKPGASVKISCKASGYSFTGYTMNWVRQAPGQGLEWIGLITPYSGASSYAQKFQGRATLTVDKSTSTAYMELSSLRSEDTAVYYCARGGYDGRGFDYWGQGTNVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 154 - protein, 216 aa, synthetic construct
  source 1..216: note = Fab CD3
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 155 - protein, 20 aa, synthetic construct
  source 1..20: note = signal peptide
METDTLLLWVLLLWVPGSTG

SEQ ID NO: 156 - protein, 23 aa, synthetic construct
  source 1..23: note = signal peptide
MRPTWAWWLFLVLLLALWAPARG

SEQ ID NO: 157 - protein, 19 aa, synthetic construct
  source 1..19: note = peptide signal
MGWSCIILFLVATATGVHS

SEQ ID NO: 158-199 - skipped (42 entries)

SEQ ID NO: 200 - protein, 111 aa, synthetic construct
  source 1..111: note = Mod. Hu_TN228 VL(D1068E)
DIQMTQSPSSLSASVGDRVTITCRASESVEYYVTSLMQWYQQKPGKAPKLLIYAASNVESGVPSRFSGSGSGTDFTLTISSLQPEDIATYYCQQSRKVPFTFGGGTKVEIK

SEQ ID NO: 201 - protein, 121 aa, synthetic construct
  source 1..121: note = Mod. Hu_TN228 VH (Y1110S) CD28
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRASGNYLYAMDYWGQGTLVTVSS

SEQ ID NO: 202 - protein, 111 aa, synthetic construct
  source 1..111: note = Mod. Hu_TN228 VL (N1066A)
DIQMTQSPSSLSASVGDRVTITCRASESVEYYVTSLMQWYQQKPGKAPKLLIYAASAVDSGVPSRFSGSGSGTDFTLTISSLQPEDIATYYCQQSRKVPFTFGGGTKVEIK

SEQ ID NO: 203 - protein, 121 aa, synthetic construct
  source 1..121: note = Mod. Hu_TN228 VH (E1080K) CD28
QVQLQESGPGLVKPSETLSLTCAVSGFSLTSYGVHWIRQPPGKGLEWLGVIWPGGGTNFNSALMSRLTISKDTSKNQVSLKLSSVTAADTAVYYCARDRAYGNYLYAMDYWGQGTLVTVSS

SEQ ID NO: 204 - protein, 121 aa, synthetic construct
  source 1..121: note = Mod. Hu_TN228 VH (P1058A)
QVQLQESGPGLVKPSETLSLTCAVSGFSLTSYGVHWIRQPPGKGLEWLGVIWAGGGTNFNSALMSRLTISEDTSKNQVSLKLSSVTAADTAVYYCARDRAYGNYLYAMDYWGQGTLVTVSS

SEQ ID NO: 205 - protein, 121 aa, synthetic construct
  source 1..121: note = Mod. Hu_TN228 VH (Y1037A)
QVQLQESGPGLVKPSETLSLTCAVSGFSLTSAGVHWIRQPPGKGLEWLGVIWPGGGTNFNSALMSRLTISEDTSKNQVSLKLSSVTAADTAVYYCARDRAYGNYLYAMDYWGQGTLVTVSS

SEQ ID NO: 206 - protein, 121 aa, synthetic construct
  source 1..121: note = Mod. Hu_TN228 VH (L1112aN)
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYNYAMDYWGQGTLVTVSS

SEQ ID NO: 207 - protein, 121 aa, synthetic construct
  source 1..121: note = Mod. Hu_TN228 VH (G1064S)
QVQLQESGPGLVKPSETLSLTCAVSGFSLTSYGVHWIRQPPGKGLEWLGVIWPGGSTNFNSALMSRLTISEDTSKNQVSLKLSSVTAADTAVYYCARDRAYGNYLYAMDYWGQGTLVTVSS

SEQ ID NO: 208 - protein, 121 aa, synthetic construct
  source 1..121: note = Mod. Hu_TN228 VH (N1111aA)
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGAYLYAMDYWGQGTLVTVSS

SEQ ID NO: 209 - protein, 111 aa, synthetic construct
  source 1..111: note = Mod. Hu_TN228 VL (V1035G) CD28
DIQMTQSPSSLSASVGDRVTITCRASESVEYYGTSLMQWYQQKPGKAPKLLIYAASNVDSGVPSRFSGSGSGTDFTLTISSLQPEDIATYYCQQSRKVPFTFGGGTKVEIK

SEQ ID NO: 210 - protein, 121 aa, synthetic construct
  source 1..121: note = VH (Y1113S)
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLSAMDYWGQGTLVTVSS

SEQ ID NO: 211 - protein, 706 aa, synthetic construct
  source 1..706: note = v29584 H CLDN 18.2/CD28
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKGGGGSGQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 212 - protein, 706 aa, synthetic construct
  source 1..706: note = v29583 H CLDN 18.2/CD28
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKGGGGSGQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGAYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 213 - protein, 706 aa, synthetic construct
  source 1..706: note = v29603 H CLDN 18.2/CD28
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKGGGGSGQVQLQESGPGLVKPSETLSLTCAVSGFSLTSYGVHWIRQPPGKGLEWLGVIWPGGSTNFNSALMSRLTISEDTSKNQVSLKLSSVTAADTAVYYCARDRAYGNYLYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 214 - protein, 706 aa, synthetic construct
  source 1..706: note = v29604 H CLDN 18.2/CD28
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKGGGGSGQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 215-299 - skipped (85 entries)

SEQ ID NO: 300 - protein, 5 aa, synthetic construct
  source 1..5: note = WT HCDR1 CD28
SYGVH

SEQ ID NO: 301 - protein, 5 aa, synthetic construct
  source 1..5: note = HCDR1 CD28
SAGVH

SEQ ID NO: 302 - protein, 5 aa, synthetic construct
  source 1..5: note = HCDR1
  VARIANT 2: note = X is Y or A
SXGVH

SEQ ID NO: 303 - protein, 16 aa, synthetic construct
  source 1..16: note = HCDR2 CD28
VIWPGGGTNFNSALMS

SEQ ID NO: 304 - protein, 16 aa, synthetic construct
  source 1..16: note = HCDR1 CD28
VIWAGGGTNFNSALMS

SEQ ID NO: 305 - protein, 16 aa, synthetic construct
  source 1..16: note = HCDR2 CD28
VIWPGGSTNFNSALMS

SEQ ID NO: 306 - protein, 16 aa, synthetic construct
  source 1..16: note = HCDR2
  VARIANT 4: note = X is P or A
  VARIANT 7: note = X is G or S
VIWXGGXTNFNSALMS

SEQ ID NO: 307 - protein, 13 aa, synthetic construct
  source 1..13: note = WT HCDR3 CD28
DRAYGNYLYAMDY

SEQ ID NO: 308 - protein, 13 aa, synthetic construct
  source 1..13: note = HCDR3 CD28
DRASGNYLYAMDY

SEQ ID NO: 309 - protein, 13 aa, synthetic construct
  source 1..13: note = HCDR3 CD28
DRAYGAYLYAMDY

SEQ ID NO: 310 - protein, 13 aa, synthetic construct
  source 1..13: note = HCDR3 CD28
DRAYGNYNYAMDY

SEQ ID NO: 311 - protein, 13 aa, synthetic construct
  source 1..13: note = HCDR3 CD28
DRAYGNYLSAMDY

SEQ ID NO: 312 - protein, 13 aa, synthetic construct
  source 1..13: note = HCDR3
  VARIANT 4: note = X is S and Y
  VARIANT 6: note = X is N or A
  VARIANT 8: note = X is L or N
  VARIANT 9: note = X is S or Y
DRAXGXYXXAMDY

SEQ ID NO: 313 - protein, 15 aa, synthetic construct
  source 1..15: note = LCDR1
RASESVEYYVTSLMQ

SEQ ID NO: 314 - protein, 15 aa, synthetic construct
  source 1..15: note = V1035G
RASESVEYYGTSLMQ

SEQ ID NO: 315 - protein, 15 aa, synthetic construct
  source 1..15: note = LCDR1
  VARIANT 10: note = X is G or V
RASESVEYYXTSLMQ

SEQ ID NO: 316 - protein, 7 aa, synthetic construct
  source 1..7: note = WT LCDR2
AASNVDS

SEQ ID NO: 317 - protein, 7 aa, synthetic construct
  source 1..7: note = D1068E
AASNVES

SEQ ID NO: 318 - protein, 7 aa, synthetic construct
  source 1..7: note = N1066A
AASAVDS

SEQ ID NO: 319 - protein, 7 aa, synthetic construct
  source 1..7: note = LCDR2
  VARIANT 4: note = X is N or A
  VARIANT 6: note = X is E or D
AASXVXS

SEQ ID NO: 320 - protein, 9 aa, synthetic construct
  source 1..9: note = WT LCDR3
QQSRKVPFT

SEQ ID NO: 321 - protein, 8 aa, synthetic construct
  source 1..8: note = HCDR1
GVTFNYYG

SEQ ID NO: 322 - protein, 8 aa, synthetic construct
  source 1..8: note = HCDR2
ITSSGGRI

SEQ ID NO: 323 - protein, 11 aa, synthetic construct
  source 1..11: note = HCDR3
TLDGRDGWVAY

SEQ ID NO: 324 - protein, 8 aa, synthetic construct
  source 1..8: note = LCDR2
TGNIGSNY

SEQ ID NO: 325 - skipped

SEQ ID NO: 326 - protein, 8 aa, synthetic construct
  source 1..8: note = LCDR3
QSYSSGFI

SEQ ID NO: 327 - protein, 5 aa, synthetic construct
  source 1..5: note = HCDR1
GYTMN

SEQ ID NO: 328 - protein, 17 aa, synthetic construct
  source 1..17: note = HCDR2
LITPYSGASSYAQKFQG

SEQ ID NO: 329 - protein, 10 aa, synthetic construct
  source 1..10: note = HCDR3
GGYDGRGFDY

SEQ ID NO: 330 - protein, 10 aa, synthetic construct
  source 1..10: note = LCDR1
SASSSVSYMH

SEQ ID NO: 331 - protein, 7 aa, synthetic construct
  source 1..7: note = LCDR2
DTSKLAS

SEQ ID NO: 332 - protein, 9 aa, synthetic construct
  source 1..9: note = LCDR3
QQWSGHPLT

SEQ ID NO: 333 - protein, 5 aa, synthetic construct
  source 1..5: note = HCDR1
SNPMI

SEQ ID NO: 334 - protein, 16 aa, synthetic construct
  source 1..16: note = HCDR2
IIDTDGSTYYADWAKG

SEQ ID NO: 335 - protein, 13 aa, synthetic construct
  source 1..13: note = HCDR3
RLHGSSNGYYDDL

SEQ ID NO: 336 - protein, 11 aa, synthetic construct
  source 1..11: note = LCDR1
QASQSIYSYLS

SEQ ID NO: 337 - protein, 7 aa, synthetic construct
  source 1..7: note = LCDR2
KASTLAS

SEQ ID NO: 338 - protein, 13 aa, synthetic construct
  source 1..13: note = LCDR3
QQGYTVTNVDKNT

SEQ ID NO: 339 - protein, 5 aa, synthetic construct
  source 1..5: note = Glutamate-alanine-lysine linker
  REGION >5: note = The entire sequence of amino acids 1-5 can be repeated one to five times
EAAAK

SEQ ID NO: 340 - protein, 5 aa, synthetic construct
  source 1..5: note = Glutamate-alanine-lysine linker
EAAAK

SEQ ID NO: 341 - protein, 10 aa, synthetic construct
  source 1..10: note = Glutamate-alanine-lysine linker
EAAAKEAAAK

SEQ ID NO: 342 - skipped

SEQ ID NO: 343 - protein, 4 aa, synthetic construct
  source 1..4: note = Proline linker
PPPP

SEQ ID NO: 344 - protein, 5 aa, synthetic construct
  source 1..5: note = Glycine-proline linker
GPPPG

SEQ ID NO: 345 - protein, 7 aa, synthetic construct
  source 1..7: note = Glycine-proline linker
GGPPPGG

SEQ ID NO: 346 - protein, 6 aa, synthetic construct
  source 1..6: note = Glycine-proline linker
GPPPPG

SEQ ID NO: 347 - protein, 8 aa, synthetic construct
  source 1..8: note = Glycine-proline linker
GGPPPPGG

SEQ ID NO: 348 - protein, 6 aa, synthetic construct
  source 1..6: note = Glycine-serine linker
  REGION 1..5: note = Up to four Gly residues may be deleted
  REGION >6: note = The entire sequence of amino acids can be repeated one to five times
GGGGGS

SEQ ID NO: 349 - protein, 9 aa, synthetic construct
  source 1..9: note = Glycine-serine linker
  REGION 1..4: note = The amino acid residues 1-4 can be repeated one to five times
GGGSGGGGS

SEQ ID NO: 350 - protein, 9 aa, synthetic construct
  source 1..9: note = Glycine-serine linker
  REGION 5..9: note = The amino acid residues 5-9 can be repeated one to five times
GGGSGGGGS

SEQ ID NO: 351 - protein, 9 aa, synthetic construct
  source 1..9: note = Glycine-serine linker
  REGION 1..4: note = The amino acid residues 1-4 can be repeated one to five times
  REGION 5..9: note = The amino acid residues 5-9 can be repeated one to five times
GGGSGGGGS

SEQ ID NO: 352 - protein, 23 aa, synthetic construct
  source 1..23: note = Glycine-serine linker
  REGION 1..20: note = Up to twenty Gly residues may be deleted
  REGION 1..21: note = The amino acid residues can be repeated one to twenty times
GGGGGGGGGGGGGGGGGGGGSGG

SEQ ID NO: 353 - protein, 21 aa, synthetic construct
  source 1..21: note = Glycine-serine linker
  REGION 2..21: note = Up to twenty Gly residues may be deleted
  REGION >21: note = The entire sequence of amino acids can be repeated one to twenty times
SGGGGGGGGGGGGGGGGGGGG

SEQ ID NO: 354 - protein, 22 aa, synthetic construct
  source 1..22: note = Glycine-serine linker
  REGION 3..22: note = Up to twenty Gly residues may be deleted
  REGION >22: note = The entire sequence of amino acids can be repeated one to twenty times
SEGGGGGGGGGGGGGGGGGGGG

SEQ ID NO: 355 - protein, 23 aa, synthetic construct
  source 1..23: note = Glycine-serine linker
  REGION 1..20: note = Up to twenty Gly residues may be deleted
  REGION 1..21: note = The amino acid residues can be repeated one to twenty times
GGGGGGGGGGGGGGGGGGGGSGG